(19) United States
(12) Patent Application Publication
(10) Pub. No.: US 2024/0425923 A1
(43) Pub. Date: Dec. 26, 2024

(54) INTESTINAL MONONUCLEAR PHAGOCYTES AS PROGNOSTIC BIOMARKER FOR CROHN'S DISEASE

(71) Applicant: CEDARS-SINAI MEDICAL CENTER, Los Angeles, CA (US)

(72) Inventors: Rebecca GONSKY, Los Angeles, CA (US); Stephan R. TARGAN, Santa Monica, CA (US)

(21) Appl. No.: 18/680,739

(22) Filed: May 31, 2024

Related U.S. Application Data
(63) Continuation of application No. PCT/US2022/051408, filed on Nov. 30, 2022.
(60) Provisional application No. 63/285,023, filed on Dec. 1, 2021.
(60) Provisional application No. 63/325,043, filed on Mar. 29, 2022.

(51) Int. Cl. C12Q 1/6883

(52) U.S. Cl. CPC C12Q 1/6883; C12Q 2600/106; C12Q 2600/112; C12Q 2600/158

(57) ABSTRACT

Described herein are systems and methods for stratifying intestinal mononuclear phagocytes (MNP) expression profiles in subjects with an inflammatory bowel disease (IBD). Further provided herein are systems and methods for determining or characterizing a Crohn's Disease (CD) subtype status in a subject having CD, selecting a treatment for a subject, or treating a subject.

### CROSS REFERENCE TO RELATED APPLICATIONS

This application is a continuation of International Application No. PCT/US2022/051408, filed Nov. 30, 2022, which claims the benefit of U.S. Provisional Application No. 63/285,023 filed Dec. 1, 2021, and U.S. Provisional Application No. 63/325,043 filed Mar. 29, 2022, each of which is incorporated herein by reference in its entirety.

### STATEMENT AS TO FEDERALLY SPONSORED RESEARCH

This invention was made with government support under grant number POT DK046763 and R01 DK056328 awarded by the National Institute of Health. The government has certain rights in the invention.

### BACKGROUND

Intestinal mononuclear phagocytes (MNP) play a key role in innate immunity, gut homeostasis and intestinal disease. Monocyte-derived macrophages display plasticity along a continuous spectrum acquiring pro-inflammatory (M1) or anti-inflammatory (M2) phenotypes in response environmental cues. In Crohn's disease (CD) MNP activation contributes to chronic inflammation and disease progression. Serotonin activates gut MNPs and enhanced serotonin receptors levels are associated with altered microbiota and disease flare.

### SUMMARY

Provided herein are methods for stratifying intestinal mononuclear phagocytes (MNP) expression profiles in subjects with an inflammatory bowel disease (IBD). In some embodiments, this may help to identify molecular pathways underlying MNP pathophysiology in treatment-resistant CD patients.

In one aspect, disclosed herein is method of determining a Crohn's Disease (CD) subtype status in a subject having CD, wherein the status comprises a CD13+ mononuclear phagocytic (MNP) subtype, the method comprising: detecting expression of one or more genes from Tables 1 or 2A-2B in a biological sample from the subject to obtain an expression profile comprising the expression levels of each of the one or more genes in the biological sample, and determining the CD subtype status of the subject based upon the expression profile, wherein an increased level of expression in the one or more genes in the biological sample as compared to a reference expression profile indicates status of CD-MNP subtype. In some embodiments, CD-MNP subtype is characterized by an inflammatory MNP transcriptomic signature. In some embodiments, the inflammatory MNP transcriptomic signature comprises the one or more genes from Table 2A. In some embodiments, the inflammatory MNP signature is associated with perianal disease or fistula, recurrence of the CD, or any combination thereof. In some embodiments, CD-MNP subtype is characterized a resident mucosal MNP transcriptomic signature. In some embodiments, the resident mucosal MNP transcriptomic signature comprises the one or more genes from Table 2B. In some embodiments, the resident mucosal MNP transcriptomic signature is associated with an increase or a decrease in a serological marker of an immune reactivity to a microbial antigen. In some embodiments, the serological marker comprises antineutrophil cytoplasmic antibodies (ANCA), antibodies (IgG) against the yeast Saccharomyces cerevisiae (ASCA), or a combination thereof. In some embodiments, the resident mucosal MNP transcriptomic signature is associated a coronavirus disease 2019 (COVID-19) transcriptomic signature. In some embodiments, the method further comprises distinguishing the CD13+MNP subtype from another CD subtype, wherein the CD13+MNP subtype is characterized by an inflammatory MNP transcriptomic signature. In some embodiments, the method further comprises distinguishing the CD13+MNP subtype from another CD subtype, wherein the CD13+MNP subtype is characterized by a resident mucosal MNP transcriptomic signature. In some embodiments, the other CD subtype comprises a subtype a CD subtype without a MNP signature. In some embodiments, the CD subtype without a MNP signature comprises a PBT subtype of CD. In some embodiments, the reference expression profile is derived from gene expression levels measured in samples obtained from one or more individuals that: (a) does not have the CD; or (b) has a different CD13+MNP subtype of the CD. In some embodiments, the increase in the level of expression of the one or more genes in the biological sample is at least 2-fold greater than in the reference expression profile. In some embodiments, the increase in the level of expression of the one or more genes in the biological sample is at least 3-fold greater than in the reference expression profile. In some embodiments, the reference expression profile comprises expression levels of the one or more genes of one or more subjects that do not have CD. In some embodiments, determining a level of expression of one or more genes comprises utilizing an assay selected from the group consisting of an RNA sequencing method, a microarray method, and quantitative polymerase chain reaction (qPCR). In some embodiments, determining a level of expression of one or more genes comprises: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, the method further comprises administering to the subject a therapeutic agent against Crohn's Disease based upon the expression profile. In some embodiments, the therapeutic agent comprises a modulator of miR-181a, miR-92a, miR-124, Tumor necrosis factor-like cytokine 1A (TL1A), Tumor necrosis factor ligand superfamily member 8 (CD30L), or any combination thereof. In some embodiments, the therapeutic agent comprises an antibody or antigen-binding fragment thereof. In some embodiments, antibody or antigen-binding fragment thereof comprises a TL1A antibody or antigen-binding fragment thereof. In some embodiments, the TL1A antibody or antigen-binding fragment thereof comprises PRA023, tulisokibart, PF-06480605, or TEV-48574. In some embodiments, the TL1A antibody or antigen-binding fragment thereof comprises PRA023. In some embodiments, the TL1A antibody or antigen-binding fragment thereof comprises tulisokibart. In some embodiments, the antibody or antigen-binding fragment thereof comprises a CD30L antibody or antigen-binding fragment thereof. In some embodiments, the CD30L antibody or antigen-binding fragment thereof comprises KPL-045. In some embodiments, the CD30L antibody or antigen-binding fragment thereof comprises PRA052. In some embodiments, the miR-181a modulator comprises an inhibitor of miR-181a. In some embodiments, the miR-92a modulator comprises an inhibitor of miR-92a. In some embodiments, the miR-124 modulator comprises an inhibitor of miR-124. In some embodiments, provided the biological sample comprises a blood sample or is purified from a blood sample of the subject. In some embodiments, the subject is not responsive to anti-TNFα therapy. In some embodiments, the subject has or is susceptible to having stricturing disease. In some embodiments, the subject has or is susceptible to having increased length of bowel resection. In some embodiments, the CD is associated with perianal disease/fistula. In some embodiments, the CD is associated with stricturing disease. In some embodiments, the CD is associated with recurrence. In some embodiments, the CD is associated with increased immune reactivity to a microbial antigen. In some embodiments, the reference expression profile comprises expression levels of the one or more genes of one or more subjects who do not have IBD or have a PBT subtype of CD. In some embodiments, the reference expression profile is stored in a database. In some embodiments, the primers and/or detectable nucleic acid probe comprises a nucleotide sequence that hybridizes to contiguous 12-45 nucleotides within the nucleic acid sequences of one or more of the genes provided in Tables 1, 2A, and/or 2B, wherein the nucleic acid sequences of the genes can be found in and are hereby incorporated by reference from the public databases using the references in Tables 1, 2A, and/or 2B. In some embodiments, the primer and/or detectable nucleic acid probe comprises a nucleotide sequence that hybridizes to a contiguous 12-45 nucleotides within the nucleic acid sequences of one or more of the genes provided in Tables 1, 2A, and/or 2B under stringent conditions. In some embodiments, the stringent conditions comprise hybridization to filter-bound DNA in 6× sodium chloride/sodium citrate (SSC) at about 45° C. followed by one or more washes in 0.2×SSC/0.1% SDS at about 50-65° C. In some embodiments, the primer and/or detectable nucleic acid probe comprises a nucleotide sequence that hybridizes to a contiguous 12-45 nucleotides within the nucleic acid sequences of one or more of the genes provided in Tables 1, 2A, and/or 2B under highly stringent conditions. In some embodiments, the highly stringent conditions comprise hybridization to filter-bound nucleic acid in 6×SSC at about 45° C. followed by one or more washes in 0.1×SSC/0.2% SDS at about 68° C. In some embodiments, the primer and/or detectable nucleic acid probe is hybridizable to contiguous 12-45 nucleotides within the reverse complement of the nucleic acid sequences of the one or more of the genes provided in Tables 1, 2A, and/or 2B. In some embodiments, the primer and/or detectable nucleic acid probe comprises a nucleotide sequence that hybridizes to a contiguous 12-45 nucleotides within the reverse complement of the nucleic acid sequences of the one or more of the genes provided in Tables 1, 2A, and/or 2B under stringent conditions. In some embodiments, the stringent conditions comprise hybridization to filter-bound DNA in 6× sodium chloride/sodium citrate (SSC) at about 45° C. followed by one or more washes in 0.2×SSC/0.1% SDS at about 50-65° C. In some embodiments, the primer and/or detectable nucleic acid probe comprises a nucleotide sequence that hybridizes to a contiguous 12-45 nucleotides within the reverse complement of the nucleic acid sequences of the one or more of the genes provided in Tables 1, 2A, and/or 2B under highly stringent conditions In some embodiments, the highly stringent conditions comprise hybridization to filter-bound nucleic acid in 6×SSC at about 45° C. followed by one or more washes in 0. IX SSC/0.2% SDS at about 68° C. In some embodiments, the contiguous 12-45 nucleotides of the nucleic acid sequences or the reverse complement of the nucleic sequences of the one or more of the genes provided in Tables 1, 2A, and/or 2B are referred to as the target hybridization nucleotides. In some embodiments of the primers and/or detectable nucleic acid probe provided herein, including in this paragraph, the target hybridization nucleotides comprise 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides.

In one aspect, disclosed herein is a method of selecting a treatment for a subject having a Crohn's Disease (CD) CD13+ mononuclear phagocytic (MNP) subtype, the method comprising: (a) determining a level of expression of one or more genes from Tables 2A-2B in a biological sample obtained from the subject having CD; (b) detecting an expression profile comprising an increase in the level of expression of the one or more genes in the biological sample, relative to a reference expression profile; and (c) identifying the subject as a candidate for treatment of Crohn's Disease based upon the expression profile that is detected in (b). In some embodiments, CD-MNP subtype is characterized by an inflammatory MNP transcriptomic signature. In some embodiments, the inflammatory MNP transcriptomic signature comprises the one or more genes from Table 2A. In some embodiments, the inflammatory MNP signature is associated with perianal disease or fistula, recurrence of the CD, or any combination thereof. In some embodiments, CD-MNP subtype is characterized a resident mucosal MNP transcriptomic signature. In some embodiments, the resident mucosal MNP transcriptomic signature comprises the one or more genes from Table 2B. In some embodiments, the resident mucosal MNP transcriptomic signature is associated with an increase or a decrease in a serological marker of an immune reactivity to a microbial antigen. In some embodiments, the serological marker comprises antineutrophil cytoplasmic antibodies (ANCA), antibodies (IgG) against the yeast Saccharomyces cerevisiae (ASCA), or a combination thereof. In some embodiments, the resident mucosal MNP transcriptomic signature is associated a coronavirus disease 2019 (COVID-19) transcriptomic signature. In some embodiments, the method further comprises distinguishing the CD13+MNP subtype from another CD subtype, wherein the CD13+MNP subtype is characterized by an inflammatory MNP transcriptomic signature. In some embodiments, the method further comprises distinguishing the CD13+MNP subtype from another CD subtype, wherein the CD13+MNP subtype is characterized by a resident mucosal MNP transcriptomic signature. In some embodiments, the other CD subtype comprises a subtype a CD subtype without a MNP signature. In some embodiments, the CD subtype without a MNP signature comprises a PBT subtype of CD. In some embodiments, the reference expression profile is derived from gene expression levels measured in samples obtained from one or more individuals that: (a) does not have the CD; or (b) has a different CD13+MNP subtype of the CD. In some embodiments, the reference expression profile comprises expression levels of the one or more genes of one or more subjects that do not have CD. In some embodiments, determining a level of expression of one or more genes comprises utilizing an assay selected from the group consisting of an RNA sequencing method, a microarray method, and quantitative polymerase chain reaction (qPCR). In some embodiments, determining a level of expression of one or more genes comprises: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, the method further comprises administering to the subject a therapeutic agent against Crohn's Disease based upon the expression profile. In some embodiments, the therapeutic agent comprises a modulator of miR-181a, miR-92a, or miR-124. In some embodiments, the miR-181a modulator comprises an inhibitor of miR-181a. In some embodiments, the miR-92a modulator comprises an inhibitor of miR-92a. In some embodiments, the miR-124 modulator comprises an inhibitor of miR-124. In some embodiments, provided the biological sample comprises a blood sample or is purified from a blood sample of the subject. In some embodiments, the subject is not responsive to anti-TNFα therapy. In some embodiments, the subject has or is susceptible to having stricturing disease. In some embodiments, the subject has or is susceptible to having increased length of bowel resection. In some embodiments, the CD is associated with perianal disease/fistula. In some embodiments, the CD is associated with stricturing disease. In some embodiments, the CD is associated with recurrence. In some embodiments, the CD is associated with increased immune reactivity to a microbial antigen. In some embodiments, the reference expression profile comprises expression levels of the one or more genes of one or more subjects who do not have IBD or have a PBT subtype of CD. In some embodiments, the reference expression profile is stored in a database. In some embodiments, the primers and/or detectable nucleic acid probe comprises a nucleotide sequence that hybridizes to contiguous 12-45 nucleotides within the nucleic acid sequences of one or more of the genes provided in Tables 1, 2A, and/or 2B, wherein the nucleic acid sequences of the genes can be found in and are hereby incorporated by reference from the public databases using the references in Tables 1, 2A, and/or 2B. In some embodiments, the primer and/or detectable nucleic acid probe comprises a nucleotide sequence that hybridizes to a contiguous 12-45 nucleotides within the nucleic acid sequences of one or more of the genes provided in Tables 1, 2A, and/or 2B under stringent conditions. In some embodiments, the stringent conditions comprise hybridization to filter-bound DNA in 6× sodium chloride/sodium citrate (SSC) at about 45° C. followed by one or more washes in 0.2×SSC/0.1% SDS at about 50-65° C. In some embodiments, the primer and/or detectable nucleic acid probe comprises a nucleotide sequence that hybridizes to a contiguous 12-45 nucleotides within the nucleic acid sequences of one or more of the genes provided in Tables 1, 2A, and/or 2B under highly stringent conditions. In some embodiments, the highly stringent conditions comprise hybridization to filter-bound nucleic acid in 6×SSC at about 45° C. followed by one or more washes in 0.1×SSC/0.2% SDS at about 68° C. In some embodiments, the primer and/or detectable nucleic acid probe is hybridizable to contiguous 12-45 nucleotides within the reverse complement of the nucleic acid sequences of the one or more of the genes provided in Tables 1, 2A, and/or 2B. In some embodiments, the primer and/or detectable nucleic acid probe comprises a nucleotide sequence that hybridizes to a contiguous 12-45 nucleotides within the reverse complement of the nucleic acid sequences of the one or more of the genes provided in Tables 1, 2A, and/or 2B under stringent conditions. In some embodiments, the stringent conditions comprise hybridization to filter-bound DNA in 6× sodium chloride/sodium citrate (SSC) at about 45° C. followed by one or more washes in 0.2×SSC/0.1% SDS at about 50-65° C. In some embodiments, the primer and/or detectable nucleic acid probe comprises a nucleotide sequence that hybridizes to a contiguous 12-45 nucleotides within the reverse complement of the nucleic acid sequences of the one or more of the genes provided in Tables 1, 2A, and/or 2B under highly stringent conditions In some embodiments, the highly stringent conditions comprise hybridization to filter-bound nucleic acid in 6×SSC at about 45° C. followed by one or more washes in 0. IX SSC/0.2% SDS at about 68° C. In some embodiments, the contiguous 12-45 nucleotides of the nucleic acid sequences or the reverse complement of the nucleic sequences of the one or more of the genes provided in Tables 1, 2A, and/or 2B are referred to as the target hybridization nucleotides. In some embodiments of the primers and/or detectable nucleic acid probe provided herein, including in this paragraph, the target hybridization nucleotides comprise 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides.

In one aspect, disclosed herein is a method of characterizing a Crohn's Disease (CD) in a subject as being a Crohn's Disease (CD) CD13+ mononuclear phagocytic (MNP) subtype (CD-MNP subtype), the method comprising: (a) determining a level of expression of one or more genes from Tables 2A-2B in a biological sample obtained from the subject having CD; (b) detecting an expression profile comprising an increase in the level of expression of the one or more genes in the biological sample, relative to a reference expression profile; and (c) characterizing the CD as being the CD-MNP subtype, based at least in part, on the expression profile that is detected in (b). In some embodiments, CD-MNP subtype is characterized by an inflammatory MNP transcriptomic signature. In some embodiments, the inflammatory MNP transcriptomic signature comprises the one or more genes from Table 2A. In some embodiments, the inflammatory MNP signature is associated with perianal disease or fistula, recurrence of the CD, or any combination thereof. In some embodiments, CD-MNP subtype is characterized a resident mucosal MNP transcriptomic signature. In some embodiments, the resident mucosal MNP transcriptomic signature comprises the one or more genes from Table 2B. In some embodiments, the resident mucosal MNP transcriptomic signature is associated with an increase or a decrease in a serological marker of an immune reactivity to a microbial antigen. In some embodiments, the serological marker comprises antineutrophil cytoplasmic antibodies (ANCA), antibodies (IgG) against the yeast Saccharomyces cerevisiae (ASCA), or a combination thereof. In some embodiments, the resident mucosal MNP transcriptomic signature is associated a coronavirus disease 2019 (COVID-19) transcriptomic signature. In some embodiments, the method further comprises distinguishing the CD13+MNP subtype from another CD subtype, wherein the CD13+MNP subtype is characterized by an inflammatory MNP transcriptomic signature. In some embodiments, the method further comprises distinguishing the CD13+MNP subtype from another CD subtype, wherein the CD13+MNP subtype is characterized by a resident mucosal MNP transcriptomic signature. In some embodiments, the other CD subtype comprises a subtype a CD subtype without a MNP signature. In some embodiments, the CD subtype without a MNP signature comprises a PBT subtype of CD. In some embodiments, the reference expression profile is derived from gene expression levels measured in samples obtained from one or more individuals that: (a) does not have the CD; or (b) has a different CD13+MNP subtype of the CD. In some embodiments, the reference expression profile comprises expression levels of the one or more genes of one or more subjects that do not have CD. In some embodiments, determining a level of expression of one or more genes comprises utilizing an assay selected from the group consisting of an RNA sequencing method, a microarray method, and quantitative polymerase chain reaction (qPCR). In some embodiments, determining a level of expression of one or more genes comprises: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, the method further comprises administering to the subject a therapeutic agent against Crohn's Disease based upon the expression profile. In some embodiments, the therapeutic agent comprises a modulator of miR-181a, miR-92a, or miR-124. In some embodiments, the miR-181a modulator comprises an inhibitor of miR-181a. In some embodiments, the miR-92a modulator comprises an inhibitor of miR-92a. In some embodiments, the miR-124 modulator comprises an inhibitor of miR-124. In some embodiments, provided the biological sample comprises a blood sample or is purified from a blood sample of the subject. In some embodiments, the subject is not responsive to anti-TNFα therapy. In some embodiments, the subject has or is susceptible to having stricturing disease. In some embodiments, the subject has or is susceptible to having increased length of bowel resection. In some embodiments, the CD is associated with perianal disease/fistula. In some embodiments, the CD is associated with stricturing disease. In some embodiments, the CD is associated with recurrence. In some embodiments, the CD is associated with increased immune reactivity to a microbial antigen. In some embodiments, the reference expression profile comprises expression levels of the one or more genes of one or more subjects who do not have IBD or have a PBT subtype of CD. In some embodiments, the reference expression profile is stored in a database.

In one aspect, disclosed herein is a treating moderate to severe Crohn's Disease (CD) in a subject, the method comprising: administering a therapeutically effective amount of a therapeutic agent for treatment of the CD, provided the subject is determined to have a CD13+ mononuclear phagocytic (MNP) subtype (CD-MNP subtype) based, at least in part, on an increased expression of one or more genes from Tables 2A-2B in a biological sample obtained from the subject, relative to a reference expression profile. In some embodiments, CD-MNP subtype is characterized by an inflammatory MNP transcriptomic signature. In some embodiments, the inflammatory MNP transcriptomic signature comprises the one or more genes from Table 2A. In some embodiments, the inflammatory MNP signature is associated with perianal disease or fistula, recurrence of the CD, or any combination thereof. In some embodiments, CD-MNP subtype is characterized a resident mucosal MNP transcriptomic signature. In some embodiments, the resident mucosal MNP transcriptomic signature comprises the one or more genes from Table 2B. In some embodiments, the resident mucosal MNP transcriptomic signature is associated with an increase or a decrease in a serological marker of an immune reactivity to a microbial antigen. In some embodiments, the serological marker comprises antineutrophil cytoplasmic antibodies (ANCA), antibodies (IgG) against the yeast Saccharomyces cerevisiae (ASCA), or a combination thereof. In some embodiments, the resident mucosal MNP transcriptomic signature is associated a coronavirus disease 2019 (COVID-19) transcriptomic signature. In some embodiments, the method further comprises distinguishing the CD13+MNP subtype from another CD subtype, wherein the CD13+MNP subtype is characterized by an inflammatory MNP transcriptomic signature. In some embodiments, the method further comprises distinguishing the CD13+MNP subtype from another CD subtype, wherein the CD13+MNP subtype is characterized by a resident mucosal MNP transcriptomic signature. In some embodiments, the other CD subtype comprises a subtype a CD subtype without a MNP signature. In some embodiments, the CD subtype without a MNP signature comprises a PBT subtype of CD. In some embodiments, the reference expression profile is derived from gene expression levels measured in samples obtained from one or more individuals that: (a) does not have the CD; or (b) has a different CD13+MNP subtype of the CD. In some embodiments, the reference expression profile comprises expression levels of the one or more genes of one or more subjects that do not have CD. In some embodiments, determining a level of expression of one or more genes comprises utilizing an assay selected from the group consisting of an RNA sequencing method, a microarray method, and quantitative polymerase chain reaction (qPCR). In some embodiments, determining a level of expression of one or more genes comprises: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, the therapeutic agent comprises a modulator of miR-181a, miR-92a, or miR-124. In some embodiments, the miR-181a modulator comprises an inhibitor of miR-181a. In some embodiments, the miR-92a modulator comprises an inhibitor of miR-92a. In some embodiments, the miR-124 modulator comprises an inhibitor of miR-124. In some embodiments, provided the biological sample comprises a blood sample or is purified from a blood sample of the subject. In some embodiments, the subject is not responsive to anti-TNFα therapy. In some embodiments, the subject has or is susceptible to having stricturing disease. In some embodiments, the subject has or is susceptible to having increased length of bowel resection. In some embodiments, the CD is associated with perianal disease/fistula. In some embodiments, the CD is associated with stricturing disease. In some embodiments, the CD is associated with recurrence. In some embodiments, the CD is associated with increased immune reactivity to a microbial antigen. In some embodiments, the reference expression profile comprises expression levels of the one or more genes of one or more subjects who do not have IBD or have a PBT subtype of CD. In some embodiments, the reference expression profile is stored in a database.

Further provided is a composition for determining a Crohn's Disease (CD) subtype status in a subject having CD, the composition comprising: a primer and/or a probe for amplifying and/or detecting the genes in Tables 1, or 2A-2B, provided the primer and/or the probe is used for identifying a CD13+ mononuclear phagocytic (MNP) subtype. In some embodiments, the primers/probe comprises a nucleotide sequence that hybridizes to contiguous 12-45 nucleotides within the nucleic acid sequences of one or more of the genes provided in Tables 1, 2A, and/or 2B, wherein the nucleic acid sequences of the genes can be found in and are hereby incorporated by reference from the public databases using the references in Tables 1, 2A, and/or 2B. In some embodiments, the primer/probe comprises a nucleotide sequence that hybridizes to a contiguous 12-45 nucleotides within the nucleic acid sequences of one or more of the genes provided in Tables 1, 2A, and/or 2B under stringent conditions. In some embodiments, the stringent conditions comprise hybridization to filter-bound DNA in 6× sodium chloride/sodium citrate (SSC) at about 45° C. followed by one or more washes in 0.2×SSC/0.1% SDS at about 50-65° C. In some embodiments, the primer/probe comprises a nucleotide sequence that hybridizes to a contiguous 12-45 nucleotides within the nucleic acid sequences of one or more of the genes provided in Tables 1, 2A, and/or 2B under highly stringent conditions. In some embodiments, the highly stringent conditions comprise hybridization to filter-bound nucleic acid in 6×SSC at about 45° C. followed by one or more washes in 0.1×SSC/0.2% SDS at about 68° C. In some embodiments, the primer/probe is hybridizable to contiguous 12-45 nucleotides within the reverse complement of the nucleic acid sequences of the one or more of the genes provided in Tables 1, 2A, and/or 2B. In some embodiments, the primer/probe comprises a nucleotide sequence that hybridizes to a contiguous 12-45 nucleotides within the reverse complement of the nucleic acid sequences of the one or more of the genes provided in Tables 1, 2A, and/or 2B under stringent conditions. In some embodiments, the stringent conditions comprise hybridization to filter-bound DNA in 6× sodium chloride/sodium citrate (SSC) at about 45° C. followed by one or more washes in 0.2×SSC/0.1% SDS at about 50-65° C. In some embodiments, the primer/probe comprises a nucleotide sequence that hybridizes to a contiguous 12-45 nucleotides within the reverse complement of the nucleic acid sequences of the one or more of the genes provided in Tables 1, 2A, and/or 2B under highly stringent conditions In some embodiments, the highly stringent conditions comprise hybridization to filter-bound nucleic acid in 6×SSC at about 45° C. followed by one or more washes in 0.1×SSC/0.2% SDS at about 68° C. In some embodiments, the contiguous 12-45 nucleotides of the nucleic acid sequences or the reverse complement of the nucleic sequences of the one or more of the genes provided in Tables 1, 2A, and/or 2B are referred to as the target hybridization nucleotides. In some embodiments of the primers/probes provided herein, including in this paragraph, the target hybridization nucleotides comprise 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides.

Further aspects disclosed herein provide a system comprising a kit for determining a Crohn's Disease (CD) subtype status in a subject having CD, the system comprising: one or more detection reagents comprising nucleic acids configured to hybridize to one or more genes in Tables 1, or 2A-2B. In some embodiments, the kit comprises reagents for use in a qPCR reaction. In some embodiments, the one or more detection reagents comprises one or more primers and/or a probe. In some embodiments, the probe comprises a quencher. In some embodiments, the probe comprises a detectable label. In some embodiments, the kit further comprises a chip comprising a solid substrate functionalized with oligonucleotides that hybridize to at least a portion of a sequence of the genes in Tables 1, and/or Table 2A or Table 2B. In some embodiments, the kit comprises sequencing library preparation reagents, wherein the sequencing library preparing reagents comprise adaptors comprising an index configured for use in demultiplexing sequencing reads when they attached to at least a portion of a sequence of the genes in Tables 1 and/or Table 2A or Table 2B, and sequenced by a sequencer.

In some embodiments, the primers and/or a probe comprises a nucleotide sequence that hybridizes to contiguous 12-45 nucleotides within the nucleic acid sequences of one or more of the genes provided in Tables 1, 2A, and/or 2B, wherein the nucleic acid sequences of the genes can be found in and are hereby incorporated by reference from the public databases using the references in Tables 1, 2A, and/or 2B. In some embodiments, the primer and/or a probe comprises a nucleotide sequence that hybridizes to a contiguous 12-45 nucleotides within the nucleic acid sequences of one or more of the genes provided in Tables 1, 2A, and/or 2B under stringent conditions. In some embodiments, the stringent conditions comprise hybridization to filter-bound DNA in 6× sodium chloride/sodium citrate (SSC) at about 45° C. followed by one or more washes in 0.2×SSC/0.1% SDS at about 50-65° C. In some embodiments, the primer and/or a probe comprises a nucleotide sequence that hybridizes to a contiguous 12-45 nucleotides within the nucleic acid sequences of one or more of the genes provided in Tables 1, 2A, and/or 2B under highly stringent conditions. In some embodiments, the highly stringent conditions comprise hybridization to filter-bound nucleic acid in 6×SSC at about 45° C. followed by one or more washes in 0.1×SSC/0.2% SDS at about 68° C. In some embodiments, the primer and/or a probe nucleic acid probe is hybridizable to contiguous 12-45 nucleotides within the reverse complement of the nucleic acid sequences of the one or more of the genes provided in Tables 1, 2A, and/or 2B. In some embodiments, the primer and/or a probe comprises a nucleotide sequence that hybridizes to a contiguous 12-45 nucleotides within the reverse complement of the nucleic acid sequences of the one or more of the genes provided in Tables 1, 2A, and/or 2B under stringent conditions. In some embodiments, the stringent conditions comprise hybridization to filter-bound DNA in 6× sodium chloride/sodium citrate (SSC) at about 45° C. followed by one or more washes in 0.2×SSC/0.1% SDS at about 50-65° C. In some embodiments, the primer and/or a probe comprises a nucleotide sequence that hybridizes to a contiguous 12-45 nucleotides within the reverse complement of the nucleic acid sequences of the one or more of the genes provided in Tables 1, 2A, and/or 2B under highly stringent conditions In some embodiments, the highly stringent conditions comprise hybridization to filter-bound nucleic acid in 6×SSC at about 45° C. followed by one or more washes in 0.1×SSC/0.2% SDS at about 68° C. In some embodiments, the contiguous 12-45 nucleotides of the nucleic acid sequences or the reverse complement of the nucleic sequences of the one or more of the genes provided in Tables 1, 2A, and/or 2B are referred to as the target hybridization nucleotides. In some embodiments of the primers and/or a probe provided herein, including in this paragraph, the target hybridization nucleotides comprise 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides.

Further provided herein is a computer-implemented platform for determining a Crohn's Disease (CD) subtype status in a subject having CD, wherein the status comprises identifying a CD13+ mononuclear phagocytic (MNP) subtype, the computer-implemented platform comprising: one or more processors collectively or individually programmed to implement a method comprising: (a) analyzing genotype data of the subject to detect a level of expression of one or more genes provided in Table 1 and/or Table 2A or Table 2B to produce an expression profile of the subject; and (b) determining the CD subtype status of the subject based upon the expression profile, wherein an increased level of the expression of the one or more genes as compared to a reference expression profile indicates that the CD subtype status of the subject comprises a CD-MNP subtype; and a database for storing the genotype data of the subject and/or the expression profile. In some embodiments, the method further comprises a genotype device configured for detecting the one or more genes from Tables 2A-2B in a biological sample obtained from the subject. In some embodiments, the genotype device comprises a microarray, sequencer, or a qPCR machine. In some embodiments, the expression profile is predictive of the CD-MNP subtype with a specificity of at least 70%, 80%, 90%, or 100%. In some embodiments, the expression profile is predictive of the CD-MNP subtype with a sensitivity of at least 70%, 80%, 90%, or 100%. In some embodiments, the expression profile is predictive of the CD-MNP subtype with an accuracy of at least 70%, 80%, 90%, or 100%. In some embodiments, the expression profile is predictive of the CD-MNP subtype with an area under the curve (AUC) of at least about 0.70, 0.80, 0.9, or 1.0. In some embodiments, the expression profile is predictive of the CD-MNP subtype with a negative predictive value (NPV) of at least 70%, 80%, 90%, or 100%. In some embodiments, the expression profile is predictive of the CD-MNP subtype with a positive predictive value (PPV) of at least 70%, 80%, 90%, or 100%. In some embodiments, CD-MNP subtype is characterized by an inflammatory MNP transcriptomic signature. In some embodiments, the inflammatory MNP transcriptomic signature comprises the one or more genes from Table 2A. In some embodiments, the inflammatory MNP signature is associated with perianal disease or fistula, recurrence of the CD, or any combination thereof. In some embodiments, CD-MNP subtype is characterized a resident mucosal MNP transcriptomic signature. In some embodiments, the resident mucosal MNP transcriptomic signature comprises the one or more genes from Table 2B. In some embodiments, the resident mucosal MNP transcriptomic signature is associated with an increase or a decrease in a serological marker of an immune reactivity to a microbial antigen. In some embodiments, the serological marker comprises antineutrophil cytoplasmic antibodies (ANCA), antibodies (IgG) against the yeast Saccharomyces cerevisiae (ASCA), or a combination thereof. In some embodiments, the resident mucosal MNP transcriptomic signature is associated a coronavirus disease 2019 (COVID-19) transcriptomic signature. In some embodiments, the method further comprises distinguishing the CD13+MNP subtype from another CD subtype, wherein the CD13+MNP subtype is characterized by an inflammatory MNP transcriptomic signature. In some embodiments, the method further comprises distinguishing the CD13+MNP subtype from another CD subtype, wherein the CD13+MNP subtype is characterized by a resident mucosal MNP transcriptomic signature. In some embodiments, the other CD subtype comprises a subtype a CD subtype without a MNP signature. In some embodiments, the CD subtype without a MNP signature comprises a PBT subtype of CD. In some embodiments, the reference expression profile is derived from gene expression levels measured in samples obtained from one or more individuals that: (a) does not have the CD; (b) has a subtype of CD that is not characterized by a CD13+MNP signature; or (c) has a different CD13+MNP subtype of the CD. In some embodiments, the method further comprises predicting whether the CD is suitable for treatment with a therapeutic agent based, at least in part, on the CD subtype status of the subject. In some embodiments, the therapeutic agent comprises a modulator of miR-181a, miR-92a, miR-124, Tumor necrosis factor-like cytokine TA (TL1A), Tumor necrosis factor ligand superfamily member 8 (CD30L), or any combination thereof. In some embodiments, the therapeutic agent comprises an antibody or antigen-binding fragment thereof. In some embodiments, antibody or antigen-binding fragment thereof comprises a TL1A antibody or antigen-binding fragment thereof. In some embodiments, the TL1A antibody or antigen-binding fragment thereof comprises PRA023, tulisokibart, PF-06480605, or TEV-48574. In some embodiments, the TL1A antibody or antigen-binding fragment thereof comprises PRA023. In some embodiments, the TL1A antibody or antigen-binding fragment thereof comprises tulisokibart. In some embodiments, the antibody or antigen-binding fragment thereof comprises a CD30L antibody or antigen-binding fragment thereof. In some embodiments, the CD30L antibody or antigen-binding fragment thereof comprises KPL-045. In some embodiments, the CD30L antibody or antigen-binding fragment thereof comprises PRA052.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the present subject matter will be obtained by reference to the following detailed description that sets forth illustrative embodiments and the accompanying drawings of which:

FIG. 1A provide a results from a principal component analysis (PCA) of RNA-seq data based on the hierarchical clustering of CD13+ subgroup versus other monocyte subgroups.

FIG. 1B provides results from a clustering analysis of total RNA-seq, which illustrate a distinct CD13+ cluster versus other monocyte clusters.

FIG. 2A is a PCA showing no batch effects in the clustered identified in FIGS. 1A-B.

FIG. 2B is a PCA showing no carry over of Crohn's disease (CD)-PBmu (CD-PBmu) subtype in the clusters identified in FIGS. 1A-B.

FIG. 3A provides the results from a PCA analysis of RNA-seq data from the CD13+ subgroup base on the hierarchical clustering shown in FIG. 3B.

FIG. 3B provides results from a clustering analysis of RNA-seq data from the CD13+ subgroup, which shows two distinct CD13+ clusters.

FIG. 4A is a heatmap illustrating the differential gene expression of CD13+ populations.

FIG. 4B is a heatmap of differential gene expression of the 952 transcripts identified in FIG. 4A having at least a 2-fold difference in gene expression, and illustrates that the CD13+ subgroup has a unique differential gene expression as compared with the other monocyte subgroups.

FIG. 5A illustrates the breakdown of the proportion of gross pathology assessment in affected tissue from samples analyzed in this study. Without being bound by any particular theory, this even distribution between cluster 1 and cluster 2 of the CD13+ subgroup suggests that gross condition is not a driver of the observed differential gene expression between the two subgroups.

FIG. 5B illustrates is a breakdown of the proportion of disease location (color or small bowel, “SB”/Ileum) of the samples analyzed in this study. Without being bound by any particular theory, this even distribution between cluster 1 and cluster 2 of the CD13+ subgroup suggests that disease location is not a driver of the observed different gene expression between the two subgroups.

FIG. 6A is a heatmap illustrating the differential gene expression of CD13+ subgroups, and shows that the differential gene expression is observed irrespective of disease location. Without being bound by any particular theory, this suggests that the differential gene expression observed in the two CD13+ subgroups is not driven by disease location. based on the clustering analyses, rather than disease location.

FIG. 6B is a Venn diagram illustrating the number of the vast majority of the differentially expressed genes observed in the two CD13+ subgroups is driven by the clustering of those subgroups, rather than disease location.

FIG. 7 illustrates the upregulation of pathways in CD13 cluster 1.

FIG. 8A illustrates an analysis using ARCHS⁴ and shows that the genes that are differentially expressed in the CD13+ cluster 1 subgroup are also reflected in other data sets related to macrophages.

FIG. 8B illustrates an analysis using ARCHS⁴ and shows that the genes that are differentially expressed in the CD13+ cluster 1 subgroup are also reflected in other data sets related to monocytes.

FIG. 8C illustrates an analysis using ARCHS⁴ and shows that the genes that are differentially expressed in the CD13+ cluster 1 subgroup are also reflected in other data sets related to the first ranked 300 genes upregulated in CD13+ cluster 1.

FIG. 9 illustrates a pathway analysis performed on RNA-seq data from CD13+ cluster 2, and shows the upregulation of certain pathways in this cluster.

FIG. 10 illustrates a pathway analysis performed on RNA-seq data from CD13+ cluster 2, and shows the upregulation of certain pathways in CD13+ cluster 2.

FIG. 11A illustrates an analysis using ARCHS⁴ and shows that the genes that are differentially expressed in the CD13+ cluster 2 subgroup are also reflected in other human data sets.

FIG. 11B illustrates an analysis using ARCHS⁴ and shows that the genes that are differentially expressed in the CD13+ cluster 2 subgroup are also reflected in other human data sets in the small bowel and colon.

FIG. 11C illustrates an analysis using ARCHS⁴ and shows that the genes that are differentially expressed in the CD13+ cluster 2 subgroup are also reflected in other mouse data sets.

FIG. 11D illustrates an analysis using ARCHS⁴ and shows that the genes that are differentially expressed in the CD13+ cluster 2 subgroup are also reflected in other mouse data sets in the small bowel and colon.

FIG. 12 is a heat map of differentially expressed genes between CD13+ cluster 1 and CD13+ cluster 2 that are targets for CD associated miRNA.

FIG. 13 is a cell type enrichment analysis of RNA-seq data to characterize the CD13+MNP cell subgroups.

FIG. 14 illustrates association of CD13+ cluster 2 cluster with elevated levels of serological markers.

FIG. 15 illustrates association of CD13+ cluster 2 cluster with increased expression of 5-hydroxytryptamine receptor 2A (HTR2A) and 5-Hydroxytryptamine Receptor 4 (HTR4).

FIG. 16A illustrates that the distribution of the CD-PBmu subtype is equally distributed between the two CD13+ clusters.

FIG. 16B illustrates the results from a pathway analysis of overlapping genes between the PBmu subtype and CD13+ cluster 1.

### DETAILED DESCRIPTION

The present disclosure provides methods and systems for characterizing and treating patients having Crohn's disease (CD). Intestinal mononuclear phagocytes (MNP) play a key role in innate immunity, gut homeostasis and intestinal disease, and MNP activation contributes to chronic inflammation and disease progression in CD. Therefore, there is a need to characterize the MNP signatures to provide more effective therapeutic strategies based on transcriptomic profiles. In particular embodiments, a CD patient is characterized as having or not having an inflammatory mononuclear phagocytes (MNP) signature or a resident mucosal MNP signature by transcriptomic profiling. A patient having either one of these signatures may express one or more genes of Tables 1, 2A-2B at a level higher than a reference subject. The reference subject may be a subject that does not have IBD. The reference subject may be a subject that does not have CD or a severe form of CD. The reference subject may be a subject that does not have a PBT subtype of CD.

Further provided herein are compositions for determining a Crohn's Disease (CD) subtype status in a subject having CD comprising a primer and/or a probe for amplifying and/or detecting the genes in Tables 1, 2A-2B. Also provided herein are systems comprising a kit for determining a Crohn's Disease (CD) subtype status in a subject having CD. The kits may comprise: one or more detection reagents comprising nucleic acids configured to hybridize to one or more genes in Tables 1, or 2A-2B. In some instances, the kit comprises qPCR, sequencing chips, or library preparations kits comprising target nucleic acid sequencing. Further provided herein are computer-implemented platforms for determining a Crohn's Disease (CD) subtype status in a subject having CD. The platform may comprise an array for determining one or more genes from Tables 2A-2B. The platforms may comprise one or more processors for detecting a level of expression of the one or more genes from the array and determining the CD subtype status of the subject based upon the expression profile. The platforms may further comprise a database for storing the one or more genes from the array, the level of expression from the one or more processors, or a combination thereof.

### Transcriptomic Signature and Profiling

Transcriptomic signatures may be used to identify genetic variations in populations. The genes identified by analyzing transcriptomic signatures may show outlier expressions or may be used to group subpopulations. In one aspect, provided herein are transcriptomic signatures associated with a subtype of IBD, including CD. In some cases, the transcriptomic signature comprises one or more genes of Table 1. In some cases, the transcriptomic signature comprises about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 1.

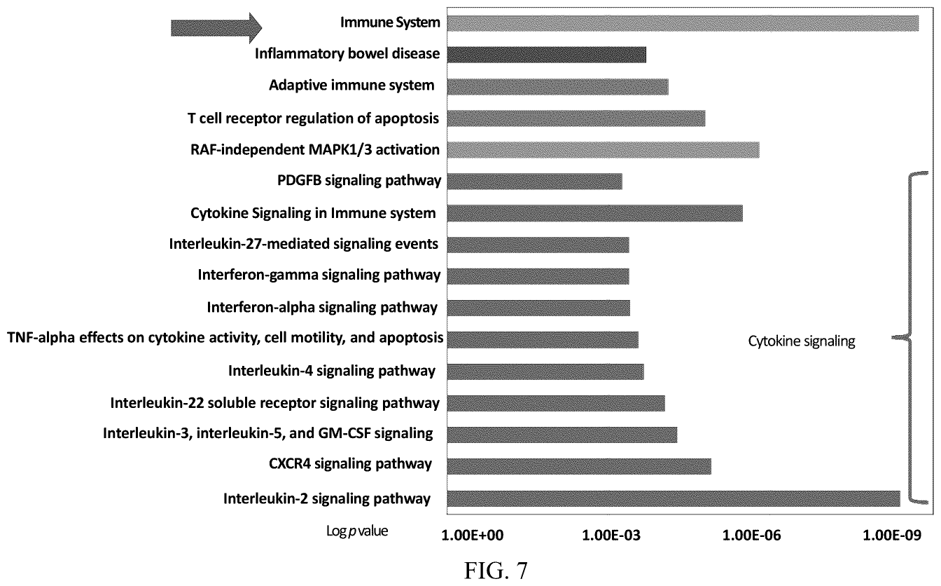

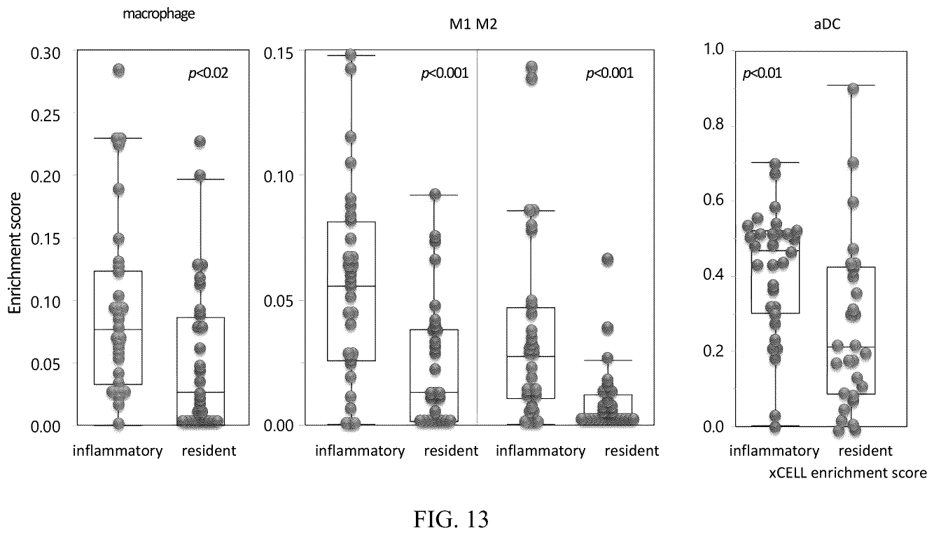

In some embodiments, the transcriptomic signature is predictive of a severe form of a disease or a condition in a subject, such as an inflammatory bowel disease (IBD). In some embodiments, the IBD comprises Crohn's disease (CD) or ulcerative colitis (UC). In some embodiments, the subtype characterized by an increase or a decrease in serological markers that display an immune reactivity to a microbial antigen. In some embodiments, the serological markers comprise antineutrophil cytoplasmic antibodies (ANCA), antibodies (IgG) against the yeast Saccharomyces cerevisiae (ASCA), or a combination thereof. In some embodiments, subtype is associated with an increase or a decrease in a quartile sum score (QSS) of such serological markers. A quartile sum score as disclosed herein may be calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699, which is hereby incorporated by reference in its entirety. In some embodiments, the subtype is associated with disease in a particular tissue ofthe GI tract, such as the large intestine or the small intestine. In some embodiments, the particular tissue comprises the colon, ileum, or ileocolonic region of the intestine, or a combination thereof. In some embodiments, the subtype is associated with an upregulation of the immune system, cytokine signaling or a combination thereof. Non-limiting examples of cytokine signaling pathways that are upregulated in the subtype disclosed herein are provided in FIG. 7. In some embodiments, the subtype is associated with an enrichment of macrophage, pro-inflammatory (Mm) or anti-inflammatory (M2) phenotypes, and activated dendric cells (“aDC”), as shown in FIG. 13. In some embodiments, the subtype is characterized by an upregulation of serotonin receptors, such as 5-hydroxytryptamine receptor 2A (HTR2A) and 5-Hydroxytryptamine Receptor 4 (HTR4).

TABLE 1
Exemplary Biomarkers of a Transcriptomic Signature

| | | | Geom | Geom | | | | | |
|---|---|---|---|---|---|---|---|---|---|
| | | Permu- | mean of | mean of | | | | | |
| Parametric | | tation | intensities | intensities | Fold | | | Entrez | |
| p-value | FDR | p-value | in class 1 | in class 2 | change | UniqueID | Name | ID | Accession |
| <1e−07 | <1e−07 | <1e−07 | 15.76 | 94.88 | 0.17 | MTCO3P12 | mitochondrially | 107075270 | |
| | | | | | | | encoded | | |
| | | | | | | | cytochrome c | | |
| | | | | | | | oxidase III | | |
| | | | | | | | pseudogene 12 | | |
| <1e−07 | <1e−07 | <1e−07 | 22.77 | 9.57 | 2.38 | IFFO1 | intermediate | 25900 | NM_001039670 |
| | | | | | | | filament family | | |
| | | | | | | | orphan 1 | | |
| <1e−07 | <1e−07 | <1e−07 | 35.37 | 9.17 | 3.86 | LILRB1 | leukocyte | 10859 | NM_001081637 |
| | | | | | | | immunoglobulin | | |
| | | | | | | | like receptor B1 | | |
| <1e−07 | <1e−07 | <1e−07 | 17.8 | 6.76 | 2.63 | S1PR2 | sphingosine-1- | 9294 | NM_004230 |
| | | | | | | | phosphate | | |
| | | | | | | | receptor 2 | | |
| <1e−07 | <1e−07 | <1e−07 | 82.81 | 24.73 | 3.35 | GGA2 | Golgi associated, | 23062 | NM_015044 |
| | | | | | | | gamma adaptin | | |
| | | | | | | | ear containing, | | |
| | | | | | | | ARF binding | | |
| | | | | | | | protein 2 | | |
| <1e−07 | <1e−07 | <1e−07 | 137.68 | 59.28 | 2.32 | ZMIZ2 | zinc finger MIZ- | 83637 | NM_001300959 |
| | | | | | | | type containing 2 | | |
| <1e−07 | <1e−07 | <1e−07 | 32.02 | 12.62 | 2.54 | AMFR | autocrine motility | 267 | NM_001144 |
| | | | | | | | factor receptor | | |
| <1e−07 | <1e−07 | <1e−07 | 80.17 | 26.55 | 3.02 | CBX6 | chromobox 6 | 23466 | NM_001303494 |
| <1e−07 | <1e−07 | <1e−07 | 70.58 | 25.68 | 2.75 | DPP7 | dipeptidyl | 29952 | NM_013379 |
| | | | | | | | peptidase 7 | | |
| <1e−07 | <1e−07 | <1e−07 | 74.23 | 32.64 | 2.27 | MSI2 | musashi RNA | 124540 | NM_001322250 |
| | | | | | | | binding protein 2 | | |
| <1e−07 | <1e−07 | <1e−07 | 117.5 | 38.48 | 3.05 | SYNE3 | spectrin repeat | 161176 | NM_024633 |
| | | | | | | | containing nuclear | | |
| | | | | | | | envelope family | | |
| | | | | | | | member 3 | | |
| <1e−07 | <1e−07 | <1e−07 | 37.22 | 15.76 | 2.36 | TJAP1 | tight junction | 93643 | NM_001146016 |
| | | | | | | | associated protein | | |
| | | | | | | | 1 | | |
| <1e−07 | <1e−07 | <1e−07 | 104.42 | 38.85 | 2.69 | WDR6 | WD repeat | 11180 | NM_001320546 |
| | | | | | | | domain 6 | | |
| <1e−07 | <1e−07 | <1e−07 | 46.63 | 11.04 | 4.22 | GNG7 | G protein subunit | 2788 | NM_052847 |
| | | | | | | | gamma 7 | | |
| <1e−07 | <1e−07 | <1e−07 | 138.37 | 54.05 | 2.56 | TNK2 | tyrosine kinase | 10188 | NM_001010938 |
| | | | | | | | non receptor 2 | | |
| <1e−07 | <1e−07 | <1e−07 | 6.85 | 31.91 | 0.21 | MTND1P23 | mitochondrially | 100887749 | |
| | | | | | | | encoded | | |
| | | | | | | | NADH: ubiquinone | | |
| | | | | | | | oxidoreductase | | |
| | | | | | | | core subunit 1 | | |
| | | | | | | | pseudogene 23 | | |
| <1e−07 | <1e−07 | <1e−07 | 89.98 | 30.41 | 2.96 | BCL2 | BCL2, apoptosis | 596 | NM_000633 |
| | | | | | | | regulator | | |
| <1e−07 | <1e−07 | <1e−07 | 15.68 | 6.73 | 2.33 | WDR45 | WD repeat | 11152 | NM_001029896 |
| | | | | | | | domain 45 | | |
| <1e−07 | <1e−07 | <1e−07 | 2535.56 | 831.48 | 3.05 | AHNAK | AHNAK | 79026 | NM_001346445 |
| | | | | | | | nucleoprotein | | |
| <1e−07 | <1e−07 | <1e−07 | 160.82 | 73.93 | 2.18 | JAK1 | Janus kinase 1 | 3716 | NM_001320923 |
| <1e−07 | <1e−07 | <1e−07 | 27.24 | 10.57 | 2.58 | DNAJC1 | DnaJ heat shock | 64215 | NM_022365 |
| | | | | | | | protein family | | |
| | | | | | | | (Hsp40) member | | |
| | | | | | | | C1 | | |
| <1e−07 | <1e−07 | <1e−07 | 55.04 | 24.58 | 2.24 | TUG1 | taurine up- | 55000 | NR_00232323 |
| | | | | | | | regulated 1 (non- | | |
| | | | | | | | protein coding) | | |
| <1e−07 | <1e−07 | <1e−07 | 186.65 | 54.42 | 3.43 | FAM214A | family with | 56204 | NM_001286495 |
| | | | | | | | sequence | | |
| | | | | | | | similarity 214 | | |
| | | | | | | | member A | | |
| <1e−07 | <1e−07 | <1e−07 | 20.68 | 8.66 | 2.39 | CFAP410 | Cilia And Flagella | 755 | |
| | | | | | | | Associated | | |
| | | | | | | | Protein 410 | | |
| <1e−07 | <1e−07 | <1e−07 | 14.2 | 5.72 | 2.48 | AL353795.3 | noncoding | | |
| | | | | | | | (Ensemble gene | | |
| | | | | | | | ID: | | |
| | | | | | | | ENSG00000279608.1) | | |
| <1e−07 | <1e−07 | <1e−07 | 7.83 | 45.2 | 0.17 | MTCO2P12 | mitochondrially | 107075310 | |
| | | | | | | | encoded | | |
| | | | | | | | cytochrome c | | |
| | | | | | | | oxidase II | | |
| | | | | | | | pseudogene 12 | | |
| <1e−07 | <1e−07 | <1e−07 | 16.77 | 6.84 | 2.45 | AC005332.4 | noncoding | | |
| | | | | | | | (Ensemble gene | | |
| | | | | | | | ID: | | |
| | | | | | | | ENSG00000274712.1) | | |
| <1e−07 | <1e−07 | <1e−07 | 48.24 | 15.04 | 3.21 | NLRP1 | NLR family pyrin | 22861 | NM_001033053 |
| | | | | | | | domain containing | | |
| | | | | | | | 1 | | |
| <1e−07 | <1e−07 | <1e−07 | 23.2 | 7.58 | 3.06 | C13orf46 | chromosome 13 | 100507747 | XM_006719994 |
| | | | | | | | open reading | | |
| | | | | | | | frame 46 | | |
| <1e−07 | <1e−07 | <1e−07 | 26.87 | 7.04 | 3.82 | TNFRSF13C | TNF receptor | 115650 | NM_052945 |
| | | | | | | | superfamily | | |
| | | | | | | | member 13C | | |
| <1e−07 | <1e−07 | <1e−07 | 35.88 | 16.49 | 2.18 | ZCCHC8 | zinc finger | 55596 | NM_001350935 |
| | | | | | | | CCHC-type | | |
| | | | | | | | containing 8 | | |
| <1e−07 | <1e−07 | <1e−07 | 14910.29 | 58582.77 | 0.25 | MT-CO2 | Mitochondrially | 4513 | |
| | | | | | | | Encoded | | |
| | | | | | | | Cytochrome C | | |
| | | | | | | | Oxidase II | | |
| <1e−07 | <1e−07 | <1e−07 | 58.75 | 22.76 | 2.58 | CCDC69 | coiled-coil | 26112 | NM_015621 |
| | | | | | | | domain containing | | |
| | | | | | | | 69 | | |
| <1e−07 | <1e−07 | <1e−07 | 36.27 | 14.74 | 2.46 | IDS | iduronate 2- | 3423 | NM_000202 |
| | | | | | | | sulfatase | | |
| <1e−07 | <1e−07 | <1e−07 | 109.03 | 33.16 | 3.29 | CLCN7 | chloride voltage- | 1186 | NM_001114331 |
| | | | | | | | gated channel 7 | | |
| <1e−07 | <1e−07 | <1e−07 | 15.6 | 5.95 | 2.62 | PARP15 | poly(ADP-ribose) | 165631 | NM_001113523 |
| | | | | | | | polymerase family | | |
| | | | | | | | member 15 | | |
| <1e−07 | <1e−07 | <1e−07 | 104.8 | 28.66 | 3.66 | LSS | lanosterol | 4047 | NM_001001438 |
| | | | | | | | synthase | | |
| <1e−07 | <1e−07 | <1e−07 | 10.38 | 45.18 | 0.23 | MTRNR2L12 | MT-RNR2 like 12 | 100463498 | |
| <1e−07 | <1e−07 | <1e−07 | 376.37 | 149.5 | 2.52 | FNBP1 | formin binding | 23048 | NM_015033 |
| | | | | | | | protein 1 | | |
| <1e−07 | <1e−07 | <1e−07 | 21.89 | 7.36 | 2.97 | N4BP3 | NEDD4 binding | 23138 | NM_015111 |
| | | | | | | | protein 3 | | |
| <1e−07 | <1e−07 | <1e−07 | 28.28 | 12.45 | 2.27 | RANBP10 | RAN binding | 57610 | NM_001320238 |
| | | | | | | | protein 10 | | |
| <1e−07 | <1e−07 | <1e−07 | 247.22 | 117.63 | 2.1 | FUS | FUS RNA binding | 2521 | NM_001010850 |
| | | | | | | | protein | | |
| <1e−07 | <1e−07 | <1e−07 | 13455.22 | 54847.16 | 0.25 | MT-ATP6 | Mitochondrially | 4508 | |
| | | | | | | | Encoded ATP | | |
| | | | | | | | Synthase | | |
| | | | | | | | Membrane | | |
| | | | | | | | Subunit 6 | | |
| <1e−07 | <1e−07 | <1e−07 | 110.11 | 41.44 | 2.66 | NFATC1 | nuclear factor of | 4772 | NM_001278669 |
| | | | | | | | activated T cells 1 | | |
| <1e−07 | <1e−07 | <1e−07 | 40.05 | 12.43 | 3.22 | TTYH2 | tweety family | 94015 | NM_001330453 |
| | | | | | | | member 2 | | |
| <1e−07 | <1e−07 | <1e−07 | 39.4 | 15.01 | 2.63 | SLC12A4 | solute carrier | 6560 | NM_001145961 |
| | | | | | | | family 12 member | | |
| | | | | | | | 4 | | |
| <1e−07 | <1e−07 | <1e−07 | 16289.89 | 64793.73 | 0.25 | MT-CO3 | Mitochondrially | 4514 | |
| | | | | | | | Encoded | | |
| | | | | | | | Cytochrome C | | |
| | | | | | | | Oxidase III | | |
| <1e−07 | <1e−07 | <1e−07 | 74.55 | 31.51 | 2.37 | DDB1 | damage specific | 1642 | NM_001923 |
| | | | | | | | DNA binding | | |
| | | | | | | | protein 1 | | |
| <1e−07 | <1e−07 | <1e−07 | 12.82 | 54.33 | 0.24 | MT-TP | Mitochondrially | 4571 | |
| | | | | | | | Encoded TRNA- | | |
| | | | | | | | Pro (CCN) | | |
| <1e−07 | <1e−07 | <1e−07 | 140.7 | 69.07 | 2.04 | PCF11 | PCF11 cleavage | 51585 | NM_001346413 |
| | | | | | | | and | | |
| | | | | | | | polyadenylation | | |
| | | | | | | | factor subunit | | |
| <1e−07 | <1e−07 | <1e−07 | 34.13 | 13.46 | 2.54 | PNMA1 | PNMA family | 9240 | NM_006029 |
| | | | | | | | member 1 | | |
| <1e−07 | <1e−07 | <1e−07 | 45.71 | 12.09 | 3.78 | SLC26A11 | solute carrier | 284129 | NM_001166347 |
| | | | | | | | family 26 member | | |
| | | | | | | | 11 | | |
| <1e−07 | <1e−07 | <1e−07 | 26.25 | 10.32 | 2.54 | SAT2 | spermidine/ | 112483 | NM_001320845 |
| | | | | | | | spermine N1- | | |
| | | | | | | | acetyltransferase | | |
| | | | | | | | family member 2 | | |
| <1e−07 | <1e−07 | <1e−07 | 57.21 | 23.51 | 2.43 | SNX29 | sorting nexin 29 | 92017 | NM_001009607 |
| <1e−07 | <1e−07 | <1e−07 | 32.35 | 11.47 | 2.82 | MAP4K1 | mitogen-activated | 11184 | NM_001042600 |
| | | | | | | | protein kinase | | |
| | | | | | | | kinase kinase | | |
| | | | | | | | kinase 1 | | |
| <1e−07 | <1e−07 | <1e−07 | 213.56 | 70.39 | 3.03 | AC004151.1 | noncoding | | |
| | | | | | | | (Ensemble gene | | |
| | | | | | | | ID: | | |
| | | | | | | | ENSG00000180448) | | |
| <1e−07 | <1e−07 | <1e−07 | 137.54 | 51.34 | 2.68 | JMY | junction | 133746 | NM_152405 |
| | | | | | | | mediating and | | |
| | | | | | | | regulatory protein, | | |
| | | | | | | | p53 cofactor | | |
| <1e−07 | <1e−07 | <1e−07 | 312.66 | 152.03 | 2.06 | SON | SON DNA | 6651 | NM_001291411 |
| | | | | | | | binding protein | | |
| <1e−07 | <1e−07 | <1e−07 | 27.87 | 11.87 | 2.35 | NSMAF | neutral | 8439 | NM_001144772 |
| | | | | | | | sphingomyelinase | | |
| | | | | | | | activation | | |
| | | | | | | | associated factor | | |
| <1e−07 | <1e−07 | <1e−07 | 227.62 | 84.45 | 2.7 | PABPC4 | poly(A) binding | 8761 | NM_001135653 |
| | | | | | | | protein | | |
| | | | | | | | cytoplasmic 4 | | |
| <1e−07 | <1e−07 | <1e−07 | 36.87 | 16.86 | 2.19 | MARCHF8 | Membrane | 220972 | |
| | | | | | | | Associated Ring- | | |
| | | | | | | | CH-Type Finger 8 | | |
| <1e−07 | <1e−07 | <1e−07 | 107.45 | 31.59 | 3.4 | ARL4C | ADP ribosylation | 10123 | NM_001282431 |
| | | | | | | | factor like GTPase | | |
| | | | | | | | 4C | | |
| <1e−07 | <1e−07 | <1e−07 | 2076.53 | 7833.8 | 0.27 | MTATP6P1 | mitochondrially | 106480796 | |
| | | | | | | | encoded ATP | | |
| | | | | | | | synthase 6 | | |
| | | | | | | | pseudogene 1 | | |
| <1e−07 | <1e−07 | <1e−07 | 105.73 | 51.21 | 2.06 | MYCBP2 | MYC binding | 23077 | NM_015057 |
| | | | | | | | protein 2, E3 | | |
| | | | | | | | ubiquitin protein | | |
| | | | | | | | ligase | | |
| <1e−07 | <1e−07 | <1e−07 | 26.87 | 12.32 | 2.18 | LIMD1 | LIM domains | 8994 | NM_014240 |
| | | | | | | | containing 1 | | |
| <1e−07 | <1e−07 | <1e−07 | 109.67 | 49.65 | 2.21 | PI4KA | phosphatidylinositol | 5297 | NM_002650 |
| | | | | | | | 4-kinase alpha | | |
| <1e−07 | <1e−07 | <1e−07 | 37.12 | 10.37 | 3.58 | RASGRP2 | RAS guanyl | 10235 | NM_001098670 |
| | | | | | | | releasing protein 2 | | |
| <1e−07 | <1e−07 | <1e−07 | 41.91 | 14.23 | 2.95 | TIAM1 | T cell lymphoma | 7074 | NM_001353684 |
| | | | | | | | invasion and | | |
| | | | | | | | metastasis 1 | | |
| <1e−07 | <1e−07 | <1e−07 | 122.59 | 54.47 | 2.25 | PLEKHM2 | pleckstrin | 23207 | NM_015164 |
| | | | | | | | homology and | | |
| | | | | | | | RUN domain | | |
| | | | | | | | containing M2 | | |
| <1e−07 | <1e−07 | <1e−07 | 66.87 | 16.87 | 3.96 | WDFY4 | WDFY family | 57705 | NM_020945 |
| | | | | | | | member 4 | | |
| <1e−07 | <1e−07 | <1e−07 | 174.09 | 75.92 | 2.29 | EHBP1L1 | EH domain | 254102 | NM_001099409 |
| | | | | | | | binding protein 1 | | |
| | | | | | | | like 1 | | |
| <1e−07 | <1e−07 | <1e−07 | 210.01 | 87.63 | 2.4 | MEF2D | myocyte enhancer | 4209 | NM_001271629 |
| | | | | | | | factor 2D | | |
| <1e−07 | <1e−07 | <1e−07 | 154.99 | 55.41 | 2.8 | RIPOR1 | RHO family | 79567 | NM_001193522 |
| | | | | | | | interacting cell | | |
| | | | | | | | polarization | | |
| | | | | | | | regulator 1 | | |
| <1e−07 | <1e−07 | <1e−07 | 24.5 | 11.33 | 2.16 | MOB2 | MOB kinase | 81532 | NM_001172223 |
| | | | | | | | activator 2 | | |
| <1e−07 | <1e−07 | <1e−07 | 225.48 | 79.68 | 2.83 | FMNL1 | formin like 1 | 752 | NM_005892 |
| <1e−07 | <1e−07 | <1e−07 | 273.91 | 104.64 | 2.62 | ERN1 | endoplasmic | 2081 | NM_001433 |
| | | | | | | | reticulum to | | |
| | | | | | | | nucleus signaling | | |
| | | | | | | | 1 | | |
| <1e−07 | <1e−07 | <1e−07 | 16.7 | 8.1 | 2.06 | ENO2 | enolase 2 | 2026 | NM_001975 |
| <1e−07 | <1e−07 | <1e−07 | 42.3 | 14.71 | 2.88 | POU2F2 | POU class 2 | 5452 | NM_00120725 |
| | | | | | | | homeobox 2 | | |
| <1e−07 | <1e−07 | <1e−07 | 28.65 | 11.47 | 2.5 | PARVB | parvin beta | 29780 | NM_001003828 |
| <1e−07 | <1e−07 | <1e−07 | 42.45 | 19.25 | 2.21 | GNPTAB | N- | 79158 | NM_024312 |
| | | | | | | | acetylglucosamine- | | |
| | | | | | | | 1-phosphate | | |
| | | | | | | | transferase alpha | | |
| | | | | | | | and beta subunits | | |
| <1e−07 | <1e−07 | <1e−07 | 318.76 | 112.6 | 2.83 | HYOU1 | hypoxia up- | 10525 | NM_001130991 |
| | | | | | | | regulated 1 | | |
| <1e−07 | <1e−07 | <1e−07 | 41.47 | 13.72 | 3.02 | PARVG | parvin gamma | 64098 | NM_001137605 |
| <1e−07 | <1e−07 | <1e−07 | 31.7 | 12.85 | 2.47 | ATP8B2 | ATPase | 57198 | NM_001005855 |
| | | | | | | | phospholipid | | |
| | | | | | | | transporting 8B2 | | |
| <1e−07 | <1e−07 | <1e−07 | 402.76 | 199.61 | 2.02 | DYNC1H1 | dynein | 1778 | NM_001376 |
| | | | | | | | cytoplasmic 1 | | |
| | | | | | | | heavy chain 1 | | |
| <1e−07 | <1e−07 | <1e−07 | 20.26 | 9.63 | 2.1 | CCM2 | CCM2 scaffold | 83605 | NM_001029835 |
| | | | | | | | protein | | |
| <1e−07 | <1e−07 | <1e−07 | 65.15 | 22.85 | 2.85 | EML3 | echinoderm | 256364 | NM_001300793 |
| | | | | | | | microtubule | | |
| | | | | | | | associated protein | | |
| | | | | | | | like 3 | | |
| <1e−07 | <1e−07 | <1e−07 | 5.58 | 18.81 | 0.3 | CPS1 | carbamoyl- | 1373 | NM_001122633 |
| | | | | | | | phosphate | | |
| | | | | | | | synthase 1 | | |
| <1e−07 | <1e−07 | <1e−07 | 191 | 85.45 | 2.24 | ATXN2L | ataxin 2 like | 11273 | NM_001308230 |
| <1e−07 | <1e−07 | <1e−07 | 193.6 | 94.76 | 2.04 | CIRBP | cold inducible | 1153 | NM_001280 |
| | | | | | | | RNA binding | | |
| | | | | | | | protein | | |
| <1e−07 | <1e−07 | <1e−07 | 55.94 | 24.01 | 2.33 | HDAC7 | histone | 51564 | NM_001098416 |
| | | | | | | | deacety lase 7 | | |
| <1e−07 | <1e−07 | <1e−07 | 48.4 | 16.48 | 2.94 | RBM38 | RNA binding | 55544 | NM_001291780 |
| | | | | | | | motif protein 38 | | |
| <1e−07 | <1e−07 | <1e−07 | 47.69 | 19.83 | 2.4 | PLEKHM1 | pleckstrin | 9842 | NM_001352825 |
| | | | | | | | homology and | | |
| | | | | | | | RUN domain | | |
| | | | | | | | containing M1 | | |
| <1e−07 | <1e−07 | <1e−07 | 6.43 | 16.78 | 0.38 | ARFGEF3 | ARFGEF family | 57221 | NM_020340 |
| | | | | | | | member 3 | | |
| <1e−07 | <1e−07 | <1e−07 | 81.75 | 40.28 | 2.03 | HNRNPA0 | heterogeneous | 10949 | NM_006805 |
| | | | | | | | nuclear | | |
| | | | | | | | ribonucleoprotein | | |
| | | | | | | | A0 | | |
| <1e−07 | <1e−07 | <1e−07 | 20.86 | 9.75 | 2.14 | UBE2J2 | ubiquitin | 118424 | NM_05816767 |
| | | | | | | | conjugating | | |
| | | | | | | | enzyme E2 J2 | | |
| <1e−07 | <1e−07 | <1e−07 | 71.28 | 20.3 | 3.51 | UAP1L1 | UDP-N- | 91373 | NM_207309 |
| | | | | | | | acetylglucosamine | | |
| | | | | | | | pyrophosphorylase | | |
| | | | | | | | 1 like 1 | | |
| <1e−07 | <1e−07 | <1e−07 | 108.35 | 50.07 | 2.16 | USP48 | ubiquitin specific | 84196 | NM_001032730 |
| | | | | | | | peptidase 48 | | |
| <1e−07 | <1e−07 | <1e−07 | 6.2 | 15.12 | 0.41 | MECOM | MDS1 and EVI1 | 2122 | NM_001105077 |
| | | | | | | | complex locus | | |
| <1e−07 | <1e−07 | <1e−07 | 98.69 | 48.95 | 2.02 | BAG6 | BCL2 associated | 7917 | NM_001098534 |
| | | | | | | | athanogene 6 | | |
| <1e−07 | <1e−07 | <1e−07 | 5.32 | 13.03 | 0.41 | SI | sucrase-isomaltase | 6476 | NM_001041 |
| <1e−07 | <1e−07 | <1e−07 | 96.42 | 38.88 | 2.48 | ULK1 | unc-51 like | 8408 | NM_003565 |
| | | | | | | | autophagy | | |
| | | | | | | | activating kinase 1 | | |
| <1e−07 | <1e−07 | <1e−07 | 62.35 | 27.69 | 2.25 | SMCR8 | Smith-Magenis | 140775 | NM_144775 |
| | | | | | | | syndrome | | |
| | | | | | | | chromosome | | |
| | | | | | | | region, candidate | | |
| | | | | | | | 8 | | |
| <1e−07 | <1e−07 | <1e−07 | 16436.22 | 59414.58 | 0.28 | MT-CYB | Mitochondrially | 4519 | |
| | | | | | | | Encoded | | |
| | | | | | | | Cytochrome B | | |
| <1e−07 | <1e−07 | <1e−07 | 127.39 | 49.76 | 2.56 | ZHX2 | zinc fingers and | 22882 | NM_014943 |
| | | | | | | | homeoboxes 2 | | |
| <1e−07 | <1e−07 | <1e−07 | 1930.16 | 7242.55 | 0.27 | MT-ATP8 | Mitochondrially | 4509 | |
| | | | | | | | Encoded ATP | | |
| | | | | | | | Synthase | | |
| | | | | | | | Membrane | | |
| | | | | | | | Subunit 8 | | |
| <1e−07 | <1e−07 | <1e−07 | 115.79 | 38.17 | 3.03 | RUBCN | RUN and cysteine | 9711 | NM_001145642 |
| | | | | | | | rich domain | | |
| | | | | | | | containing beclin | | |
| | | | | | | | 1 interacting | | |
| | | | | | | | protein | | |
| <1e−07 | <1e−07 | <1e−07 | 79.7 | 289.95 | 0.27 | MTCO1P12 | mitochondrially | 107075141 | |
| | | | | | | | encoded | | |
| | | | | | | | cytochrome c | | |
| | | | | | | | oxidase I | | |
| | | | | | | | pseudogene 12 | | |
| <1e−07 | <1e−07 | <1e−07 | 82.43 | 24.9 | 3.31 | AP5Z1 | adaptor related | 9907 | NM_014855 |
| | | | | | | | protein complex 5 | | |
| | | | | | | | zeta 1 subunit | | |
| <1e−07 | <1e−07 | <1e−07 | 61.29 | 15.71 | 3.9 | SLC17A9 | solute carrier | 63910 | NM_001302643 |
| | | | | | | | family 17 member | | |
| | | | | | | | 9 | | |
| <1e−07 | <1e−07 | <1e−07 | 32.25 | 12.29 | 2.62 | MBD3 | methyl-CpG | 53615 | NM_001281453 |
| | | | | | | | binding domain | | |
| | | | | | | | protein 3 | | |
| <1e−07 | <1e−07 | <1e−07 | 19.63 | 8.67 | 2.26 | TCP11L2 | t-complex 11 like | 255394 | NM_001286262 |
| | | | | | | | 2 | | |
| <1e−07 | <1e−07 | <1e−07 | 15.57 | 5.51 | 2.83 | AC092068.3 | noncoding | | |
| | | | | | | | (Ensemble gene | | |
| | | | | | | | ID: | | |
| | | | | | | | ENSG00000267749.1) | | |
| <1e−07 | <1e−07 | <1e−07 | 70.24 | 27.9 | 2.52 | MTA2 | metastasis | 9219 | NM_001330292 |
| | | | | | | | associated 1 | | |
| | | | | | | | family member 2 | | |
| <1e−07 | <1e−07 | <1e−07 | 6.19 | 18.56 | 0.33 | AGR2 | anterior gradient | 10551 | NM_006408 |
| | | | | | | | 2, protein | | |
| | | | | | | | disulphide | | |
| | | | | | | | isomerase family | | |
| | | | | | | | member | | |
| <1e−07 | <1e−07 | <1e−07 | 56.48 | 22.05 | 2.56 | TRPM7 | transient receptor | 54822 | NM_001301212 |
| | | | | | | | potential cation | | |
| | | | | | | | channel subfamily | | |
| | | | | | | | M member 7 | | |
| <1e−07 | <1e−07 | <1e−07 | 60.26 | 29.68 | 2.03 | SEC24C | SEC24 homolog | 9632 | NM_004922 |
| | | | | | | | C, COPII coat | | |
| | | | | | | | complex | | |
| | | | | | | | component | | |
| <1e−07 | <1e−07 | <1e−07 | 62.46 | 27.39 | 2.28 | PLEKHO1 | pleckstrin | 51177 | NM_001304722 |
| | | | | | | | homology domain | | |
| | | | | | | | containing O1 | | |
| <1e−07 | <1e−07 | <1e−07 | 46.11 | 21.35 | 2.16 | FAM13B | family with | 51306 | NM_001101800 |
| | | | | | | | sequence | | |
| | | | | | | | similarity 13 | | |
| | | | | | | | member B | | |
| <1e−07 | <1e−07 | <1e−07 | 67.84 | 24.86 | 2.73 | VAC14 | Vac14, PIKFYVE | 55697 | NM_001351157 |
| | | | | | | | complex | | |
| | | | | | | | component | | |
| <1e−07 | <1e−07 | <1e−07 | 457.87 | 162.39 | 2.82 | RAPGEF1 | Rap guanine | 2889 | NM_001304275 |
| | | | | | | | nucleotide | | |
| | | | | | | | exchange factor 1 | | |
| <1e−07 | <1e−07 | <1e−07 | 863.28 | 324.84 | 2.66 | FLNA | filamin A | 2316 | NM_001110556 |
| <1e−07 | <1e−07 | <1e−07 | 54.14 | 21.69 | 2.5 | ZNF460 | zinc finger protein | 10794 | NM_001330622 |
| | | | | | | | 460 | | |
| <1e−07 | <1e−07 | <1e−07 | 5.43 | 13.29 | 0.41 | ADGRV1 | adhesion G | 84059 | NM_032119 |
| | | | | | | | protein-coupled | | |
| | | | | | | | receptor V1 | | |
| <1e−07 | <1e−07 | <1e−07 | 178.83 | 71.85 | 2.49 | IL10RA | interleukin 10 | 3587 | NM_001558 |
| | | | | | | | receptor subunit | | |
| | | | | | | | alpha | | |
| <1e−07 | <1e−07 | <1e−07 | 117.18 | 46.8 | 2.5 | KDM2B | lysine | 84678 | NM_001005366 |
| | | | | | | | demethylase 2B | | |
| <1e−07 | <1e−07 | <1e−07 | 55.82 | 18.14 | 3.08 | SH3TC1 | SH3 domain and | 54436 | NM_001318480 |
| | | | | | | | tetratricopeptide | | |
| | | | | | | | repeats 1 | | |
| <1e−07 | <1e−07 | <1e−07 | 158.26 | 75.84 | 2.09 | ASXL1 | additional sex | 171023 | NM_001164603 |
| | | | | | | | combs like 1, | | |
| | | | | | | | transcriptional | | |
| | | | | | | | regulator | | |
| <1e−07 | <1e−07 | <1e−07 | 117.72 | 51.63 | 2.28 | KMT2A | lysine | 4297 | NM_001197104 |
| | | | | | | | methyltransferase | | |
| | | | | | | | 2A | | |
| <1e−07 | <1e−07 | <1e−07 | 94.46 | 41.88 | 2.26 | LPIN1 | lipin 1 | 23175 | NM_001261427 |
| <1e−07 | <1e−07 | <1e−07 | 53.14 | 21.88 | 2.43 | SLC41A1 | solute carrier | 254428 | NM_173854 |
| | | | | | | | family 41 member | | |
| | | | | | | | 1 | | |
| <1e−07 | <1e−07 | <1e−07 | 6.72 | 18.97 | 0.35 | PCLO | piccolo | 27445 | NM_014510 |
| | | | | | | | presynaptic | | |
| | | | | | | | cytomatrix protein | | |
| <1e−07 | <1e−07 | <1e−07 | 62 | 23.09 | 2.69 | IL6R | interleukin 6 | 3570 | NM_000565 |
| | | | | | | | receptor | | |
| <1e−07 | <1e−07 | <1e−07 | 46.92 | 16.1 | 2.91 | FADS3 | fatty acid | 3995 | NM_021727 |
| | | | | | | | desaturase 3 | | |
| <1e−07 | <1e−07 | <1e−07 | 129.15 | 49.13 | 2.63 | PHF1 | PHD finger | 5252 | NM_002636 |
| | | | | | | | protein 1 | | |
| <1e−07 | <1e−07 | <1e−07 | 20.02 | 9.1 | 2.2 | LRWD1 | leucine rich | 222229 | NM_001317721 |
| | | | | | | | repeats and WD | | |
| | | | | | | | repeat domain | | |
| | | | | | | | containing 1 | | |
| <1e−07 | <1e−07 | <1e−07 | 111.77 | 40.15 | 2.78 | INPP5D | inositol | 3635 | NM_001017915 |
| | | | | | | | polyphosphate-5- | | |
| | | | | | | | phosphatase D | | |
| <1e−07 | <1e−07 | <1e−07 | 45.08 | 20.1 | 2.24 | MTMR14 | myotubularin | 64419 | NM_001077525 |
| | | | | | | | related protein 14 | | |
| <1e−07 | <1e−07 | <1e−07 | 20773.16 | 74286.26 | 0.28 | MT-ND1 | Mitochondrially | 4535 | |
| | | | | | | | Encoded | | |
| | | | | | | | NADH: Ubiquinone | | |
| | | | | | | | Oxidoreductase | | |
| | | | | | | | Core Subunit 1 | | |
| <1e−07 | <1e−07 | <1e−07 | 26.13 | 12.51 | 2.09 | DGCR8 | DGCR8, | 54487 | NM_001190326 |
| | | | | | | | microprocessor | | |
| | | | | | | | complex subunit | | |
| <1e−07 | <1e−07 | <1e−07 | 20.43 | 8.58 | 2.38 | ZCWPW2 | zinc finger CW- | 152098 | NM_001040432 |
| | | | | | | | type and PWWP | | |
| | | | | | | | domain containing | | |
| | | | | | | | 2 | | |
| <1e−07 | <1e−07 | <1e−07 | 268.86 | 130.79 | 2.06 | CYTH1 | cytohesin 1 | 9267 | NM_001292018 |
| <1e−07 | <1e−07 | <1e−07 | 134.97 | 63.88 | 2.11 | ASH1L | ASH 1 like histone | 55870 | NM_018489 |
| | | | | | | | lysine | | |
| | | | | | | | methyltransferase | | |
| <1e−07 | <1e−07 | <1e−07 | 73.92 | 27.98 | 2.64 | GALNT2 | polypeptide N- | 2590 | NM_001291866 |
| | | | | | | | acetyl- | | |
| | | | | | | | galactosaminyl- | | |
| | | | | | | | transferase 2 | | |
| <1e−07 | <1e−07 | <1e−07 | 31.16 | 14.79 | 2.11 | SNX13 | sorting nexin 13 | 23161 | NM_001350862 |
| <1e−07 | <1e−07 | <1e−07 | 56.34 | 20.57 | 2.74 | TMC6 | transmembrane | 11322 | NM_001127198 |
| | | | | | | | channel like 6 | | |
| <1e−07 | <1e−07 | <1e−07 | 5.34 | 11.07 | 0.48 | CFTR | cystic fibrosis | 1080 | NM_000492 |
| | | | | | | | transmembrane | | |
| | | | | | | | conductance | | |
| | | | | | | | regulator | | |
| <1e−07 | <1e−07 | <1e−07 | 26.34 | 9.62 | 2.74 | H1FX-AS1 | H1FX antisense | 339942 | NM_001025468 |
| | | | | | | | RNA 1 | | |
| <1e−07 | <1e−07 | <1e−07 | 28870.19 | 101887.14 | 0.28 | MT-ND4 | Mitochondrially | 4538 | |
| | | | | | | | Encoded | | |
| | | | | | | | NADH: Ubiquinone | | |
| | | | | | | | Oxidoreductase | | |
| | | | | | | | Core Subunit 4 | | |
| <1e−07 | <1e−07 | <1e−07 | 29.01 | 12.01 | 2.42 | SGSH | N- | 6448 | NM_000199 |
| | | | | | | | sulfoglucosamine | | |
| | | | | | | | sulfohydrolase | | |
| <1e−07 | <1e−07 | <1e−07 | 8.98 | 26.26 | 0.34 | MT-TS1 | Mitochondrially | 4574 | |
| | | | | | | | Encoded TRNA- | | |
| | | | | | | | Ser (UCN) 1 | | |
| <1e−07 | <1e−07 | <1e−07 | 114878.01 | 448424.87 | 0.26 | MT-RNR2 | Mitochondrially | 4550 | |
| | | | | | | | Encoded 16S | | |
| | | | | | | | RRNA | | |
| <1e−07 | <1e−07 | <1e−07 | 41.25 | 14.2 | 2.91 | ARMC5 | armadillo repeat | 79798 | NM_001105247 |
| | | | | | | | containing 5 | | |
| <1e−07 | <1e−07 | <1e−07 | 96.18 | 42.7 | 2.25 | ZNF335 | zinc finger protein | 63925 | NM_022095 |
| | | | | | | | 335 | | |
| <1e−07 | <1e−07 | <1e−07 | 38.93 | 17.46 | 2.23 | STARD3 | StAR related lipid | 10948 | NM_001165937 |
| | | | | | | | transfer domain | | |
| | | | | | | | containing 3 | | |
| <1e−07 | <1e−07 | <1e−07 | 73.43 | 19.14 | 3.84 | AC004687.1 | Homo sapiens | | |
| | | | | | | | transcribed RNA | | |
| | | | | | | | MIR142 gene for | | |
| | | | | | | | microRNA 142, | | |
| | | | | | | | complete | | |
| | | | | | | | sequence | | |
| | | | | | | | (EnsEMBL Gene ID | | |
| | | | | | | | (ENST00000579003.1) | | |
| <1e−07 | <1e−07 | <1e−07 | 129.71 | 59.45 | 2.18 | FBRSL1 | fibrosin like 1 | 57666 | NM_001142641 |
| <1e−07 | <1e−07 | <1e−07 | 63.77 | 26.09 | 2.44 | TTC7A | tetratricopeptide | 57217 | NM_001288951 |
| | | | | | | | repeat domain 7A | | |
| <1e−07 | <1e−07 | <1e−07 | 24.52 | 10.45 | 2.35 | PUS1 | pseudouridy late | 80324 | NM_001002019 |
| | | | | | | | synthase 1 | | |
| <1e−07 | <1e−07 | <1e−07 | 105.09 | 37.36 | 2.81 | RFTN1 | raftlin, lipid raft | 23180 | NM_015150 |
| | | | | | | | linker 1 | | |
| <1e−07 | <1e−07 | <1e−07 | 59.37 | 25.79 | 2.3 | RB1CC1 | RB1 inducible | 9821 | NM_001083617 |
| | | | | | | | coiled-coil 1 | | |
| <1e−07 | <1e−07 | <1e−07 | 749.5 | 319.65 | 2.34 | HNRNPH1 | heterogeneous | 3187 | NM_001257293 |
| | | | | | | | nuclear | | |
| | | | | | | | ribonucleoprotein | | |
| | | | | | | | H1 | | |
| <1e−07 | <1e−07 | <1e−07 | 88.01 | 37.26 | 2.36 | RASA3 | RAS p21 protein | 22821 | NM_001320821 |
| | | | | | | | activator 3 | | |
| <1e−07 | <1e−07 | <1e−07 | 27.64 | 13 | 2.13 | TEPSIN | TEPSIN, adaptor | 146705 | NM_144679 |
| | | | | | | | related protein | | |
| | | | | | | | complex 4 | | |
| | | | | | | | accessory protein | | |
| <1e−07 | <1e−07 | <1e−07 | 48.11 | 18.73 | 2.57 | SMG1P1 | SMG1 | 641298 | NM_199284 |
| | | | | | | | pseudogene 1 | | |
| <1e−07 | <1e−07 | <1e−07 | 41.77 | 19.41 | 2.15 | FAM168B | family with | 130074 | NM_001009993 |
| | | | | | | | sequence | | |
| | | | | | | | similarity 168 | | |
| | | | | | | | member B | | |
| <1e−07 | <1e−07 | <1e−07 | 46.04 | 18.05 | 2.55 | MGAT2 | mannosyl (alpha- | 4247 | NM_001015883 |
| | | | | | | | 1,6-)-glycoprotein | | |
| | | | | | | | beta-1,2-N- | | |
| | | | | | | | acetylglucos- | | |
| | | | | | | | aminyltransferase | | |
| <1e−07 | <1e−07 | <1e−07 | 37.26 | 13.74 | 2.71 | ADCY7 | adenylate cyclase | 113 | NM_001114 |
| | | | | | | | 7 | | |
| <1e−07 | <1e−07 | <1e−07 | 110.17 | 49.4 | 2.23 | DOCK2 | dedicator of | 1794 | NM_004946 |
| | | | | | | | cytokinesis 2 | | |
| <1e−07 | <1e−07 | <1e−07 | 20.64 | 9.09 | 2.27 | AC004980.1 | noncoding | | |
| | | | | | | | (Ensemble gene | | |
| | | | | | | | ID: | | |
| | | | | | | | ENSG00000214243) | | |
| <1e−07 | <1e−07 | <1e−07 | 30.18 | 13.26 | 2.28 | NGLY1 | N-glycanase 1 | 55768 | NM_001145293 |
| <1e−07 | <1e−07 | <1e−07 | 48.29 | 19.98 | 2.42 | SRSF6 | serine and | 6431 | NM_006275 |
| | | | | | | | arginine rich | | |
| | | | | | | | splicing factor 6 | | |
| <1e−07 | <1e−07 | <1e−07 | 14.07 | 6.34 | 2.22 | MB21D2 | Mab-21 domain | 151963 | NM_178496 |
| | | | | | | | containing 2 | | |
| <1e−07 | <1e−07 | <1e−07 | 280.83 | 107.12 | 2.62 | AKNA | AT-hook | 80709 | NM_001317950 |
| | | | | | | | transcription | | |
| | | | | | | | factor | | |
| <1e−07 | <1e−07 | <1e−07 | 40.19 | 14.73 | 2.73 | NCAPH2 | non-SMC | 29781 | NM_001185011 |
| | | | | | | | condensin II | | |
| | | | | | | | complex subunit | | |
| | | | | | | | H2 | | |
| <1e−07 | <1e−07 | <1e−07 | 24.76 | 11.35 | 2.18 | GID8 | GID complex | 54994 | NM_017896 |
| | | | | | | | subunit 8 homobg | | |
| <1e−07 | <1e−07 | <1e−07 | 128.56 | 57.59 | 2.23 | SIK3 | SIK family kinase | 23387 | NM_001281748 |
| | | | | | | | 3 | | |
| <1e−07 | 5.39E−06 | <1e−07 | 61.45 | 29.63 | 2.07 | KHNYN | KH and NYN | 23351 | NM_001290256 |
| | | | | | | | domain containing | | |
| <1e−07 | 5.39E−06 | <1e−07 | 12.58 | 6.15 | 2.05 | QPCTL | glutaminyl- | 54814 | NM_001163377 |
| | | | | | | | peptide | | |
| | | | | | | | cyclotransferase | | |
| | | | | | | | like | | |
| <1e−07 | 5.39E−06 | <1e−07 | 37.5 | 16.54 | 2.27 | FAM3C | family with | 10447 | NM_001040020 |
| | | | | | | | sequence | | |
| | | | | | | | similarity 3 | | |
| | | | | | | | member C | | |
| <1e−07 | 5.39E−06 | <1e−07 | 5.07 | 10.85 | 0.47 | ABCA13 | ATP binding | 154664 | NM_152701 |
| | | | | | | | cassette subfamily | | |
| | | | | | | | A member 13 | | |
| <1e−07 | 5.39E−06 | <1e−07 | 32.28 | 13.76 | 2.35 | USP16 | ubiquitin specific | 10600 | NM_001001992 |
| | | | | | | | peptidase 16 | | |
| <1e−07 | 5.39E−06 | <1e−07 | 34.4 | 14.21 | 2.42 | CYBC1 | cytochrome b-245 | 79415 | NM_001033046 |
| | | | | | | | chaperone 1 | | |
| <1e−07 | 5.39E−06 | <1e−07 | 16.74 | 8.12 | 2.06 | FBXO33 | F-box protein 33 | 254170 | NM_203301 |
| <1e−07 | 5.39E−06 | <1e−07 | 73.93 | 25.42 | 2.91 | ARFGAP1 | ADP ribosylation | 55738 | NM_001281482 |
| | | | | | | | factor GTPase | | |
| | | | | | | | activating protein | | |
| | | | | | | | 1 | | |
| <1e−07 | 5.39E−06 | <1e−07 | 68.72 | 27.35 | 2.51 | CARS1 | Cysteinyl-TRNA | 833 | |
| | | | | | | | Synthetase 1 | | |
| <1e−07 | 5.39E−06 | <1e−07 | 29.99 | 14.29 | 2.1 | FPGS | folylpolyglutamate | 2356 | NM_001018078 |
| | | | | | | | synthase | | |
| <1e−07 | 5.39E−06 | <1e−07 | 31.09 | 11.68 | 2.66 | AC044849.1 | noncoding | | |
| | | | | | | | (Ensemble gene | | |
| | | | | | | | ID: | | |
| | | | | | | | ENSG00000272256.1) | | |
| <1e−07 | 5.39E−06 | <1e−07 | 38.11 | 17.19 | 2.22 | FCHSD2 | FCH and double | 9873 | NM_014824 |
| | | | | | | | SH3 domains 2 | | |
| <1e−07 | 5.39E−06 | <1e−07 | 924.07 | 449.77 | 2.05 | DDX5 | DEAD-box | 1655 | NM_001320595 |
| | | | | | | | helicase 5 | | |
| <1e−07 | 5.39E−06 | <1e−07 | 227.23 | 111.15 | 2.04 | UBE2D3 | ubiquitin | 7323 | NM_001300795 |
| | | | | | | | conjugating | | |
| | | | | | | | enzyme E2 D3 | | |
| <1e−07 | 5.39E−06 | <1e−07 | 47.63 | 22.44 | 2.12 | LDB1 | LIM domain | 8861 | NM_001113407 |
| | | | | | | | binding 1 | | |
| <1e−07 | 5.39E−06 | <1e−07 | 20.65 | 9.91 | 2.08 | CERS4 | ceramide synthase | 79603 | NM_024552 |
| | | | | | | | 4 | | |
| <1e−07 | 5.39E−06 | <1e−07 | 35.79 | 12.94 | 2.77 | FKBP11 | FK506 binding | 51303 | NM_001143781 |
| | | | | | | | protein 11 | | |
| <1e−07 | 5.39E−06 | <1e−07 | 68.2 | 33.87 | 2.01 | CPSF7 | cleavage and | 79869 | NM_001136040 |
| | | | | | | | polyadenylation | | |
| | | | | | | | specific factor 7 | | |
| <1e−07 | 5.39E−06 | <1e−07 | 57.88 | 22.38 | 2.59 | IARS1 | Isoleucyl-TRNA | 3376 | |
| | | | | | | | Synthetase 1 | | |
| <1e−07 | 5.39E−06 | <1e−07 | 47.35 | 22.59 | 2.1 | ZBTB40 | zinc finger and | 9923 | NM_001083621 |
| | | | | | | | BTB domain | | |
| | | | | | | | containing 40 | | |
| <1e−07 | 5.39E−06 | <1e−07 | 37.3 | 16.67 | 2.24 | DMXL1 | Dmx like 1 | 1657 | NM_001290321 |
| <1e−07 | 5.39E−06 | <1e−07 | 162.04 | 80.19 | 2.02 | PRPF38B | pre-m RNA | 55119 | NM_001349757 |
| | | | | | | | processing factor | | |
| | | | | | | | 38B | | |
| <1e−07 | 5.39E−06 | <1e−07 | 313.62 | 145.41 | 2.16 | SRSF5 | serine and | 6430 | NM_001039465 |
| | | | | | | | arginine rich | | |
| | | | | | | | splicing factor 5 | | |
| <1e−07 | 5.39E−06 | <1e−07 | 47869.56 | 161215.08 | 0.3 | MT-CO1 | Mitochondrially | 4512 | |
| | | | | | | | Encoded | | |
| | | | | | | | Cytochrome C | | |
| | | | | | | | Oxidase I | | |
| <1e−07 | 5.39E−06 | <1e−07 | 84.71 | 31.25 | 2.71 | PKD1 | polycystin 1, | 5310 | NM_000296 |
| | | | | | | | transient receptor | | |
| | | | | | | | potential channel | | |
| | | | | | | | interacting | | |
| <1e−07 | 5.39E−06 | <1e−07 | 216.88 | 104.05 | 2.08 | SF3B1 | splicing factor 3b | 23451 | NM_001005526 |
| | | | | | | | subunit 1 | | |
| <1e−07 | 5.39E−06 | <1e−07 | 86.58 | 21.54 | 4.02 | NPIPB5 | nuclear pore | 100132247 | NM_001135865 |
| | | | | | | | complex | | |
| | | | | | | | interacting protein | | |
| | | | | | | | family member | | |
| | | | | | | | B5 | | |
| <1e−07 | 5.39E−06 | <1e−07 | 15.41 | 7.58 | 2.03 | PTOV1- | PTOV1 antisense | 101928378 | NR_110730 |
| | | | | | | AS2 | RNA 2 | | |
| <1e−07 | 5.39E−06 | <1e−07 | 139.07 | 55.65 | 2.5 | CRTC2 | CREB regulated | 200186 | NM_181715 |
| | | | | | | | transcription | | |
| | | | | | | | coactivator 2 | | |
| <1e−07 | 5.39E−06 | <1e−07 | 5.26 | 11.52 | 0.46 | DNAH7 | dynein axonemal | 56171 | NM_018897 |
| | | | | | | | heavy chain 7 | | |
| <1e−07 | 5.39E−06 | <1e−07 | 21.54 | 9.66 | 2.23 | INTS6L | integrator | 203522 | NM_001351601 |
| | | | | | | | complex subunit 6 | | |
| | | | | | | | like | | |
| 0.0000001 | 5.39E−06 | <1e−07 | 85.33 | 34.51 | 2.47 | AARS1 | Alanyl-TRNA | 16 | |
| | | | | | | | Synthetase 1 | | |
| 0.0000001 | 5.39E−06 | <1e−07 | 30.11 | 14.15 | 2.13 | ABCD4 | ATP binding | 5826 | NM_001353591 |
| | | | | | | | cassette subfamily | | |
| | | | | | | | D member 4 | | |
| 0.0000001 | 5.39E−06 | <1e−07 | 5.96 | 12.23 | 0.49 | PROM1 | prominin 1 | 8842 | NM_001145847 |
| 0.0000001 | 5.39E−06 | <1e−07 | 193.66 | 88.84 | 2.18 | NXF1 | nuclear RNA | 10482 | NM_001081491 |
| | | | | | | | export factor 1 | | |
| 0.0000001 | 5.39E−06 | <1e−07 | 127.08 | 60.34 | 2.11 | SLC38A10 | solute carrier | 124565 | NM_001037984 |
| | | | | | | | family 38 member | | |
| | | | | | | | 10 | | |
| 0.0000001 | 5.39E−06 | <1e−07 | 46.06 | 18.97 | 2.43 | SHMT2 | serine | 6472 | NM_001166356 |
| | | | | | | | hydroxymeth- | | |
| | | | | | | | yltransferase 2 | | |
| 0.0000001 | 5.39E−06 | <1e−07 | 110.55 | 49.15 | 2.25 | VPS37B | VPS37B, ESCRT- | 79720 | NM_024667 |
| | | | | | | | I subunit | | |
| 0.0000001 | 5.39E−06 | <1e−07 | 11.07 | 40.47 | 0.27 | MTND4P12 | mitochondrially | 100293090 | |
| | | | | | | | encoded | | |
| | | | | | | | NADH: ubiquinone | | |
| | | | | | | | oxidoreductase | | |
| | | | | | | | core subunit 4 | | |
| | | | | | | | pseudogene 12 | | |
| 0.0000001 | 5.39E−06 | <1e−07 | 6.8 | 16.2 | 0.42 | SEMA6A | semaphorin 6A | 57556 | NM_001300780 |
| 0.0000001 | 5.39E−06 | <1e−07 | 47.95 | 20.38 | 2.35 | ARFGAP2 | ADP ribosylation | 84364 | NM_001242832 |
| | | | | | | | factor GTPase | | |
| | | | | | | | activating protein | | |
| | | | | | | | 2 | | |
| 0.0000001 | 5.39E−06 | <1e−07 | 49.71 | 22.85 | 2.18 | RNF216 | ring finger protein | 54476 | NM_019011 |
| | | | | | | | 216 | | |
| 0.0000001 | 5.39E−06 | <1e−07 | 44.05 | 12.22 | 3.6 | BACH2 | BTB domain and | 60468 | NM_001170794 |
| | | | | | | | CNC homolog 2 | | |
| 0.0000001 | 5.39E−06 | <1e−07 | 65.96 | 20.31 | 3.25 | PTPRS | protein tyrosine | 5802 | NM_0028505 |
| | | | | | | | phosphatase, | | |
| | | | | | | | receptor type S | | |
| 0.0000001 | 5.39E−06 | <1e−07 | 23.85 | 11.4 | 2.09 | PSMC6 | proteasome 26S | 5706 | NM_00280606 |
| | | | | | | | subunit, ATPase 6 | | |
| 0.0000001 | 5.39E−06 | <1e−07 | 39.82 | 17.9 | 2.23 | M6PR | mannose-6- | 4074 | NM_001207024 |
| | | | | | | | phosphate | | |
| | | | | | | | receptor, cation | | |
| | | | | | | | dependent | | |
| 0.0000001 | 5.39E−06 | <1e−07 | 20.52 | 9.41 | 2.18 | TSR2 | TSR2, ribosome | 90121 | NM_001346789 |
| | | | | | | | maturation factor | | |
| 0.0000001 | 5.39E−06 | <1e−07 | 65.4 | 29.63 | 2.21 | KCNAB2 | potassium | 8514 | NM_001199860 |
| | | | | | | | voltage-gated | | |
| | | | | | | | channel subfamily | | |
| | | | | | | | A regulatory beta | | |
| | | | | | | | subunit 2 | | |
| 0.0000001 | 5.39E−06 | <1e−07 | 134.13 | 49.38 | 2.72 | STAT2 | signal transducer | 6773 | NM_005419 |
| | | | | | | | and activator of | | |
| | | | | | | | transcription 2 | | |
| 0.0000001 | 5.39E−06 | <1e−07 | 51.9 | 24.01 | 2.16 | PTOV1 | prostate tumor | 53635 | NM_001305105 |
| | | | | | | | overexpressed 1 | | |
| 0.0000001 | 5.39E−06 | <1e−07 | 18.57 | 76.2 | 0.24 | MT-TT | Mitochondrially | 4576 | |
| | | | | | | | Encoded TRNA- | | |
| | | | | | | | Thr (ACN) | | |
| 0.0000001 | 5.39E−06 | <1e−07 | 75.54 | 34.1 | 2.22 | USP11 | ubiquitin specific | 8237 | NM_004651 |
| | | | | | | | peptidase 11 | | |
| 0.0000001 | 5.39E−06 | <1e−07 | 174.93 | 32.55 | 5.37 | STAB1 | stabilin 1 | 23166 | NM_015136 |
| 0.0000001 | 5.39E−06 | <1e−07 | 6.36 | 18.11 | 0.35 | SLC4A4 | solute carrier | 8671 | NM_001098484 |
| | | | | | | | family 4 member | | |
| | | | | | | | 4 | | |
| 0.0000001 | 5.39E−06 | <1e−07 | 44.89 | 21.83 | 2.06 | UBR3 | ubiquitin protein | 130507 | NM_172070 |
| | | | | | | | ligase E3 | | |
| | | | | | | | component n- | | |
| | | | | | | | recognin 3 | | |
| | | | | | | | (putative) | | |
| 0.0000001 | 5.39E−06 | <1e−07 | 26.28 | 9.91 | 2.65 | BHLHE41 | basic helix-loop- | 79365 | NM_030762 |
| | | | | | | | helix family | | |
| | | | | | | | member e41 | | |
| 0.0000001 | 5.39E−06 | <1e−07 | 111.77 | 47.73 | 2.34 | ATP2B1 | ATPase plasma | 490 | NM_001001323 |
| | | | | | | | membrane Ca2+ | | |
| | | | | | | | transporting 1 | | |
| 0.0000001 | 5.39E−06 | <1e−07 | 83.12 | 31.74 | 2.62 | IFFO2 | intermediate | 126917 | NM_001136265 |
| | | | | | | | filament family | | |
| | | | | | | | orphan 2 | | |
| 0.0000001 | 5.39E−06 | <1e−07 | 65.87 | 32.91 | 2 | BTAF1 | B-TFIID TATA- | 9044 | NM_003972 |
| | | | | | | | box binding | | |
| | | | | | | | protein associated | | |
| | | | | | | | factor 1 | | |
| 0.0000001 | 5.39E−06 | <1e−07 | 105.24 | 41.37 | 2.54 | SYVN1 | synoviolin 1 | 84447 | NM_032431 |
| 0.0000001 | 5.39E−06 | <1e−07 | 19.8 | 8.61 | 2.3 | TRIM39 | tripartite motif | 56658 | NM_021253 |
| | | | | | | | containing 39 | | |
| 0.0000001 | 5.39E−06 | <1e−07 | 151.14 | 65.2 | 2.32 | SUN2 | Sad1 and UNC84 | 25777 | NM_001199579 |
| | | | | | | | domain containing | | |
| | | | | | | | 2 | | |
| 0.0000001 | 5.39E−06 | <1e−07 | 18968.37 | 64548.84 | 0.29 | MT-ND5 | Mitochondrially | 4540 | |
| | | | | | | | Encoded | | |
| | | | | | | | NADH: Ubiquinone | | |
| | | | | | | | Oxidoreductase | | |
| | | | | | | | Core Subunit 5 | | |
| 0.0000001 | 5.39E−06 | <1e−07 | 26.17 | 12.38 | 2.11 | TOPORS | TOP1 binding | 10210 | NM_001195622 |
| | | | | | | | arginine/serine | | |
| | | | | | | | rich protein | | |
| 0.0000001 | 5.39E−06 | <1e−07 | 239.51 | 102.26 | 2.34 | CCNL1 | cyclin L1 | 57018 | NM_001308185 |
| 0.0000002 | 9.26E−06 | <1e−07 | 2193.07 | 6970.15 | 0.31 | MT-ND4L | Mitochondrially | 4539 | |
| | | | | | | | Encoded | | |
| | | | | | | | NADH: Ubiquinone | | |
| | | | | | | | Oxidoreductase | | |
| | | | | | | | Core Subunit 4L | | |
| 0.0000002 | 9.26E−06 | <1e−07 | 32.66 | 15.63 | 2.09 | KLC2 | kinesin light chain | 64837 | NM_001134774 |
| | | | | | | | 2 | | |
| 0.0000002 | 9.26E−06 | <1e−07 | 72.57 | 22.74 | 3.19 | ACAP1 | ArfGAP with | 9744 | NM_014716 |
| | | | | | | | coiled-coil, | | |
| | | | | | | | ankyrin repeat and | | |
| | | | | | | | PH domains 1 | | |
| 0.0000002 | 9.26E−06 | <1e−07 | 18.62 | 8.76 | 2.13 | IFNAR2 | interferon alpha | 3455 | NM_000874 |
| | | | | | | | and beta receptor | | |
| | | | | | | | subunit 2 | | |
| 0.0000002 | 9.26E−06 | <1e−07 | 154.16 | 62.36 | 2.47 | FNBP4 | formin binding | 23360 | NM_001318339 |
| | | | | | | | protein 4 | | |
| 0.0000002 | 9.26E−06 | <1e−07 | 29.07 | 14.28 | 2.04 | USF1 | upstream | 7391 | NM_001276373 |
| | | | | | | | transcription | | |
| | | | | | | | factor 1 | | |
| 0.0000002 | 9.26E−06 | <1e−07 | 26.76 | 10.99 | 2.43 | PPP3CC | protein | 5533 | NM_001243974 |
| | | | | | | | phosphatase 3 | | |
| | | | | | | | catalytic subunit | | |
| | | | | | | | gamma | | |
| 0.0000002 | 9.26E−06 | <1e−07 | 64.7 | 23.67 | 2.73 | EMILIN2 | elastin microfibril | 84034 | NM_032048 |
| | | | | | | | interfacer 2 | | |
| 0.0000002 | 9.26E−06 | <1e−07 | 166.82 | 59.88 | 2.79 | TNFRSF14 | TNF receptor | 8764 | NM_001297605 |
| | | | | | | | superfamily | | |
| | | | | | | | member 14 | | |
| 0.0000002 | 9.26E−06 | <1e−07 | 5.56 | 11.53 | 0.48 | PLA2R1 | phospholipase A2 | 22925 | NM_001007267 |
| | | | | | | | receptor 1 | | |
| 0.0000002 | 9.26E−06 | <1e−07 | 54.53 | 21.17 | 2.58 | UBE2Z | ubiquitin | 65264 | NM_023079 |
| | | | | | | | conjugating | | |
| | | | | | | | enzyme E2 Z | | |
| 0.0000002 | 9.26E−06 | <1e−07 | 93.97 | 33.2 | 2.83 | LPXN | leupaxin | 9404 | NM_001143995 |
| 0.0000002 | 9.26E−06 | <1e−07 | 48.85 | 21.8 | 2.24 | MAN2B1 | mannosidase | 4125 | NM_000528 |
| | | | | | | | alpha class 2B | | |
| | | | | | | | member 1 | | |
| 0.0000002 | 9.26E−06 | <1e−07 | 1485 | 620.94 | 2.39 | VIM | vimentin | 7431 | NM_003380 |
| 0.0000002 | 9.26E−06 | <1e−07 | 68.4 | 34.06 | 2.01 | TCF3 | transcription | 6929 | NM_001136139 |
| | | | | | | | factor 3 | | |
| 0.0000002 | 9.26E−06 | <1e−07 | 92.92 | 35.11 | 2.65 | PBXIP1 | PBX homeobox | 57326 | NM_001317734 |
| | | | | | | | interacting protein | | |
| | | | | | | | 1 | | |
| 0.0000002 | 9.26E−06 | <1e−07 | 140.91 | 54.91 | 2.57 | TRA2A | transformer 2 | 29896 | NM_001282757 |
| | | | | | | | alpha homolog | | |
| 0.0000002 | 9.26E−06 | <1e−07 | 241.08 | 105.55 | 2.28 | ARHGEF1 | Rho guanine | 9138 | NM_004706 |
| | | | | | | | nucleotide | | |
| | | | | | | | exchange factor 1 | | |
| 0.0000002 | 9.26E−06 | <1e−07 | 14920.08 | 48937.58 | 0.3 | MT-ND2 | Mitochondrially | 4536 | |
| | | | | | | | Encoded | | |
| | | | | | | | NADH: Ubiquinone | | |
| | | | | | | | Oxidoreductase | | |
| | | | | | | | Core Subunit 2 | | |
| 0.0000002 | 9.26E−06 | <1e−07 | 79.3 | 32.26 | 2.46 | PLXND1 | plexin D1 | 23129 | NM_015103 |
| 0.0000002 | 9.26E−06 | <1e−07 | 108 | 34.99 | 3.09 | PLXNA1 | plexin A1 | 5361 | NM_032242 |
| 0.0000002 | 9.26E−06 | <1e−07 | 68.2 | 29.35 | 2.32 | PKN1 | protein kinase N1 | 5585 | NM_002741 |
| 0.0000002 | 9.26E−06 | <1e−07 | 7.53 | 25.38 | 0.3 | ATP10B | ATPase | 23120 | NM_025153 |
| | | | | | | | phospholipid | | |
| | | | | | | | transporting 10B | | |
| | | | | | | | (putative) | | |
| 0.0000002 | 9.26E−06 | <1e−07 | 247.91 | 120.55 | 2.06 | PIEZO1 | piezo type | 9780 | NM_001142864 |
| | | | | | | | mechanosensitive | | |
| | | | | | | | ion channel | | |
| | | | | | | | component 1 | | |
| 0.0000002 | 9.26E−06 | <1e−07 | 43.22 | 21.46 | 2.01 | ARMCX3 | armadillo repeat | 51566 | NM_016607 |
| | | | | | | | containing, X- | | |
| | | | | | | | linked 3 | | |
| 0.0000002 | 9.26E−06 | <1e−07 | 5.46 | 12.04 | 0.45 | PRELID2 | PRLI domain | 153768 | NM_138492 |
| | | | | | | | containing 2 | | |
| 0.0000002 | 9.26E−06 | <1e−07 | 80.01 | 33.52 | 2.39 | PCNX3 | pecanexhomolog | 399909 | NM_032223 |
| | | | | | | | 3 | | |
| 0.0000002 | 9.26E−06 | <1e−07 | 21.49 | 9.57 | 2.24 | XOSC6 | exosome | 118460 | NM_058219 |
| | | | | | | | component 6 | | |
| 0.0000002 | 9.26E−06 | <1e−07 | 24.32 | 11.1 | 2.19 | DCAF8 | DDB1 and CUL4 | 50717 | NM_015726 |
| | | | | | | | associated factor 8 | | |
| 0.0000002 | 9.26E−06 | <1e−07 | 183.31 | 81.13 | 2.26 | SBF1 | SET binding | 6305 | NM_002972 |
| | | | | | | | factor 1 | | |
| 0.0000002 | 9.26E−06 | <1e−07 | 5.65 | 11.86 | 0.48 | FOXP2 | forkhead box P2 | 93986 | NM_001172766 |
| 0.0000002 | 9.26E−06 | <1e−07 | 33.62 | 14.12 | 2.38 | MAP3K3 | mitogen-activated | 4215 | NM_001330431 |
| | | | | | | | protein kinase | | |
| | | | | | | | kinases kinase 3 | | |
| 0.0000002 | 9.26E−06 | <1e−07 | 280.37 | 124.16 | 2.26 | UBR4 | ubiquitin protein | 23352 | NM_020765 |
| | | | | | | | ligase E3 | | |
| | | | | | | | component n- | | |
| | | | | | | | recognin 4 | | |
| 0.0000002 | 9.26E−06 | <1e−07 | 50.08 | 21.05 | 2.38 | NR1H2 | nuclear receptor | 7376 | NM_001256647 |
| | | | | | | | subfamily 1 group | | |
| | | | | | | | H member 2 | | |
| 0.0000002 | 9.26E−06 | <1e−07 | 71.44 | 35.27 | 2.03 | SCAF4 | SR-related CTD | 57466 | NM_001145444 |
| | | | | | | | associated factor 4 | | |
| 0.0000002 | 9.26E−06 | <1e−07 | 27.47 | 10.73 | 2.56 | CARMIL2 | capping protein | 146206 | NM_001013838 |
| | | | | | | | regulator and | | |
| | | | | | | | myosin 1 linker 2 | | |
| 0.0000003 | 1.23E−05 | <1e−07 | 33.45 | 15.46 | 2.16 | RETREG2 | reticulophagy | 79137 | NM_001321109 |
| | | | | | | | regulator family | | |
| | | | | | | | member 2 | | |
| 0.0000003 | 1.23E−05 | <1e−07 | 50.24 | 18.24 | 2.75 | SLC1A4 | solute carrier | 6509 | NM_001135581 |
| | | | | | | | family 1 member | | |
| | | | | | | | 4 | | |
| 0.0000003 | 1.23E−05 | <1e−07 | 107.15 | 50.13 | 2.14 | CYLD | CYLD lysine 63 | 1540 | NM_001042355 |
| | | | | | | | deubiquitinase | | |
| 0.0000003 | 1.23E−05 | <1e−07 | 52.96 | 18.76 | 2.82 | SNX8 | sorting nexin 8 | 29886 | NM_013321 |
| 0.0000003 | 1.23E−05 | <1e−07 | 59.37 | 26.27 | 2.26 | GATAD2B | GATA zinc finger | 57459 | NM_020699 |
| | | | | | | | domain containing | | |
| | | | | | | | 2B | | |
| 0.0000003 | 1.23E−05 | <1e−07 | 39.92 | 17.7 | 2.26 | DUSP10 | dual specificity | 11221 | NM_007207 |
| | | | | | | | phosphatase 10 | | |
| 0.0000003 | 1.23E−05 | <1e−07 | 31 | 14.03 | 2.21 | HMBOX1 | homeobox | 79618 | NM_001135726 |
| | | | | | | | containing 1 | | |
| 0.0000003 | 1.23E−05 | <1e−07 | 215.32 | 87.92 | 2.45 | MGAT1 | mannosyl (alpha- | 4245 | NM_001114617 |
| | | | | | | | 1,3-)-glycoprotein | | |
| | | | | | | | beta-1,2-N- | | |
| | | | | | | | acetylglucos- | | |
| | | | | | | | aminyltransferase | | |
| 0.0000003 | 1.23E−05 | <1e−07 | 44.39 | 14.81 | 3 | RASGRP3 | RAS guanyl | 25780 | NM_001139488 |
| | | | | | | | releasing protein 3 | | |
| 0.0000003 | 1.23E−05 | <1e−07 | 123.46 | 60.08 | 2.05 | HGS | hepatocyte growth | 9146 | NM_004712 |
| | | | | | | | factor-regulated | | |
| | | | | | | | tyrosine kinase | | |
| | | | | | | | substrate | | |
| 0.0000003 | 1.23E−05 | <1e−07 | 44.68 | 13.71 | 3.26 | LY9 | lymphocyte | 4063 | NM_001033667 |
| | | | | | | | antigen 9 | | |
| 0.0000003 | 1.23E−05 | <1e−07 | 132.49 | 63.62 | 2.08 | TRRAP | transformation/ | 8295 | NM_001244580 |
| | | | | | | | transcription domain | | |
| | | | | | | | associated protein | | |
| 0.0000003 | 1.23E−05 | <1e−07 | 309.57 | 139.73 | 2.22 | AC022966.1 | ubiquitin specific | | |
| | | | | | | | peptidase 36 | | |
| | | | | | | | (EnsMBL Gene | | |
| | | | | | | | ID | | |
| | | | | | | | ENSG00000055483) | | |
| 0.0000003 | 1.23E−05 | <1e−07 | 22.33 | 10.89 | 2.05 | TENT2 | Terminal | 167153 | |
| | | | | | | | Nucleotidyl- | | |
| | | | | | | | transferase 2 | | |
| 0.0000003 | 1.23E−05 | <1e−07 | 26.27 | 12.37 | 2.12 | TFEB | transcription | 7942 | NM_001167827 |
| | | | | | | | factor EB | | |
| 0.0000003 | 1.23E−05 | <1e−07 | 91.89 | 42.49 | 2.16 | ESYT2 | extended | 57488 | NM_02072828 |
| | | | | | | | synaptotagmin 2 | | |
| 0.0000003 | 1.23E−05 | <1e−07 | 19.2 | 8.19 | 2.34 | ZNRF1 | zinc and ring | 84937 | NM_032268 |
| | | | | | | | finger 1 | | |
| 0.0000003 | 1.23E−05 | <1e−07 | 72.97 | 29.1 | 2.51 | MARS1 | Methionyl-TRNA | 4141 | |
| | | | | | | | Synthetase 1 | | |
| 0.0000003 | 1.23E−05 | <1e−07 | 42.37 | 20.93 | 2.02 | TPP2 | tripeptidyl | 7174 | NM_001330588 |
| | | | | | | | peptidase 2 | | |
| 0.0000003 | 1.23E−05 | <1e−07 | 25.84 | 11.26 | 2.29 | STK17A | serine/threonine | 9263 | NM_004760 |
| | | | | | | | kinase 17a | | |
| 0.0000003 | 1.23E−05 | <1e−07 | 30.58 | 13.47 | 2.27 | CRTC1 | CREB regulated | 23373 | NM_001098482 |
| | | | | | | | transcription | | |
| | | | | | | | coactivator 1 | | |
| 0.0000003 | 1.23E−05 | <1e−07 | 310.63 | 113.39 | 2.74 | PLEKHG2 | pleckstrin | 64857 | NM_001351693 |
| | | | | | | | homology and | | |
| | | | | | | | RhoGF domain | | |
| | | | | | | | containing G2 | | |
| 0.0000003 | 1.23E−05 | <1e−07 | 28.34 | 12.37 | 2.29 | CNPPD1 | cyclin | 27013 | NM_001321389 |
| | | | | | | | Pas1/PHO80 | | |
| | | | | | | | domain containing | | |
| | | | | | | | 1 | | |
| 0.0000003 | 1.23E−05 | <1e−07 | 120.38 | 51.1 | 2.36 | STAT6 | signal transducer | 6778 | NM_001178078 |
| | | | | | | | and activator of | | |
| | | | | | | | transcription 6 | | |
| 0.0000003 | 1.23E−05 | <1e−07 | 16.41 | 7.64 | 2.15 | IRF5 | interferon | 3663 | NM_001098627 |
| | | | | | | | regulatory factor 5 | | |
| 0.0000003 | 1.23E−05 | <1e−07 | 28.42 | 11.69 | 2.43 | LINC00342 | long intergenic | 150759 | NR_103734 |
| | | | | | | | non-protein | | |
| | | | | | | | coding RNA 342 | | |
| 0.0000003 | 1.23E−05 | <1e−07 | 8.59 | 24.91 | 0.34 | MYO5C | myosin VC | 55930 | NM_018728 |
| 0.0000003 | 1.23E−05 | <1e−07 | 10.5 | 48.36 | 0.22 | LGALS4 | galectin 4 | 3960 | NM_006149 |
| 0.0000003 | 1.23E−05 | <1e−07 | 65.89 | 27.83 | 2.37 | PNKP | polynucleotide | 11284 | NM_007254 |
| | | | | | | | kinase 3′- | | |
| | | | | | | | phosphatase | | |
| 0.0000003 | 1.23E−05 | <1e−07 | 114.12 | 25.59 | 4.46 | SNORD89 | small nucleolar | 692205 | NR_003070 |
| | | | | | | | RNA, C/D box 89 | | |
| 0.0000003 | 1.23E−05 | <1e−07 | 4623.59 | 14461.63 | 0.32 | MT-ND3 | Mitochondrially | 4537 | |
| | | | | | | | Encoded | | |
| | | | | | | | NADH: Ubiquinone | | |
| | | | | | | | Oxidoreductase | | |
| | | | | | | | Core Subunit 3 | | |
| 0.0000003 | 1.23E−05 | <1e−07 | 250.77 | 107.35 | 2.34 | PER1 | period circadian | 5187 | NM_002616 |
| | | | | | | | regulator 1 | | |
| 0.0000004 | 0.000015 | <1e−07 | 29.75 | 14.26 | 2.09 | CAPN10 | calpain 10 | 11132 | NM_021251 |
| 0.0000004 | 0.000015 | <1e−07 | 454.39 | 168.51 | 2.7 | TSC22D3 | TSC22 domain | 1831 | NM_001015881 |
| | | | | | | | family member 3 | | |
| 0.0000004 | 0.000015 | <1e−07 | 70.27 | 32.4 | 2.17 | GPBP1 | GC-rich promoter | 65056 | NM_001127235 |
| | | | | | | | binding protein 1 | | |
| 0.0000004 | 0.000015 | <1e−07 | 369.52 | 182.63 | 2.02 | POLR2A | RNA polymerase | 5430 | NM_000937 |
| | | | | | | | II subunit A | | |
| 0.0000004 | 0.000015 | <1e−07 | 49.47 | 19.81 | 2.5 | ENTPD4 | ectonucleoside | 9583 | NM_001128930 |
| | | | | | | | triphosphate | | |
| | | | | | | | diphosphohydrolase | | |
| | | | | | | | 4 | | |
| 0.0000004 | 0.000015 | <1e−07 | 21.88 | 8.01 | 2.73 | FAM30A | family with | 9834 | NM_01415151 |
| | | | | | | | sequence | | |
| | | | | | | | similarity 30 | | |
| | | | | | | | member A | | |
| 0.0000004 | 0.000015 | <1e−07 | 107.07 | 38.82 | 2.76 | ADAM19 | ADAM | 8728 | NM_023038 |
| | | | | | | | metallopeptidase | | |
| | | | | | | | domain 19 | | |
| 0.0000004 | 0.000015 | <1e−07 | 139.26 | 45.3 | 3.07 | MICAL1 | microtubule | 64780 | NM_001159291 |
| | | | | | | | associated | | |
| | | | | | | | monooxygenase, | | |
| | | | | | | | calponin and LIM | | |
| | | | | | | | domain containing | | |
| | | | | | | | 1 | | |
| 0.0000004 | 0.000015 | <1e−07 | 47.77 | 21.85 | 2.19 | TENT4A | Terminal | 11044 | |
| | | | | | | | Nucleotidyl- | | |
| | | | | | | | transferase 4A | | |
| 0.0000004 | 0.000015 | <1e−07 | 5.25 | 13.91 | 0.38 | B3GALT5 | beta-1,3- | 10317 | NM_001278650 |
| | | | | | | | galactosyl- | | |
| | | | | | | | transferase 5 | | |
| 0.0000004 | 0.000015 | <1e−07 | 33.87 | 11.15 | 3.04 | TNFRSF18 | TNF receptor | 8784 | NM_004195 |
| | | | | | | | superfamily | | |
| | | | | | | | member 18 | | |
| 0.0000004 | 0.000015 | <1e−07 | 50.23 | 22.66 | 2.22 | PIKFYVE | phosphoinositide | 200576 | NM_001002881 |
| | | | | | | | kinase, FYVE- | | |
| | | | | | | | type zinc finger | | |
| | | | | | | | containing | | |
| 0.0000004 | 0.000015 | <1e−07 | 26036.47 | 89741.76 | 0.29 | MT-RNR1 | Mitochondrially | 4549 | |
| | | | | | | | Encoded 12S | | |
| | | | | | | | RRNA | | |
| 0.0000004 | 0.000015 | <1e−07 | 101.86 | 50.36 | 2.02 | KANSL1 | KAT8 regulatory | 284058 | NM_001193465 |
| | | | | | | | NSL complex | | |
| | | | | | | | subunit 1 | | |
| 0.0000004 | 0.000015 | <1e−07 | 376.27 | 157.9 | 2.38 | TGFB1 | transforming | 7040 | NM_000660 |
| | | | | | | | growth factor beta | | |
| | | | | | | | 1 | | |
| 0.0000004 | 0.000015 | <1e−07 | 116.78 | 57.21 | 2.04 | IQGAP2 | IQ motif | 10788 | NM_001285460 |
| | | | | | | | containing | | |
| | | | | | | | GTPase activating | | |
| | | | | | | | protein 2 | | |
| 0.0000004 | 0.000015 | <1e−07 | 38.65 | 17.18 | 2.25 | AUP1 | AUP1, lipid | 550 | NM_181575 |
| | | | | | | | droplet regulating | | |
| | | | | | | | VLDL assembly | | |
| | | | | | | | factor | | |
| 0.0000004 | 0.000015 | <1e−07 | 24.21 | 7.63 | 3.17 | CD72 | CD72 molecule | 97 | NM_001782 |
| 0.0000005 | 1.78E−05 | <1e−07 | 55.35 | 22.16 | 2.5 | MAP3K1 | mitogen-activated | 4214 | NM_005921 |
| | | | | | | | protein kinase | | |
| | | | | | | | kinase kinase 1 | | |
| 0.0000005 | 1.78E−05 | <1e−07 | 64.21 | 31.48 | 2.04 | TASOR2 | Transcription | 54906 | |
| | | | | | | | Activation | | |
| | | | | | | | Suppressor | | |
| | | | | | | | Family Member 2 | | |
| 0.0000005 | 1.78E−05 | <1e−07 | 31.25 | 9.95 | 3.14 | TNFRSF13B | TNF receptor | 23495 | NM_012452 |
| | | | | | | | superfamily | | |
| | | | | | | | member 13B | | |
| 0.0000005 | 1.78E−05 | <1e−07 | 5.28 | 10.9 | 0.48 | SEMA4G | semaphorin 4G | 57715 | NM_001203244 |
| 0.0000005 | 1.78E−05 | <1e−07 | 113.4 | 49.23 | 2.3 | ITPR1 | inositol 1,4,5- | 3708 | NM_001099952 |
| | | | | | | | trisphosphate | | |
| | | | | | | | receptor type 1 | | |
| 0.0000005 | 1.78E−05 | <1e−07 | 127.28 | 43.04 | 2.96 | OGT | O-linked N- | 8473 | NM_003605 |
| | | | | | | | acetylglucosamine | | |
| | | | | | | | (GlcNAc) | | |
| | | | | | | | transferase | | |
| 0.0000005 | 1.78E−05 | <1e−07 | 67.3 | 25 | 2.69 | ATF5 | activating | 22809 | NM_001193646 |
| | | | | | | | transcription | | |
| | | | | | | | factor 5 | | |
| 0.0000005 | 1.78E−05 | <1e−07 | 24.37 | 11.94 | 2.04 | LRRC41 | leucine rich repeat | 10489 | NM_006369 |
| | | | | | | | containing 41 | | |
| 0.0000005 | 1.78E−05 | <1e−07 | 5.83 | 15.21 | 0.38 | MUC19 | mucin 19, | 283463 | NM_173600 |
| | | | | | | | oligomeric | | |
| 0.0000005 | 1.78E−05 | <1e−07 | 24.46 | 9.17 | 2.67 | DTX1 | deltex E3 | 1840 | NM_004416 |
| | | | | | | | ubiquitin ligase 1 | | |
| 0.0000005 | 1.78E−05 | <1e−07 | 15.77 | 6.48 | 2.43 | ADM2 | adrenomedullin 2 | 79924 | NM_001253845 |
| 0.0000006 | 2.07E−05 | <1e−07 | 18.73 | 8.48 | 2.21 | VAMP2 | vesicle associated | 6844 | NM_001330125 |
| | | | | | | | membrane protein | | |
| | | | | | | | 2 | | |
| 0.0000006 | 2.07E−05 | <1e−07 | 59.59 | 26.53 | 2.25 | ATG4B | autophagy related | 23192 | NM_013325 |
| | | | | | | | 4B cysteine | | |
| | | | | | | | peptidase | | |
| 0.0000006 | 2.07E−05 | <1e−07 | 36.17 | 18.06 | 2 | CLCN6 | chloride voltage- | 1185 | NM_001256959 |
| | | | | | | | gated channel 6 | | |
| 0.0000006 | 2.07E−05 | <1e−07 | 29.55 | 12.61 | 2.34 | MTSS1 | MTSS1, I-BAR | 9788 | NM_001282971 |
| | | | | | | | domain containing | | |
| 0.0000006 | 2.07E−05 | <1e−07 | 5.69 | 12.62 | 0.45 | PRSS8 | serine protease 8 | 5652 | NM_002773 |
| 0.0000006 | 2.07E−05 | <1e−07 | 48.92 | 20.88 | 2.34 | KDM6A | lysine | 7403 | NM_001291415 |
| | | | | | | | demethylase 6A | | |
| 0.0000006 | 2.07E−05 | <1e−07 | 55.88 | 25.28 | 2.21 | PACS1 | phosphofurin | 55690 | NM_018026 |
| | | | | | | | acidic cluster | | |
| | | | | | | | sorting protein 1 | | |
| 0.0000006 | 2.07E−05 | <1e−07 | 124.69 | 56.5 | 2.21 | STX4 | syntaxin 4 | 6810 | NM_001272095 |
| 0.0000007 | 2.34E−05 | <1e−07 | 142.42 | 54.81 | 2.6 | TYK2 | tyrosine kinase 2 | 7297 | NM_003331 |
| 0.0000007 | 2.34E−05 | <1e−07 | 476.75 | 232.01 | 2.05 | CD44 | CD44 molecule | 960 | NM_000610 |
| | | | | | | | (Indian blood | | |
| | | | | | | | group) | | |
| 0.0000007 | 2.34E−05 | <1e−07 | 103.54 | 42.09 | 2.46 | EDEM1 | ER degradation | 9695 | NM_014674 |
| | | | | | | | enhancing alpha- | | |
| | | | | | | | mannosidase like | | |
| | | | | | | | protein 1 | | |
| 0.0000007 | 2.34E−05 | <1e−07 | 114.75 | 56.88 | 2.02 | PCNX1 | pecanexhomolog | 22990 | NM_001308160 |
| | | | | | | | 1 | | |
| 0.0000007 | 2.34E−05 | <1e−07 | 22.95 | 10.01 | 2.29 | AMZ2 | archaelysin family | 51321 | NM_001033569 |
| | | | | | | | metallopeptidase | | |
| | | | | | | | 2 | | |
| 0.0000007 | 2.34E−05 | <1e−07 | 24.91 | 11.72 | 2.13 | CBX7 | chromobox 7 | 23492 | NM_001346743 |
| 0.0000007 | 2.34E−05 | <1e−07 | 19.73 | 8.91 | 2.21 | SRCAP | Snf2 related | 10847 | NM_006662 |
| | | | | | | | CREBBP | | |
| | | | | | | | activator protein | | |
| 0.0000007 | 2.34E−05 | <1e−07 | 144.19 | 68.04 | 2.12 | HSPA9 | heat shock protein | 3313 | NM_004134 |
| | | | | | | | family A (Hsp70) | | |
| | | | | | | | member 9 | | |
| 0.0000007 | 2.34E−05 | <1e−07 | 130.38 | 60.21 | 2.17 | PPP6R1 | protein | 22870 | NM_014931 |
| | | | | | | | phosphatase 6 | | |
| | | | | | | | regulatory subunit | | |
| | | | | | | | 1 | | |
| 0.0000007 | 2.34E−05 | <1e−07 | 38.87 | 18.64 | 2.09 | GNPTG | N- | 84572 | NM_032520 |
| | | | | | | | acetylglucosamine- | | |
| | | | | | | | 1-phosphate | | |
| | | | | | | | transferase | | |
| | | | | | | | gamma subunit | | |
| 0.0000008 | 2.61E−05 | <1e−07 | 5.47 | 12.96 | 0.42 | REG4 | regenerating | 83998 | NM_001159352 |
| | | | | | | | family member 4 | | |
| 0.0000008 | 2.61E−05 | <1e−07 | 148.8 | 55.75 | 2.67 | HCLS1 | hematopoietic | 3059 | NM_001292041 |
| | | | | | | | cell-specific Lyn | | |
| | | | | | | | substrate 1 | | |
| 0.0000008 | 2.61E−05 | <1e−07 | 175.09 | 86.77 | 2.02 | CLK1 | CDC like kinase 1 | 1195 | NM_001024646 |
| 0.0000008 | 2.61E−05 | <1e−07 | 50.88 | 22.4 | 2.27 | ZFYVE26 | zinc finger FYVE- | 23503 | NM_015346 |
| | | | | | | | type containing 26 | | |
| 0.0000008 | 2.61E−05 | <1e−07 | 31.8 | 13.65 | 2.33 | WASHC4 | WASH complex | 23325 | NM_001293640 |
| | | | | | | | subunit 4 | | |
| 0.0000008 | 2.61E−05 | <1e−07 | 29.42 | 13.52 | 2.18 | ZC3H7A | zinc finger | 29066 | NM_014153 |
| | | | | | | | CCCH-type | | |
| | | | | | | | containing 7A | | |
| 0.0000008 | 2.61E−05 | <1e−07 | 44.19 | 19.96 | 2.21 | ARHGAP31 | Rho GTPase | 57514 | NM_020754 |
| | | | | | | | activating protein | | |
| | | | | | | | 31 | | |
| 0.0000009 | 2.83E−05 | <1e−07 | 14.03 | 6.71 | 2.09 | TEC | tec protein | 7006 | NM_003215 |
| | | | | | | | tyrosine kinase | | |
| 0.0000009 | 2.83E−05 | <1e−07 | 104.93 | 52.35 | 2 | GDI1 | GDP dissociation | 2664 | NM_0014939 |
| | | | | | | | inhibitor 1 | | |
| 0.0000009 | 2.83E−05 | <1e−07 | 766.53 | 2237 | 0.34 | MT-ND6 | Mitochondrially | 4541 | |
| | | | | | | | Encoded | | |
| | | | | | | | NADH: Ubiquinone | | |
| | | | | | | | Oxidoreductase | | |
| | | | | | | | Core Subunit 6 | | |
| 0.0000009 | 2.83E−05 | <1e−07 | 122.78 | 54.71 | 2.24 | PTPN1 | protein tyrosine | 5770 | NM_001278618 |
| | | | | | | | phosphatase, non- | | |
| | | | | | | | receptor type 1 | | |
| 0.0000009 | 2.83E−05 | <1e−07 | 40.29 | 18.55 | 2.17 | PDE7A | phosphodiesterase | 5150 | NM_001242318 |
| | | | | | | | 7A | | |
| 0.0000009 | 2.83E−05 | <1e−07 | 25.09 | 11.79 | 2.13 | AC008105.3 | noncoding | | |
| | | | | | | | (Ensemble gene | | |
| | | | | | | | ID: | | |
| | | | | | | | ENSG00000272256.1) | | |
| 0.0000009 | 2.83E−05 | <1e−07 | 35.11 | 14.74 | 2.38 | ERO1B | endoplasmic | 56605 | NM_019891 |
| | | | | | | | reticulum | | |
| | | | | | | | oxidoreductase 1 | | |
| | | | | | | | beta | | |
| 0.0000009 | 2.83E−05 | <1e−07 | 46.48 | 22.25 | 2.09 | MED25 | mediator complex | 81857 | NM_030973 |
| | | | | | | | subunit 25 | | |
| 0.0000009 | 2.83E−05 | <1e−07 | 43.06 | 18.87 | 2.28 | CENPC | centromere | 1060 | NM_001812 |
| | | | | | | | protein C | | |
| 0.0000009 | 2.83E−05 | <1e−07 | 50.87 | 22.71 | 2.24 | WASHC2C | WASH complex | 253725 | NM_001169106 |
| | | | | | | | subunit 2C | | |
| 0.0000009 | 2.83E−05 | <1e−07 | 70.25 | 33.53 | 2.1 | KLHDC4 | kelch domain | 54758 | NM_001184854 |
| | | | | | | | containing 4 | | |
| 0.0000009 | 2.83E−05 | <1e−07 | 99.42 | 40.05 | 2.48 | ZNF331 | zinc finger protein | 55422 | NM_001079906 |
| | | | | | | | 331 | | |
| 0.000001 | 3.07E−05 | <1e−07 | 87.38 | 36.76 | 2.38 | LTBP3 | latent | 4054 | NM_001130144 |
| | | | | | | | transforming | | |
| | | | | | | | growth factor beta | | |
| | | | | | | | binding protein 3 | | |
| 0.000001 | 3.07E−05 | <1e−07 | 27.49 | 9.61 | 2.86 | IGLL5 | immunoglobulin | 100423062 | NM_001178126 |
| | | | | | | | lambda like | | |
| | | | | | | | polypeptide 5 | | |
| 0.000001 | 3.07E−05 | <1e−07 | 51.55 | 24.34 | 2.12 | NAGK | N- | 55577 | NM_001330425 |
| | | | | | | | acetylglucosamine | | |
| | | | | | | | kinase | | |
| 0.000001 | 3.07E−05 | <1e−07 | 366.93 | 173.78 | 2.11 | ILF3 | interleukin | 3609 | NM_001137673 |
| | | | | | | | enhancer binding | | |
| | | | | | | | factor 3 | | |
| 0.000001 | 3.07E−05 | <1e−07 | 71.05 | 31.44 | 2.26 | STX5 | syntaxin 5 | 6811 | NM_001244666 |
| 0.000001 | 3.07E−05 | <1e−07 | 24.69 | 11.64 | 2.12 | POLG2 | DNA polymerase | 11232 | NM_007215 |
| | | | | | | | gamma 2, | | |
| | | | | | | | accessory subunit | | |
| 0.000001 | 3.07E−05 | <1e−07 | 39.41 | 19.38 | 2.03 | DNAJB2 | Dna J heat shock | 3300 | NM_001039550 |
| | | | | | | | protein family | | |
| | | | | | | | (Hsp40) member | | |
| | | | | | | | B2 | | |
| 0.000001 | 3.07E−05 | <1e−07 | 103.08 | 48.68 | 2.12 | IFI16 | interferon gamma | 3428 | NM_001206567 |
| | | | | | | | inducible protein | | |
| | | | | | | | 16 | | |
| 0.000001 | 3.07E−05 | <1e−07 | 143.22 | 52.46 | 2.73 | LINC-PINT | long intergenic | 378805 | NM_001085379 |
| | | | | | | | non-protein | | |
| | | | | | | | coding RNA, p53 | | |
| | | | | | | | induced transcript | | |
| 0.0000011 | 3.33E−05 | <1e−07 | 47.79 | 22.21 | 2.15 | NCKAP5L | NCK associated | 57701 | NM_001037806 |
| | | | | | | | protein 5 like | | |
| 0.0000011 | 3.33E−05 | <1e−07 | 150.04 | 65.03 | 2.31 | CSNKID | casein kinase 1 | 1453 | NM_001893 |
| | | | | | | | delta | | |
| 0.0000011 | 3.33E−05 | <1e−07 | 572.98 | 273.21 | 2.1 | MYO9B | myosin IXB | 4650 | NM_001130065 |
| 0.0000011 | 3.33E−05 | <1e−07 | 22.9 | 9.8 | 2.34 | FCHO1 | FCH domain only | 23149 | NM_001161357 |
| | | | | | | | 1 | | |
| 0.0000012 | 3.52E−05 | <1e−07 | 6.89 | 21.33 | 0.32 | PLEKHH1 | pleckstrin | 57475 | NM_020715 |
| | | | | | | | homology, | | |
| | | | | | | | MyTH4 and | | |
| | | | | | | | FERM domain | | |
| | | | | | | | containing H1 | | |
| 0.0000012 | 3.52E−05 | <1e−07 | 33.53 | 16.13 | 2.08 | FYTTD1 | forty-two-three | 84248 | NM_001011537 |
| | | | | | | | domain containing | | |
| | | | | | | | 1 | | |
| 0.0000012 | 3.52E−05 | <1e−07 | 29.72 | 13.99 | 2.12 | SECISBP2L | SECIS binding | 9728 | NM_001193489 |
| | | | | | | | protein 2 like | | |
| 0.0000012 | 3.52E−05 | <1e−07 | 42.39 | 16.96 | 2.5 | ITGB7 | integrin subunit | 3695 | NM_000889 |
| | | | | | | | beta 7 | | |
| 0.0000012 | 3.52E−05 | <1e−07 | 37.32 | 16.75 | 2.23 | AHDC1 | AT-hook DNA | 27245 | NM_001029882 |
| | | | | | | | binding motif | | |
| | | | | | | | containing 1 | | |
| 0.0000012 | 3.52E−05 | <1e−07 | 60.96 | 25.03 | 2.44 | SLC7A1 | solute carrier | 6541 | NM_003045 |
| | | | | | | | family 7 member | | |
| | | | | | | | 1 | | |
| 0.0000012 | 3.52E−05 | <1e−07 | 6.91 | 18.49 | 0.37 | CACNA1D | calcium voltage- | 776 | NM_000720 |
| | | | | | | | gated channel | | |
| | | | | | | | subunit alpha 1 D | | |
| 0.0000012 | 3.52E−05 | <1e−07 | 6.18 | 13.15 | 0.47 | C1orf115 | chromosome 1 | 79762 | NM_024709 |
| | | | | | | | open reading | | |
| | | | | | | | frame 115 | | |
| 0.0000013 | 3.73E−05 | <1e−07 | 118.96 | 51.23 | 2.32 | ARHGAP4 | Rho GTPase | 393 | NM_001164741 |
| | | | | | | | activating protein | | |
| | | | | | | | 4 | | |
| 0.0000013 | 3.73E−05 | <1e−07 | 5.25 | 11.38 | 0.46 | PBLD | phenazine | 64081 | NM_001033083 |
| | | | | | | | biosynthesis like | | |
| | | | | | | | protein domain | | |
| | | | | | | | containing | | |
| 0.0000013 | 3.73E−05 | <1e−07 | 58.05 | 22.69 | 2.56 | SLC8B1 | solute carrier | 80024 | NM_001330466 |
| | | | | | | | family 8 member | | |
| | | | | | | | B1 | | |
| 0.0000013 | 3.73E−05 | <1e−07 | 30.06 | 13.56 | 2.22 | CHPF2 | chondroitin | 54480 | NM_001284295 |
| | | | | | | | polymerizing | | |
| | | | | | | | factor 2 | | |
| 0.0000014 | 3.93E−05 | <1e−07 | 52.48 | 24.11 | 2.18 | ZBTB1 | zinc finger and | 22890 | NM_001123329 |
| | | | | | | | BTB domain | | |
| | | | | | | | containing 1 | | |
| 0.0000014 | 3.93E−05 | <1e−07 | 22.07 | 11.02 | 2 | ADNP2 | ADNP homeobox | 22850 | NM_014913 |
| | | | | | | | 2 | | |
| 0.0000014 | 3.93E−05 | <1e−07 | 72.03 | 30.54 | 2.36 | EMP3 | epithelial | 2014 | NM_001313905 |
| | | | | | | | membrane protein | | |
| | | | | | | | 3 | | |
| 0.0000014 | 3.93E−05 | <1e−07 | 5.31 | 11.52 | 0.46 | MT-TY | Mitochondrially | 4579 | |
| | | | | | | | Encoded TRNA- | | |
| | | | | | | | Tyr (UAU/C) | | |
| 0.0000014 | 3.93E−05 | <1e−07 | 91.75 | 37.9 | 2.42 | RHBDF2 | rhomboid 5 | 79651 | NM_001005498 |
| | | | | | | | homolog 2 | | |
| 0.0000014 | 3.93E−05 | <1e−07 | 83.57 | 38.26 | 2.18 | SEC61A1 | Sec61 translocon | 29927 | NM_013336 |
| | | | | | | | alpha 1 subunit | | |
| 0.0000014 | 3.93E−05 | <1e−07 | 115.65 | 50.94 | 2.27 | GCN1 | GCN1, eIF2 alpha | 10985 | NM_006836 |
| | | | | | | | kinase activator | | |
| | | | | | | | homolog | | |
| 0.0000015 | 0.000041 | <1e−07 | 23.13 | 11.12 | 2.08 | ATF2 | activating | 1386 | NM_001256090 |
| | | | | | | | transcription | | |
| | | | | | | | factor 2 | | |
| 0.0000015 | 0.000041 | <1e−07 | 6.01 | 12.75 | 0.47 | WWC1 | WW and C2 | 23286 | NM_001161661 |
| | | | | | | | domain containing | | |
| | | | | | | | 1 | | |
| 0.0000015 | 0.000041 | <1e−07 | 51.89 | 22.12 | 2.35 | GMPPB | GDP-mannose | 29925 | NM_013334 |
| | | | | | | | pyrophosphorylase | | |
| | | | | | | | B | | |
| 0.0000015 | 0.000041 | <1e−07 | 101.63 | 47.69 | 2.13 | AGAP3 | ArfGAP with | 116988 | NM_001042535 |
| | | | | | | | GTPase domain, | | |
| | | | | | | | ankyrin repeat and | | |
| | | | | | | | PH domain 3 | | |
| 0.0000015 | 0.000041 | <1e−07 | 54.99 | 25.64 | 2.14 | IRF3 | interferon | 3661 | NM_001197122 |
| | | | | | | | regulatory factor 3 | | |
| 0.0000016 | 4.31E−05 | <1e−07 | 77.29 | 34.98 | 2.21 | TRABD | Tra B domain | 80305 | NM_001320484 |
| | | | | | | | containing | | |
| 0.0000016 | 4.31E−05 | <1e−07 | 34.74 | 17.14 | 2.03 | RIC1 | RIC1 homolog, | 57589 | NM_001135920 |
| | | | | | | | RAB6A GF | | |
| | | | | | | | complex partner 1 | | |
| 0.0000016 | 4.31E−05 | <1e−07 | 5.59 | 14.63 | 0.38 | DDC | dopa | 1644 | NM_000790 |
| | | | | | | | decarboxylase | | |
| 0.0000016 | 4.31E−05 | <1e−07 | 87.2 | 37.82 | 2.31 | STK17B | serine/threonine | 9262 | NM_004226 |
| | | | | | | | kinase 17b | | |
| 0.0000017 | 4.51E−05 | <1e−07 | 6.94 | 19.68 | 0.35 | MT-TQ | Mitochondrially | 4572 | |
| | | | | | | | Encoded TRNA- | | |
| | | | | | | | Gln (CAA/G) | | |
| 0.0000017 | 4.51E−05 | <1e−07 | 44.98 | 21.69 | 2.07 | UBALD1 | UBA like domain | 124402 | NM_001330467 |
| | | | | | | | containing 1 | | |
| 0.0000018 | 4.73E−05 | <1e−07 | 38.93 | 15.4 | 2.53 | TPRA1 | transmembrane | 131601 | NM_001136053 |
| | | | | | | | protein adipocyte | | |
| | | | | | | | associated 1 | | |
| 0.0000018 | 4.73E−05 | <1e−07 | 18.36 | 8 | 2.29 | VASH1 | vasohibin 1 | 22846 | NM_014909 |
| 0.0000018 | 4.73E−05 | <1e−07 | 41.38 | 20.18 | 2.05 | TBC1D17 | TBC1 domain | 79735 | NM_001168222 |
| | | | | | | | family member 17 | | |
| 0.0000019 | 0.000049 | <1e−07 | 51.33 | 24.55 | 2.09 | RFX1 | regulatory factor | 5989 | NM_002918 |
| | | | | | | | X1 | | |
| 0.0000019 | 0.000049 | <1e−07 | 119.78 | 58.74 | 2.04 | TRIM28 | tripartite motif | 10155 | NM_005762 |
| | | | | | | | containing 28 | | |
| 0.0000019 | 0.000049 | <1e−07 | 286.42 | 138.79 | 2.06 | HIPK2 | homeodomain | 28996 | NM_001113239 |
| | | | | | | | interacting protein | | |
| | | | | | | | kinase 2 | | |
| 0.0000019 | 0.000049 | <1e−07 | 42.82 | 19 | 2.25 | POLDIP3 | DNA polymerase | 84271 | NM_001278657 |
| | | | | | | | delta interacting | | |
| | | | | | | | protein 3 | | |
| 0.0000019 | 0.000049 | <1e−07 | 23.38 | 10.4 | 2.25 | TRAM2 | translocation | 9697 | NM_012288 |
| | | | | | | | associated | | |
| | | | | | | | membrane protein | | |
| | | | | | | | 2 | | |
| 0.000002 | 5.12E−05 | 0.0001 | 41.73 | 19.87 | 2.1 | MAP2K7 | mitogen-activated | 5609 | NM_001297555 |
| | | | | | | | protein kinase | | |
| | | | | | | | kinase 7 | | |
| 0.000002 | 5.12E−05 | <1e−07 | 41.78 | 20.86 | 2 | DDHD1 | DDHD domain | 80821 | NM_001160147 |
| | | | | | | | containing 1 | | |
| 0.0000021 | 5.27E−05 | <1e−07 | 32.71 | 15.43 | 2.12 | ABTB1 | ankyrin repeat and | 80325 | NM_032548 |
| | | | | | | | BTB domain | | |
| | | | | | | | containing 1 | | |
| 0.0000021 | 5.27E−05 | <1e−07 | 30.73 | 12.75 | 2.41 | AC139887.2 | noncoding | | |
| | | | | | | | (Ensemble gene | | |
| | | | | | | | ID: | | |
| | | | | | | | ENSG00000249592.6) | | |
| 0.0000021 | 5.27E−05 | <1e−07 | 22.52 | 10.53 | 2.14 | PPM1K | protein | 152926 | NM_152542 |
| | | | | | | | phosphatase, | | |
| | | | | | | | Mg2+/Mn2+ | | |
| | | | | | | | dependent 1K | | |
| 0.0000021 | 5.27E−05 | <1e−07 | 49.05 | 21.62 | 2.27 | ADAM17 | ADAM | 6868 | NM_003183 |
| | | | | | | | metallopeptidase | | |
| | | | | | | | domain 17 | | |
| 0.0000021 | 5.27E−05 | <1e−07 | 71 | 34.4 | 2.06 | CCDC88A | coiled-coil | 55704 | NM_001135597 |
| | | | | | | | domain containing | | |
| | | | | | | | 88A | | |
| 0.0000021 | 5.27E−05 | <1e−07 | 23.25 | 11.06 | 2.1 | TARS3 | Threonyl-TRNA | 123283 | |
| | | | | | | | Synthetase 3 | | |
| 0.0000021 | 5.27E−05 | <1e−07 | 572.37 | 282.86 | 2.02 | KLF6 | Kruppel like | 1316 | NM_001008490 |
| | | | | | | | factor 6 | | |
| 0.0000021 | 5.27E−05 | <1e−07 | 142.13 | 61.48 | 2.31 | STK4 | serine/threonine | 6789 | NM_001352385 |
| | | | | | | | kinase 4 | | |
| 0.0000022 | 5.47E−05 | <1e−07 | 31.56 | 15.21 | 2.07 | CCDC97 | coiled-coil | 90324 | NM_001346100 |
| | | | | | | | domain containing | | |
| | | | | | | | 97 | | |
| 0.0000022 | 5.47E−05 | <1e−07 | 6.39 | 15.03 | 0.43 | MAOA | monoamine | 4128 | NM_000240 |
| | | | | | | | oxidase A | | |
| 0.0000023 | 5.65E−05 | 0.0001 | 19.14 | 7.75 | 2.47 | SIK1B | salt inducible | 102724428 | NM_001320643 |
| | | | | | | | kinase 1B | | |
| | | | | | | | (putative) | | |
| 0.0000023 | 5.65E−05 | <1e−07 | 173.56 | 71.44 | 2.43 | PIM1 | Pim-1 proto- | 5292 | NM_001243186 |
| | | | | | | | oncogene, | | |
| | | | | | | | serine/threonine | | |
| | | | | | | | kinase | | |
| 0.0000023 | 5.65E−05 | <1e−07 | 38.41 | 19.01 | 2.02 | RGL2 | ral guanine | 5863 | NM_001243738 |
| | | | | | | | nucleotide | | |
| | | | | | | | dissociation | | |
| | | | | | | | stimulator like 2 | | |
| 0.0000023 | 5.65E−05 | <1e−07 | 65.83 | 26.56 | 2.48 | OSBPL3 | oxysterol binding | 26031 | NM_015550 |
| | | | | | | | protein like 3 | | |
| 0.0000023 | 5.65E−05 | <1e−07 | 36.83 | 17.49 | 2.11 | RING1 | ring finger protein | 6015 | NM_002931 |
| | | | | | | | 1 | | |
| 0.0000023 | 5.65E−05 | <1e−07 | 30.99 | 13.16 | 2.35 | NPIPB4 | nuclear pore | 440345 | NM_001310148 |
| | | | | | | | complex | | |
| | | | | | | | interacting protein | | |
| | | | | | | | family member | | |
| | | | | | | | B4 | | |
| 0.0000024 | 5.78E−05 | <1e−07 | 30.46 | 12.75 | 2.39 | SYTL1 | synaptotagmin | 84958 | NM_001193308 |
| | | | | | | | like 1 | | |
| 0.0000024 | 5.78E−05 | <1e−07 | 114.99 | 57.03 | 2.02 | GNL1 | G protein | 2794 | NM_005275 |
| | | | | | | | nucleolar 1 | | |
| | | | | | | | (putative) | | |
| 0.0000024 | 5.78E−05 | <1e−07 | 45.02 | 19.2 | 2.34 | ARL8B | ADP ribosylation | 55207 | NM_018184 |
| | | | | | | | factor like GTPase | | |
| | | | | | | | 8B | | |
| 0.0000024 | 5.78E−05 | <1e−07 | 61.81 | 22.19 | 2.79 | TBC1D9 | TBC1 domain | 23158 | NM_015130 |
| | | | | | | | family member 9 | | |
| 0.0000024 | 5.78E−05 | <1e−07 | 70.57 | 29.89 | 2.36 | CD37 | CD37 molecule | 951 | NM_001040031 |
| 0.0000024 | 5.78E−05 | <1e−07 | 24.89 | 11.31 | 2.2 | IRF2BP1 | interferon | 26145 | NM_015649 |
| | | | | | | | regulatory factor 2 | | |
| | | | | | | | binding protein 1 | | |
| 0.0000024 | 5.78E−05 | <1e−07 | 24.61 | 8.74 | 2.82 | BLK | BLK proto- | 640 | NM_001330465 |
| | | | | | | | oncogene, Src | | |
| | | | | | | | family tyrosine | | |
| | | | | | | | kinase | | |
| 0.0000025 | 0.00006 | <1e−07 | 25.52 | 12.04 | 2.12 | ZNF655 | zinc finger protein | 79027 | NM_001009956 |
| | | | | | | | 655 | | |
| 0.0000025 | 0.00006 | <1e−07 | 95.7 | 40.09 | 2.39 | NKTR | natural killer cell | 4820 | NM_001012651 |
| | | | | | | | triggering receptor | | |
| 0.0000026 | 6.19E−05 | <1e−07 | 14.83 | 7.4 | 2.01 | TMEM268 | transmembrane | 203197 | NM_001330760 |
| | | | | | | | protein 268 | | |
| 0.0000026 | 6.19E−05 | <1e−07 | 33.09 | 16.03 | 2.06 | FCHSD1 | FCH and double | 89848 | NM_033449 |
| | | | | | | | SH3 domains 1 | | |
| 0.0000026 | 6.19E−05 | <1e−07 | 66.97 | 29.78 | 2.25 | DOCK10 | dedicator of | 55619 | NM_001290263 |
| | | | | | | | cytokinesis 10 | | |
| 0.0000027 | 6.33E−05 | <1e−07 | 48.11 | 22.55 | 2.13 | ABCF3 | ATP binding | 55324 | NM_001351298 |
| | | | | | | | cassette subfamily | | |
| | | | | | | | F member 3 | | |
| 0.0000027 | 6.33E−05 | <1e−07 | 27.03 | 13.42 | 2.01 | CHMP7 | charged | 91782 | NM_001317899 |
| | | | | | | | multivesicular | | |
| | | | | | | | body protein 7 | | |
| 0.0000027 | 6.33E−05 | <1e−07 | 118.24 | 47.53 | 2.49 | NCOA3 | nuclear receptor | 8202 | NM_001174087 |
| | | | | | | | coactivator 3 | | |
| 0.0000027 | 6.33E−05 | <1e−07 | 67.94 | 32.49 | 2.09 | MYO5A | myosin VA | 4644 | NM_000259 |
| 0.0000027 | 6.33E−05 | <1e−07 | 138.79 | 67.32 | 2.06 | BDP1 | B double prime 1, | 55814 | NM_018429 |
| | | | | | | | subunit of RNA | | |
| | | | | | | | polymerase III | | |
| | | | | | | | transcription | | |
| | | | | | | | initiation factor | | |
| | | | | | | | IIIB | | |
| 0.0000028 | 0.000065 | <1e−07 | 31.98 | 15.02 | 2.13 | SEMA4C | semaphorin 4C | 54910 | NM_017789 |
| 0.0000028 | 0.000065 | <1e−07 | 158.71 | 69.3 | 2.29 | ARID5A | AT-rich | 10865 | NM_001319085 |
| | | | | | | | interaction | | |
| | | | | | | | domain 5A | | |
| 0.0000028 | 0.000065 | <1e−07 | 34.06 | 15.79 | 2.16 | SFI1 | SFI1 centrin | 9814 | NM_001007467 |
| | | | | | | | binding protein | | |
| 0.0000028 | 0.000065 | 0.0001 | 34.54 | 14.78 | 2.34 | SNHG12 | small nucleolar | 85028 | NR_024127 |
| | | | | | | | RNA host gene 12 | | |
| 0.0000029 | 6.68E−05 | <1e−07 | 217.85 | 106.46 | 2.05 | TRA2B | transformer 2 beta | 6434 | NM_001243879 |
| | | | | | | | homolog | | |
| 0.0000029 | 6.68E−05 | <1e−07 | 164.98 | 70.66 | 2.33 | ATP2A3 | ATPase | 489 | NM_005173 |
| | | | | | | | sarcoplasmic/ | | |
| | | | | | | | endoplasmic | | |
| | | | | | | | reticulum Ca2+ | | |
| | | | | | | | transporting 3 | | |
| 0.000003 | 6.83E−05 | <1e−07 | 5.87 | 14.72 | 0.4 | XDH | xanthine | 7498 | NM_000379 |
| | | | | | | | dehydrogenase | | |
| 0.000003 | 6.83E−05 | <1e−07 | 207.97 | 100.75 | 2.06 | IGF2R | insulin like | 3482 | NM_000876 |
| | | | | | | | growth factor 2 | | |
| | | | | | | | receptor | | |
| 0.000003 | 6.83E−05 | <1e−07 | 35.01 | 11.03 | 3.17 | MIR155HG | MIR155 host gene | 114614 | NR_001458 |
| 0.000003 | 6.83E−05 | <1e−07 | 7.75 | 16.29 | 0.48 | UACA | uvealautoantigen | 55075 | NM_001008224 |
| | | | | | | | with coiled-coil | | |
| | | | | | | | domains and | | |
| | | | | | | | ankyrin repeats | | |
| 0.0000031 | 6.98E−05 | <1e−07 | 21.31 | 9.33 | 2.29 | ESR2 | estrogen receptor | 2100 | NM_001040275 |
| | | | | | | | 2 | | |
| 0.0000031 | 6.98E−05 | <1e−07 | 39702.89 | 17624.61 | 2.25 | MALAT1 | metastasis | 378938 | NR_002819 |
| | | | | | | | associated lung | | |
| | | | | | | | adenocarcinoma | | |
| | | | | | | | transcript 1 (non- | | |
| | | | | | | | protein coding) | | |
| 0.0000031 | 6.98E−05 | <1e−07 | 33.69 | 15.7 | 2.15 | IL18BP | interleukin 18 | 10068 | NM_001039659 |
| | | | | | | | binding protein | | |
| 0.0000032 | 7.14E−05 | <1e−07 | 8.18 | 31.27 | 0.26 | CLCA1 | chloride channel | 1179 | NM_001285 |
| | | | | | | | accessory 1 | | |
| 0.0000032 | 7.14E−05 | <1e−07 | 90.44 | 30.45 | 2.97 | LAMA5 | laminin subunit | 3911 | NM_005560 |
| | | | | | | | alpha 5 | | |
| 0.0000032 | 7.14E−05 | <1e−07 | 52.75 | 25.95 | 2.03 | ZNF394 | zinc finger protein | 84124 | NM_001345967 |
| | | | | | | | 394 | | |
| 0.0000032 | 7.14E−05 | <1e−07 | 222.31 | 99.96 | 2.22 | TMEM259 | transmembrane | 91304 | NM_001033026 |
| | | | | | | | protein 259 | | |
| 0.0000032 | 7.14E−05 | <1e−07 | 44.65 | 22.25 | 2.01 | EDC4 | enhancer of | 23644 | NM_014329 |
| | | | | | | | mRNA decapping | | |
| | | | | | | | 4 | | |
| 0.0000033 | 7.31E−05 | <1e−07 | 27.22 | 11.84 | 2.3 | PNPLA7 | patatin like | 375775 | NM_001098537 |
| | | | | | | | phospholipase | | |
| | | | | | | | domain containing | | |
| | | | | | | | 7 | | |
| 0.0000033 | 7.31E−05 | <1e−07 | 143.89 | 65.66 | 2.19 | PNPLA2 | patatin like | 57104 | NM_020376 |
| | | | | | | | phospholipase | | |
| | | | | | | | domain containing | | |
| | | | | | | | 2 | | |
| 0.0000034 | 7.49E−05 | <1e−07 | 24.79 | 10.33 | 2.4 | ST6GALNAC4 | ST6 N-acetyl- | 27090 | NM_014403 |
| | | | | | | | galactosaminide | | |
| | | | | | | | alpha-2,6- | | |
| | | | | | | | sialyltransferase 4 | | |
| 0.0000034 | 7.49E−05 | <1e−07 | 66.67 | 33.02 | 2.02 | CCDC93 | coiled-coil | 54520 | NM_019044 |
| | | | | | | | domain containing | | |
| | | | | | | | 93 | | |
| 0.0000035 | 7.64E−05 | <1e−07 | 46.31 | 20.81 | 2.23 | ARRDC2 | arrestin domain | 27106 | NM_001025604 |
| | | | | | | | containing 2 | | |
| 0.0000035 | 7.64E−05 | <1e−07 | 25.97 | 10.41 | 2.49 | IL4I1 | interleukin 4 | 259307 | NM_001258017 |
| | | | | | | | induced 1 | | |
| 0.0000035 | 7.64E−05 | <1e−07 | 1863.62 | 461.63 | 4.04 | SNORD17 | small nucleolar | 692086 | NR_003045 |
| | | | | | | | RNA, C/D box 17 | | |
| 0.0000035 | 7.64E−05 | <1e−07 | 50.42 | 20.59 | 2.45 | ITGA4 | integrin subunit | 3676 | NM_000885 |
| | | | | | | | alpha 4 | | |
| 0.0000036 | 7.74E−05 | <1e−07 | 24.93 | 11.74 | 2.12 | DEF8 | differentially | 54849 | NM_001242816 |
| | | | | | | | expressed in | | |
| | | | | | | | FDCP 8 homolog | | |
| 0.0000036 | 7.74E−05 | <1e−07 | 46.65 | 18.24 | 2.56 | JAK3 | Janus kinase 3 | 3718 | NM_000215 |
| 0.0000036 | 7.74E−05 | <1e−07 | 87.22 | 37.34 | 2.34 | TIPARP | TCDD inducible | 25976 | NM_001184717 |
| | | | | | | | poly(ADP-ribose) | | |
| | | | | | | | polymerase | | |
| 0.0000036 | 7.74E−05 | <1e−07 | 62.42 | 30.09 | 2.07 | CLPTM1L | CLPTM1 like | 81037 | NM_030782 |
| 0.0000036 | 7.74E−05 | <1e−07 | 101.94 | 47.35 | 2.15 | OTUD5 | OTU | 55593 | NM_001136157 |
| | | | | | | | deubiquitinase 5 | | |
| 0.0000036 | 7.74E−05 | <1e−07 | 47.24 | 18.62 | 2.54 | RHOH | ras homolog | 399 | NM_001278359 |
| | | | | | | | family member H | | |
| 0.0000036 | 7.74E−05 | <1e−07 | 39.86 | 18.72 | 2.13 | UNKL | unkempt family | 64718 | NM_001037125 |
| | | | | | | | like zinc finger | | |
| 0.0000037 | 0.000079 | <1e−07 | 103.09 | 45.07 | 2.29 | ANKLE2 | ankyrin repeat and | 23141 | NM_015114 |
| | | | | | | | LEM domain | | |
| | | | | | | | containing 2 | | |
| 0.0000037 | 0.000079 | <1e−07 | 55.21 | 25.62 | 2.16 | UNC13D | unc-13 homolog | 201294 | NM_19924242 |
| | | | | | | | D | | |
| 0.0000037 | 0.000079 | <1e−07 | 25.22 | 11.56 | 2.18 | SLC2A4RG | SLC2A4 regulator | 56731 | NM_020062 |
| 0.0000038 | 7.99E−05 | <1e−07 | 62.44 | 28.76 | 2.17 | CHFR | checkpoint with | 55743 | NM_001161344 |
| | | | | | | | forkhead and ring | | |
| | | | | | | | finger domains | | |
| 0.0000038 | 7.99E−05 | <1e−07 | 6.15 | 12.82 | 0.48 | PLCB4 | phospholipase C | 5332 | NM_000933 |
| | | | | | | | beta 4 | | |
| 0.0000038 | 7.99E−05 | <1e−07 | 43.93 | 18.31 | 2.4 | ST6GAL1 | ST6 beta- | 6480 | NM_001353916 |
| | | | | | | | galactoside alpha- | | |
| | | | | | | | 2,6- | | |
| | | | | | | | sialyltransferase 1 | | |
| 0.0000038 | 7.99E−05 | <1e−07 | 40.51 | 18.19 | 2.23 | FLI1 | Fli-1 proto- | 2313 | NM_001167681 |
| | | | | | | | oncogene, TS | | |
| | | | | | | | transcription | | |
| | | | | | | | factor | | |
| 0.0000038 | 7.99E−05 | <1e−07 | 53.19 | 25.6 | 2.08 | SWAP70 | switching B cell | 23075 | NM_001297714 |
| | | | | | | | complex subunit | | |
| | | | | | | | SWAP70 | | |
| 0.0000039 | 8.11E−05 | <1e−07 | 576.51 | 270.33 | 2.13 | JUND | JunD proto- | 3727 | NM_001286968 |
| | | | | | | | oncogene, AP-1 | | |
| | | | | | | | transcription | | |
| | | | | | | | factor subunit | | |
| 0.0000039 | 8.11E−05 | <1e−07 | 56.13 | 25.55 | 2.2 | ORAI2 | ORAI calcium | 80228 | NM_001126340 |
| | | | | | | | release-activated | | |
| | | | | | | | calcium | | |
| | | | | | | | modulator 2 | | |
| 0.000004 | 8.27E−05 | <1e−07 | 36.12 | 17.41 | 2.07 | UBE2D2 | ubiquitin | 7322 | NM_003339 |
| | | | | | | | conjugating | | |
| | | | | | | | enzyme E2 D2 | | |
| 0.000004 | 8.27E−05 | <1e−07 | 6.72 | 13.6 | 0.49 | LIPG | lipase G, | 9388 | NM_001308006 |
| | | | | | | | endothelial type | | |
| 0.0000041 | 8.41E−05 | <1e−07 | 20.19 | 9.3 | 2.17 | AC138932.1 | noncoding | | |
| | | | | | | | (Ensemble gene | | |
| | | | | | | | ID: | | |
| | | | | | | | ENSG00000183458.14) | | |
| 0.0000041 | 8.41E−05 | <1e−07 | 120.86 | 56.92 | 2.12 | CREBRF | CRB3 regulatory | 153222 | NM_001168393 |
| | | | | | | | factor | | |
| 0.0000043 | 8.74E−05 | <1e−07 | 23.76 | 11.63 | 2.04 | TSSC4 | tumor suppressing | 10078 | NM_001297658 |
| | | | | | | | subtransferable | | |
| | | | | | | | candidate 4 | | |
| 0.0000043 | 8.74E−05 | <1e−07 | 63.49 | 30.69 | 2.07 | GANAB | glucosidase II | 23193 | NM_001278192 |
| | | | | | | | alpha subunit | | |
| 0.0000044 | 8.87E−05 | <1e−07 | 26.44 | 13.21 | 2 | SLC12A9 | solute carrier | 56996 | NM_001267812 |
| | | | | | | | family 12 member | | |
| | | | | | | | 9 | | |
| 0.0000044 | 8.87E−05 | <1e−07 | 129.36 | 56.95 | 2.27 | DDX39A | DExD-box | 10212 | NM_001204057 |
| | | | | | | | helicase 39A | | |
| 0.0000045 | 8.96E−05 | <1e−07 | 66.24 | 28.88 | 2.29 | PRNP | prion protein | 5621 | NM_000311 |
| 0.0000045 | 8.96E−05 | <1e−07 | 112.32 | 45.97 | 2.44 | ITPKB | inositol- | 3707 | NM_002221 |
| | | | | | | | trisphosphate 3- | | |
| | | | | | | | kinase B | | |
| 0.0000045 | 8.96E−05 | <1e−07 | 44.05 | 21.13 | 2.09 | GNA12 | G protein subunit | 2768 | NM_001282440 |
| | | | | | | | alpha 12 | | |
| 0.0000045 | 8.96E−05 | <1e−07 | 19.54 | 9.23 | 2.12 | THOC6 | THO complex 6 | 79228 | NM_001142350 |
| 0.0000048 | 9.47E−05 | <1e−07 | 104.95 | 49.32 | 2.13 | HIVEP1 | human | 3096 | NM_002114 |
| | | | | | | | immunodeficiency | | |
| | | | | | | | virus type I | | |
| | | | | | | | enhancer binding | | |
| | | | | | | | protein 1 | | |
| 0.0000049 | 9.58E−05 | <1e−07 | 308.61 | 140.81 | 2.19 | PPP1R15B | protein | 84919 | NM_032833 |
| | | | | | | | phosphatase 1 | | |
| | | | | | | | regulatory subunit | | |
| | | | | | | | 15B | | |
| 0.0000049 | 9.58E−05 | <1e−07 | 94.43 | 40.55 | 2.33 | MED12 | mediator complex | 9968 | NM_00512020 |
| | | | | | | | subunit 12 | | |
| 0.0000049 | 9.58E−05 | <1e−07 | 36.57 | 14.82 | 2.47 | SP140 | SP140 nuclear | 11262 | NM_001005176 |
| | | | | | | | body protein | | |
| 0.0000049 | 9.58E−05 | <1e−07 | 46.24 | 21.41 | 2.16 | TLNRD1 | talin rod domain | 59274 | NM_022566 |
| | | | | | | | containing 1 | | |
| 0.000005 | 9.72E−05 | <1e−07 | 26.14 | 12.58 | 2.08 | CPNE1 | copine 1 | 8904 | NM_001198863 |
| 0.000005 | 9.72E−05 | <1e−07 | 64.66 | 31.24 | 2.07 | MTCH1 | mitochondrial | 23787 | NM_001271641 |
| | | | | | | | carrier 1 | | |
| 0.0000051 | 9.85E−05 | 0.0001 | 62.17 | 26.83 | 2.32 | GARS1 | Glycyl-TRNA | 2617 | |
| | | | | | | | Synthetase 1 | | |
| 0.0000052 | 0.0001 | <1e−07 | 40.42 | 16.4 | 2.47 | RASAL3 | RAS protein | 64926 | NM_001348027 |
| | | | | | | | activator like 3 | | |
| 0.0000053 | 0.000101 | <1e−07 | 5.51 | 12.79 | 0.43 | PRDM16 | PR/SET domain | 63976 | NM_022114 |
| | | | | | | | 16 | | |
| 0.0000053 | 0.000101 | 0.0001 | 61.48 | 30.07 | 2.04 | MAZ | MYC associated | 4150 | NM_001042539 |
| | | | | | | | zinc finger protein | | |
| 0.0000053 | 0.000101 | <1e−07 | 17.6 | 8.65 | 2.03 | CLN8 | CLN8, | 2055 | NM_001034061 |
| | | | | | | | transmembrane | | |
| | | | | | | | ER and ERGIC | | |
| | | | | | | | protein | | |
| 0.0000054 | 0.000103 | <1e−07 | 82.72 | 38.2 | 2.17 | NOP56 | NOP56 | 10528 | NM_006392 |
| | | | | | | | ribonucleoprotein | | |
| 0.0000055 | 0.000104 | <1e−07 | 47.27 | 19.49 | 2.43 | SNED1 | sushi, nidogen and | 25992 | NM_001080437 |
| | | | | | | | GF like domains | | |
| | | | | | | | 1 | | |
| 0.0000055 | 0.000104 | <1e−07 | 44.29 | 22.08 | 2.01 | TIMM44 | translocase of | 10469 | NM_006351 |
| | | | | | | | inner | | |
| | | | | | | | mitochondrial | | |
| | | | | | | | membrane 44 | | |
| 0.0000056 | 0.000105 | <1e−07 | 49.12 | 19.28 | 2.55 | AMDHD2 | amidohydrolase | 51005 | NM_001145815 |
| | | | | | | | domain containing | | |
| | | | | | | | 2 | | |
| 0.0000057 | 0.000106 | <1e−07 | 18.65 | 7.09 | 2.63 | HLA-DMB | major | 3109 | NM_002118 |
| | | | | | | | histocompatibility | | |
| | | | | | | | complex, class II, | | |
| | | | | | | | DM beta | | |
| 0.0000059 | 0.000109 | <1e−07 | 41.21 | 132.4 | 0.31 | MT-TM | Mitochondrially | 4569 | |
| | | | | | | | Encoded TRNA- | | |
| | | | | | | | Met (AUA/G) | | |
| 0.0000059 | 0.000109 | <1e−07 | 57.7 | 21.51 | 2.68 | CARD11 | caspa se | 84433 | NM_001324281 |
| | | | | | | | recruitment | | |
| | | | | | | | domain family | | |
| | | | | | | | member 11 | | |
| 0.000006 | 0.00011 | <1e−07 | 48.06 | 22.88 | 2.1 | WHAMM | WAS protein | 123720 | NM_001080435 |
| | | | | | | | homolog | | |
| | | | | | | | associated with | | |
| | | | | | | | actin, golgi | | |
| | | | | | | | membranes and | | |
| | | | | | | | microtubules | | |
| 0.0000062 | 0.000113 | <1e−07 | 34.16 | 16.61 | 2.06 | TAF4B | TATA-box | 6875 | NM_001293725 |
| | | | | | | | binding protein | | |
| | | | | | | | associated factor | | |
| | | | | | | | 4b | | |
| 0.0000062 | 0.000113 | <1e−07 | 37.91 | 13.92 | 2.72 | CD40 | CD40 molecule | 958 | NM_001250 |
| 0.0000063 | 0.000115 | <1e−07 | 90.88 | 37.59 | 2.42 | SIPA1 | signal-induced | 6494 | NM_006747 |
| | | | | | | | proliferation- | | |
| | | | | | | | associated 1 | | |
| 0.0000063 | 0.000115 | 0.0001 | 24.28 | 10.61 | 2.29 | ADAMTSL4- | ADAMTSL4 | 574406 | NM_001025493 |
| | | | | | | AS1 | antisense RNA 1 | | |
| 0.0000064 | 0.000116 | <1e−07 | 9.83 | 39.58 | 0.25 | VIL1 | villin 1 | 7429 | NM_007127 |
| 0.0000064 | 0.000116 | <1e−07 | 9.06 | 21.99 | 0.41 | CREB3L1 | cAMP responsive | 90993 | NM_052854 |
| | | | | | | | element binding | | |
| | | | | | | | protein 3 like 1 | | |
| 0.0000064 | 0.000116 | 0.0001 | 22.9 | 9.59 | 2.39 | A4GALT | alpha 1,4- | 53947 | NM_001318038 |
| | | | | | | | galactosyl- | | |
| | | | | | | | transferase | | |
| | | | | | | | (P blood group) | | |
| 0.00000064 | 0.000116 | <1e−07 | 62.93 | 26.13 | 2.41 | WDR74 | WD repeat | 54663 | NM_001307977 |
| | | | | | | | domain 74 | | |
| 0.0000065 | 0.000117 | <1e−07 | 52.45 | 23.79 | 2.21 | PAG1 | phosphoprotein | 55824 | NM_018440 |
| | | | | | | | membrane anchor | | |
| | | | | | | | with | | |
| | | | | | | | glycosphingolipid | | |
| | | | | | | | microdomains 1 | | |
| 0.0000065 | 0.000117 | <1e−07 | 47.62 | 15.64 | 3.05 | TNRC18P1 | trinucleotide | 644962 | NR_077215 |
| | | | | | | | repeat containing | | |
| | | | | | | | 18 pseudogene 1 | | |
| 0.0000065 | 0.000117 | <1e−07 | 80.15 | 24.98 | 3.21 | WHRN | whirlin | 25861 | NM_001083885 |
| 0.0000066 | 0.000118 | <1e−07 | 165.71 | 62.26 | 2.66 | KLF2 | Kruppel like | 10365 | NM_016270 |
| | | | | | | | factor 2 | | |
| 0.0000066 | 0.000118 | <1e−07 | 82.73 | 36.66 | 2.26 | DOCK8 | dedicator of | 81704 | NM_001190458 |
| | | | | | | | cytokinesis 8 | | |
| 0.0000067 | 0.00012 | <1e−07 | 51.58 | 24.08 | 2.14 | WDR81 | WD repeat | 124997 | NM_001163673 |
| | | | | | | | domain 81 | | |
| 0.0000068 | 0.000121 | <1e−07 | 80.91 | 39.22 | 2.06 | ACAP3 | ArfGAP with | 116983 | NM_030649 |
| | | | | | | | coiled-coil, | | |
| | | | | | | | ankyrin repeat and | | |
| | | | | | | | PH domains 3 | | |
| 0.0000068 | 0.000121 | <1e−07 | 73.8 | 34.78 | 2.12 | ANXA6 | annexin A6 | 309 | NM_001155 |
| 0.0000068 | 0.000121 | <1e−07 | 67.13 | 25.62 | 2.62 | GRASP | general receptor | 160622 | NM_001271856 |
| | | | | | | | for | | |
| | | | | | | | phosphoinositides | | |
| | | | | | | | 1 associated | | |
| | | | | | | | scaffold protein | | |
| 0.0000069 | 0.000122 | <1e−07 | 23.94 | 11.82 | 2.03 | SLC35C2 | solute carrier | 51006 | NM_001281457 |
| | | | | | | | family 35 member | | |
| | | | | | | | C2 | | |
| 0.000007 | 0.000124 | <1e−07 | 6.46 | 19.64 | 0.33 | ALDOB | aldolase, fructose- | 229 | NM_000035 |
| | | | | | | | bisphosphate B | | |
| 0.0000071 | 0.000125 | <1e−07 | 7.09 | 14.97 | 0.47 | PLCE1 | phospholipase C | 51196 | NM_001165979 |
| | | | | | | | epsilon 1 | | |
| 0.0000072 | 0.000126 | <1e−07 | 49.01 | 17.35 | 2.83 | SNHG22 | small nucleolar | 103091864 | NR_117096 |
| | | | | | | | RNA host gene 22 | | |
| 0.00000072 | 0.000126 | <1e−07 | 38.43 | 15.38 | 2.5 | FGD2 | FYVE, RhoGF | 221472 | NM_173558 |
| | | | | | | | and PH domain | | |
| | | | | | | | containing 2 | | |
| 0.0000072 | 0.000126 | <1e−07 | 21.23 | 9.3 | 2.28 | CABLES1 | Cdk5 and Abl | 91768 | NM_001100619 |
| | | | | | | | enzyme substrate | | |
| | | | | | | | 1 | | |
| 0.0000072 | 0.000126 | <1e−07 | 6.48 | 15.71 | 0.41 | FREM1 | FRAS1 related | 158326 | NM_001177704 |
| | | | | | | | extracellular | | |
| | | | | | | | matrix 1 | | |
| 0.0000073 | 0.000127 | <1e−07 | 80.01 | 30.4 | 2.63 | GPR132 | G protein-coupled | 29933 | NM_001278694 |
| | | | | | | | receptor 132 | | |
| 0.0000074 | 0.000128 | <1e−07 | 159.49 | 61.33 | 2.6 | IRF4 | interferon | 3662 | NM_001195286 |
| | | | | | | | regulatory factor 4 | | |
| 0.0000075 | 0.00013 | <1e−07 | 81.41 | 40.05 | 2.03 | KDM5A | lysine | 5927 | NM_001042603 |
| | | | | | | | demethylase 5A | | |
| 0.0000077 | 0.000133 | <1e−07 | 94.09 | 43.02 | 2.19 | PPRC1 | peroxisome | 23082 | NM_001288727 |
| | | | | | | | proliferator- | | |
| | | | | | | | activated receptor | | |
| | | | | | | | gamma, | | |
| | | | | | | | coactivator-related | | |
| | | | | | | | 1 | | |
| 0.0000079 | 0.000136 | <1e−07 | 104.95 | 46.46 | 2.26 | SMG1P3 | SMG1 | 100271836 | NR_027155 |
| | | | | | | | pseudogene 3 | | |
| 0.000008 | 0.000137 | <1e−07 | 14.79 | 5.89 | 2.51 | LINC00926 | long intergenic | 283663 | NR_024433 |
| | | | | | | | non-protein | | |
| | | | | | | | coding RNA 926 | | |
| 0.000008 | 0.000137 | <1e−07 | 26.01 | 12.59 | 2.07 | CCDC85B | coiled-coil | 11007 | NM_006848 |
| | | | | | | | domain containing | | |
| | | | | | | | 85B | | |
| 0.0000082 | 0.00014 | <1e−07 | 33.65 | 15.07 | 2.23 | RNF166 | ring finger protein | 115992 | NM_001171815 |
| | | | | | | | 166 | | |
| 0.0000082 | 0.00014 | <1e−07 | 51.05 | 23.06 | 2.21 | NPC1 | NPC intra cellular | 4864 | NM_000271 |
| | | | | | | | cholesterol | | |
| | | | | | | | transporter 1 | | |
| 0.0000085 | 0.000142 | <1e−07 | 24.52 | 11.26 | 2.18 | SETDB2 | SET domain | 83852 | NM_001160308 |
| | | | | | | | bifurcated 2 | | |
| 0.0000085 | 0.000142 | 0.0001 | 220.89 | 106.72 | 2.07 | HIVEP2 | human | 3097 | NM_006734 |
| | | | | | | | immunodeficiency | | |
| | | | | | | | virus type I | | |
| | | | | | | | enhancer binding | | |
| | | | | | | | protein 2 | | |
| 0.0000085 | 0.000142 | <1e−07 | 123.22 | 55.79 | 2.21 | SH3BP5 | SH3 domain | 9467 | NM_001018009 |
| | | | | | | | binding protein 5 | | |
| 0.0000086 | 0.000144 | <1e−07 | 60.93 | 29.65 | 2.05 | PPP2R5C | protein | 5527 | NM_001161725 |
| | | | | | | | phosphatase 2 | | |
| | | | | | | | regulatory subunit | | |
| | | | | | | | B'gamma | | |
| 0.0000087 | 0.000145 | <1e−07 | 34.23 | 16.86 | 2.03 | TESK1 | testis associated | 7016 | NM_001318230 |
| | | | | | | | actin remodelling | | |
| | | | | | | | kinase 1 | | |
| 0.0000087 | 0.000145 | <1e−07 | 76.28 | 36.09 | 2.11 | SEPTIN6 | Septin 6 | 23157 | |
| 0.0000088 | 0.000146 | <1e−07 | 41.61 | 15.65 | 2.66 | P2RX1 | purinergic | 5023 | NM_002558 |
| | | | | | | | receptor P2X 1 | | |
| 0.0000089 | 0.000147 | <1e−07 | 20.2 | 8.14 | 2.48 | RNVU1-27 | RNA, Variant U1 | 115409988 | |
| | | | | | | | Small Nuclear 27 | | |
| 0.0000091 | 0.00015 | <1e−07 | 106.08 | 45.85 | 2.31 | NIBAN1 | Niban Apoptosis | 116496 | |
| | | | | | | | Regulator 1 | | |
| 0.0000092 | 0.000151 | <1e−07 | 21.56 | 57.17 | 0.38 | KLF5 | Kruppel like | 688 | NM_001286818 |
| | | | | | | | factor 5 | | |
| 0.0000094 | 0.000154 | <1e−07 | 33.73 | 16.29 | 2.07 | PSMA3- | PSMA3 antisense | 379025 | NR_029434 |
| | | | | | | AS1 | RNA 1 | | |
| 0.0000095 | 0.000154 | <1e−07 | 119.95 | 54.71 | 2.19 | RSRP1 | arginine and | 57035 | NM_001321772 |
| | | | | | | | serine rich protein | | |
| | | | | | | | 1 | | |
| 0.0000095 | 0.000154 | <1e−07 | 5.88 | 13.73 | 0.43 | MT-TW | Mitochondrially | 4578 | |
| | | | | | | | Encoded TRNA- | | |
| | | | | | | | Trp (UGA/G) | | |
| 0.0000097 | 0.000156 | <1e−07 | 857.82 | 410.38 | 2.09 | MCL1 | MCL1, BCL2 | 4170 | NM_001197320 |
| | | | | | | | family apoptosis | | |
| | | | | | | | regulator | | |
| 0.0000099 | 0.000159 | <1e−07 | 7.24 | 18.55 | 0.39 | CDH17 | cadherin 17 | 1015 | NM_001144663 |
| 0.0000099 | 0.000159 | 0.0001 | 29.54 | 14.08 | 2.1 | MAGT1 | magnesium | 84061 | NM_032121 |
| | | | | | | | transporter 1 | | |
| 0.00001 | 0.000159 | <1e−07 | 24.6 | 10.99 | 2.24 | QSOX2 | quiescin | 169714 | NM_181701 |
| | | | | | | | sulfhydryloxidase | | |
| | | | | | | | 2 | | |
| 0.00001 | 0.000159 | <1e−07 | 20.27 | 9.72 | 2.09 | RNF122 | ring finger protein | 79845 | NM_024787 |
| | | | | | | | 122 | | |
| 0.00001 | 0.000159 | <1e−07 | 26.99 | 13.1 | 2.06 | XPOT | exportin for tRNA | 11260 | NM_007235 |
| 0.00001 | 0.000159 | <1e−07 | 62.4 | 25.52 | 2.44 | ST3GAL1 | ST3 beta- | 6482 | NM_003033 |
| | | | | | | | galactoside alpha- | | |
| | | | | | | | 2,3- | | |
| | | | | | | | sialyltransferase 1 | | |
| 0.00001 | 0.000159 | <1e−07 | 27.37 | 13.21 | 2.07 | E4F1 | E4F transcription | 1877 | NM_001288776 |
| | | | | | | | factor 1 | | |
| 0.0000101 | 0.00016 | <1e−07 | 70.78 | 33.39 | 2.12 | REXO1 | RNA exonuclease | 57455 | NM_020695 |
| | | | | | | | 1 homolog | | |
| 0.0000103 | 0.000163 | <1e−07 | 29 | 12.63 | 2.3 | AC117382.1 | noncoding (Clone | | |
| | | | | | | | Fragment ID: | | |
| | | | | | | | AC117382.10) | | |
| 0.0000106 | 0.000167 | <1e−07 | 39.83 | 10.75 | 3.71 | SNORD3B- | small nucleolar | 26851 | NR_003271 |
| | | | | | | 1 | RNA, C/D box | | |
| | | | | | | | 3B-1 | | |
| 0.0000106 | 0.000167 | <1e−07 | 20.07 | 8.51 | 2.36 | AC109326.1 | noncoding | | |
| | | | | | | | (Ensemble gene | | |
| | | | | | | | ID: | | |
| | | | | | | | ENSG00000225584) | | |
| 0.0000106 | 0.000167 | <1e−07 | 85.01 | 38.82 | 2.19 | ATP6V0D1 | ATPase H+ | 9114 | NM_004691 |
| | | | | | | | transporting V0 | | |
| | | | | | | | subunit d1 | | |
| 0.0000106 | 0.000167 | <1e−07 | 32.72 | 13.96 | 2.34 | ABCG1 | ATP binding | 9619 | NM_004915 |
| | | | | | | | cassette subfamily | | |
| | | | | | | | G member 1 | | |
| 0.0000107 | 0.000168 | <1e−07 | 3340.15 | 1176.19 | 2.84 | NEAT1 | nuclear | 283131 | NR_002802 |
| | | | | | | | paraspeckle | | |
| | | | | | | | assembly | | |
| | | | | | | | transcript 1 (non- | | |
| | | | | | | | protein coding) | | |
| 0.0000107 | 0.000168 | <1e−07 | 5.71 | 12.19 | 0.47 | MTND5P11 | mitochondrially | 100506169 | |
| | | | | | | | encoded | | |
| | | | | | | | NADH: ubiquinone | | |
| | | | | | | | oxidoreductase | | |
| | | | | | | | core subunit 5 | | |
| | | | | | | | pseudogene 11 | | |
| 0.0000107 | 0.000168 | <1e−07 | 95.66 | 35.11 | 2.73 | ITGA5 | integrin subunit | 3678 | NM_002205 |
| | | | | | | | alpha 5 | | |
| 0.0000108 | 0.000169 | <1e−07 | 58.69 | 17.64 | 3.33 | CCR7 | C-C motif | 1236 | NM_001301714 |
| | | | | | | | chemokine | | |
| | | | | | | | receptor 7 | | |
| 0.0000113 | 0.000175 | 0.0001 | 464.93 | 227.95 | 2.04 | DDX3X | DAD-box | 1654 | NM_001193416 |
| | | | | | | | helicase 3, X- | | |
| | | | | | | | linked | | |
| 0.0000113 | 0.000175 | <1e−07 | 52.51 | 23.02 | 2.28 | ANKRD44 | ankyrin repeat | 91526 | NM_001195144 |
| | | | | | | | domain 44 | | |
| 0.0000114 | 0.000177 | <1e−07 | 44.75 | 20.6 | 2.17 | MAML2 | mastermind like | 84441 | NM_032427 |
| | | | | | | | transcriptional | | |
| | | | | | | | coactivator 2 | | |
| 0.0000117 | 0.000181 | <1e−07 | 5.93 | 12.53 | 0.47 | DHRS11 | dehydrogenase/ | 79154 | NM_024308 |
| | | | | | | | reductase 11 | | |
| 0.000012 | 0.000184 | <1e−07 | 176.46 | 81.74 | 2.16 | PTK2B | protein tyrosine | 2185 | NM_004103 |
| | | | | | | | kinase 2 beta | | |
| 0.0000121 | 0.000185 | <1e−07 | 47.41 | 22.64 | 2.09 | BMF | Bcl2 modifying | 90427 | NM_001003940 |
| | | | | | | | factor | | |
| 0.0000121 | 0.000185 | <1e−07 | 81.41 | 39.73 | 2.05 | CTBP1 | C-terminal | 1487 | NM_001012614 |
| | | | | | | | binding protein 1 | | |
| 0.0000121 | 0.000185 | <1e−07 | 29.92 | 11.65 | 2.57 | AC018445.3 | noncoding | | |
| | | | | | | | (Ensemble gene | | |
| | | | | | | | ID: | | |
| | | | | | | | ENSG00000279637.1) | | |
| 0.0000122 | 0.000186 | <1e−07 | 37.91 | 17.96 | 2.11 | ACOT9 | acyl-CoA | 23597 | NM_001033583 |
| | | | | | | | thioesterase 9 | | |
| 0.0000124 | 0.000188 | <1e−07 | 33.49 | 13.67 | 2.45 | CHPF | chondroitin | 79586 | NM_001195731 |
| | | | | | | | polymerizing | | |
| | | | | | | | factor | | |
| 0.0000127 | 0.000192 | <1e−07 | 54.73 | 25.66 | 2.13 | CDK17 | cyclin dependent | 5128 | NM_001170464 |
| | | | | | | | kinase 17 | | |
| 0.0000128 | 0.000192 | <1e−07 | 42.73 | 20.17 | 2.12 | RDX | radixin | 5962 | NM_001260492 |
| 0.0000128 | 0.000192 | <1e−07 | 35.11 | 11 | 3.19 | IFITM10 | interferon induced | 402778 | NM_001170820 |
| | | | | | | | transmembrane | | |
| | | | | | | | protein 10 | | |
| 0.0000128 | 0.000192 | <1e−07 | 35.32 | 16.9 | 2.09 | FBXW4 | F-box and WD | 6468 | NM_001323541 |
| | | | | | | | repeat domain | | |
| | | | | | | | containing 4 | | |
| 0.0000134 | 0.000199 | <1e−07 | 70.92 | 33.98 | 2.09 | MAN2C1 | mannosidase | 4123 | NM_001256494 |
| | | | | | | | alpha class 2C | | |
| | | | | | | | member 1 | | |
| 0.0000136 | 0.000202 | <1e−07 | 70.66 | 32.03 | 2.21 | SNX9 | sorting nexin 9 | 51429 | NM_016224 |
| 0.0000138 | 0.000203 | <1e−07 | 24.6 | 10.5 | 2.34 | ISYNA1 | inositol-3- | 51477 | NM_001170938 |
| | | | | | | | phosphate | | |
| | | | | | | | synthase 1 | | |
| 0.0000138 | 0.000203 | <1e−07 | 6.9 | 17.65 | 0.39 | CACNA1A | calcium voltage- | 773 | NM_000068 |
| | | | | | | | gated channel | | |
| | | | | | | | subunit alpha 1 A | | |
| 0.000014 | 0.000205 | <1e−07 | 23.63 | 11.46 | 2.06 | PRKD3 | protein kinase D3 | 23683 | NM_005813 |
| 0.0000144 | 0.00021 | <1e−07 | 1985.17 | 991 | 2 | SRRM2 | serine/arginine | 23524 | NM_016333 |
| | | | | | | | repetitive matrix 2 | | |
| 0.0000146 | 0.000211 | <1e−07 | 96.23 | 36.13 | 2.66 | PIK3CG | phosphatidylinositol- | 5294 | NM_001282426 |
| | | | | | | | 4,5- | | |
| | | | | | | | bisphosphate 3- | | |
| | | | | | | | kinase catalytic | | |
| | | | | | | | subunit gamma | | |
| 0.0000146 | 0.000211 | <1e−07 | 108.18 | 36.83 | 2.94 | SEMA4A | semaphorin 4A | 64218 | NM_001193300 |
| 0.0000146 | 0.000211 | 0.0001 | 29.98 | 12.04 | 2.49 | AC016831.1 | noncoding | | |
| | | | | | | | (Ensemble gene | | |
| | | | | | | | ID: | | |
| | | | | | | | ENSG00000226380.9) | | |
| 0.0000149 | 0.000215 | <1e−07 | 37.97 | 16.63 | 2.28 | PITPNC1 | phosphatidylinositol | 26207 | NM_012417 |
| | | | | | | | transfer protein | | |
| | | | | | | | cytoplasmic 1 | | |
| 0.0000151 | 0.000217 | 0.0001 | 148.09 | 56.65 | 2.61 | EIF2AK3 | eukaryotic | 9451 | NM_001313915 |
| | | | | | | | translation | | |
| | | | | | | | initiation factor 2 | | |
| | | | | | | | alpha kinase 3 | | |
| 0.0000152 | 0.000218 | <1e−07 | 130.02 | 64.42 | 2.02 | FMNL3 | formin like 3 | 91010 | NM_175736 |
| 0.0000154 | 0.00022 | <1e−07 | 105.31 | 49.86 | 2.11 | CCNL2 | cyclin L2 | 81669 | NM_001039577 |
| 0.0000156 | 0.000222 | <1e−07 | 47.4 | 22.52 | 2.1 | ADCY3 | adenylate cyclase | 109 | NM_001320613 |
| | | | | | | | 3 | | |
| 0.00000156 | 0.000222 | <1e−07 | 176.37 | 83.89 | 2.1 | WIPF1 | WAS/WASL | 7456 | NM_001077269 |
| | | | | | | | interacting protein | | |
| | | | | | | | family member 1 | | |
| 0.00000156 | 0.000222 | <1e−07 | 27.21 | 12.11 | 2.25 | HEXA | hexosaminidase | 3073 | NM_000520 |
| | | | | | | | subunit alpha | | |
| 0.00000158 | 0.000224 | 0.0001 | 10.18 | 27.39 | 0.37 | HELLPAR | HELLP associated | 101101692 | |
| | | | | | | | long non-coding | | |
| | | | | | | | RNA | | |
| 0.0000159 | 0.000225 | <1e−07 | 172.38 | 58.73 | 2.93 | HLA-DRB1 | major | 3123 | NM_001243965 |
| | | | | | | | histocompatibility | | |
| | | | | | | | complex, class II, | | |
| | | | | | | | DR beta 1 | | |
| 0.0000162 | 0.000228 | <1e−07 | 29.49 | 14.2 | 2.08 | KLF16 | Kruppel like | 83855 | NM_031918 |
| | | | | | | | factor 16 | | |
| 0.0000163 | 0.000229 | <1e−07 | 79.64 | 36.78 | 2.17 | TP53INP1 | tumor protein p53 | 94241 | NM_001135733 |
| | | | | | | | inducible nuclear | | |
| | | | | | | | protein 1 | | |
| 0.0000165 | 0.000231 | <1e−07 | 134.74 | 61.54 | 2.19 | OGA | O-GlcNAcase | 10724 | |
| 0.0000169 | 0.000235 | <1e−07 | 45.46 | 20.63 | 2.2 | FMNL2 | formin like 2 | 114793 | NM_001004417 |
| 0.0000169 | 0.000235 | <1e−07 | 5.91 | 14.4 | 0.41 | NGEF | neuronal guanine | 25791 | NM_001114090 |
| | | | | | | | nucleotide | | |
| | | | | | | | exchange factor | | |
| 0.0000171 | 0.000237 | <1e−07 | 18.55 | 7.69 | 2.41 | EBF1 | early B cell factor | 1879 | NM_001290360 |
| | | | | | | | 1 | | |
| 0.0000172 | 0.000237 | <1e−07 | 74.99 | 36.47 | 2.06 | SLC6A6 | solute carrier | 6533 | NM_001134367 |
| | | | | | | | family 6 member | | |
| | | | | | | | 6 | | |
| 0.0000173 | 0.000238 | <1e−07 | 63.44 | 31.49 | 2.01 | DGKD | diacylglycerol | 8527 | NM_003648 |
| | | | | | | | kinase delta | | |
| 0.0000173 | 0.000238 | 0.0001 | 74.43 | 34.14 | 2.18 | HBP1 | HMG-box | 26959 | NM_001244262 |
| | | | | | | | transcription | | |
| | | | | | | | factor 1 | | |
| 0.0000173 | 0.000238 | <1e−07 | 49.42 | 23.71 | 2.08 | ELMO1 | engulfment and | 9844 | NM_001039459 |
| | | | | | | | cell motility 1 | | |
| 0.0000174 | 0.000239 | 0.0001 | 17.92 | 7.9 | 2.27 | AL021155.5 | noncoding (Clone | | |
| | | | | | | | Fragment ID: | | |
| | | | | | | | AL021155.1) | | |
| 0.0000173 | 0.00025 | <1e−07 | 130.03 | 61.66 | 2.11 | TUBGCP6 | tubulin gamma | 85378 | NM_001008658 |
| | | | | | | | complex | | |
| | | | | | | | associated protein | | |
| | | | | | | | 6 | | |
| 0.0000187 | 0.000253 | 0.0002 | 201.17 | 85.69 | 2.35 | ZBTB10 | zinc finger and | 65986 | NM_001105539 |
| | | | | | | | BTB domain | | |
| | | | | | | | containing 10 | | |
| 0.0000187 | 0.000253 | <1e−07 | 34.49 | 15.53 | 2.22 | LIMD2 | LIM domain | 80774 | NM_030576 |
| | | | | | | | containing 2 | | |
| 0.000019 | 0.000256 | <1e−07 | 7.19 | 14.6 | 0.49 | SOX6 | SRY-box 6 | 55553 | NM_001145811 |
| 0.0000194 | 0.00026 | <1e−07 | 64.95 | 23.04 | 2.82 | POU2AF1 | POU class 2 | 5450 | NM_006235 |
| | | | | | | | associating factor | | |
| | | | | | | | 1 | | |
| 0.0000195 | 0.000261 | <1e−07 | 50.45 | 17.95 | 2.81 | DUSP4 | dual specificity | 1846 | NM_001394 |
| | | | | | | | phosphatase 4 | | |
| 0.0000199 | 0.000265 | <1e−07 | 87.68 | 37.13 | 2.36 | YPEL5 | yippee like 5 | 51646 | NM_001127399 |
| 0.00002 | 0.000266 | <1e−07 | 320.11 | 148.83 | 2.15 | EHMT1 | euchromatic | 79813 | NM_001039765 |
| | | | | | | | histone lysine | | |
| | | | | | | | methyltransferase | | |
| | | | | | | | 1 | | |
| 0.0000204 | 0.000269 | <1e−07 | 20.03 | 9.88 | 2.03 | KLHL5 | kelch like family | 51088 | NM_001007075 |
| | | | | | | | member 5 | | |
| 0.0000204 | 0.000269 | 0.0001 | 7.38 | 20.07 | 0.37 | SPIRE2 | spire type actin | 84501 | NM_032451 |
| | | | | | | | nucleation factor 2 | | |
| 0.0000205 | 0.00027 | <1e−07 | 5.68 | 11.43 | 0.5 | CHDH | choline | 55349 | NM_018397 |
| | | | | | | | dehydrogenase | | |
| 0.0000207 | 0.000272 | <1e−07 | 44.26 | 20.84 | 2.12 | TAF1C | TATA-box | 9013 | NM_001243156 |
| | | | | | | | binding protein | | |
| | | | | | | | associated factor, | | |
| | | | | | | | RNA polymerase | | |
| | | | | | | | I subunit C | | |
| 0.0000209 | 0.000274 | <1e−07 | 6.65 | 20.03 | 0.33 | MYO1A | myosin IA | 4640 | NM_001256041 |
| 0.000021 | 0.000275 | <1e−07 | 10.44 | 55.9 | 0.19 | DMBT1 | deleted in | 1755 | NM_001320644 |
| | | | | | | | malignant brain | | |
| | | | | | | | tumors 1 | | |
| 0.0000211 | 0.000276 | <1e−07 | 5.62 | 11.96 | 0.47 | NR1I2 | nuclear receptor | 8856 | NM_003889 |
| | | | | | | | subfamily 1 group | | |
| | | | | | | | I member 2 | | |
| 0.0000216 | 0.00028 | <1e−07 | 77.2 | 31 | 2.49 | ARHGAP9 | Rho GTPase | 64333 | NM_001080156 |
| | | | | | | | activating protein | | |
| | | | | | | | 9 | | |
| 0.0000217 | 0.000281 | 0.0002 | 68.73 | 29.16 | 2.36 | MYO1G | myosin IG | 64005 | NM_033054 |
| 0.0000217 | 0.000281 | 0.0001 | 351.25 | 171.2 | 2.05 | ADGR5 | adhesion G | 976 | NM_001025160 |
| | | | | | | | protein-coupled | | |
| | | | | | | | receptor 5 | | |
| 0.0000218 | 0.000282 | <1e−07 | 130.23 | 59.14 | 2.2 | PIK3R5 | phosphoinositide- | 23533 | NM_001142633 |
| | | | | | | | 3-kinase | | |
| | | | | | | | regulatory subunit | | |
| | | | | | | | 5 | | |
| 0.0000221 | 0.000285 | <1e−07 | 15.48 | 7.56 | 2.05 | TNFRSF4 | TNF receptor | 7293 | NM_003327 |
| | | | | | | | superfamily | | |
| | | | | | | | member 4 | | |
| 0.0000224 | 0.000288 | 0.0001 | 18.35 | 9.1 | 2.02 | AC013394.1 | noncoding | | |
| | | | | | | | (Ensemble gene | | |
| | | | | | | | ID: | | |
| | | | | | | | ENSG00000272888.1) | | |
| 0.0000231 | 0.000296 | <1e−07 | 6.33 | 14.5 | 0.44 | MYRF | myelin regulatory | 745 | NM_001127392 |
| | | | | | | | factor | | |
| 0.0000233 | 0.000297 | 0.0001 | 12.75 | 28.33 | 0.45 | NFIB | nuclear factor I B | 4781 | NM_001190737 |
| 0.0000233 | 0.000297 | <1e−07 | 5.94 | 13.06 | 0.46 | RAP1GAP | RAPI GTPase | 5909 | NM_001145657 |
| | | | | | | | activating protein | | |
| 0.0000234 | 0.000297 | <1e−07 | 6.14 | 12.93 | 0.48 | MGAM2 | maltase- | 93432 | NM_001008748 |
| | | | | | | | glucoamylase 2 | | |
| | | | | | | | (putative) | | |
| 0.0000235 | 0.000298 | 0.0001 | 56.98 | 15.81 | 3.6 | SNORD3B- | small nucleolar | 780852 | NR_003924 |
| | | | | | | 2 | RNA, C/D box | | |
| | | | | | | | 3B-2 | | |
| 0.0000238 | 0.000301 | <1e−07 | 6.9 | 15.36 | 0.45 | PARP3 | poly (ADP-ribose) | 10039 | NM_001003931 |
| | | | | | | | polymerase family | | |
| | | | | | | | member 3 | | |
| 0.0000241 | 0.000304 | <1e−07 | 12.72 | 5.79 | 2.2 | AC090617.10 | noncoding | | |
| | | | | | | | (Ensemble gene | | |
| | | | | | | | ID: | | |
| | | | | | | | ENSG00000287553.1) | | |
| 0.0000245 | 0.000308 | <1e−07 | 5.43 | 10.96 | 0.5 | AL356534.1 | noncoding | | |
| | | | | | | | (Ensemble gene | | |
| | | | | | | | ID: | | |
| | | | | | | | ENSG00000286215.1) | | |
| 0.0000252 | 0.000314 | <1e−07 | 133.58 | 38.97 | 3.43 | SEMA6B | semaphorin 6B | 10501 | NM_020241 |
| 0.0000254 | 0.000316 | <1e−07 | 27.3 | 12.21 | 2.24 | BMP6 | bone | 654 | NM_001718 |
| | | | | | | | morphogenetic | | |
| | | | | | | | protein 6 | | |
| 0.0000258 | 0.00032 | <1e−07 | 22.6 | 10.7 | 2.11 | RABGEF1 | RAB guanine | 27342 | NM_001287060 |
| | | | | | | | nucleotide | | |
| | | | | | | | exchange factor 1 | | |
| 0.0000263 | 0.000324 | <1e−07 | 49.83 | 22.01 | 2.26 | CHST2 | carbohydrate | 9435 | NM_004267 |
| | | | | | | | sulfotransferase 2 | | |
| 0.0000264 | 0.000325 | <1e−07 | 5.67 | 13.57 | 0.42 | ENPEP | glutamyl | 2028 | NM_001977 |
| | | | | | | | aminopeptidase | | |
| 0.0000266 | 0.000327 | <1e−07 | 29.9 | 13.76 | 2.17 | METTL7A | methyltransferase | 25840 | NM_014033 |
| | | | | | | | like 7A | | |
| 0.0000269 | 0.000329 | <1e−07 | 43.46 | 21.31 | 2.04 | CHST11 | carbohydrate | 50515 | NM_001173982 |
| | | | | | | | sulfotransferase | | |
| | | | | | | | 11 | | |
| 0.0000271 | 0.000331 | <1e−07 | 58.32 | 25.33 | 2.3 | PIK3CD | phosphatidylinositol- | 5293 | NM_001350234 |
| | | | | | | | 4,5- | | |
| | | | | | | | bisphosphate 3- | | |
| | | | | | | | kinase catalytic | | |
| | | | | | | | subunit delta | | |
| 0.0000274 | 0.000334 | 0.0001 | 36.25 | 14.01 | 2.59 | SPAG4 | sperm associated | 6676 | NM_001317931 |
| | | | | | | | antigen 4 | | |
| 0.0000274 | 0.000334 | <1e−07 | 43.55 | 13.81 | 3.15 | CD22 | CD22 molecule | 933 | NM_001185099 |
| 0.0000275 | 0.000335 | <1e−07 | 96.44 | 38.99 | 2.47 | HIVEP3 | human | 59269 | NM_001127714 |
| | | | | | | | immunodeficiency | | |
| | | | | | | | virus type I | | |
| | | | | | | | enhancer binding | | |
| | | | | | | | protein 3 | | |
| 0.0000278 | 0.00038 | <1e−07 | 233.44 | 713.53 | 0.33 | MTND2P28 | mitochondrially | 100652939 | |
| | | | | | | | encoded | | |
| | | | | | | | NADH: ubiquinone | | |
| | | | | | | | oxidoreductase | | |
| | | | | | | | core subunit 2 | | |
| | | | | | | | pseudogene 28 | | |
| 0.0000278 | 0.000338 | <1e−07 | 21.05 | 8.44 | 2.49 | ADGRB1 | adhesion G | 575 | NM_001702 |
| | | | | | | | protein-coupled | | |
| | | | | | | | receptor B1 | | |
| 0.0000278 | 0.000342 | 0.0001 | 24.21 | 10.62 | 2.28 | HLA-DMA | major | 3108 | NM_006120 |
| | | | | | | | histocompatibility | | |
| | | | | | | | complex, class II, | | |
| | | | | | | | DM alpha | | |
| 0.0000282 | 0.000342 | 0.0001 | 27.27 | 12.85 | 2.12 | CBFA2T3 | CBFA2/RUNX1 | 863 | NM_005187 |
| | | | | | | | translocation | | |
| | | | | | | | partner 3 | | |
| 0.0000283 | 0.000343 | <1e−07 | 180.5 | 89.17 | 2.02 | GOLGA3 | golgin A3 | 2802 | NM_001172557 |
| 0.0000284 | 0.000343 | <1e−07 | 8.53 | 20.08 | 0.43 | MDK | midkine | 4192 | NM_001012333 |
| 0.0000285 | 0.000343 | <1e−07 | 31.55 | 14.83 | 2.13 | CTC1 | CST telomere | 80169 | NM_025099 |
| | | | | | | | replication | | |
| | | | | | | | complex | | |
| | | | | | | | component 1 | | |
| 0.0000286 | 0.000344 | <1e−07 | 19.1 | 9.12 | 2.1 | FAM53B | family with | 9679 | NM_014661 |
| | | | | | | | sequence | | |
| | | | | | | | similarity 53 | | |
| | | | | | | | member B | | |
| 0.0000288 | 0.000346 | 0.0001 | 72.53 | 21.39 | 3.39 | TRPV3 | transient receptor | 162514 | NM_001258205 |
| | | | | | | | potential cation | | |
| | | | | | | | channel subfamily | | |
| | | | | | | | V member 3 | | |
| 0.0000292 | 0.00035 | <1e−07 | 11.11 | 32.01 | 0.35 | CRACD | Capping Protein | 57482 | |
| | | | | | | | Inhibiting | | |
| | | | | | | | Regulator Of | | |
| | | | | | | | Actin Dynamics | | |
| 0.0000298 | 0.000354 | 0.0001 | 52.07 | 25.94 | 2.01 | ARHGAP25 | Rho GTPase | 9938 | NM_001007231 |
| | | | | | | | activating protein | | |
| | | | | | | | 25 | | |
| 0.0000301 | 0.000356 | <1e−07 | 31.03 | 13.6 | 2.28 | TMEM131L | transmembrane | 23240 | NM_001131007 |
| | | | | | | | 131 like | | |
| 0.0000305 | 0.00036 | 0.0001 | 82.04 | 39.79 | 2.06 | FAM102A | family with | 399665 | NM_001035254 |
| | | | | | | | sequence | | |
| | | | | | | | similarity 102 | | |
| | | | | | | | member A | | |
| 0.0000313 | 0.000367 | <1e−07 | 79.56 | 35.63 | 2.23 | ISG20 | interferon | 3669 | NM_001303233 |
| | | | | | | | stimulated | | |
| | | | | | | | exonuclease gene | | |
| | | | | | | | 20 | | |
| 0.0000313 | 0.000367 | 0.0001 | 47.08 | 21.08 | 2.23 | NCF4 | neutrophil | 4689 | NM_000631 |
| | | | | | | | cytosolic factor 4 | | |
| 0.0000324 | 0.000377 | <1e−07 | 23.2 | 10.94 | 2.12 | TMEM273 | transmembrane | 170371 | NM_001010863 |
| | | | | | | | protein 273 | | |
| 0.0000331 | 0.000384 | <1e−07 | 43.02 | 11.88 | 3.62 | IGHG4 | immunoglobulin | 3503 | |
| | | | | | | | heavy constant | | |
| | | | | | | | gamma 4 (G4m | | |
| | | | | | | | marker) | | |
| 0.0000332 | 0.000384 | <1e−07 | 23.3 | 11.36 | 2.05 | ORMDL3 | ORMDL | 94103 | NM_001320801 |
| | | | | | | | sphingolipid | | |
| | | | | | | | biosynthesis | | |
| | | | | | | | regulator 3 | | |
| 0.0000334 | 0.000386 | <1e−07 | 63.09 | 28.83 | 2.19 | MIB2 | mindbomb E3 | 142678 | NM_001170686 |
| | | | | | | | ubiquitin protein | | |
| | | | | | | | ligase 2 | | |
| 0.0000335 | 0.000386 | <1e−07 | 609.29 | 282.44 | 2.16 | SQSTM1 | sequestosome 1 | 8878 | NM_001142298 |
| 0.0000336 | 0.000387 | <1e−07 | 23.46 | 10.54 | 2.23 | KCNA3 | potassium | 3738 | NM_002232 |
| | | | | | | | voltage-gated | | |
| | | | | | | | channel subfamily | | |
| | | | | | | | A member 3 | | |
| 0.0000339 | 0.000389 | 0.0001 | 68.78 | 28.31 | 2.43 | SESN2 | sestrin 2 | 83667 | NM_031459 |
| 0.0000346 | 0.000395 | <1e−07 | 208.68 | 85.11 | 2.45 | GAK | cyclin G | 2580 | NM_001286833 |
| | | | | | | | associated kinase | | |
| 0.0000347 | 0.000396 | 0.0002 | 71.01 | 26.27 | 2.7 | AC131212.3 | noncoding | | |
| | | | | | | | (Ensemble gene | | |
| | | | | | | | ID: | | |
| | | | | | | | ENSG00000280287.1) | | |
| 0.0000349 | 0.000397 | <1e−07 | 50.95 | 24.35 | 2.09 | DNAJB11 | DnaJ heat shock | 51726 | NM_016306 |
| | | | | | | | protein family | | |
| | | | | | | | (Hsp40) member | | |
| | | | | | | | B11 | | |
| 0.0000353 | 0.000401 | <1e−07 | 24.71 | 12.11 | 2.04 | RRN3P3 | RRN3 homolog, | 100131998 | NR_027460 |
| | | | | | | | RNA polymerase | | |
| | | | | | | | I transcription | | |
| | | | | | | | factor pseudo gene | | |
| | | | | | | | 3 | | |
| 0.0000356 | 0.000404 | <1e−07 | 212.26 | 88.74 | 2.39 | MAPK8IP3 | mitogen-activated | 23162 | NM_001040439 |
| | | | | | | | protein kinase 8 | | |
| | | | | | | | interacting protein | | |
| | | | | | | | 3 | | |
| 0.0000359 | 0.000406 | <1e−07 | 64.01 | 29.15 | 2.2 | RASGEF1B | RasGEF domain | 153020 | NM_001300735 |
| | | | | | | | family member | | |
| | | | | | | | 1B | | |
| 0.0000363 | 0.000409 | <1e−07 | 87.11 | 35.61 | 2.45 | OTUD1 | OTU | 220213 | NM_001145373 |
| | | | | | | | deubiquitinase 1 | | |
| 0.0000363 | 0.000409 | <1e−07 | 19.53 | 9.73 | 2.01 | SRA1 | steroid receptor | 10011 | NM_001035235 |
| | | | | | | | RNA activator 1 | | |
| 0.0000364 | 0.00041 | <1e−07 | 105.02 | 42.09 | 2.5 | AC007384.1 | noncoding | | |
| | | | | | | | (Ensemble gene | | |
| | | | | | | | ID: | | |
| | | | | | | | ENSG00000237513.2) | | |
| 0.000037 | 0.000415 | <1e−07 | 11.63 | 30.79 | 0.38 | SHROOM3 | shroom family | 57619 | NM_020859 |
| | | | | | | | member 3 | | |
| 0.0000379 | 0.000424 | <1e−07 | 54.59 | 24.59 | 2.22 | SYNGAP1 | synaptic Ras | 8831 | NM_001130066 |
| | | | | | | | GTPase activating | | |
| | | | | | | | protein 1 | | |
| 0.0000383 | 0.000426 | <1e−07 | 104.35 | 52.06 | 2 | CD81 | CD81 molecule | 975 | NM_001297649 |
| 0.0000389 | 0.000431 | <1e−07 | 241.79 | 87.51 | 2.76 | ABCA1 | ATP binding | 19 | NM_005502 |
| | | | | | | | cassette subfamily | | |
| | | | | | | | A member 1 | | |
| 0.00004 | 0.00044 | <1e−07 | 10.06 | 23.15 | 0.43 | PPARGC1B | PPARG | 133522 | NM_001172698 |
| | | | | | | | coactivator 1 beta | | |
| 0.0000403 | 0.000442 | 0.0001 | 22.03 | 10.35 | 2.13 | AC122718.1 | noncoding (Clone | | |
| | | | | | | | Fragment ID: | | |
| | | | | | | | AC122718.2) | | |
| 0.0000403 | 0.000442 | <1e−07 | 41.45 | 17.55 | 2.36 | TOR3A | torsin family 3 | 64222 | NM_022371 |
| | | | | | | | member A | | |
| 0.0000404 | 0.000442 | <1e−07 | 446.98 | 188.67 | 2.37 | HERPUD1 | homocysteine | 9709 | NM_001010989 |
| | | | | | | | inducible ER | | |
| | | | | | | | protein with | | |
| | | | | | | | ubiquitin like | | |
| | | | | | | | domain 1 | | |
| 0.0000406 | 0.000444 | <1e−07 | 151.87 | 75.86 | 2 | PARP14 | poly(ADP-ribose) | 54625 | NM_017554 |
| | | | | | | | polymerase family | | |
| | | | | | | | member 14 | | |
| 0.0000409 | 0.000446 | <1e−07 | 145 | 61.5 | 2.36 | KLHL21 | kelch like family | 9903 | NM_001324309 |
| | | | | | | | member 21 | | |
| 0.000042 | 0.000456 | <1e−07 | 11.49 | 26.47 | 0.43 | SHANK2 | SH3 and multiple | 22941 | NM_012309 |
| | | | | | | | ankyrin repeat | | |
| | | | | | | | domains 2 | | |
| 0.0000428 | 0.000463 | <1e−07 | 7.27 | 17.5 | 0.42 | ERBB3 | erb-b2 receptor | 2065 | NM_001005915 |
| | | | | | | | tyrosine kinase 3 | | |
| 0.0000435 | 0.000469 | <1e−07 | 24.96 | 12.31 | 2.03 | ATP6V0B | ATPase H+ | 533 | NM_001039457 |
| | | | | | | | transporting V0 | | |
| | | | | | | | subunit b | | |
| 0.0000447 | 0.000479 | <1e−07 | 25.61 | 12.24 | 2.09 | BBC3 | BCL2 binding | 27113 | NM_001127240 |
| | | | | | | | component 3 | | |
| 0.0000457 | 0.000488 | 0.0001 | 18.71 | 9.28 | 2.02 | COL4A4 | collagen type IV | 1286 | NM_000092 |
| | | | | | | | alpha 4 chain | | |
| 0.0000459 | 0.000489 | <1e−07 | 80.44 | 32.48 | 2.48 | RGCC | regulator of cell | 28984 | NM_014059 |
| | | | | | | | cycle | | |
| 0.000046 | 0.00049 | <1e−07 | 51.66 | 24.72 | 2.09 | ABCA7 | ATP binding | 10347 | NM_019112 |
| | | | | | | | cassette subfamily | | |
| | | | | | | | A member 7 | | |
| 0.0000464 | 0.000492 | <1e−07 | 21.12 | 9.74 | 2.17 | CPNE5 | copine 5 | 57699 | NM_001314017 |
| 0.0000464 | 0.000492 | <1e−07 | 6.85 | 17.08 | 0.4 | INAVA | innate immunity | 55765 | NM_001142569 |
| | | | | | | | activator | | |
| 0.0000473 | 0.000499 | 0.0001 | 15.32 | 71.71 | 0.21 | MUC3A | mucin 3A, cell | 4584 | NM_005960 |
| | | | | | | | surface associated | | |
| 0.0000481 | 0.000506 | <1e−07 | 6.98 | 18.94 | 0.37 | BAIAP2L2 | BAI1 associated | 80115 | NM_025045 |
| | | | | | | | protein 2 like 2 | | |
| 0.0000484 | 0.000508 | 0.0002 | 21.58 | 10.65 | 2.03 | ZNF275 | zinc finger protein | 10838 | NM_001080485 |
| | | | | | | | 275 | | |
| 0.0000484 | 0.000508 | <1e−07 | 10.63 | 31.02 | 0.34 | HNF4A | hepatocyte | 3172 | NM_000457 |
| | | | | | | | nuclear factor 4 | | |
| | | | | | | | alpha | | |
| 0.0000486 | 0.000509 | <1e−07 | 8.72 | 24.03 | 0.36 | PKP2 | plakophilin 2 | 5318 | NM_001005242 |
| 0.0000491 | 0.000512 | <1e−07 | 55.06 | 26.58 | 2.07 | ATP2B4 | ATPase plasma | 493 | NM_001001396 |
| | | | | | | | membrane Ca2+ | | |
| | | | | | | | transporting 4 | | |
| 0.0000491 | 0.000512 | <1e−07 | 30.39 | 14.79 | 2.06 | CDC25B | cell division cycle | 994 | NM_001287516 |
| | | | | | | | 25B | | |
| 0.0000494 | 0.000514 | <1e−07 | 6.88 | 24.52 | 0.28 | MALRD1 | MAM and LDL | 340895 | NM_001142308 |
| | | | | | | | receptor class A | | |
| | | | | | | | domain containing | | |
| | | | | | | | 1 | | |
| 0.0000498 | 0.000518 | <1e−07 | 10.04 | 28.24 | 0.36 | TCEA3 | transcription | 6920 | NM_003196 |
| | | | | | | | elongation factor | | |
| | | | | | | | A3 | | |
| 0.0000506 | 0.000524 | <1e−07 | 7.37 | 17.29 | 0.43 | HMGN5 | high mobility | 79366 | NM_030763 |
| | | | | | | | group nucleosome | | |
| | | | | | | | binding domain 5 | | |
| 0.0000506 | 0.000524 | 0.0001 | 22.79 | 10.35 | 2.2 | PI4K2A | phosphatidylinositol | 55361 | NM_018425 |
| | | | | | | | 4-kinasetype 2 | | |
| | | | | | | | alpha | | |
| 0.000052 | 0.000536 | 0.0001 | 23.83 | 10.09 | 2.36 | CHST7 | carbohydrate | 56548 | NM_019886 |
| | | | | | | | sulfotransferase 7 | | |
| 0.0000523 | 0.000538 | 0.0001 | 5.54 | 11.27 | 0.49 | CCDC26 | CCDC26 long | 137196 | NR_130917 |
| | | | | | | | non-coding RNA | | |
| 0.0000525 | 0.000539 | <1e−07 | 94.06 | 41.9 | 2.24 | MIR29B2CHG | MIR29B2 and | 100128537 | NR_135298 |
| | | | | | | | MIR29C host | | |
| | | | | | | | gene | | |
| 0.0000526 | 0.00054 | 0.0002 | 37.03 | 15.91 | 2.33 | NRP2 | neuropilin 2 | 8828 | NM_003872 |
| 0.0000527 | 0.00054 | <1e−07 | 101.86 | 47.8 | 2.13 | TNKS1BP1 | tankyrase 1 | 85456 | NM_033396 |
| | | | | | | | binding protein 1 | | |
| 0.0000528 | 0.000541 | <1e−07 | 8.84 | 30.09 | 0.29 | CDHR5 | cadherin related | 53841 | NM_001171968 |
| | | | | | | | family member 5 | | |
| 0.0000534 | 0.000544 | <1e−07 | 20.24 | 8.1 | 2.5 | FCMR | Fc fragment of | 9214 | NM_001142472 |
| | | | | | | | IgM receptor | | |
| 0.0000536 | 0.000545 | 0.0002 | 125.25 | 49.25 | 2.54 | SCARNA7 | small Cajal body- | 677767 | NR_003001 |
| | | | | | | | specific RNA 7 | | |
| 0.0000562 | 0.000565 | 0.0001 | 31.56 | 15.02 | 2.1 | TRPS1 | transcriptional | 7227 | NM_001282902 |
| | | | | | | | repressor GATA | | |
| | | | | | | | binding 1 | | |
| 0.0000567 | 0.000569 | <1e−07 | 134.99 | 47.21 | 2.86 | AEBP1 | A binding | 165 | NM_001129 |
| | | | | | | | protein 1 | | |
| 0.0000568 | 0.00057 | <1e−07 | 56.04 | 21.29 | 2.63 | SYTL3 | synaptotagmin | 94120 | NM_001009991 |
| | | | | | | | like 3 | | |
| 0.0000575 | 0.000575 | <1e−07 | 5.8 | 12.17 | 0.48 | TMEM184A | transmembrane | 202915 | NM_001097620 |
| | | | | | | | protein 184A | | |
| 0.000059 | 0.000585 | <1e−07 | 5.9 | 12.59 | 0.47 | PMFBP1 | polyamine | 83449 | NM_001160213 |
| | | | | | | | modulated factor | | |
| | | | | | | | 1 binding protein | | |
| | | | | | | | 1 | | |
| 0.0000619 | 0.000609 | <1e−07 | 745.77 | 347.5 | 2.15 | DUSP1 | dual specificity | 1843 | NM_004417 |
| | | | | | | | phosphatase 1 | | |
| 0.000062 | 0.000609 | <1e−07 | 394.01 | 166.87 | 2.36 | LENG8 | leukocyte receptor | 114823 | NM_052925 |
| | | | | | | | cluster member 8 | | |
| 0.0000625 | 0.000612 | <1e−07 | 6.2 | 14.08 | 0.44 | MT-TI | Mitochondrially | 4565 | |
| | | | | | | | Encoded TRNA- | | |
| | | | | | | | Ile (AUU/C) | | |
| 0.0000626 | 0.000612 | 0.0001 | 6.46 | 14.08 | 0.46 | PLEKHG6 | pleckstrin | 55200 | NM_001144856 |
| | | | | | | | homology and | | |
| | | | | | | | RhoGF domain | | |
| | | | | | | | containing G6 | | |
| 0.0000626 | 0.000612 | 0.0001 | 53.41 | 23.17 | 2.3 | SLC5A3 | solute carrier | 6526 | NM_006933 |
| | | | | | | | family 5 member | | |
| | | | | | | | 3 | | |
| 0.0000639 | 0.000621 | <1e−07 | 34.41 | 16.47 | 2.09 | FLCN | folliculin | 201163 | NM_001353229 |
| 0.0000645 | 0.000625 | <1e−07 | 27.95 | 13.47 | 2.07 | PIK3IP1 | phosphoinositide- | 113791 | NM_001135911 |
| | | | | | | | 3-kinase | | |
| | | | | | | | interacting protein | | |
| | | | | | | | 1 | | |
| 0.0000647 | 0.000626 | <1e−07 | 93.5 | 45.7 | 2.05 | FNDC3A | fibronectin type | 22862 | NM_001079673 |
| | | | | | | | III domain | | |
| | | | | | | | containing 3A | | |
| 0.0000648 | 0.000626 | <1e−07 | 20.04 | 9.94 | 2.02 | C16orf54 | chromosome 16 | 283897 | NM_175900 |
| | | | | | | | open reading | | |
| | | | | | | | frame 54 | | |
| 0.0000672 | 0.000643 | <1e−07 | 135.1 | 60.93 | 2.22 | DUSP2 | dual specificity | 1844 | NM_004418 |
| | | | | | | | phosphatase 2 | | |
| 0.0000675 | 0.000645 | <1e−07 | 88.19 | 39.89 | 2.21 | SLC1A5 | solute carrier | 6510 | NM_001145144 |
| | | | | | | | family 1 member | | |
| | | | | | | | 5 | | |
| 0.0000675 | 0.000645 | <1e−07 | 33.64 | 16.52 | 2.04 | HLA-L | major | 3139 | NR_027822 |
| | | | | | | | histocompatibility | | |
| | | | | | | | complex, class I, | | |
| | | | | | | | L (pseudogene) | | |
| 0.0000691 | 0.000657 | <1e−07 | 192.25 | 91.43 | 2.1 | SLC3A2 | solute carrier | 6520 | NM_001012661 |
| | | | | | | | family 3 member | | |
| | | | | | | | 2 | | |
| 0.0000693 | 0.000659 | <1e−07 | 6.21 | 15.01 | 0.41 | SH3RF2 | SH3 domain | 153769 | NM_152550 |
| | | | | | | | containing ring | | |
| | | | | | | | finger 2 | | |
| 0.0000695 | 0.00066 | <1e−07 | 44.18 | 16.54 | 2.67 | MZB1 | marginal zone B | 51237 | NM_016459 |
| | | | | | | | and B1 cell | | |
| | | | | | | | specific protein | | |
| 0.00007 | 0.000664 | 0.0001 | 127.13 | 58.34 | 2.18 | RELB | RLB proto- | 5971 | NM_006509 |
| | | | | | | | oncogene, NF-kB | | |
| | | | | | | | subunit | | |
| 0.0000702 | 0.000665 | <1e−07 | 6.27 | 14.99 | 0.42 | MT-TA | Mitochondrially | 4553 | |
| | | | | | | | Encoded TRNA- | | |
| | | | | | | | Ala (GCN) | | |
| 0.0000716 | 0.000676 | 0.0001 | 19.65 | 9.41 | 2.09 | STARD8 | StAR related lipid | 9754 | NM_001142503 |
| | | | | | | | transfer domain | | |
| | | | | | | | containing 8 | | |
| 0.0000719 | 0.000678 | 0.0001 | 61.26 | 29.83 | 2.05 | VAV1 | vav guanine | 7409 | NM_001258206 |
| | | | | | | | nucleotide | | |
| | | | | | | | exchange factor 1 | | |
| 0.0000725 | 0.000682 | 0.0001 | 37.53 | 18.43 | 2.04 | GNS | glucosamine (N- | 2799 | NM_002076 |
| | | | | | | | acetyl)-6-sulfatase | | |
| 0.0000725 | 0.000682 | 0.0001 | 42.92 | 12.82 | 3.35 | SNORA20 | small nucleolar | 677806 | NR_002960 |
| | | | | | | | RNA, H/ACA box | | |
| | | | | | | | 20 | | |
| 0.0000735 | 0.000688 | 0.0001 | 14.36 | 40.21 | 0.36 | ITGB4 | integrin subunit | 3691 | NM_000213 |
| | | | | | | | beta 4 | | |
| 0.0000739 | 0.00069 | <1e−07 | 75.52 | 36.73 | 2.06 | NABP1 | nucleic acid | 64859 | NM_001031716 |
| | | | | | | | binding protein 1 | | |
| 0.0000766 | 0.000711 | 0.0001 | 8.28 | 26.98 | 0.31 | KRT20 | keratin 20 | 54474 | NM_019010 |
| 0.0000772 | 0.000715 | <1e−07 | 38.2 | 18.73 | 2.04 | REC8 | REC8 meiotic | 9985 | NM_001048205 |
| | | | | | | | recombination | | |
| | | | | | | | protein | | |
| 0.0000776 | 0.000717 | <1e−07 | 64.93 | 24.22 | 2.68 | IKZF3 | IKAROS family | 22806 | NM_001257408 |
| | | | | | | | zinc finger 3 | | |
| 0.0000781 | 0.00072 | 0.0001 | 66.87 | 31.15 | 2.15 | NEU1 | neuraminidase 1 | 4758 | NM_000434 |
| 0.0000786 | 0.000723 | <1e−07 | 108.85 | 45.89 | 2.37 | THEMIS2 | thymocyte | 9473 | NM_001039477 |
| | | | | | | | selection | | |
| | | | | | | | associated family | | |
| | | | | | | | member 2 | | |
| 0.0000787 | 0.000723 | 0.0001 | 18.07 | 63.37 | 0.29 | CGN | cingulin | 57530 | NM_020770 |
| 0.0000805 | 0.000737 | 0.0002 | 25.97 | 12.38 | 2.1 | RALGPS2 | Ral GF with PH | 55103 | NM_001286247 |
| | | | | | | | domain and SH3 | | |
| | | | | | | | binding motif 2 | | |
| 0.0000808 | 0.000739 | 0.0004 | 104.84 | 46.74 | 2.24 | FNIP2 | folliculin | 57600 | NM_001323916 |
| | | | | | | | interacting protein | | |
| | | | | | | | 2 | | |
| 0.0000835 | 0.00076 | 0.0002 | 122.86 | 49.12 | 2.5 | NFKBID | NFKB inhibitor | 84807 | NM_001321831 |
| | | | | | | | delta | | |
| 0.000085 | 0.000769 | 0.0001 | 34.66 | 14.72 | 2.35 | TMC8 | transmembrane | 147138 | NM_152468 |
| | | | | | | | channel like 8 | | |
| 0.0000857 | 0.000774 | 0.0001 | 44.14 | 12.83 | 3.44 | CD163L1 | CD163 molecule | 283316 | NM_001297650 |
| | | | | | | | like 1 | | |
| 0.0000864 | 0.000779 | 0.0002 | 41.71 | 16.66 | 2.5 | ADAM28 | ADAM | 10863 | NM_001304351 |
| | | | | | | | metallopeptidase | | |
| | | | | | | | domain 28 | | |
| 0.0000871 | 0.000784 | 0.0003 | 7.41 | 20.05 | 0.37 | ST6GALNAC1 | ST6 N-acetyl- | 55808 | NM_001289107 |
| | | | | | | | galactosaminide | | |
| | | | | | | | alpha-2,6- | | |
| | | | | | | | sialyltransferase 1 | | |
| 0.000088 | 0.00079 | 0.0001 | 138.15 | 59.47 | 2.32 | GRN | granulin precursor | 2896 | NM_001012479 |
| 0.0000883 | 0.000793 | 0.0001 | 79.02 | 35.96 | 2.2 | SLC38A1 | solute carrier | 81539 | NM_001077484 |
| | | | | | | | family 38 member | | |
| | | | | | | | 1 | | |
| 0.000089 | 0.000797 | 0.0002 | 8.3 | 21.17 | 0.39 | KIAA1324 | KIAA1324 | 57535 | NM_001267048 |
| 0.0000899 | 0.000803 | 0.0001 | 46.65 | 22.85 | 2.04 | CSGALNACT2 | chondroitin | 55454 | NM_001319654 |
| | | | | | | | sulfate N-acetyl- | | |
| | | | | | | | galactosaminyl- | | |
| | | | | | | | transferase 2 | | |
| 0.0000907 | 0.000808 | 0.0001 | 68.87 | 342.37 | 0.2 | PIGR | polymeric | 5284 | NM_002644 |
| | | | | | | | immunoglobulin | | |
| | | | | | | | receptor | | |
| 0.000091 | 0.00081 | 0.0001 | 66.54 | 22.25 | 2.99 | CHAC1 | ChaC glutathione | 79094 | NM_001142776 |
| | | | | | | | specific gamma- | | |
| | | | | | | | glutamylcyclo- | | |
| | | | | | | | transferase 1 | | |
| 0.0000931 | 0.000825 | <1e−07 | 6.33 | 14.11 | 0.45 | TMC4 | transmembrane | 147798 | NM_001145303 |
| | | | | | | | channel like 4 | | |
| 0.0000949 | 0.000836 | <1e−07 | 36.8 | 18.26 | 2.01 | DOCK11 | dedicator of | 139818 | NM_144658 |
| | | | | | | | cytokinesis 11 | | |
| 0.0000964 | 0.000846 | 0.0002 | 81.53 | 39.65 | 2.06 | ATP1B3 | ATPase Na+/K+ | 483 | NM_001679 |
| | | | | | | | transporting | | |
| | | | | | | | subunit beta 3 | | |
| 0.0000976 | 0.000853 | <1e−07 | 31.91 | 14.15 | 2.26 | P2RY8 | P2Y receptor | 286530 | NM_178129 |
| | | | | | | | family member 8 | | |
| 0.000098 | 0.000855 | 0.0003 | 20.19 | 9.69 | 2.08 | SNHG1 | small nucleolar | 23642 | NR_003098 |
| | | | | | | | RNA host gene 1 | | |
| 0.0000988 | 0.000861 | 0.0001 | 31.69 | 15.36 | 2.06 | RTL5 | retrotransposon | 340526 | NM_001024455 |
| | | | | | | | Gag like 5 | | |
| 0.0001008 | 0.000874 | <1e−07 | 46.66 | 19.41 | 2.4 | ITGAL | integrin subunit | 3683 | NM_001114380 |
| | | | | | | | alpha L | | |
| 0.0001031 | 0.00089 | 0.0001 | 35.52 | 17.55 | 2.02 | TEDC1 | tubulin epsilon | 283643 | NM_001134875 |
| | | | | | | | and delta complex | | |
| | | | | | | | 1 | | |
| 0.0001058 | 0.000908 | 0.0003 | 53.75 | 297.88 | 0.18 | MUC2 | mucin 2, | 4583 | NM_002457 |
| | | | | | | | oligomeric | | |
| | | | | | | | mucus/gel- | | |
| | | | | | | | forming | | |
| 0.0001068 | 0.000916 | 0.0002 | 35.23 | 17.18 | 2.05 | KMT2E- | KMT2E antisense | 100216545 | NR_024586 |
| | | | | | | AS1 | RNA 1 | | |
| 0.0001082 | 0.000927 | 0.0002 | 19.36 | 9.45 | 2.05 | AC116366.2 | noncoding | | |
| | | | | | | | (Ensemble gene | | |
| | | | | | | | ID: | | |
| | | | | | | | ENSG00000238160.1) | | |
| 0.0001085 | 0.000929 | <1e−07 | 17.62 | 8.4 | 2.1 | GCNA | germ cell nuclear | 93953 | NM_052957 |
| | | | | | | | acidic peptidase | | |
| 0.0001091 | 0.000933 | 0.0001 | 21.73 | 10.54 | 2.06 | AQP3 | aquaporin 3 (Gill | 360 | NM_001318144 |
| | | | | | | | blood group) | | |
| 0.0001105 | 0.000941 | <1e−07 | 19.62 | 9.55 | 2.05 | SMOX | spermine oxidase | 54498 | NM_001270691 |
| 0.0001106 | 0.000941 | <1e−07 | 34.74 | 17.04 | 2.04 | ZFYVE16 | zinc finger FYVE- | 9765 | NM_001105251 |
| | | | | | | | type containing 16 | | |
| 0.0001106 | 0.000941 | 0.0001 | 88.33 | 35.29 | 2.5 | FYN | FYN proto- | 2534 | NM_001242779 |
| | | | | | | | oncogene, Src | | |
| | | | | | | | family tyrosine | | |
| | | | | | | | kinase | | |
| 0.0001129 | 0.000955 | 0.0002 | 32.37 | 15.23 | 2.13 | FGD3 | FYVE, RhoGEF | 89846 | NM_001083536 |
| | | | | | | | and PH domain | | |
| | | | | | | | containing 3 | | |
| 0.0001134 | 0.000958 | <1e−07 | 128.14 | 63.49 | 2.02 | SEL1L | SEL1L ERAD E3 | 6400 | NM_001244984 |
| | | | | | | | ligase adaptor | | |
| | | | | | | | subunit | | |
| 0.0001176 | 0.000989 | <1e−07 | 30.37 | 14.23 | 2.13 | SLC35A2 | solute carrier | 7355 | NM_001032289 |
| | | | | | | | family 35 member | | |
| | | | | | | | A2 | | |
| 0.0001181 | 0.000991 | 0.0002 | 155.18 | 74.09 | 2.09 | ATG2A | autophagy related | 23130 | NM_015104 |
| | | | | | | | 2A | | |
| 0.000122 | 0.00101 | <1e−07 | 57.21 | 25.77 | 2.22 | FGFR1 | fibroblast growth | 2260 | NM_001174063 |
| | | | | | | | factor receptor 1 | | |
| 0.0001242 | 0.00103 | 0.0001 | 91.51 | 43.69 | 2.09 | TRANK1 | tetratricopeptide | 9881 | NM_001329998 |
| | | | | | | | repeat and ankyrin | | |
| | | | | | | | repeat containing | | |
| | | | | | | | 1 | | |
| 0.0001284 | 0.00105 | <1e−07 | 59.38 | 29.43 | 2.02 | U2SURP | U2 snRNP | 23350 | NM_001080415 |
| | | | | | | | associated SURP | | |
| | | | | | | | domain containing | | |
| 0.0001291 | 0.00106 | 0.0002 | 166.78 | 71.78 | 2.32 | TRAF1 | TNF receptor | 7185 | NM_001190945 |
| | | | | | | | associated factor 1 | | |
| 0.0001312 | 0.00107 | 0.0002 | 23.22 | 10.49 | 2.21 | MCOLN2 | mucolipin 2 | 255231 | NM_001330647 |
| 0.0001335 | 0.00109 | 0.0001 | 27.5 | 13.3 | 2.07 | CASTOR2 | cytosolic arginine | 729438 | NM_001145064 |
| | | | | | | | sensor for | | |
| | | | | | | | mTORC1 subunit | | |
| | | | | | | | 2 | | |
| 0.0001408 | 0.00113 | 0.0001 | 84.8 | 40.18 | 2.11 | MROH1 | maestro heat like | 727957 | NM_001099280 |
| | | | | | | | repeat family | | |
| | | | | | | | member 1 | | |
| 0.0001413 | 0.00114 | 0.0001 | 576.48 | 257.2 | 2.24 | NFKB2 | nuclear factor | 4791 | NM_001077494 |
| | | | | | | | kappa B subunit 2 | | |
| 0.0001419 | 0.00114 | 0.0001 | 154.78 | 63.14 | 2.45 | ANKRD28 | ankyrin repeat | 23243 | NM_001195098 |
| | | | | | | | domain 28 | | |
| 0.0001429 | 0.00115 | 0.0001 | 1152.66 | 405.22 | 2.84 | AC020916.1 | noncoding | | |
| | | | | | | | (Ensemble gene | | |
| | | | | | | | ID: | | |
| | | | | | | | ENSG00000267519.6) | | |
| 0.0001466 | 0.00117 | 0.0001 | 30.61 | 14.93 | 2.05 | DNAAF2 | dynein axonemal | 55172 | NM_001083908 |
| | | | | | | | assembly factor 2 | | |
| 0.000148 | 0.00118 | <1e−07 | 218.23 | 84.92 | 2.57 | PNISR | PNN interacting | 25957 | NM_001322405 |
| | | | | | | | serine and | | |
| | | | | | | | arginine rich | | |
| | | | | | | | protein | | |
| 0.0001488 | 0.00119 | 0.0002 | 102.58 | 51.02 | 2.01 | MAFK | MAF bZIP | 7975 | NM_002360 |
| | | | | | | | transcription | | |
| | | | | | | | factor K | | |
| 0.0001502 | 0.00119 | 0.0001 | 12.06 | 36.1 | 0.33 | CLDN3 | claudin 3 | 1365 | NM_001306 |
| 0.0001547 | 0.00122 | 0.0001 | 37.81 | 18.74 | 2.02 | ARFGAP3 | ADP ribosylation | 26286 | NM_001142293 |
| | | | | | | | factor GTPase | | |
| | | | | | | | activating protein | | |
| | | | | | | | 3 | | |
| 0.0001551 | 0.00122 | <1e−07 | 47.96 | 19.97 | 2.4 | GM2A | GM2 ganglioside | 2760 | NM_000405 |
| | | | | | | | activator | | |
| 0.0001554 | 0.00122 | 0.0001 | 15.68 | 32.45 | 0.48 | SH3D19 | SH3 domain | 152503 | NM_001009555 |
| | | | | | | | containing 19 | | |
| 0.000156 | 0.00123 | 0.0001 | 109.55 | 47.59 | 2.3 | FR1L4 | fer-1 like family | 80307 | NR_001442 |
| | | | | | | | member 4, | | |
| | | | | | | | pseudogene | | |
| 0.000161 | 0.00126 | 0.0002 | 134.22 | 65.73 | 2.04 | ARID5B | AT-rich | 84159 | NM_001244638 |
| | | | | | | | interaction | | |
| | | | | | | | domain 5B | | |
| 0.0001638 | 0.00127 | 0.0002 | 132.71 | 62.15 | 2.14 | TTYH3 | tweety family | 80727 | NM_025250 |
| | | | | | | | member 3 | | |
| 0.0001645 | 0.00128 | <1e−07 | 7.31 | 17.08 | 0.43 | TMC5 | transmembrane | 79838 | NM_001105248 |
| | | | | | | | channel like 5 | | |
| 0.0001649 | 0.00128 | <1e−07 | 30.55 | 13.49 | 2.27 | CD4 | CD4 molecule | 920 | NM_000616 |
| 0.0001668 | 0.00129 | 0.0001 | 35.74 | 11.52 | 3.1 | IGLV3-21 | immunoglobulin | 28796 | |
| | | | | | | | lambda variable 3- | | |
| | | | | | | | 21 | | |
| 0.0001705 | 0.00131 | <1e−07 | 93.92 | 42.55 | 2.21 | STAT5A | signal transducer | 6776 | NM_001288718 |
| | | | | | | | and activator of | | |
| | | | | | | | transcription 5A | | |
| 0.0001705 | 0.00131 | 0.0002 | 58.32 | 28.54 | 2.04 | CNOT6L | CCR4-NOT | 246175 | NM_001286790 |
| | | | | | | | transcription | | |
| | | | | | | | complex subunit 6 | | |
| | | | | | | | like | | |
| 0.0001741 | 0.00133 | 0.0001 | 15.27 | 7.41 | 2.06 | NUGGC | nuclear GTPase, | 389643 | NM_001010906 |
| | | | | | | | germinal center | | |
| | | | | | | | associated | | |
| 0.0001746 | 0.00133 | <1e−07 | 376.32 | 162.48 | 2.32 | PSAP | prosaposin | 5660 | NM_001042465 |
| 0.0001764 | 0.00134 | 0.0001 | 177.99 | 69.22 | 2.57 | PLCG2 | phospholipase C | 5336 | NM_002661 |
| | | | | | | | gamma 2 | | |
| 0.0001771 | 0.00135 | 0.0001 | 2498.29 | 999.47 | 2.5 | FOSB | FosB proto- | 2354 | NM_001114171 |
| | | | | | | | oncogene, AP-1 | | |
| | | | | | | | transcription | | |
| | | | | | | | factor subunit | | |
| 0.0001812 | 0.00137 | 0.0003 | 21.99 | 9.39 | 2.34 | NPIPB3 | nuclear pore | 23117 | NM_130464 |
| | | | | | | | complex | | |
| | | | | | | | interactingprotein | | |
| | | | | | | | family member | | |
| | | | | | | | B3 | | |
| 0.0001858 | 0.0014 | 0.0003 | 8.1 | 19.37 | 0.42 | TJP3 | tight junction | 27134 | NM_001267560 |
| | | | | | | | protein 3 | | |
| 0.0001875 | 0.00141 | 0.0001 | 97.19 | 32.77 | 2.97 | SPOCK2 | SPARC | 9806 | NM_001134434 |
| | | | | | | | (osteonectin), | | |
| | | | | | | | cwcv and kazal | | |
| | | | | | | | like domains | | |
| | | | | | | | proteoglycan 2 | | |
| 0.0001889 | 0.00141 | 0.0001 | 255.89 | 107.22 | 2.39 | LTBP4 | latent | 8425 | NM_001042544 |
| | | | | | | | transforming | | |
| | | | | | | | growth factor beta | | |
| | | | | | | | binding protein 4 | | |
| 0.0001968 | 0.00146 | 0.0002 | 51.97 | 24.64 | 2.11 | PRPF3 | pre-m RNA | 9129 | NM_001350529 |
| | | | | | | | processing factor | | |
| | | | | | | | 3 | | |
| 0.0001987 | 0.00147 | 0.0002 | 15.28 | 6.82 | 2.24 | CCL22 | C-C motif | 6367 | NM_002990 |
| | | | | | | | chemokine ligand | | |
| | | | | | | | 22 | | |
| 0.0001989 | 0.00147 | 0.0004 | 20.15 | 9.98 | 2.02 | APBB3 | amyloid beta | 10307 | NM_006051 |
| | | | | | | | precursor protein | | |
| | | | | | | | binding family B | | |
| | | | | | | | member 3 | | |
| 0.0002053 | 0.00151 | 0.0003 | 51.34 | 24.47 | 2.1 | CHST15 | carbohydrate | 51363 | NM_001270764 |
| | | | | | | | sulfotransferase | | |
| | | | | | | | 15 | | |
| 0.0002056 | 0.00151 | 0.0002 | 256.22 | 122.01 | 2.1 | CSRNP1 | cysteine and | 64651 | NM_001320559 |
| | | | | | | | serine rich nuclear | | |
| | | | | | | | protein 1 | | |
| 0.0002076 | 0.00152 | 0.0003 | 13.29 | 6.31 | 2.1 | GPR15 | G protein-coupled | 2838 | NM_005290 |
| | | | | | | | receptor 15 | | |
| 0.0002084 | 0.00152 | 0.0003 | 34.58 | 143.37 | 0.24 | MYH14 | myosin heavy | 79784 | NM_001077186 |
| | | | | | | | chain 14 | | |
| 0.000217 | 0.00157 | 0.0004 | 52.49 | 20.72 | 2.53 | HLA-DQA1 | major | 3117 | NM_002122 |
| | | | | | | | histocompatibility | | |
| | | | | | | | complex, class II, | | |
| | | | | | | | DQ alpha 1 | | |
| 0.0002213 | 0.00159 | <1e−07 | 29.19 | 13.4 | 2.18 | BAIAP3 | BAI1 associated | 8938 | NM_001199096 |
| | | | | | | | protein 3 | | |
| 0.0002217 | 0.00159 | 0.0001 | 11.04 | 33.5 | 0.33 | EPCAM | epithelial cell | 4072 | NM_002354 |
| | | | | | | | adhesion molecule | | |
| 0.0002236 | 0.0016 | 0.0001 | 8.39 | 36.39 | 0.23 | DEFA5 | defensin alpha 5 | 1670 | NM_021010 |
| 0.0002256 | 0.00161 | 0.0001 | 9.07 | 19.91 | 0.46 | CAMSAP3 | calmodulin | 57662 | NM_001080429 |
| | | | | | | | regulated spectrin | | |
| | | | | | | | associated protein | | |
| | | | | | | | family member 3 | | |
| 0.0002337 | 0.00166 | 0.0001 | 5.73 | 13.05 | 0.44 | SMIM24 | small integral | 284422 | NM_001136503 |
| | | | | | | | membrane protein | | |
| | | | | | | | 24 | | |
| 0.0002341 | 0.00166 | 0.0005 | 59.43 | 25.5 | 2.33 | SPHK1 | sphingosine | 8877 | NM_001142601 |
| | | | | | | | kinase 1 | | |
| 0.0002365 | 0.00168 | 0.0001 | 65.94 | 32.65 | 2.02 | TCF4 | transcription | 6925 | NM_001083962 |
| | | | | | | | factor 4 | | |
| 0.0002415 | 0.0017 | 0.0002 | 52.55 | 23.52 | 2.23 | IQCN | IQ motif | 80726 | NM_001145304 |
| | | | | | | | containing N | | |
| 0.0002421 | 0.00171 | 0.0002 | 30.46 | 15.07 | 2.02 | BTN2A2 | butyrophilin | 10385 | NM_001197237 |
| | | | | | | | subfamily 2 | | |
| | | | | | | | member A2 | | |
| 0.0002465 | 0.00173 | 0.0003 | 7.45 | 15.89 | 0.47 | EPHX2 | epoxide hydrolase | 2053 | NM_001256482 |
| | | | | | | | 2 | | |
| 0.0002518 | 0.00175 | 0.0001 | 5.97 | 12.01 | 0.5 | FAM83E | family with | 54854 | NM_017708 |
| | | | | | | | sequence | | |
| | | | | | | | similarity 83 | | |
| | | | | | | | member E | | |
| 0.0002529 | 0.00176 | 0.0003 | 6.61 | 14.5 | 0.46 | FOXQ1 | forkhead box Q1 | 94234 | NM_033260 |
| 0.0002539 | 0.00176 | 0.0004 | 20.34 | 9.85 | 2.06 | TSPAN33 | tetraspanin 33 | 340348 | NM_178562 |
| 0.0002546 | 0.00177 | 0.0003 | 18.02 | 9 | 2 | AC011939.1 | noncoding (Clone | | |
| | | | | | | | Fragment ID: | | |
| | | | | | | | AC011939.9) | | |
| 0.0002549 | 0.00177 | <1e−07 | 137.94 | 66.66 | 2.07 | ABCC1 | ATP binding | 4363 | NM_004996 |
| | | | | | | | cassette subfamily | | |
| | | | | | | | C member 1 | | |
| 0.0002555 | 0.00177 | 0.0004 | 19.62 | 9.07 | 2.16 | SNORA14B | small nucleolar | 677802 | NR_002956 |
| | | | | | | | RNA, H/ACA box | | |
| | | | | | | | 14B | | |
| 0.0002566 | 0.00177 | 0.0005 | 19.01 | 46.06 | 0.41 | MAP7 | microtubule | 9053 | NM_001198608 |
| | | | | | | | associated protein | | |
| | | | | | | | 7 | | |
| 0.0002568 | 0.00177 | 0.0006 | 33.61 | 16.01 | 2.1 | TUBB6 | tubulin beta 6 | 84617 | NM_001303524 |
| | | | | | | | class V | | |
| 0.0002626 | 0.0018 | 0.0001 | 18.31 | 45.05 | 0.41 | PCSK5 | proprotein | 5125 | NM_001190482 |
| | | | | | | | convertase | | |
| | | | | | | | subtilisin/kexin | | |
| | | | | | | | type 5 | | |
| 0.0002639 | 0.00181 | 0.0002 | 43.73 | 20.47 | 2.14 | FAM118A | family with | 55007 | NM_001104595 |
| | | | | | | | sequence | | |
| | | | | | | | similarity 118 | | |
| | | | | | | | member A | | |
| 0.0002685 | 0.00184 | 0.0003 | 11.15 | 43.01 | 0.26 | MUC17 | mucin 17, cell | 140453 | NM_001004430 |
| | | | | | | | surface associated | | |
| 0.0002734 | 0.00187 | <1e−07 | 84.78 | 41.38 | 2.05 | BCL6 | B cell | 604 | NM_001130845 |
| | | | | | | | CLL/lymphoma 6 | | |
| 0.0002765 | 0.00188 | 0.0001 | 69.61 | 32.4 | 2.15 | CORO1A | coronin 1A | 11151 | NM_001193333 |
| 0.0002801 | 0.0019 | 0.0002 | 12.94 | 31.08 | 0.42 | MYO5B | myosin VB | 4645 | NM_001080467 |
| 0.0002842 | 0.00192 | 0.0005 | 8.81 | 18.71 | 0.47 | MT-TF | Mitochondrially | 4558 | |
| | | | | | | | Encoded TRNA- | | |
| | | | | | | | Phe (UUU/C) | | |
| 0.0002857 | 0.00193 | 0.0001 | 48.18 | 23.21 | 2.08 | MIAT | myocardial | 440823 | NR_003491 |
| | | | | | | | infarction | | |
| | | | | | | | associated | | |
| | | | | | | | transcript (non- | | |
| | | | | | | | protein coding) | | |
| 0.0002912 | 0.00196 | 0.0004 | 14.62 | 61.57 | 0.24 | OLFM4 | olfactomedin 4 | 10562 | NM_006418 |
| 0.0002985 | 0.002 | 0.0001 | 85.93 | 38.61 | 2.23 | SCARNA5 | small Cajal body- | 677775 | NR_003008 |
| | | | | | | | specific RNA 5 | | |
| 0.0003062 | 0.00204 | <1e−07 | 71.25 | 35.56 | 2 | DAPK1 | death associated | 1612 | NM_001288729 |
| | | | | | | | protein kinase 1 | | |
| 0.0003078 | 0.00205 | 0.0003 | 68.54 | 32.7 | 2.1 | TMEM63B | transmembrane | 55362 | NM_001318792 |
| | | | | | | | protein 63B | | |
| 0.000314 | 0.00208 | 0.0001 | 290.72 | 139.92 | 2.08 | PIM3 | Pim-3 proto- | 415116 | NM_001001852 |
| | | | | | | | oncogene, | | |
| | | | | | | | serine/threonine | | |
| | | | | | | | kinase | | |
| 0.0003145 | 0.00208 | 0.0006 | 90.26 | 37.92 | 2.38 | RRAD | RRAD, Ras | 6236 | NM_001128850 |
| | | | | | | | related glycolysis | | |
| | | | | | | | inhibitor and | | |
| | | | | | | | calcium channel | | |
| | | | | | | | regulator | | |
| 0.0003235 | 0.00213 | 0.0001 | 8.08 | 19.28 | 0.42 | CLDN7 | claudin 7 | 1366 | NM_001185022 |
| 0.0003281 | 0.00215 | 0.0003 | 21.43 | 10.01 | 2.14 | IL21R | interleukin 21 | 50615 | NM_021798 |
| | | | | | | | receptor | | |
| 0.0003297 | 0.00216 | 0.0003 | 11.52 | 26.91 | 0.43 | TRIM2 | tripartite motif | 23321 | NM_001130067 |
| | | | | | | | containing 2 | | |
| 0.0003328 | 0.00217 | 0.0002 | 96.06 | 45 | 2.13 | ETS1 | ETS proto- | 2113 | NM_001143820 |
| | | | | | | | oncogene 1, | | |
| | | | | | | | transcription | | |
| | | | | | | | factor | | |
| 0.0003501 | 0.00226 | 0.0004 | 44.38 | 15.85 | 2.8 | CD79A | CD79a molecule | 973 | NM_001783 |
| 0.0003501 | 0.00226 | 0.0004 | 13.17 | 26.64 | 0.49 | ANXA4 | annexin A4 | 307 | NM_001153 |
| 0.0003538 | 0.00228 | 0.0004 | 46.07 | 16.42 | 2.81 | CD163 | CD163 molecule | 9332 | NM_004244 |
| 0.0003705 | 0.00235 | 0.0003 | 135.12 | 60.53 | 2.23 | RGS1 | regulator of G | 5996 | NM_002922 |
| | | | | | | | protein signaling 1 | | |
| 0.0003781 | 0.00239 | 0.0009 | 55.49 | 20.2 | 2.75 | AXL | AXL receptor | 558 | NM_001278599 |
| | | | | | | | tyrosine kinase | | |
| 0.0003872 | 0.00243 | 0.0003 | 40.37 | 15.83 | 2.55 | ACP5 | acid phosphatase | 54 | NM_001111034 |
| | | | | | | | 5, tartrate resistant | | |
| 0.0004003 | 0.00249 | 0.0003 | 145.53 | 68.82 | 2.11 | RELT | RLT, TNF | 84957 | NM_032871 |
| | | | | | | | receptor | | |
| 0.0004093 | 0.00254 | 0.0005 | 501.83 | 234.59 | 2.14 | TSPYL2 | TSPY like 2 | 64061 | NM_022117 |
| 0.0004119 | 0.00255 | 0.0005 | 52.43 | 19.94 | 2.63 | MMP19 | matrix | 4327 | NM_001032360 |
| | | | | | | | metallopeptidase | | |
| | | | | | | | 19 | | |
| 0.0004168 | 0.00257 | 0.0006 | 27.29 | 12.7 | 2.15 | LONRF3 | LON peptidase N- | 79836 | NM_001031855 |
| | | | | | | | terminal domain | | |
| | | | | | | | and ring finger 3 | | |
| 0.000419 | 0.00259 | 0.0006 | 8.73 | 25.28 | 0.35 | AC103691.1 | noncoding | | |
| | | | | | | | (Ensemble gene | | |
| | | | | | | | ID: | | |
| | | | | | | | ENSG00000274383.1) | | |
| 0.000421 | 0.00259 | 0.0008 | 88.68 | 32.13 | 2.76 | MEG3 | maternally | 55384 | NR_002766 |
| | | | | | | | expressed 3 (non- | | |
| | | | | | | | protein coding) | | |
| 0.0004398 | 0.00269 | 0.0005 | 10.24 | 20.65 | 0.5 | PARD3B | par-3 family cell | 117583 | NM_001302769 |
| | | | | | | | polarity regulator | | |
| | | | | | | | beta | | |
| 0.000445 | 0.00271 | 0.0004 | 94.79 | 40.34 | 2.35 | SLAMF7 | SLAM family | 57823 | NM_001282588 |
| | | | | | | | member 7 | | |
| 0.000468 | 0.00282 | 0.0007 | 8.34 | 19.79 | 0.42 | ATP8B1 | ATPase | 5205 | NM_005603 |
| | | | | | | | phospholipid | | |
| | | | | | | | transporting 8B1 | | |
| 0.0004761 | 0.00286 | 0.0004 | 6.53 | 13.34 | 0.49 | PTPRH | protein tyrosine | 5794 | NM_001161440 |
| | | | | | | | phosphatase, | | |
| | | | | | | | receptor type H | | |
| 0.0004774 | 0.00286 | 0.0003 | 351.96 | 166.48 | 2.11 | SLC7A5 | solute carrier | 8140 | NM_003486 |
| | | | | | | | family 7 member | | |
| | | | | | | | 5 | | |
| 0.0004871 | 0.00291 | 0.0001 | 25.25 | 11.19 | 2.26 | SNORD13 | small nucleolar | 692084 | NR_003041 |
| | | | | | | | RNA, C/D box 13 | | |
| 0.0004946 | 0.00294 | 0.0006 | 49.98 | 19.01 | 2.63 | ZBTB16 | zinc finger and | 7704 | NM_001018011 |
| | | | | | | | BTB domain | | |
| | | | | | | | containing 16 | | |
| 0.0004987 | 0.00296 | 0.0002 | 44.98 | 21.99 | 2.05 | MEF2C | myocyte enhancer | 4208 | NM_001131005 |
| | | | | | | | factor 2C | | |
| 0.0005009 | 0.00297 | 0.0006 | 8.24 | 16.83 | 0.49 | LPIN3 | lipin 3 | 64900 | NM_001301860 |
| 0.0005157 | 0.00304 | 0.0005 | 127.22 | 48.09 | 2.65 | ANKRD36BP2 | ankyrin repeat | 645784 | NR_015424 |
| | | | | | | | domain 36B | | |
| | | | | | | | pseudogene 2 | | |
| 0.0005284 | 0.00309 | 0.0002 | 113.02 | 53.83 | 2.1 | TENT5C | Terminal | 54855 | |
| | | | | | | | Nucleotidyl- | | |
| | | | | | | | transferase 5C | | |
| 0.0005291 | 0.0031 | 0.0005 | 23.31 | 10.24 | 2.28 | STAP1 | signal transducing | 26228 | NM_001317769 |
| | | | | | | | adaptor family | | |
| | | | | | | | member 1 | | |
| 0.0005686 | 0.00329 | 0.0004 | 6.82 | 13.96 | 0.49 | GATA6 | GATA binding | 2627 | NM_005257 |
| | | | | | | | protein 6 | | |
| 0.0005854 | 0.00337 | 0.0008 | 162 | 74.56 | 2.17 | CD83 | CD83 molecule | 9308 | NM_001040280 |
| 0.0005921 | 0.0034 | 0.001 | 17.68 | 7.47 | 2.37 | CD19 | CD19 molecule | 930 | NM_001178098 |
| 0.0005958 | 0.00342 | 0.0007 | 25.07 | 11.43 | 2.19 | FBLN2 | fibulin 2 | 2199 | NM_001004019 |
| 0.0005971 | 0.00343 | 0.0003 | 135.05 | 54.88 | 2.46 | DUSP5 | dual specificity | 1847 | NM_004419 |
| | | | | | | | phosphatase 5 | | |
| 0.0006081 | 0.00348 | 0.0005 | 50.65 | 23.85 | 2.12 | PLD3 | phospholipase D | 23646 | NM_001031696 |
| | | | | | | | family member 3 | | |
| 0.0006296 | 0.00357 | 0.0005 | 16.1 | 7.14 | 2.26 | CXCR5 | C-X-C motif | 643 | NM_001716 |
| | | | | | | | chemokine | | |
| | | | | | | | receptor 5 | | |
| 0.0006423 | 0.00363 | 0.0007 | 87.62 | 34.35 | 2.55 | ADAMTS1 | ADAM | 9510 | NM_006988 |
| | | | | | | | metallopeptidase | | |
| | | | | | | | with | | |
| | | | | | | | thrombospondin | | |
| | | | | | | | type 1 motif 1 | | |
| 0.0006456 | 0.00364 | 0.0006 | 22.45 | 47.82 | 0.47 | H1-0 | H1.0 Linker | 3005 | |
| | | | | | | | Histone | | |
| 0.0006915 | 0.00384 | 0.0009 | 19.14 | 40.14 | 0.48 | CCND1 | cyclin D1 | 595 | NM_053056 |
| 0.0007359 | 0.00404 | 0.0003 | 63.99 | 29.75 | 2.15 | DOCK4 | dedicator of | 9732 | NM_014705 |
| | | | | | | | cytokinesis 4 | | |
| 0.0007413 | 0.00407 | 0.0011 | 10.69 | 25.42 | 0.42 | SELENBP1 | selenium binding | 8991 | NM_001258288 |
| | | | | | | | protein 1 | | |
| 0.000746 | 0.00409 | 0.0012 | 8.11 | 22.62 | 0.36 | MLXIPL | MLX interacting | 51085 | NM_032951 |
| | | | | | | | protein like | | |
| 0.0007536 | 0.00412 | 0.0006 | 8 | 18.87 | 0.42 | CDHR2 | cadherin related | 54825 | NM_001171976 |
| | | | | | | | family member 2 | | |
| 0.0007625 | 0.00416 | 0.0006 | 34.35 | 16.65 | 2.06 | TRPM2 | transient receptor | 7226 | NM_001001188 |
| | | | | | | | potential cation | | |
| | | | | | | | channel subfamily | | |
| | | | | | | | M member 2 | | |
| 0.0007643 | 0.00417 | 0.0013 | 8 | 17.26 | 0.46 | CYP3A5 | cytochrome P450 | 1577 | NM_000777 |
| | | | | | | | family 3 | | |
| | | | | | | | subfamily A | | |
| | | | | | | | member 5 | | |
| 0.0007846 | 0.00425 | 0.0006 | 56.58 | 27.58 | 2.05 | CTSS | cathepsin S | 1520 | NM_001199739 |
| 0.0007912 | 0.00428 | 0.0004 | 13.48 | 6.26 | 2.15 | NIBAN3 | Niban Apoptosis | 199786 | |
| | | | | | | | Regulator 3 | | |
| 0.0007975 | 0.0043 | 0.0009 | 8.06 | 17.51 | 0.46 | TMPRSS2 | transmembrane | 7113 | NM_001135099 |
| | | | | | | | serine protease 2 | | |
| 0.0007997 | 0.00431 | 0.0006 | 1006.5 | 496.11 | 2.03 | LMNA | lamin A/C | 4000 | NM_001257374 |
| 0.0008113 | 0.00435 | 0.001 | 36.08 | 14.57 | 2.48 | DNASE1L3 | deoxyribonuclease | 1776 | NM_001256560 |
| | | | | | | | 1 like 3 | | |
| 0.0008179 | 0.00438 | 0.0008 | 85.08 | 35.57 | 2.39 | SSR4 | signal sequence | 6748 | NM_001204526 |
| | | | | | | | receptor subunit 4 | | |
| 0.0008296 | 0.00443 | 0.0005 | 9.39 | 20.77 | 0.45 | CARD10 | caspase | 29775 | NM_014550 |
| | | | | | | | recruitment | | |
| | | | | | | | domain family | | |
| | | | | | | | member 10 | | |
| 0.0008483 | 0.00451 | 0.0014 | 37.75 | 138.21 | 0.27 | KRT8 | keratin 8 | 3856 | NM_001256282 |
| 0.000858 | 0.00455 | 0.0011 | 12.55 | 25.71 | 0.49 | NHSL1 | NHS like 1 | 57224 | NM_001144060 |
| 0.0008593 | 0.00455 | 0.001 | 292.02 | 132.74 | 2.2 | HLA-DPB1 | major | 3115 | NM_002121 |
| | | | | | | | histocompatibility | | |
| | | | | | | | complex, class II, | | |
| | | | | | | | DP beta 1 | | |
| 0.0008684 | 0.00459 | 0.0017 | 14.02 | 28.15 | 0.5 | MAGI1 | membrane | 9223 | NM_001033057 |
| | | | | | | | associated | | |
| | | | | | | | guany late kinase, | | |
| | | | | | | | WW and PDZ | | |
| | | | | | | | domain containing | | |
| | | | | | | | 1 | | |
| 0.0008767 | 0.00462 | 0.0007 | 15.35 | 39.79 | 0.39 | LRATD1 | LRAT Domain | 151354 | |
| | | | | | | | Containing 1 | | |
| 0.0009106 | 0.00476 | 0.0012 | 23.6 | 11.23 | 2.1 | STAT4 | signal transducer | 6775 | NM_001243835 |
| | | | | | | | and activator of | | |
| | | | | | | | transcription 4 | | |
| 0.0009638 | 0.00499 | 0.0004 | 44.22 | 21.3 | 2.08 | COBLL1 | cordon-bleu WH2 | 22837 | NM_001278458 |
| | | | | | | | repeat protein like | | |
| | | | | | | | 1 | | |
| 0.0009744 | 0.00503 | 0.0011 | 10.88 | 25.78 | 0.42 | CDH1 | cadherin 1 | 999 | NM_001317184 |
| 0.0009985 | 0.00514 | 0.0009 | 80.07 | 38.2 | 2.1 | SH3D21 | SH3 domain | | NM_001162530 |
| | | | | | | | containing 21 | 79729 | |

In some embodiments, the methods involve detecting in a biological sample from a subject expression levels of one or more genes of a transcriptomic signature to obtain an expression profile comprising the expression levels of each of the one or more genes in the signature. In some embodiments, the transcriptomic signature comprises one or more biomarkers listed in Tables 1. In some embodiments, the transcriptomic signature comprises any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 5, 60, 65, 70, 75, 80, 90, 100, or more of the genes of Tables 1.

In some embodiments, gene expression profiling may be used as a research tool to identify new markers for diagnosis and/or classification of an IBD disease or condition, to monitor the effect of drugs or candidate drugs on biological samples and/or patients, to uncover new pathways for IBD treatment, or any combination thereof. In some embodiments, the treatment comprises a modulator of miR-181a, miR-92a, and miR-124.

The expression profile of atranscriptomic signature in a subject may be determined by analyzing genetic material obtained from a subject. The subject may be human. In some embodiments, the genetic material is obtained from a subject having an inflammatory disease, such as inflammatory bowel disease, or specifically, Crohn's Disease. Although the methods described herein are generally referenced for use with Crohn's Disease patients, in some cases the methods and transcriptomic signatures are applicable to other inflammatory diseases, including, ulcerative colitis.

In some embodiments, the genetic material is obtained from blood, serum, plasma, sweat, hair, tears, urine, or tissue. Techniques for obtaining samples from a subject include, for example, obtaining samples by a mouth swab or a mouth wash, drawing blood, and obtaining a biopsy. In some cases, the genetic material is obtained from a biopsy, e.g., from the intestinal track of the subject. Isolating components of fluid or tissue samples (e.g., cells or RNA or DNA) may be accomplished using a variety of techniques. After the sample is obtained, it may be further processed to enrich for or purify genomic material.

In some embodiments, the expression level of a biomarker in a sample from a subject is compared to a reference expression level (or reference expression profile). In some cases, the reference expression level is from a subject that does not comprise IBD. In some cases, the reference expression level is from a subject that does not comprises a severe form of IBD.

In some cases, the reference expression level is from a subject that comprises a non-inflammatory MNP signature of CD. In some cases, the reference expression level is from a subject that comprises an inflammatory MNP signature (e.g., “CD13 cluster 1 subtype” in Example 1). In some cases, the reference expression level is from a subject that comprises a non-resident mucosal MNP signature of CD. In some cases, the reference expression level is from a subject that comprises a resident mucosal MNP signature (e.g., “CD13 cluster 2 subtype” in Example 1). In some cases, a patient having an inflammatory MNP signature has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who does not have IBD or has a resident mucosal MNP signature). In some cases, a patient having a resident mucosal MNP signature has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who does not have IBD or has an inflammatory MNP signature). Table 2A provides non-limiting examples of increased expression fold of biomarkers in an inflammatory MNP signature as compared to a resident mucosal MNP signature. Table 2B provides non-limiting examples of increased expression fold of biomarkers in a resident mucosal MNP signature as compared to an inflammatory MNP signature.

TABLE 2A
Exemplary Biomarkers of a Transcriptomic Signature

| | | | Geom | Geom | | | | | |
|---|---|---|---|---|---|---|---|---|---|
| | | | mean of | mean of | | | | | |
| Parametric | | Permutation | intensities | intensities | Fold | | | Entrez | |
| p-value | FDR | p-value | in class 1 | in class 2 | change | UniqueID | Name | ID | Accession |
| 0.0000001 | 5.39E−06 | <1e−07 | 65.87 | 32.91 | 2 | BTAF1 | B-TFIID TATA-box | 9044 | NM_003972 |
| | | | | | | | binding protein | | |
| | | | | | | | associated factor 1 | | |
| 0.0000006 | 2.07E−05 | <1e−07 | 36.17 | 18.06 | 2 | CLCN6 | chloride voltage- | 1185 | NM_001256959 |
| | | | | | | | gated channel 6 | | |
| 0.0000009 | 2.83E−05 | <1e−07 | 104.93 | 52.35 | 2 | GDI1 | GDP dissociation | 2664 | NM_001493 |
| | | | | | | | inhibitor 1 | | |
| 0.0000014 | 3.93E−05 | <1e−07 | 22.07 | 11.02 | 2 | ADNP2 | ADNP homeobox 2 | 22850 | NM_014913 |
| 0.000002 | 5.12E−05 | <1e−07 | 41.78 | 20.86 | 2 | DDHD1 | DDHD domain | 80821 | NM_001160147 |
| | | | | | | | containing 1 | | |
| 0.0000044 | 8.87E−05 | <1e−07 | 26.44 | 13.21 | 2 | SLC12A9 | solute carrier family | 56996 | NM_001267812 |
| | | | | | | | 12 member 9 | | |
| 0.0000144 | 0.00021 | <1e−07 | 1985.17 | 991 | 2 | SRRM2 | serine/arginine | 23524 | NM_016333 |
| | | | | | | | repetitive matrix 2 | | |
| 0.0000383 | 0.000426 | <1e−07 | 104.35 | 52.06 | 2 | CD81 | CD81 molecule | 975 | NM_001297649 |
| 0.0000406 | 0.000444 | <1e−07 | 151.87 | 75.86 | 2 | PARP14 | poly (ADP-ribose) | 54625 | NM_017554 |
| | | | | | | | polymerase family | | |
| | | | | | | | member 14 | | |
| | | | | | | | noncoding (Clone | | |
| | | | | | | | Fragment ID: | | |
| | | | | | | | AC011939.9) | | |
| 0.0003062 | 0.00204 | <1e−07 | 71.25 | 35.56 | 2 | DAPK1 | death associated | 1612 | NM_001288729 |
| | | | | | | | protein kinase 1 | | |
| <1e−07 | 5.39E−06 | <1e−07 | 68.2 | 33.87 | 2.01 | CPSF7 | cleavage and | 79869 | NM_001136040 |
| | | | | | | | polyadenylation | | |
| | | | | | | | specific factor 7 | | |
| 0.0000002 | 9.26E−06 | <1e−07 | 68.4 | 34.06 | 2.01 | TCF3 | transcription factor | 6929 | NM_001136139 |
| | | | | | | | 3 | | |
| 0.0000002 | 9.26E−06 | <1e−07 | 43.22 | 21.46 | 2.01 | ARMCX3 | armadillo repeat | 51566 | NM_016607 |
| | | | | | | | containing, X-linked | | |
| | | | | | | | 3 | | |
| 0.0000026 | 6.19E−05 | <1e−07 | 14.83 | 7.4 | 2.01 | TMEM268 | transmembrane | 203197 | NM_001330760 |
| | | | | | | | protein 268 | | |
| 0.0000027 | 6.33E−05 | <1e−07 | 27.03 | 13.42 | 2.01 | CHMP7 | charged | 91782 | NM_001317899 |
| | | | | | | | multivesicular body | | |
| | | | | | | | protein 7 | | |
| 0.0000032 | 7.14E−05 | <1e−07 | 44.65 | 22.25 | 2.01 | EDC4 | enhancer of mRNA | 23644 | NM_014329 |
| | | | | | | | decapping 4 | | |
| 0.0000055 | 0.000104 | <1e−07 | 44.29 | 22.08 | 2.01 | TIMM44 | translocase of inner | 10469 | NM_006351 |
| | | | | | | | mitochondrial | | |
| | | | | | | | membrane 44 | | |
| 0.0000173 | 0.000238 | <1e−07 | 63.44 | 31.49 | 2.01 | DGKD | diacylglycerol | 8527 | NM_003648 |
| | | | | | | | kinase delta | | |
| 0.0000298 | 0.000354 | 0.0001 | 52.07 | 25.94 | 2.01 | ARHGAP25 | Rho GTPase | 9938 | NM_001007231 |
| | | | | | | | activating protein 25 | | |
| 0.0000363 | 0.000409 | <1e−07 | 19.53 | 9.73 | 2.01 | SRA1 | steroid receptor | 10011 | NM_001035235 |
| | | | | | | | RNA activator 1 | | |
| 0.0000949 | 0.000836 | <1e−07 | 36.8 | 18.26 | 2.01 | DOCK11 | dedicator of | 139818 | NM_144658 |
| | | | | | | | cytokinesis 11 | | |
| 0.0001488 | 0.00119 | 0.0002 | 102.58 | 51.02 | 2.01 | MAFK | MAF bZIP | 7975 | NM_002360 |
| | | | | | | | transcription factor | | |
| | | | | | | | K | | |
| <1e−07 | <1e−07 | <1e−07 | 402.76 | 199.61 | 2.02 | DYNC1H1 | dynein cytoplasmic | 1778 | NM_001376 |
| | | | | | | | 1 heavy chain 1 | | |
| <1e−07 | <1e−07 | <1e−07 | 98.69 | 48.95 | 2.02 | BAG6 | BCL2 associated | 7917 | NM_001098534 |
| | | | | | | | athanogene 6 | | |
| <1e−07 | 5.39E−06 | <1e−07 | 162.04 | 80.19 | 2.02 | PRPF38B | pre-mRNA | 55119 | NM_001349757 |
| | | | | | | | processing factor | | |
| | | | | | | | 38B | | |
| 0.0000003 | 1.23E−05 | <1e−07 | 42.37 | 20.93 | 2.02 | TPP2 | tripeptidyl peptidase | 7174 | NM_001330588 |
| | | | | | | | 2 | | |
| 0.0000004 | 0.000015 | <1e−07 | 369.52 | 182.63 | 2.02 | POLR2A | RNA polymerase II | 5430 | NM_000937 |
| | | | | | | | subunit A | | |
| 0.0000004 | 0.000015 | <1e−07 | 101.86 | 50.36 | 2.02 | KANSLI | KAT8 regulatory | 284058 | NM_001193465 |
| | | | | | | | NSL complex | | |
| | | | | | | | subunit 1 | | |
| 0.0000007 | 2.34E−05 | <1e−07 | 114.75 | 56.88 | 2.02 | PCNX1 | pecanex homolog 1 | 22990 | NM_001308160 |
| 0.0000008 | 2.61E−05 | <1e−07 | 175.09 | 86.77 | 2.02 | CLK1 | CDC like kinase 1 | 1195 | NM_001024646 |
| 0.0000021 | 5.27E−05 | <1e−07 | 572.37 | 282.86 | 2.02 | KLF6 | Kruppel like factor 6 | 1316 | NM_001008490 |
| 0.0000023 | 5.65E−05 | <1e−07 | 38.41 | 19.01 | 2.02 | RGL2 | ral guanine | 5863 | NM_001243738 |
| | | | | | | | nucleotide | | |
| | | | | | | | dissociation | | |
| | | | | | | | stimulator like 2 | | |
| 0.0000024 | 5.78E−05 | <1e−07 | 114.99 | 57.03 | 2.02 | GNL1 | G protein nucleolar | 2794 | NM_005275 |
| | | | | | | | 1 (putative) | | |
| 0.0000034 | 7.49E−05 | <1e−07 | 66.67 | 33.02 | 2.02 | CCDC93 | coiled-coil domain | 54520 | NM_019044 |
| | | | | | | | containing 93 | | |
| 0.0000152 | 0.000218 | <1e−07 | 130.02 | 64.42 | 2.02 | FMNL3 | form in like 3 | 91010 | NM_175736 |
| 0.0000224 | 0.000288 | 0.0001 | 18.35 | 9.1 | 2.02 | AC013394.1 | noncoding | | |
| | | | | | | | (Ensemble gene ID: | | |
| | | | | | | | ENSG00000272888.1) | | |
| 0.0000283 | 0.000343 | <1e−07 | 180.5 | 89.17 | 2.02 | GOLGA3 | golgin A3 | 2802 | NM_001172557 |
| 0.0000457 | 0.000488 | 0.0001 | 18.71 | 9.28 | 2.02 | COL4A4 | colla gen type IV | 1286 | NM_000092 |
| | | | | | | | alpha 4 chain | | |
| 0.0000648 | 0.000626 | <1e−07 | 20.04 | 9.94 | 2.02 | C16orf54 | chromosome 16 | 283897 | NM_175900 |
| | | | | | | | open reading frame | | |
| | | | | | | | 54 | | |
| 0.0001031 | 0.00089 | 0.0001 | 35.52 | 17.55 | 2.02 | TEDC1 | tubulin epsilon and | 283643 | NM_001134875 |
| | | | | | | | delta complex 1 | | |
| 0.0001134 | 0.000958 | <1e−07 | 128.14 | 63.49 | 2.02 | SEL1L | SEL1L ERAD E3 | 6400 | NM_001244984 |
| | | | | | | | ligase adaptor | | |
| | | | | | | | subunit | | |
| 0.0001284 | 0.00105 | <1e−07 | 59.38 | 29.43 | 2.02 | U2SURP | U2 snRNP | 23350 | NM_001080415 |
| | | | | | | | associated SURP | | |
| | | | | | | | domain containing | | |
| 0.0001547 | 0.00122 | 0.0001 | 37.81 | 18.74 | 2.02 | ARFGAP3 | ADP ribosy lation | 26286 | NM_001142293 |
| | | | | | | | factor GTPase | | |
| | | | | | | | activating protein 3 | | |
| 0.0001989 | 0.00147 | 0.0004 | 20.15 | 9.98 | 2.02 | APBB3 | amyloid beta | 10307 | NM_006051 |
| | | | | | | | precursor protein | | |
| | | | | | | | binding family B | | |
| | | | | | | | member 3 | | |
| 0.0002365 | 0.00168 | 0.0001 | 65.94 | 32.65 | 2.02 | TCF4 | transcription factor | 6925 | NM_001083962 |
| | | | | | | | 4 | | |
| 0.0002421 | 0.00171 | 0.0002 | 30.46 | 15.07 | 2.02 | BTN2A2 | butyrophilin | 10385 | NM_001197237 |
| | | | | | | | subfamily 2 member | | |
| | | | | | | | A2 | | |
| <1e−07 | <1e−07 | <1e−07 | 81.75 | 40.28 | 2.03 | HNRNPA0 | heterogeneous | 10949 | NM_006805 |
| | | | | | | | nuclear | | |
| | | | | | | | ribonucleoprotein | | |
| | | | | | | | A0 | | |
| <1e−07 | <1e−07 | <1e−07 | 60.26 | 29.68 | 2.03 | SEC24C | SEC24 homolog C, | 9632 | NM_004922 |
| | | | | | | | COPII coat complex | | |
| | | | | | | | component | | |
| <1e−07 | 5.39E−06 | <1e−07 | 15.41 | 7.58 | 2.03 | PTOV1-AS2 | PTOV1 antisense | 101928378 | NR_110730 |
| | | | | | | | RNA 2 | | |
| 0.0000002 | 9.26E−06 | <1e−07 | 71.44 | 35.27 | 2.03 | SCAF4 | SR-related CTD | 57466 | NM_001145444 |
| | | | | | | | associated factor 4 | | |
| 0.000001 | 3.07E−05 | <1e−07 | 39.41 | 19.38 | 2.03 | DNAJB2 | DnaJ heat shock | 3300 | NM_001039550 |
| | | | | | | | protein family | | |
| | | | | | | | (Hsp40) member B2 | | |
| 0.0000016 | 4.31E−05 | <1e−07 | 34.74 | 17.14 | 2.03 | RIC1 | RIC1 homolog, | 57589 | NM_001135920 |
| | | | | | | | RAB6A GEF | | |
| | | | | | | | complex partner 1 | | |
| 0.0000032 | 7.14E−05 | <1e−07 | 52.75 | 25.95 | 2.03 | ZNF394 | zinc finger protein | 84124 | NM_001345967 |
| | | | | | | | 394 | | |
| 0.0000053 | 0.000101 | <1e−07 | 17.6 | 8.65 | 2.03 | CLN8 | CLN8, | 2055 | NM_001034061 |
| | | | | | | | transmembrane ER | | |
| | | | | | | | and ERGIC protein | | |
| 0.0000069 | 0.000122 | <1e−07 | 23.94 | 11.82 | 2.03 | SLC35C2 | solute carrier family | 51006 | NM_001281457 |
| | | | | | | | 35 member C2 | | |
| 0.0000075 | 0.00013 | <1e−07 | 81.41 | 40.05 | 2.03 | KDM5A | lysine demethylase | 5927 | NM_001042603 |
| | | | | | | | 5A | | |
| 0.0000087 | 0.000145 | <1e−07 | 34.23 | 16.86 | 2.03 | TESK1 | testis associated | 7016 | NM_001318230 |
| | | | | | | | actin remodelling | | |
| | | | | | | | kinase 1 | | |
| 0.0000204 | 0.000269 | <1e−07 | 20.03 | 9.88 | 2.03 | KLHL5 | kelch like family | 51088 | NM_001007075 |
| | | | | | | | member 5 | | |
| 0.0000435 | 0.000469 | <1e−07 | 24.96 | 12.31 | 2.03 | ATP6V0B | ATPase H+ | 533 | NM_001039457 |
| | | | | | | | transporting V0 | | |
| | | | | | | | subunit b | | |
| 0.0000484 | 0.000508 | 0.0002 | 21.58 | 10.65 | 2.03 | ZNF275 | zinc finger protein | 10838 | NM_001080485 |
| | | | | | | | 275 | | |
| 0.0007997 | 0.00431 | 0.0006 | 1006.5 | 496.11 | 2.03 | LMNA | lamin A/C | 4000 | NM_001257374 |
| <1e−07 | <1e−07 | <1e−07 | 140.7 | 69.07 | 2.04 | PCF11 | PCF11 cleavage and | 51585 | NM_001346413 |
| | | | | | | | polyadenylation | | |
| | | | | | | | factor subunit | | |
| <1e−07 | <1e−07 | <1e−07 | 193.6 | 94.76 | 2.04 | CIRBP | cold inducible RNA | 1153 | NM_001280 |
| | | | | | | | binding protein | | |
| <1e−07 | 5.39E−06 | <1e−07 | 227.23 | 111.15 | 2.04 | UBE2D3 | ubiquitin | 7323 | NM_001300795 |
| | | | | | | | conjugating enzyme | | |
| | | | | | | | E2 D3 | | |
| 0.0000002 | 9.26E−06 | <1e−07 | 29.07 | 14.28 | 2.04 | USF1 | upstream | 7391 | NM_001276373 |
| | | | | | | | transcription factor | | |
| | | | | | | | 1 | | |
| 0.0000004 | 0.000015 | <1e−07 | 116.78 | 57.21 | 2.04 | IQGAP2 | IQmotif containing | 10788 | NM_001285460 |
| | | | | | | | GTPase activating | | |
| | | | | | | | protein 2 | | |
| 0.0000005 | 1.78E−05 | <1e−07 | 64.21 | 31.48 | 2.04 | TASOR2 | Transcription | 54906 | |
| | | | | | | | Activation | | |
| | | | | | | | Suppressor Family | | |
| | | | | | | | Member 2 | | |
| 0.0000005 | 1.78E−05 | <1e−07 | 24.37 | 11.94 | 2.04 | LRRC41 | leucine rich repeat | 10489 | NM_006369 |
| | | | | | | | containing 41 | | |
| 0.0000019 | 0.000049 | <1e−07 | 119.78 | 58.74 | 2.04 | TRIM28 | tripartite motif | 10155 | NM_005762 |
| | | | | | | | containing 28 | | |
| 0.0000043 | 8.74E−05 | <1e−07 | 23.76 | 11.63 | 2.04 | TSSC4 | tumor suppressing | 10078 | NM_001297658 |
| | | | | | | | subtransferable | | |
| | | | | | | | candidate 4 | | |
| 0.0000053 | 0.000101 | 0.0001 | 61.48 | 30.07 | 2.04 | MAZ | MYC associated | 4150 | NM_001042539 |
| | | | | | | | zinc finger protein | | |
| 0.0000113 | 0.000175 | 0.0001 | 464.93 | 227.95 | 2.04 | DDX3X | DEAD-box helicase | 1654 | NM_001193416 |
| | | | | | | | 3, X-linked | | |
| 0.0000269 | 0.000329 | <1e−07 | 43.46 | 21.31 | 2.04 | CHST11 | carbohydrate | 50515 | NM_001173982 |
| | | | | | | | sulfotransferase 11 | | |
| 0.0000353 | 0.000401 | <1e−07 | 24.71 | 12.11 | 2.04 | RRN3P3 | RRN3 homolog, | 100131998 | NR_027460 |
| | | | | | | | RNA polymerase I | | |
| | | | | | | | transcription factor | | |
| | | | | | | | pseudogene 3 | | |
| 0.0000675 | 0.000645 | <1e−07 | 33.64 | 16.52 | 2.04 | HLA-L | major | 3139 | NR_027822 |
| | | | | | | | histocompatibility | | |
| | | | | | | | complex, class I, L | | |
| | | | | | | | (pseudogene) | | |
| 0.0000725 | 0.000682 | 0.0001 | 37.53 | 18.43 | 2.04 | GNS | glucosamine (N- | 2799 | NM_002076 |
| | | | | | | | acetyl)-6-sulfatase | | |
| 0.0000772 | 0.000715 | <1e−07 | 38.2 | 18.73 | 2.04 | REC8 | REC8 meiotic | 9985 | NM_001048205 |
| | | | | | | | recombination | | |
| | | | | | | | protein | | |
| 0.0000899 | 0.000803 | 0.0001 | 46.65 | 22.85 | 2.04 | CSGALNACT2 | chondroitin sulfate | 55454 | NM_001319654 |
| | | | | | | | N- | | |
| | | | | | | | acetylgalactosaminyl- | | |
| | | | | | | | transferase 2 | | |
| 0.0001106 | 0.000941 | <1e−07 | 34.74 | 17.04 | 2.04 | ZFYVE16 | zinc finger FYVE- | 9765 | NM_001105251 |
| | | | | | | | type containing 16 | | |
| 0.000161 | 0.00126 | 0.0002 | 134.22 | 65.73 | 2.04 | ARID5B | AT-rich interaction | 84159 | NM_001244638 |
| | | | | | | | domain 5B | | |
| 0.0001705 | 0.00131 | 0.0002 | 58.32 | 28.54 | 2.04 | CNOT6L | CCR4-NOT | 246175 | NM_001286790 |
| | | | | | | | transcription | | |
| | | | | | | | complex subunit 6 | | |
| | | | | | | | like | | |
| <1e−07 | 5.39E−06 | <1e−07 | 12.58 | 6.15 | 2.05 | QPCTL | glutaminyl-peptide | 54814 | NM_001163377 |
| | | | | | | | cyclotransferase like | | |
| <1e−07 | 5.39E−06 | <1e−07 | 924.07 | 449.77 | 2.05 | DDX5 | DEAD-box helicase | 1655 | NM_001320595 |
| | | | | | | | 5 | | |
| 0.0000003 | 1.23E−05 | <1e−07 | 123.46 | 60.08 | 2.05 | HGS | hepatocyte growth | 9146 | NM_004712 |
| | | | | | | | factor-regulated | | |
| | | | | | | | tyrosine kinase | | |
| | | | | | | | substrate | | |
| 0.0000003 | 1.23E−05 | <1e−07 | 22.33 | 10.89 | 2.05 | TENT2 | Terminal | 167153 | |
| | | | | | | | Nucleotidyltransferase | | |
| | | | | | | | 2 | | |
| 0.0000007 | 2.34E−05 | <1e−07 | 476.75 | 232.01 | 2.05 | CD44 | CD44 molecule | 960 | NM_000610 |
| | | | | | | | (Indian blood group) | | |
| 0.0000018 | 4.73E−05 | <1e−07 | 41.38 | 20.18 | 2.05 | TBC1D17 | TBC1 domain | 79735 | NM_001168222 |
| | | | | | | | family member 17 | | |
| 0.0000029 | 6.68E−05 | <1e−07 | 217.85 | 106.46 | 2.05 | TRA2B | transformer 2 beta | 6434 | NM_001243879 |
| | | | | | | | homolog | | |
| 0.0000086 | 0.000144 | <1e−07 | 60.93 | 29.65 | 2.05 | PPP2R5C | protein phosphatase | 5527 | NM_001161725 |
| | | | | | | | 2 regulatory subunit | | |
| | | | | | | | B'gamma | | |
| 0.0000121 | 0.000185 | <1e−07 | 81.41 | 39.73 | 2.05 | CTBP1 | C-terminal binding | 1487 | NM_001012614 |
| | | | | | | | protein 1 | | |
| 0.0000217 | 0.000281 | 0.0001 | 351.25 | 171.2 | 2.05 | ADGRE5 | adhesion G protein- | 976 | NM_001025160 |
| | | | | | | | coupled receptor E5 | | |
| 0.0000221 | 0.000285 | <1e−07 | 15.48 | 7.56 | 2.05 | TNFRSF4 | TNF receptor | 7293 | NM_003327 |
| | | | | | | | superfamily member | | |
| | | | | | | | 4 | | |
| 0.0000332 | 0.000384 | <1e−07 | 23.3 | 11.36 | 2.05 | ORMDL3 | ORMDL | 94103 | NM_001320801 |
| | | | | | | | sphingolipid | | |
| | | | | | | | biosynthesis | | |
| | | | | | | | regulator 3 | | |
| 0.0000647 | 0.000626 | <1e−07 | 93.5 | 45.7 | 2.05 | FNDC3A | fibronectin type III | 22862 | NM_001079673 |
| | | | | | | | domain containing | | |
| | | | | | | | 3A | | |
| 0.0000719 | 0.000678 | 0.0001 | 61.26 | 29.83 | 2.05 | VAVI | vav guanine | 7409 | NM_001258206 |
| | | | | | | | nucleotide exchange | | |
| | | | | | | | factor 1 | | |
| 0.0001068 | 0.000916 | 0.0002 | 35.23 | 17.18 | 2.05 | KMT2E-AS1 | KMT2E antisense | 100216545 | NR_024586 |
| | | | | | | | RNA 1 | | |
| | | | | | | | noncoding | | |
| 0.0001082 | 0.000927 | 0.0002 | 19.36 | 9.45 | 2.05 | AC116366.2 | (Ensemble gene ID: | | |
| | | | | | | | ENSG00000238160.1) | | |
| 0.0001105 | 0.000941 | <1e−07 | 19.62 | 9.55 | 2.05 | SMOX | spermine oxidase | 54498 | NM_001270691 |
| 0.0001466 | 0.00117 | 0.0001 | 30.61 | 14.93 | 2.05 | DNAAF2 | dynein axonemal | 55172 | NM_001083908 |
| | | | | | | | assembly factor 2 | | |
| 0.0002734 | 0.00187 | <1e−07 | 84.78 | 41.38 | 2.05 | BCL6 | B cell | 604 | NM_001130845 |
| | | | | | | | CLL/lymphoma 6 | | |
| 0.0004987 | 0.00296 | 0.0002 | 44.98 | 21.99 | 2.05 | MEF2C | myocyte enhancer | 4208 | NM_001131005 |
| | | | | | | | factor 2C | | |
| 0.0007846 | 0.00425 | 0.0006 | 56.58 | 27.58 | 2.05 | CTSS | cathepsin S | 1520 | NM_001199739 |
| <1e−07 | <1e−07 | <1e−07 | 312.66 | 152.03 | 2.06 | SON | SON DNA binding | 6651 | NM_001291411 |
| | | | | | | | protein | | |
| <1e−07 | <1e−07 | <1e−07 | 105.73 | 51.21 | 2.06 | MYCBP2 | MYC binding | 23077 | NM_015057 |
| | | | | | | | protein 2, E3 | | |
| | | | | | | | ubiquitin protein | | |
| | | | | | | | ligase | | |
| <1e−07 | <1e−07 | <1e−07 | 16.7 | 8.1 | 2.06 | ENO2 | enolase 2 | 2026 | NM_001975 |
| <1e−07 | <1e−07 | <1e−07 | 268.86 | 130.79 | 2.06 | CYTH1 | cytohesin 1 | 9267 | NM_001292018 |
| <1e−07 | 5.39E−06 | <1e−07 | 16.74 | 8.12 | 2.06 | FBXO33 | F-box protein 33 | 254170 | NM_203301 |
| 0.0000001 | 5.39E−06 | <1e−07 | 44.89 | 21.83 | 2.06 | UBR3 | ubiquitin protein | 130507 | NM_172070 |
| | | | | | | | ligase E3 | | |
| | | | | | | | component n- | | |
| | | | | | | | recognin 3 | | |
| | | | | | | | (putative) | | |
| 0.0000002 | 9.26E−06 | <1e−07 | 247.91 | 120.55 | 2.06 | PIEZO1 | piezo type | 9780 | NM_001142864 |
| | | | | | | | mechanosensitive | | |
| | | | | | | | ion channel | | |
| | | | | | | | component 1 | | |
| 0.0000019 | 0.000049 | <1e−07 | 286.42 | 138.79 | 2.06 | HIPK2 | homeodomain | 28996 | NM_001113239 |
| | | | | | | | interacting protein | | |
| | | | | | | | kinase 2 | | |
| 0.0000021 | 5.27E−05 | <1e−07 | 71 | 34.4 | 2.06 | CCDC88A | coiled-coil domain | 55704 | NM_001135597 |
| | | | | | | | containing 88A | | |
| 0.0000026 | 6.19E−05 | <1e−07 | 33.09 | 16.03 | 2.06 | FCHSD1 | FCH and double | 89848 | NM_033449 |
| | | | | | | | SH3 domains 1 | | |
| 0.0000027 | 6.33E−05 | <1e−07 | 138.79 | 67.32 | 2.06 | BDP1 | B double prime 1, | 55814 | NM_018429 |
| | | | | | | | subunit of RNA | | |
| | | | | | | | polymerase III | | |
| | | | | | | | transcription | | |
| | | | | | | | initiation factor IIIB | | |
| 0.000003 | 6.83E−05 | <1e−07 | 207.97 | 100.75 | 2.06 | IGF2R | insulin like growth | 3482 | NM_000876 |
| | | | | | | | factor 2 receptor | | |
| 0.0000062 | 0.000113 | <1e−07 | 34.16 | 16.61 | 2.06 | TAF4B | TATA-box binding | 6875 | NM_001293725 |
| | | | | | | | protein associated | | |
| | | | | | | | factor 4b | | |
| 0.0000068 | 0.000121 | <1e−07 | 80.91 | 39.22 | 2.06 | ACAP3 | ArfGAP with | 116983 | NM_030649 |
| | | | | | | | coiled-coil, ankyrin | | |
| | | | | | | | repeat and PH | | |
| | | | | | | | domains 3 | | |
| 0.00001 | 0.000159 | <1e−07 | 26.99 | 13.1 | 2.06 | XPOT | exportin for tRNA | 11260 | NM_007235 |
| 0.000014 | 0.000205 | <1e−07 | 23.63 | 11.46 | 2.06 | PRKD3 | protein kinase D3 | 23683 | NM_005813 |
| 0.0000172 | 0.000237 | <1e−07 | 74.99 | 36.47 | 2.06 | SLC6A6 | solute carrier family | 6533 | NM_001134367 |
| | | | | | | | 6 member 6 | | |
| 0.0000305 | 0.00036 | 0.0001 | 82.04 | 39.79 | 2.06 | FAM102A | family with | 399665 | NM_001035254 |
| | | | | | | | sequence similarity | | |
| | | | | | | | 102 member A | | |
| 0.0000491 | 0.000512 | <1e−07 | 30.39 | 14.79 | 2.06 | CDC25B | cell division cycle | 994 | NM_001287516 |
| | | | | | | | 25B | | |
| 0.0000739 | 0.00069 | <1e−07 | 75.52 | 36.73 | 2.06 | NABP1 | nucleic acid binding | 64859 | NM_001031716 |
| | | | | | | | protein 1 | | |
| 0.0000964 | 0.000846 | 0.0002 | 81.53 | 39.65 | 2.06 | ATP1B3 | ATPase Na+/K+ | 483 | NM_001679 |
| | | | | | | | transporting subunit | | |
| | | | | | | | beta 3 | | |
| 0.0000988 | 0.000861 | 0.0001 | 31.69 | 15.36 | 2.06 | RTL5 | retrotransposon Gag | 340526 | NM_001024455 |
| | | | | | | | like 5 | | |
| 0.0001091 | 0.000933 | 0.0001 | 21.73 | 10.54 | 2.06 | AQP3 | aquaporin 3 (Gill | 360 | NM_001318144 |
| | | | | | | | blood group) | | |
| 0.0001741 | 0.00133 | 0.0001 | 15.27 | 7.41 | 2.06 | NUGGC | nuclear GTPase, | 389643 | NM_001010906 |
| | | | | | | | germinal center | | |
| | | | | | | | associated | | |
| 0.0002539 | 0.00176 | 0.0004 | 20.34 | 9.85 | 2.06 | TSPAN33 | tetraspanin 33 | 340348 | NM_178562 |
| 0.0007625 | 0.00416 | 0.0006 | 34.35 | 16.65 | 2.06 | TRPM2 | transient receptor | 7226 | NM_001001188 |
| | | | | | | | potential cation | | |
| | | | | | | | channel subfamily | | |
| | | | | | | | M member 2 | | |
| <1e−07 | 5.39E−06 | <1e−07 | 61.45 | 29.63 | 2.07 | KHNYN | KH and NYN | 23351 | NM_001290256 |
| | | | | | | | domain containing | | |
| 0.0000017 | 4.51E−05 | <1e−07 | 44.98 | 21.69 | 2.07 | UBALDI | UBA like domain | 124402 | NM_001330467 |
| | | | | | | | containing 1 | | |
| 0.0000022 | 5.47E−05 | <1e−07 | 31.56 | 15.21 | 2.07 | CCDC97 | coiled-coil domain | 90324 | NM_001346100 |
| | | | | | | | containing 97 | | |
| 0.0000036 | 7.74E−05 | <1e−07 | 62.42 | 30.09 | 2.07 | CLPTM1L | CLPTM1 like | 81037 | NM_030782 |
| 0.000004 | 8.27E−05 | <1e−07 | 36.12 | 17.41 | 2.07 | UBE2D2 | ubiquitin | 7322 | NM_003339 |
| | | | | | | | conjugating enzyme | | |
| | | | | | | | E2 D2 | | |
| 0.0000043 | 8.74E−05 | <1e−07 | 63.49 | 30.69 | 2.07 | GANAB | glucosidase II alpha | 23193 | NM_001278192 |
| | | | | | | | subunit | | |
| 0.000005 | 9.72E−05 | <1e−07 | 64.66 | 31.24 | 2.07 | MTCH1 | mitochondrial | 23787 | NM_001271641 |
| | | | | | | | carrier 1 | | |
| 0.000008 | 0.000137 | <1e−07 | 26.01 | 12.59 | 2.07 | CCDC85B | coiled-coil domain | 11007 | NM_006848 |
| | | | | | | | containing 85B | | |
| 0.0000085 | 0.000142 | 0.0001 | 220.89 | 106.72 | 2.07 | HIVEP2 | human | 3097 | NM_006734 |
| | | | | | | | immunodeficiency | | |
| | | | | | | | virus type I | | |
| | | | | | | | enhancer binding | | |
| | | | | | | | protein 2 | | |
| 0.0000094 | 0.000154 | <1e−07 | 33.73 | 16.29 | 2.07 | PSMA3-AS1 | PSMA3 antisense | 379025 | NR_029434 |
| | | | | | | | RNA 1 | | |
| 0.00001 | 0.000159 | <1e−07 | 27.37 | 13.21 | 2.07 | E4F1 | E4F transcription | 1877 | NM_001288776 |
| | | | | | | | factor 1 | | |
| 0.0000491 | 0.000512 | <1e−07 | 55.06 | 26.58 | 2.07 | ATP2B4 | ATPase plasma | 493 | NM_001001396 |
| | | | | | | | membrane Ca2+ | | |
| | | | | | | | transporting 4 | | |
| 0.0000645 | 0.000625 | <1e−07 | 27.95 | 13.47 | 2.07 | PIK3IP1 | phosphoinositide-3- | 113791 | NM_001135911 |
| | | | | | | | kinase interacting | | |
| | | | | | | | protein 1 | | |
| 0.0001335 | 0.00109 | 0.0001 | 27.5 | 13.3 | 2.07 | CASTOR2 | cytosolic arginine | 729438 | NM_001145064 |
| | | | | | | | sensorform TORC1 | | |
| | | | | | | | subunit 2 | | |
| 0.0002549 | 0.00177 | <1e−07 | 137.94 | 66.66 | 2.07 | ABCC1 | ATP binding | 4363 | NM_004996 |
| | | | | | | | cassette subfamily C | | |
| | | | | | | | member 1 | | |
| <1e−07 | 5.39E−06 | <1e−07 | 20.65 | 9.91 | 2.08 | CERS4 | ceramide synthase 4 | 79603 | NM_024552 |
| <1e−07 | 5.39E−06 | <1e−07 | 216.88 | 104.05 | 2.08 | SF3B1 | splicing factor 3b | 23451 | NM_001005526 |
| | | | | | | | subunit 1 | | |
| 0.0000003 | 1.23E−05 | <1e−07 | 132.49 | 63.62 | 2.08 | TRRAP | transformation/ | 8295 | NM_001244580 |
| | | | | | | | transcription domain | | |
| | | | | | | | associated protein | | |
| 0.0000012 | 3.52E−05 | <1e−07 | 33.53 | 16.13 | 2.08 | FYTTD1 | forty-two-three | 84248 | NM_001011537 |
| | | | | | | | domain containing 1 | | |
| 0.0000015 | 0.000041 | <1e−07 | 23.13 | 11.12 | 2.08 | ATF2 | activating | 1386 | NM_001256090 |
| | | | | | | | transcription factor | | |
| | | | | | | | 2 | | |
| 0.0000038 | 7.99E−05 | <1e−07 | 53.19 | 25.6 | 2.08 | SWAP70 | switching B cell | 23075 | NM_001297714 |
| | | | | | | | complex subunit | | |
| | | | | | | | SWAP70 | | |
| 0.000005 | 9.72E−05 | <1e−07 | 26.14 | 12.58 | 2.08 | CPNE1 | copine 1 | 8904 | NM_001198863 |
| 0.0000162 | 0.000228 | <1e−07 | 29.49 | 14.2 | 2.08 | KLF16 | Kruppel like factor | 83855 | NM_031918 |
| | | | | | | | 16 | | |
| 0.0000173 | 0.000238 | <1e−07 | 49.42 | 23.71 | 2.08 | ELMO1 | engulfment and cell | 9844 | NM_001039459 |
| | | | | | | | motility 1 | | |
| 0.000098 | 0.000855 | 0.0003 | 20.19 | 9.69 | 2.08 | SNHG1 | small nucleolar | 23642 | NR_003098 |
| | | | | | | | RNA host gene 1 | | |
| 0.0002857 | 0.00193 | 0.0001 | 48.18 | 23.21 | 2.08 | MIAT | myocardial | 440823 | NR_003491 |
| | | | | | | | infarction associated | | |
| | | | | | | | transcript (non- | | |
| | | | | | | | protein coding) | | |
| 0.000314 | 0.00208 | 0.0001 | 290.72 | 139.92 | 2.08 | PIM3 | Pim-3 proto- | 415116 | NM_001001852 |
| | | | | | | | oncogene, | | |
| | | | | | | | serine/threonine | | |
| | | | | | | | kinase | | |
| 0.0009638 | 0.00499 | 0.0004 | 44.22 | 21.3 | 2.08 | COBLL1 | cordon-bleu WH2 | 22837 | NM_001278458 |
| | | | | | | | repeat protein like 1 | | |
| <1e−07 | <1e−07 | <1e−07 | 158.26 | 75.84 | 2.09 | ASXL1 | additional sex | 171023 | NM_001164603 |
| | | | | | | | combs like 1, | | |
| | | | | | | | transcriptional | | |
| | | | | | | | regulator | | |
| <1e−07 | <1e−07 | <1e−07 | 26.13 | 12.51 | 2.09 | DGCR8 | DGCR8, | 54487 | NM_001190326 |
| | | | | | | | microprocessor | | |
| | | | | | | | complex subunit | | |
| 0.0000001 | 5.39E−06 | <1e−07 | 23.85 | 11.4 | 2.09 | PSMC6 | protea some 26S | 5706 | NM_002806 |
| | | | | | | | subunit, ATPase 6 | | |
| 0.0000002 | 9.26E−06 | <1e−07 | 32.66 | 15.63 | 2.09 | KLC2 | kinesin light chain 2 | 64837 | NM_001134774 |
| 0.0000004 | 0.000015 | <1e−07 | 29.75 | 14.26 | 2.09 | CAPN10 | calpain 10 | 11132 | NM_021251 |
| 0.0000007 | 2.34E−05 | <1e−07 | 38.87 | 18.64 | 2.09 | GNPTG | N- | 84572 | NM_032520 |
| | | | | | | | acetylglucosamine- | | |
| | | | | | | | 1-phosphate | | |
| | | | | | | | transferase gamma | | |
| | | | | | | | subunit | | |
| 0.0000009 | 2.83E−05 | <1e−07 | 14.03 | 6.71 | 2.09 | TEC | tec protein tyrosine | 7006 | NM_003215 |
| | | | | | | | kinase | | |
| 0.0000009 | 2.83E−05 | <1e−07 | 46.48 | 22.25 | 2.09 | MED25 | mediator complex | 81857 | NM_030973 |
| | | | | | | | subunit 25 | | |
| 0.0000019 | 0.000049 | <1e−07 | 51.33 | 24.55 | 2.09 | RFX1 | regulatory factor X1 | 5989 | NM_002918 |
| 0.0000027 | 6.33E−05 | <1e−07 | 67.94 | 32.49 | 2.09 | MYO5A | myosin VA | 4644 | NM_000259 |
| 0.0000045 | 8.96E−05 | <1e−07 | 44.05 | 21.13 | 2.09 | GNA12 | G protein subunit | 2768 | NM_001282440 |
| | | | | | | | alpha 12 | | |
| 0.0000097 | 0.000156 | <1e−07 | 857.82 | 410.38 | 2.09 | MCL1 | MCL1, BCL2 | 4170 | NM_001197320 |
| | | | | | | | family apoptosis | | |
| | | | | | | | regulator | | |
| 0.00001 | 0.000159 | <1e−07 | 20.27 | 9.72 | 2.09 | RNF122 | ring finger protein | 79845 | NM_024787 |
| | | | | | | | 122 | | |
| 0.0000121 | 0.000185 | <1e−07 | 47.41 | 22.64 | 2.09 | BMF | Bcl2 modifying | 90427 | NM_001003940 |
| | | | | | | | factor | | |
| 0.0000128 | 0.000192 | <1e−07 | 35.32 | 16.9 | 2.09 | FBXW4 | F-box and WD | 6468 | NM_001323541 |
| | | | | | | | repeat domain | | |
| | | | | | | | containing 4 | | |
| 0.0000134 | 0.000199 | <1e−07 | 70.92 | 33.98 | 2.09 | MAN2C1 | mannosidase alpha | 4123 | NM_001256494 |
| | | | | | | | class 2C member 1 | | |
| 0.0000349 | 0.000397 | <1e−07 | 50.95 | 24.35 | 2.09 | DNAJB11 | DnaJ heat shock | 51726 | NM_016306 |
| | | | | | | | protein family | | |
| | | | | | | | (Hsp40) member | | |
| | | | | | | | B11 | | |
| 0.0000447 | 0.000479 | <1e−07 | 25.61 | 12.24 | 2.09 | BBC3 | BCL2 binding | 27113 | NM_001127240 |
| | | | | | | | component 3 | | |
| 0.000046 | 0.00049 | <1e−07 | 51.66 | 24.72 | 2.09 | ABCA7 | ATP binding | 10347 | NM_019112 |
| | | | | | | | cassette subfamily A | | |
| | | | | | | | member 7 | | |
| 0.0000639 | 0.000621 | <1e−07 | 34.41 | 16.47 | 2.09 | FLCN | folliculin | 201163 | NM_001353229 |
| 0.0000716 | 0.000676 | 0.0001 | 19.65 | 9.41 | 2.09 | STARD8 | StAR related lipid | 9754 | NM_001142503 |
| | | | | | | | transfer domain | | |
| | | | | | | | containing 8 | | |
| 0.0001181 | 0.000991 | 0.0002 | 155.18 | 74.09 | 2.09 | ATG2A | autophagy related | 23130 | NM_015104 |
| | | | | | | | 2A | | |
| 0.0001242 | 0.00103 | 0.0001 | 91.51 | 43.69 | 2.09 | TRANK1 | tetratricopeptide | 9881 | NM_001329998 |
| | | | | | | | repeat and ankyrin | | |
| | | | | | | | repeat containing 1 | | |
| <1e−07 | <1e−07 | <1e−07 | 247.22 | 117.63 | 2.1 | FUS | FUS RNA binding | 2521 | NM_001010850 |
| | | | | | | | protein | | |
| <1e−07 | <1e−07 | <1e−07 | 20.26 | 9.63 | 2.1 | CCM2 | CCM2 scaffold | 83605 | NM_001029835 |
| | | | | | | | protein | | |
| <1e−07 | 5.39E−06 | <1e−07 | 29.99 | 14.29 | 2.1 | FPGS | folylpoly glutamate | 2356 | NM_001018078 |
| | | | | | | | synthase | | |
| <1e−07 | 5.39E−06 | <1e−07 | 47.35 | 22.59 | 2.1 | ZBTB40 | zinc finger and BTB | 9923 | NM_001083621 |
| | | | | | | | domain containing | | |
| | | | | | | | 40 | | |
| 0.0000009 | 2.83E−05 | <1e−07 | 70.25 | 33.53 | 2.1 | KLHDC4 | kelch domain | 54758 | NM_001184854 |
| | | | | | | | containing 4 | | |
| 0.0000011 | 3.33E−05 | <1e−07 | 572.98 | 273.21 | 2.1 | MYO9B | myosin IXB | 4650 | NM_001130065 |
| 0.000002 | 5.12E−05 | 0.0001 | 41.73 | 19.87 | 2.1 | MAP2K7 | mitogen-activated | 5609 | NM_001297555 |
| | | | | | | | protein kinase | | |
| | | | | | | | kinase 7 | | |
| 0.0000021 | 5.27E−05 | <1e−07 | 23.25 | 11.06 | 2.1 | TARS3 | Threonyl-TRNA | | |
| | | | | | | | Synthetase 3 | | |
| 0.000006 | 0.00011 | <1e−07 | 48.06 | 22.88 | 2.1 | WHAMM | WAS protein | 123720 | NM_001080435 |
| | | | | | | | homologassociated | | |
| | | | | | | | with actin, golgi | | |
| | | | | | | | membranes and | | |
| | | | | | | | microtubules | | |
| 0.0000099 | 0.000159 | 0.0001 | 29.54 | 14.08 | 2.1 | MAGT1 | magnesium | 84061 | NM_032121 |
| | | | | | | | transporter 1 | | |
| 0.0000156 | 0.000222 | <1e−07 | 47.4 | 22.52 | 2.1 | ADCY3 | adenylate cyclase 3 | 109 | NM_001320613 |
| 0.0000156 | 0.000222 | <1e−07 | 176.37 | 83.89 | 2.1 | WIPF1 | WAS/WASL | 7456 | NM_001077269 |
| | | | | | | | interacting protein | | |
| | | | | | | | family member 1 | | |
| 0.0000286 | 0.000344 | <1e−07 | 19.1 | 9.12 | 2.1 | FAM53B | family with | 9679 | NM_014661 |
| | | | | | | | sequence similarity | | |
| | | | | | | | 53 member B | | |
| 0.0000562 | 0.000565 | 0.0001 | 31.56 | 15.02 | 2.1 | TRPS1 | transcriptional | 7227 | NM_001282902 |
| | | | | | | | repressor GATA | | |
| | | | | | | | binding 1 | | |
| 0.0000691 | 0.000657 | <1e−07 | 192.25 | 91.43 | 2.1 | SLC3A2 | solute carrier family | 6520 | NM_001012661 |
| | | | | | | | 3 member 2 | | |
| 0.0000805 | 0.000737 | 0.0002 | 25.97 | 12.38 | 2.1 | RALGPS2 | Ral GEF with PH | 55103 | NM_001286247 |
| | | | | | | | domain and SH3 | | |
| | | | | | | | binding motif 2 | | |
| 0.0001085 | 0.000929 | <1e−07 | 17.62 | 8.4 | 2.1 | GCNA | germ cell nuclear | 93953 | NM_052957 |
| | | | | | | | acidic peptidase | | |
| 0.0002053 | 0.00151 | 0.0003 | 51.34 | 24.47 | 2.1 | CHST15 | carbohydrate | 51363 | NM_001270764 |
| | | | | | | | sulfotransferase 15 | | |
| 0.0002056 | 0.00151 | 0.0002 | 256.22 | 122.01 | 2.1 | CSRNP1 | cysteine and serine | 64651 | NM_001320559 |
| | | | | | | | rich nuclear protein | | |
| | | | | | | | 1 | | |
| 0.0002076 | 0.00152 | 0.0003 | 13.29 | 6.31 | 2.1 | GPR15 | G protein-coupled | 2838 | NM_005290 |
| | | | | | | | receptor 15 | | |
| 0.0002568 | 0.00177 | 0.0006 | 33.61 | 16.01 | 2.1 | TUBB6 | tubulin beta 6 class | 84617 | NM_001303524 |
| | | | | | | | V | | |
| 0.0003078 | 0.00205 | 0.0003 | 68.54 | 32.7 | 2.1 | TMEM63B | transmembrane | 55362 | NM_001318792 |
| | | | | | | | protein 63B | | |
| 0.0005284 | 0.00309 | 0.0002 | 113.02 | 53.83 | 2.1 | TENT5C | Terminal | 54855 | |
| | | | | | | | Nucleotidyltransferase | | |
| | | | | | | | 5C | | |
| 0.0009106 | 0.00476 | 0.0012 | 23.6 | 11.23 | 2.1 | STAT4 | signal transducer | 6775 | NM_001243835 |
| | | | | | | | and activator of | | |
| | | | | | | | transcription 4 | | |
| 0.0009985 | 0.00514 | 0.0009 | 80.07 | 38.2 | 2.1 | SH3D21 | SH3 domain | 79729 | NM_001162530 |
| | | | | | | | containing 21 | | |
| <1e−07 | <1e−07 | <1e−07 | 134.97 | 63.88 | 2.11 | ASH1L | ASH1 like histone | 55870 | NM_018489 |
| | | | | | | | lysine | | |
| | | | | | | | methyltransferase | | |
| <1e−07 | <1e−07 | <1e−07 | 31.16 | 14.79 | 2.11 | SNX13 | sorting nexin 13 | 23161 | NM_001350862 |
| 0.0000001 | 5.39E−06 | <1e−07 | 127.08 | 60.34 | 2.11 | SLC38A10 | solute carrier family | 124565 | NM_001037984 |
| | | | | | | | 38 member 10 | | |
| 0.0000001 | 5.39E−06 | <1e−07 | 26.17 | 12.38 | 2.11 | TOPORS | TOP1 binding | 10210 | NM_001195622 |
| | | | | | | | arginine/serine rich | | |
| | | | | | | | protein | | |
| 0.000001 | 3.07E−05 | <1e−07 | 366.93 | 173.78 | 2.11 | ILF3 | interleukin enhancer | 3609 | NM_001137673 |
| | | | | | | | binding factor 3 | | |
| 0.0000023 | 5.65E−05 | <1e−07 | 36.83 | 17.49 | 2.11 | RING1 | ring finger protein 1 | 6015 | NM_002931 |
| 0.0000087 | 0.000145 | <1e−07 | 76.28 | 36.09 | 2.11 | SEPTIN6 | Septin 6 | 23157 | |
| 0.0000122 | 0.000186 | <1e−07 | 37.91 | 17.96 | 2.11 | ACOT9 | acyl-CoA | 23597 | NM_001033583 |
| | | | | | | | thioesterase 9 | | |
| 0.0000154 | 0.00022 | <1e−07 | 105.31 | 49.86 | 2.11 | CCNL2 | cyclin L2 | 81669 | NM_001039577 |
| 0.0000183 | 0.00025 | <1e−07 | 130.03 | 61.66 | 2.11 | TUBGCP6 | tubulin gamma | 85378 | NM_001008658 |
| | | | | | | | complex associated | | |
| | | | | | | | protein 6 | | |
| 0.0000258 | 0.00032 | <1e−07 | 22.6 | 10.7 | 2.11 | RABGEF1 | RAB guanine | 27342 | NM_001287060 |
| | | | | | | | nucleotide exchange | | |
| | | | | | | | factor 1 | | |
| 0.0001408 | 0.00113 | 0.0001 | 84.8 | 40.18 | 2.11 | MROH1 | maestro heat like | 727957 | NM_001099280 |
| | | | | | | | repeat family | | |
| | | | | | | | member 1 | | |
| 0.0001968 | 0.00146 | 0.0002 | 51.97 | 24.64 | 2.11 | PRPF3 | pre-m RNA | 9129 | NM_001350529 |
| | | | | | | | processing factor 3 | | |
| 0.0004003 | 0.00249 | 0.0003 | 145.53 | 68.82 | 2.11 | RELT | RELT, TNF | 84957 | NM_032871 |
| | | | | | | | receptor | | |
| 0.0004774 | 0.00286 | 0.0003 | 351.96 | 166.48 | 2.11 | SLC7A5 | solute carrier family | 8140 | NM_003486 |
| | | | | | | | 7 member 5 | | |
| <1e−07 | 5.39E−06 | <1e−07 | 47.63 | 22.44 | 2.12 | LDB1 | LIM domain | 8861 | NM_001113407 |
| | | | | | | | binding 1 | | |
| 0.0000003 | 1.23E−05 | <1e−07 | 26.27 | 12.37 | 2.12 | TFEB | transcription factor | 7942 | NM_001167827 |
| | | | | | | | EB | | |
| 0.0000007 | 2.34E−05 | <1e−07 | 144.19 | 68.04 | 2.12 | HSPA9 | heat shock protein | 3313 | NM_004134 |
| | | | | | | | family A (Hsp70) | | |
| | | | | | | | member 9 | | |
| 0.000001 | 3.07E−05 | <1e−07 | 51.55 | 24.34 | 2.12 | NAGK | N- | 55577 | NM_001330425 |
| | | | | | | | acetylglucosamine | | |
| | | | | | | | kinase | | |
| 0.000001 | 3.07E−05 | <1e−07 | 24.69 | 11.64 | 2.12 | POLG2 | DNA polymerase | 11232 | NM_007215 |
| | | | | | | | gamma 2, accessory | | |
| | | | | | | | subunit | | |
| 0.000001 | 3.07E−05 | <1e−07 | 103.08 | 48.68 | 2.12 | IFI16 | interferon gamma | 3428 | NM_001206567 |
| | | | | | | | inducible protein 16 | | |
| 0.0000012 | 3.52E−05 | <1e−07 | 29.72 | 13.99 | 2.12 | SECISBP2L | SECIS binding | 9728 | NM_001193489 |
| | | | | | | | protein 2 like | | |
| 0.0000021 | 5.27E−05 | <1e−07 | 32.71 | 15.43 | 2.12 | ABTB1 | ankyrin repeat and | 80325 | NM_032548 |
| | | | | | | | BTB domain | | |
| | | | | | | | containing 1 | | |
| 0.0000025 | 0.00006 | <1e−07 | 25.52 | 12.04 | 2.12 | ZNF655 | zinc finger protein | 79027 | NM_001009956 |
| | | | | | | | 655 | | |
| 0.0000036 | 7.74E−05 | <1e−07 | 24.93 | 11.74 | 2.12 | DEF8 | differentially | 54849 | NM_001242816 |
| | | | | | | | expressed in FDCP | | |
| | | | | | | | 8 homolog | | |
| 0.0000041 | 8.41E−05 | <1e−07 | 120.86 | 56.92 | 2.12 | CREBRF | CREB3 regulatory | 153222 | NM_001168393 |
| | | | | | | | factor | | |
| 0.0000045 | 8.96E−05 | <1e−07 | 19.54 | 9.23 | 2.12 | THOC6 | THO complex 6 | 79228 | NM_001142350 |
| 0.0000068 | 0.000121 | <1e−07 | 73.8 | 34.78 | 2.12 | ANXA6 | annexin A6 | 309 | NM_001155 |
| 0.0000101 | 0.00016 | <1e−07 | 70.78 | 33.39 | 2.12 | REXO1 | RNA exonuclease 1 | 57455 | NM_020695 |
| | | | | | | | homolog | | |
| 0.0000128 | 0.000192 | <1e−07 | 42.73 | 20.17 | 2.12 | RDX | radixin | 5962 | NM_001260492 |
| 0.0000207 | 0.000272 | <1e−07 | 44.26 | 20.84 | 2.12 | TAF1C | TATA-box binding | 9013 | NM_001243156 |
| | | | | | | | protein associated | | |
| | | | | | | | factor, RNA | | |
| | | | | | | | polymerase I | | |
| | | | | | | | subunit C | | |
| 0.0000282 | 0.000342 | 0.0001 | 27.27 | 12.85 | 2.12 | CBFA2T3 | CBFA2/RUNX1 | 863 | NM_005187 |
| | | | | | | | translocation partner | | |
| | | | | | | | 3 | | |
| 0.0000324 | 0.000377 | <1e−07 | 23.2 | 10.94 | 2.12 | TMEM273 | transmembrane | 170371 | NM_001010863 |
| | | | | | | | protein 273 | | |
| 0.0006081 | 0.00348 | 0.0005 | 50.65 | 23.85 | 2.12 | PLD3 | phospholipase D | 23646 | NM_001031696 |
| | | | | | | | family member 3 | | |
| <1e−07 | <1e−07 | <1e−07 | 27.64 | 13 | 2.13 | TEPSIN | TEPSIN, adaptor | 146705 | NM_144679 |
| | | | | | | | related protein | | |
| | | | | | | | complex 4 accessory | | |
| | | | | | | | protein | | |
| 0.0000001 | 5.39E−06 | <1e−07 | 30.11 | 14.15 | 2.13 | ABCD4 | ATP binding | 5826 | NM_001353591 |
| | | | | | | | cassette subfamily D | | |
| | | | | | | | member 4 | | |
| 0.0000002 | 9.26E−06 | <1e−07 | 18.62 | 8.76 | 2.13 | IFNAR2 | interferon alpha and | 3455 | NM_000874 |
| | | | | | | | beta receptor | | |
| | | | | | | | subunit 2 | | |
| 0.0000007 | 2.34E−05 | <1e−07 | 24.91 | 11.72 | 2.13 | CBX7 | chromobox 7 | 23492 | NM_001346743 |
| 0.0000009 | 2.83E−05 | <1e−07 | 25.09 | 11.79 | 2.13 | AC008105.3 | noncoding | | |
| | | | | | | | (Ensemble gene ID: | | |
| | | | | | | | ENSG00000272256.1) | | |
| 0.0000015 | 0.000041 | <1e−07 | 101.63 | 47.69 | 2.13 | AGAP3 | ArfGAP with | 116988 | NM_001042535 |
| | | | | | | | GTPase domain, | | |
| | | | | | | | ankyrin repeat and | | |
| | | | | | | | PH domain 3 | | |
| 0.0000027 | 6.33E−05 | <1e−07 | 48.11 | 22.55 | 2.13 | ABCF3 | ATP binding | 55324 | NM_001351298 |
| | | | | | | | cassette subfamily F | | |
| | | | | | | | member 3 | | |
| 0.0000028 | 0.000065 | <1e−07 | 31.98 | 15.02 | 2.13 | SEMA4C | semaphorin 4C | 54910 | NM_017789 |
| 0.0000036 | 7.74E−05 | <1e−07 | 39.86 | 18.72 | 2.13 | UNKL | unkempt family like | 64718 | NM_001037125 |
| | | | | | | | zinc finger | | |
| 0.0000039 | 8.11E−05 | <1e−07 | 576.51 | 270.33 | 2.13 | JUND | JunD proto- | 3727 | NM_001286968 |
| | | | | | | | oncogene, AP-1 | | |
| | | | | | | | transcription factor | | |
| | | | | | | | subunit | | |
| 0.0000048 | 9.47E−05 | <1e−07 | 104.95 | 49.32 | 2.13 | HIVEP1 | human | 3096 | NM_002114 |
| | | | | | | | immunodeficiency | | |
| | | | | | | | virus type I | | |
| | | | | | | | enhancer binding | | |
| | | | | | | | protein 1 | | |
| 0.0000127 | 0.000192 | <1e−07 | 54.73 | 25.66 | 2.13 | CDK17 | cyclin dependent | 5128 | NM_001170464 |
| | | | | | | | kinase 17 | | |
| 0.0000285 | 0.000343 | <1e−07 | 31.55 | 14.83 | 2.13 | CTC1 | CST telomere | 80169 | NM_025099 |
| | | | | | | | replication complex | | |
| | | | | | | | component 1 | | |
| 0.0000403 | 0.000442 | 0.0001 | 22.03 | 10.35 | 2.13 | AC122718.1 | noncoding (Clone | | |
| | | | | | | | Fragment ID: | | |
| | | | | | | | AC122718.2) | | |
| 0.0000527 | 0.00054 | <1e−07 | 101.86 | 47.8 | 2.13 | TNKS1BP1 | tankyrase 1 binding | 85456 | NM_033396 |
| | | | | | | | protein 1 | | |
| 0.0001129 | 0.000955 | 0.0002 | 32.37 | 15.23 | 2.13 | FGD3 | FYVE, RhoGEF and | 89846 | NM_001083536 |
| | | | | | | | PH domain | | |
| | | | | | | | containing 3 | | |
| 0.0001176 | 0.000989 | <1e−07 | 30.37 | 14.23 | 2.13 | SLC35A2 | solute carrier family | 7355 | NM_001032289 |
| | | | | | | | 35 member A2 | | |
| 0.0003328 | 0.00217 | 0.0002 | 96.06 | 45 | 2.13 | ETS1 | ETS proto-oncogene | 2113 | NM_001143820 |
| | | | | | | | 1, transcription | | |
| | | | | | | | factor | | |
| <1e−07 | <1e−07 | <1e−07 | 20.86 | 9.75 | 2.14 | UBE2J2 | ubiquitin | 118424 | NM_058167 |
| | | | | | | | conjugating enzyme | | |
| | | | | | | | E2 J2 | | |
| 0.0000003 | 1.23E−05 | <1e−07 | 107.15 | 50.13 | 2.14 | CYLD | CYLD lysine 63 | 1540 | NM_001042355 |
| | | | | | | | deubiquitinase | | |
| 0.0000015 | 0.000041 | <1e−07 | 54.99 | 25.64 | 2.14 | IRF3 | interferon regulatory | 3661 | NM_001197122 |
| | | | | | | | factor 3 | | |
| 0.0000021 | 5.27E−05 | <1e−07 | 22.52 | 10.53 | 2.14 | PPM1K | protein phosphatase, | 152926 | NM_152542 |
| | | | | | | | Mg2+/Mn2+ | | |
| | | | | | | | dependent 1K | | |
| 0.0000067 | 0.00012 | <1e−07 | 51.58 | 24.08 | 2.14 | WDR81 | WD repeat domain | 124997 | NM_001163673 |
| | | | | | | | 81 | | |
| 0.0001638 | 0.00127 | 0.0002 | 132.71 | 62.15 | 2.14 | TTYH3 | tweety family | 80727 | NM_025250 |
| | | | | | | | member 3 | | |
| 0.0002639 | 0.00181 | 0.0002 | 43.73 | 20.47 | 2.14 | FAM118A | family with | 55007 | NM_001104595 |
| | | | | | | | sequence similarity | | |
| | | | | | | | 118 member A | | |
| 0.0003281 | 0.00215 | 0.0003 | 21.43 | 10.01 | 2.14 | IL21R | interleukin 21 | 50615 | NM_021798 |
| | | | | | | | receptor | | |
| 0.0004093 | 0.00254 | 0.0005 | 501.83 | 234.59 | 2.14 | TSPYL2 | TSPY like 2 | 64061 | NM_022117 |
| <1e−07 | <1e−07 | <1e−07 | 41.77 | 19.41 | 2.15 | FAM168B | family with | 130074 | NM_001009993 |
| | | | | | | | sequence similarity | | |
| | | | | | | | 168 member B | | |
| 0.0000003 | 1.23E−05 | <1e−07 | 16.41 | 7.64 | 2.15 | IRF5 | interferon regulatory | 3663 | NM_001098627 |
| | | | | | | | factor 5 | | |
| 0.0000011 | 3.33E−05 | <1e−07 | 47.79 | 22.21 | 2.15 | NCKAP5L | NCK associated | 57701 | NM_001037806 |
| | | | | | | | protein 5 like | | |
| 0.0000031 | 6.98E−05 | <1e−07 | 33.69 | 15.7 | 2.15 | IL18BP | interleukin 18 | 10068 | NM_001039659 |
| | | | | | | | binding protein | | |
| 0.0000036 | 7.74E−05 | <1e−07 | 101.94 | 47.35 | 2.15 | OTUD5 | OTU deubiquitinase | 55593 | NM_001136157 |
| | | | | | | | 5 | | |
| 0.00002 | 0.000266 | <1e−07 | 320.11 | 148.83 | 2.15 | EHMT1 | euchromatic histone | 79813 | NM_001039765 |
| | | | | | | | lysine | | |
| | | | | | | | methyltransferase 1 | | |
| 0.0000619 | 0.000609 | <1e−07 | 745.77 | 347.5 | 2.15 | DUSP1 | dual specificity | 1843 | NM_004417 |
| | | | | | | | phosphatase 1 | | |
| 0.0000781 | 0.00072 | 0.0001 | 66.87 | 31.15 | 2.15 | NEU1 | neuraminidase 1 | 4758 | NM_000434 |
| 0.0002765 | 0.00188 | 0.0001 | 69.61 | 32.4 | 2.15 | CORO1A | coronin 1A | 11151 | NM_001193333 |
| 0.0004168 | 0.00257 | 0.0006 | 27.29 | 12.7 | 2.15 | LONRF3 | LON peptidase N- | 79836 | NM_001031855 |
| | | | | | | | terminal domain and | | |
| | | | | | | | ring finger 3 | | |
| 0.0007359 | 0.00404 | 0.0003 | 63.99 | 29.75 | 2.15 | DOCK4 | dedicator of | 9732 | NM_014705 |
| | | | | | | | cytokinesis 4 | | |
| 0.0007912 | 0.00428 | 0.0004 | 13.48 | 6.26 | 2.15 | NIBAN3 | Niban Apoptosis | 199786 | |
| | | | | | | | Regulator 3 | | |
| <1e−07 | <1e−07 | <1e−07 | 24.5 | 11.33 | 2.16 | MOB2 | MOB kinase | 81532 | NM_001172223 |
| | | | | | | | activator 2 | | |
| <1e−07 | <1e−07 | <1e−07 | 108.35 | 50.07 | 2.16 | USP48 | ubiquitin specific | 84196 | NM_001032730 |
| | | | | | | | peptidase 48 | | |
| <1e−07 | <1e−07 | <1e−07 | 46.11 | 21.35 | 2.16 | FAM13B | family with | 51306 | NM_001101800 |
| | | | | | | | sequence similarity | | |
| | | | | | | | 13 member B | | |
| <1e−07 | 5.39E−06 | <1e−07 | 313.62 | 145.41 | 2.16 | SRSF5 | serine and arginine | 6430 | NM_001039465 |
| | | | | | | | rich splicing factor 5 | | |
| 0.0000001 | 5.39E−06 | <1e−07 | 51.9 | 24.01 | 2.16 | PTOV1 | prostate tumor | 53635 | NM_001305105 |
| | | | | | | | overexpressed 1 | | |
| 0.0000003 | 1.23E−05 | <1e−07 | 33.45 | 15.46 | 2.16 | RETREG2 | reticulophagy | 79137 | NM_001321109 |
| | | | | | | | regulator family | | |
| | | | | | | | member 2 | | |
| 0.0000003 | 1.23E−05 | <1e−07 | 91.89 | 42.49 | 2.16 | ESYT2 | extended | 57488 | NM_020728 |
| | | | | | | | synaptotagmin 2 | | |
| 0.0000028 | 0.000065 | <1e−07 | 34.06 | 15.79 | 2.16 | SFI1 | SFI1 centrin binding | 9814 | NM_001007467 |
| | | | | | | | protein | | |
| 0.0000037 | 0.000079 | <1e−07 | 55.21 | 25.62 | 2.16 | UNC13D | unc-13 homolog D | 201294 | NM_199242 |
| 0.0000049 | 9.58E−05 | <1e−07 | 46.24 | 21.41 | 2.16 | TLNRD1 | talin rod domain | 59274 | NM_022566 |
| | | | | | | | containing 1 | | |
| 0.000012 | 0.000184 | <1e−07 | 176.46 | 81.74 | 2.16 | PTK2B | protein tyrosine | 2185 | NM_004103 |
| | | | | | | | kinase 2 beta | | |
| 0.0000335 | 0.000386 | <1e−07 | 609.29 | 282.44 | 2.16 | SQSTM1 | sequestosome 1 | 8878 | NM_001142298 |
| 0.0002555 | 0.00177 | 0.0004 | 19.62 | 9.07 | 2.16 | SNORA14B | small nucleolar | 677802 | NR_002956 |
| | | | | | | | RNA, H/ACA box | | |
| | | | | | | | 14B | | |
| 0.0000004 | 0.000015 | <1e−07 | 70.27 | 32.4 | 2.17 | GPBP1 | GC-rich promoter | 65056 | NM_001127235 |
| | | | | | | | binding protein 1 | | |
| 0.0000007 | 2.34E−05 | <1e−07 | 130.38 | 60.21 | 2.17 | PPP6R1 | protein phosphatase | 22870 | NM_014931 |
| | | | | | | | 6 regulatory subunit | | |
| | | | | | | | 1 | | |
| 0.0000009 | 2.83E−05 | <1e−07 | 40.29 | 18.55 | 2.17 | PDE7A | phosphodiesterase | 5150 | NM_001242318 |
| | | | | | | | 7A | | |
| 0.0000038 | 7.99E−05 | <1e−07 | 62.44 | 28.76 | 2.17 | CHFR | checkpoint with | 55743 | NM_001161344 |
| | | | | | | | forkhead and ring | | |
| | | | | | | | finger domains | | |
| 0.0000041 | 8.41E−05 | <1e−07 | 20.19 | 9.3 | 2.17 | AC138932.1 | noncoding | | |
| | | | | | | | (Ensemble gene ID: | | |
| | | | | | | | ENSG00000183458.14) | | |
| 0.0000054 | 0.000103 | <1e−07 | 82.72 | 38.2 | 2.17 | NOP56 | NOP56 | 10528 | NM_006392 |
| | | | | | | | ribonucleoprotein | | |
| 0.0000114 | 0.000177 | <1e−07 | 44.75 | 20.6 | 2.17 | MAML2 | mastermind like | 84441 | NM_032427 |
| | | | | | | | transcriptional | | |
| | | | | | | | coactivator 2 | | |
| 0.0000163 | 0.000229 | <1e−07 | 79.64 | 36.78 | 2.17 | TP53INP1 | tumor protein p53 | 94241 | NM_001135733 |
| | | | | | | | inducible nuclear | | |
| | | | | | | | protein 1 | | |
| 0.0000266 | 0.000327 | <1e−07 | 29.9 | 13.76 | 2.17 | METTL7A | methyltransferase | 25840 | NM_014033 |
| | | | | | | | like 7A | | |
| 0.0000464 | 0.000492 | <1e−07 | 21.12 | 9.74 | 2.17 | CPNE5 | copine 5 | 57699 | NM_001314017 |
| 0.0005854 | 0.00337 | 0.0008 | 162 | 74.56 | 2.17 | CD83 | CD83 molecule | 9308 | NM_001040280 |
| <1e−07 | <1e−07 | <1e−07 | 160.82 | 73.93 | 2.18 | JAK1 | Janus kinase 1 | 3716 | NM_001320923 |
| <1e−07 | <1e−07 | <1e−07 | 35.88 | 16.49 | 2.18 | ZCCHC8 | zinc finger CCHC- | 55596 | NM_001350935 |
| | | | | | | | type containing 8 | | |
| <1e−07 | <1e−07 | <1e−07 | 26.87 | 12.32 | 2.18 | LIMD1 | LIM domains | 8994 | NM_014240 |
| | | | | | | | containing 1 | | |
| <1e−07 | <1e−07 | <1e−07 | 129.71 | 59.45 | 2.18 | FBRSL1 | fibrosin like 1 | 57666 | NM_001142641 |
| <1e−07 | <1e−07 | <1e−07 | 24.76 | 11.35 | 2.18 | GID8 | GID complex | 54994 | NM_017896 |
| | | | | | | | subunit 8 homolog | | |
| 0.0000001 | 5.39E−06 | <1e−07 | 193.66 | 88.84 | 2.18 | NXF1 | nuclear RNA export | 10482 | NM_001081491 |
| | | | | | | | factor 1 | | |
| 0.0000001 | 5.39E−06 | <1e−07 | 49.71 | 22.85 | 2.18 | RNF216 | ring finger protein | 54476 | NM_019011 |
| | | | | | | | 216 | | |
| 0.0000001 | 5.39E−06 | <1e−07 | 20.52 | 9.41 | 2.18 | TSR2 | TSR2, ribosome | 90121 | NM_001346789 |
| | | | | | | | maturation factor | | |
| 0.0000008 | 2.61E−05 | <1e−07 | 29.42 | 13.52 | 2.18 | ZC3H7A | zinc finger CCCH- | 29066 | NM_014153 |
| | | | | | | | type containing 7A | | |
| 0.0000014 | 3.93E−05 | <1e−07 | 52.48 | 24.11 | 2.18 | ZBTB1 | zinc finger and BTB | 22890 | NM_001123329 |
| | | | | | | | domain containing 1 | | |
| 0.0000014 | 3.93E−05 | <1e−07 | 83.57 | 38.26 | 2.18 | SEC61A1 | Sec61 translocon | 29927 | NM_013336 |
| | | | | | | | alpha 1 subunit | | |
| 0.0000037 | 0.000079 | <1e−07 | 25.22 | 11.56 | 2.18 | SLC2A4RG | SLC2A4 regulator | 56731 | NM_020062 |
| 0.0000085 | 0.000142 | <1e−07 | 24.52 | 11.26 | 2.18 | SETDB2 | SET domain | 83852 | NM_001160308 |
| | | | | | | | bifurcated 2 | | |
| 0.0000173 | 0.000238 | 0.0001 | 74.43 | 34.14 | 2.18 | HBP1 | HMG-box | 26959 | NM_001244262 |
| | | | | | | | transcription factor | | |
| | | | | | | | 1 | | |
| 0.00007 | 0.000664 | 0.0001 | 127.13 | 58.34 | 2.18 | RELB | RELB proto- | 5971 | NM_006509 |
| | | | | | | | oncogene, NF-kB | | |
| | | | | | | | subunit | | |
| 0.0002213 | 0.00159 | <1e−07 | 29.19 | 13.4 | 2.18 | BAIAP3 | BAI1 associated | 8938 | NM_001199096 |
| | | | | | | | protein 3 | | |
| <1e−07 | <1e−07 | <1e−07 | 36.87 | 16.86 | 2.19 | MARCHF8 | Membrane | 220972 | |
| | | | | | | | Associated Ring- | | |
| | | | | | | | CH-Type Finger 8 | | |
| 0.0000002 | 9.26E−06 | <1e−07 | 24.32 | 11.1 | 2.19 | DCAF8 | DDB1 and CUL4 | 50717 | NM_015726 |
| | | | | | | | associated factor 8 | | |
| | | | | | | | Terminal | | |
| | | | | | | | Nucleotidyltransferase | | |
| | | | | | | | 4A | | |
| 0.0000033 | 7.31E−05 | <1e−07 | 143.89 | 65.66 | 2.19 | PNPLA2 | patatin like | 57104 | NM_020376 |
| | | | | | | | phospholipase | | |
| | | | | | | | domain containing 2 | | |
| 0.0000049 | 9.58E−05 | <1e−07 | 308.61 | 140.81 | 2.19 | PPP1R15B | protein phosphatase | 84919 | NM_032833 |
| | | | | | | | 1 regulatory subunit | | |
| | | | | | | | 15B | | |
| 0.0000077 | 0.000133 | <1e−07 | 94.09 | 43.02 | 2.19 | PPRC1 | peroxisome | 23082 | NM_001288727 |
| | | | | | | | proliferator- | | |
| | | | | | | | activated receptor | | |
| | | | | | | | gamma, coactivator- | | |
| | | | | | | | related 1 | | |
| 0.0000095 | 0.000154 | <1e−07 | 119.95 | 54.71 | 2.19 | RSRP1 | arginine and serine | 57035 | NM_001321772 |
| | | | | | | | rich protein 1 | | |
| 0.0000106 | 0.000167 | <1e−07 | 85.01 | 38.82 | 2.19 | ATP6V0D1 | ATPase H+ | 9114 | NM_004691 |
| | | | | | | | transporting V0 | | |
| | | | | | | | subunit d1 | | |
| 0.0000165 | 0.000231 | <1e−07 | 134.74 | 61.54 | 2.19 | OGA | O-GlcNAcase | 10724 | |
| 0.0000334 | 0.000386 | <1e−07 | 63.09 | 28.83 | 2.19 | MIB2 | mindbomb E3 | 142678 | NM_001170686 |
| | | | | | | | ubiquitin protein | | |
| | | | | | | | ligase 2 | | |
| 0.0005958 | 0.00342 | 0.0007 | 25.07 | 11.43 | 2.19 | FBLN2 | fibulin 2 | 2199 | NM_001004019 |
| <1e−07 | <1e−07 | <1e−07 | 20.02 | 9.1 | 2.2 | LRWD1 | leucine rich repeats | 222229 | NM_001317721 |
| | | | | | | | and WD repeat | | |
| | | | | | | | domain containing 1 | | |
| 0.0000024 | 5.78E−05 | <1e−07 | 24.89 | 11.31 | 2.2 | IRF2BP1 | interferon regulatory | 26145 | NM_015649 |
| | | | | | | | factor 2 binding | | |
| | | | | | | | protein 1 | | |
| 0.0000039 | 8.11E−05 | <1e−07 | 56.13 | 25.55 | 2.2 | ORAI2 | ORAI calcium | 80228 | NM_001126340 |
| | | | | | | | release-activated | | |
| | | | | | | | calcium modulator 2 | | |
| 0.0000169 | 0.000235 | <1e−07 | 45.46 | 20.63 | 2.2 | FMNL2 | form in like 2 | 114793 | NM_001004417 |
| 0.0000218 | 0.000282 | <1e−07 | 130.23 | 59.14 | 2.2 | PIK3R5 | phosphoinositide-3- | 23533 | NM_001142633 |
| | | | | | | | kinase regulatory | | |
| | | | | | | | subunit 5 | | |
| 0.0000241 | 0.000304 | <1e−07 | 12.72 | 5.79 | 2.2 | AC090617.10 | noncoding | | |
| | | | | | | | (Ensemble gene ID: | | |
| | | | | | | | ENSG00000287553.1) | | |
| 0.0000359 | 0.000406 | <1e−07 | 64.01 | 29.15 | 2.2 | RASGEF1B | RasGEF domain | 153020 | NM_001300735 |
| | | | | | | | family member 1B | | |
| 0.0000506 | 0.000524 | 0.0001 | 22.79 | 10.35 | 2.2 | PI4K2A | phosphatidylinositol | 55361 | NM_018425 |
| | | | | | | | 4-kina se type 2 | | |
| | | | | | | | alpha | | |
| 0.0000883 | 0.000793 | 0.0001 | 79.02 | 35.96 | 2.2 | SLC38A1 | solute carrier family | 81539 | NM_001077484 |
| | | | | | | | 38 member 1 | | |
| 0.0008593 | 0.00455 | 0.001 | 292.02 | 132.74 | 2.2 | HLA-DPB1 | major | 3115 | NM_002121 |
| | | | | | | | histocompatibility | | |
| | | | | | | | complex, class II, | | |
| | | | | | | | DP beta 1 | | |
| <1e−07 | <1e−07 | <1e−07 | 109.67 | 49.65 | 2.21 | PI4KA | phosphatidylinositol | 5297 | NM_002650 |
| | | | | | | | 4-kina se alpha | | |
| <1e−07 | <1e−07 | <1e−07 | 42.45 | 19.25 | 2.21 | GNPTAB | N- | 79158 | NM_024312 |
| | | | | | | | acetylglucosamine- | | |
| | | | | | | | 1-phosphate | | |
| | | | | | | | transferase alpha | | |
| | | | | | | | and beta subunits | | |
| 0.0000001 | 5.39E−06 | <1e−07 | 65.4 | 29.63 | 2.21 | KCNAB2 | potassium voltage- | 8514 | NM_001199860 |
| | | | | | | | gated channel | | |
| | | | | | | | subfamily A | | |
| | | | | | | | regulatory beta | | |
| | | | | | | | subunit 2 | | |
| 0.0000003 | 1.23E−05 | <1e−07 | 31 | 14.03 | 2.21 | HMBOX1 | homeobox | 79618 | NM_001135726 |
| | | | | | | | containing 1 | | |
| 0.0000006 | 2.07E−05 | <1e−07 | 18.73 | 8.48 | 2.21 | VAMP2 | vesicle associated | 6844 | NM_001330125 |
| | | | | | | | membrane protein 2 | | |
| 0.0000006 | 2.07E−05 | <1e−07 | 55.88 | 25.28 | 2.21 | PACS1 | phosphofurin acidic | 55690 | NM_018026 |
| | | | | | | | cluster sorting | | |
| | | | | | | | protein 1 | | |
| 0.0000006 | 2.07E−05 | <1e−07 | 124.69 | 56.5 | 2.21 | STX4 | syntaxin 4 | 6810 | NM_001272095 |
| 0.0000007 | 2.34E−05 | <1e−07 | 19.73 | 8.91 | 2.21 | SRCAP | Snf2 related | 10847 | NM_006662 |
| | | | | | | | CREBBP activator | | |
| | | | | | | | protein | | |
| 0.0000008 | 2.61E−05 | <1e−07 | 44.19 | 19.96 | 2.21 | ARHGAP31 | Rho GTPase | 57514 | NM_020754 |
| | | | | | | | activating protein 31 | | |
| 0.0000016 | 4.31E−05 | <1e−07 | 77.29 | 34.98 | 2.21 | TRABD | TraB domain | 80305 | NM_001320484 |
| | | | | | | | containing | | |
| 0.0000065 | 0.000117 | <1e−07 | 52.45 | 23.79 | 2.21 | PAG1 | phosphoprotein | 55824 | NM_018440 |
| | | | | | | | membrane anchor | | |
| | | | | | | | with | | |
| | | | | | | | glycosphingolipid | | |
| | | | | | | | microdomains 1 | | |
| 0.0000082 | 0.00014 | <1e−07 | 51.05 | 23.06 | 2.21 | NPC1 | NPC intracellular | 4864 | NM_000271 |
| | | | | | | | cholesterol | | |
| | | | | | | | transporter 1 | | |
| 0.0000085 | 0.000142 | <1e−07 | 123.22 | 55.79 | 2.21 | SH3BP5 | SH3 domain binding | 9467 | NM_001018009 |
| | | | | | | | protein 5 | | |
| 0.0000136 | 0.000202 | <1e−07 | 70.66 | 32.03 | 2.21 | SNX9 | sorting nexin 9 | 51429 | NM_016224 |
| 0.0000675 | 0.000645 | <1e−07 | 88.19 | 39.89 | 2.21 | SLC1A5 | solute carrier family | 6510 | NM_001145144 |
| | | | | | | | 1 member 5 | | |
| 0.0001312 | 0.00107 | 0.0002 | 23.22 | 10.49 | 2.21 | MCOLN2 | mucolipin 2 | 255231 | NM_001330647 |
| 0.0001705 | 0.00131 | <1e−07 | 93.92 | 42.55 | 2.21 | STAT5A | signal transducer | 6776 | NM_001288718 |
| | | | | | | | and activator of | | |
| | | | | | | | transcription 5A | | |
| <1e−07 | <1e−07 | <1e−07 | 14.07 | 6.34 | 2.22 | MB21D2 | Mab-21 domain | 151963 | NM_178496 |
| | | | | | | | containing 2 | | |
| <1e−07 | 5.39E−06 | <1e−07 | 38.11 | 17.19 | 2.22 | FCHSD2 | FCH and double | 9873 | NM_014824 |
| | | | | | | | SH3 domains 2 | | |
| 0.0000001 | 5.39E−06 | <1e−07 | 75.54 | 34.1 | 2.22 | USP11 | ubiquitin specific | 8237 | NM_004651 |
| | | | | | | | peptidase 11 | | |
| 0.0000003 | 1.23E−05 | <1e−07 | 309.57 | 139.73 | 2.22 | AC022966.1 | ubiquitin specific | | |
| | | | | | | | peptidase 36 | | |
| | | | | | | | (EnsEMBL Gene ID | | |
| | | | | | | | ENSG00000055483) | | |
| 0.0000004 | 0.000015 | <1e−07 | 50.23 | 22.66 | 2.22 | PIKFYVE | phosphoinositide | 200576 | NM_001002881 |
| | | | | | | | kinase, FYVE-type | | |
| | | | | | | | zinc finger | | |
| | | | | | | | containing | | |
| 0.0000013 | 3.73E−05 | <1e−07 | 30.06 | 13.56 | 2.22 | CHPF2 | chondroitin | 54480 | NM_001284295 |
| | | | | | | | polymerizing factor | | |
| | | | | | | | 2 | | |
| 0.0000032 | 7.14E−05 | <1e−07 | 222.31 | 99.96 | 2.22 | TMEM259 | transmembrane | 91304 | NM_001033026 |
| | | | | | | | protein 259 | | |
| 0.0000187 | 0.000253 | <1e−07 | 34.49 | 15.53 | 2.22 | LIMD2 | LIM domain | 80774 | NM_030576 |
| | | | | | | | containing 2 | | |
| 0.0000379 | 0.000424 | <1e−07 | 54.59 | 24.59 | 2.22 | SYNGAP1 | synaptic Ras | 8831 | NM_001130066 |
| | | | | | | | GTPase activating | | |
| | | | | | | | protein 1 | | |
| 0.0000672 | 0.000643 | <1e−07 | 135.1 | 60.93 | 2.22 | DUSP2 | dual specificity | 1844 | NM_004418 |
| | | | | | | | phosphatase 2 | | |
| 0.000122 | 0.00101 | <1e−07 | 57.21 | 25.77 | 2.22 | FGFR1 | fibroblast growth | 2260 | NM_001174063 |
| | | | | | | | factor receptor 1 | | |
| <1e−07 | <1e−07 | <1e−07 | 38.93 | 17.46 | 2.23 | STARD3 | StAR related lipid | 10948 | NM_001165937 |
| | | | | | | | transfer domain | | |
| | | | | | | | containing 3 | | |
| <1e−07 | <1e−07 | <1e−07 | 110.17 | 49.4 | 2.23 | DOCK2 | dedicator of | 1794 | NM_004946 |
| | | | | | | | cytokinesis 2 | | |
| <1e−07 | <1e−07 | <1e−07 | 128.56 | 57.59 | 2.23 | SIK3 | SIK family kinase 3 | 23387 | NM_001281748 |
| <1e−07 | 5.39E−06 | <1e−07 | 21.54 | 9.66 | 2.23 | INTS6L | integrator complex | 203522 | NM_001351601 |
| | | | | | | | subunit 6 like | | |
| 0.0000001 | 5.39E−06 | <1e−07 | 39.82 | 17.9 | 2.23 | M6PR | mannose-6- | 4074 | NM_001207024 |
| | | | | | | | phosphate receptor, | | |
| | | | | | | | cation dependent | | |
| 0.0000012 | 3.52E−05 | <1e−07 | 37.32 | 16.75 | 2.23 | AHDC1 | AT-hook DNA | 27245 | NM_001029882 |
| | | | | | | | binding motif | | |
| | | | | | | | containing 1 | | |
| 0.0000035 | 7.64E−05 | <1e−07 | 46.31 | 20.81 | 2.23 | ARRDC2 | arrestin domain | 27106 | NM_001025604 |
| | | | | | | | containing 2 | | |
| 0.0000038 | 7.99E−05 | <1e−07 | 40.51 | 18.19 | 2.23 | FLI1 | Fli-1 proto- | 2313 | NM_001167681 |
| | | | | | | | oncogene, ETS | | |
| | | | | | | | transcription factor | | |
| 0.0000082 | 0.00014 | <1e−07 | 33.65 | 15.07 | 2.23 | RNF166 | ring finger protein | 115992 | NM_001171815 |
| | | | | | | | 166 | | |
| 0.0000313 | 0.000367 | <1e−07 | 79.56 | 35.63 | 2.23 | ISG20 | interferon stimulated | 3669 | NM_001303233 |
| | | | | | | | exonuclease gene 20 | | |
| 0.0000313 | 0.000367 | 0.0001 | 47.08 | 21.08 | 2.23 | NCF4 | neutrophil cytosolic | 4689 | NM_000631 |
| | | | | | | | factor 4 | | |
| 0.0000336 | 0.000387 | <1e−07 | 23.46 | 10.54 | 2.23 | KCNA3 | potassium voltage- | 3738 | NM_002232 |
| | | | | | | | gated channel | | |
| | | | | | | | subfamily A | | |
| | | | | | | | member 3 | | |
| 0.0002415 | 0.0017 | 0.0002 | 52.55 | 23.52 | 2.23 | IQCN | IQmotif containing | 80726 | NM_001145304 |
| | | | | | | | N | | |
| 0.0002985 | 0.002 | 0.0001 | 85.93 | 38.61 | 2.23 | SCARNA5 | small Cajal body- | 677775 | NR_003008 |
| | | | | | | | specific RNA 5 | | |
| 0.0003705 | 0.00235 | 0.0003 | 135.12 | 60.53 | 2.23 | RGS1 | regulator of G | 5996 | NM_002922 |
| | | | | | | | protein signaling 1 | | |
| <1e−07 | <1e−07 | <1e−07 | 55.04 | 24.58 | 2.24 | TUG1 | taurine up-regulated | 55000 | NR_002323 |
| | | | | | | | 1 (non-protein | | |
| | | | | | | | coding) | | |
| <1e−07 | <1e−07 | <1e−07 | 191 | 85.45 | 2.24 | ATXN2L | ataxin 2 like | 11273 | NM_001308230 |
| <1e−07 | <1e−07 | <1e−07 | 45.08 | 20.1 | 2.24 | MTMR14 | myotubularin related | 64419 | NM_001077525 |
| | | | | | | | protein 14 | | |
| <1e−07 | 5.39E−06 | <1e−07 | 37.3 | 16.67 | 2.24 | DMXL1 | Dmx like 1 | 1657 | NM_001290321 |
| 0.0000002 | 9.26E−06 | <1e−07 | 48.85 | 21.8 | 2.24 | MAN2B1 | mannosidase alpha | 4125 | NM_000528 |
| | | | | | | | class 2B member 1 | | |
| 0.0000002 | 9.26E−06 | <1e−07 | 21.49 | 9.57 | 2.24 | EXOSC6 | exosome component | 118460 | NM_058219 |
| | | | | | | | 6 | | |
| 0.0000009 | 2.83E−05 | <1e−07 | 122.78 | 54.71 | 2.24 | PTPN1 | protein tyrosine | 5770 | NM_001278618 |
| | | | | | | | phosphatase, non- | | |
| | | | | | | | receptor type 1 | | |
| 0.0000009 | 2.83E−05 | <1e−07 | 50.87 | 22.71 | 2.24 | WASHC2C | WASH complex | 253725 | NM_001169106 |
| | | | | | | | subunit 2C | | |
| 0.00001 | 0.000159 | <1e−07 | 24.6 | 10.99 | 2.24 | QSOX2 | quiescin sulfhydryl | 169714 | NM_181701 |
| | | | | | | | oxidase 2 | | |
| 0.0000254 | 0.000316 | <1e−07 | 27.3 | 12.21 | 2.24 | BMP6 | bone morphogenetic | 654 | NM_001718 |
| | | | | | | | protein 6 | | |
| 0.0000525 | 0.000539 | <1e−07 | 94.06 | 41.9 | 2.24 | MIR29B2CHG | MIR29B2 and | 100128537 | NR_135298 |
| | | | | | | | MIR29C host gene | | |
| 0.0000808 | 0.000739 | 0.0004 | 104.84 | 46.74 | 2.24 | FNIP2 | folliculin interacting | 57600 | NM_001323916 |
| | | | | | | | protein 2 | | |
| 0.0001413 | 0.00114 | 0.0001 | 576.48 | 257.2 | 2.24 | NFKB2 | nuclear factor kappa | 4791 | NM_001077494 |
| | | | | | | | B subunit 2 | | |
| 0.0001987 | 0.00147 | 0.0002 | 15.28 | 6.82 | 2.24 | CCL22 | C-C motif | 6367 | NM_002990 |
| | | | | | | | chemokine ligand | | |
| | | | | | | | 22 | | |
| <1e−07 | <1e−07 | <1e−07 | 122.59 | 54.47 | 2.25 | PLEKHM2 | pleckstrin homology | 23207 | NM_015164 |
| | | | | | | | and RUN domain | | |
| | | | | | | | containing M2 | | |
| <1e−07 | <1e−07 | <1e−07 | 62.35 | 27.69 | 2.25 | SMCR8 | Smith-Magenis | 140775 | NM_144775 |
| | | | | | | | syndrome | | |
| | | | | | | | chromosome region, | | |
| | | | | | | | candidate 8 | | |
| <1e−07 | <1e−07 | <1e−07 | 96.18 | 42.7 | 2.25 | ZNF335 | zinc finger protein | 63925 | NM_022095 |
| | | | | | | | 335 | | |
| 0.0000001 | 5.39E−06 | <1e−07 | 110.55 | 49.15 | 2.25 | VPS37B | VPS37B, ESCRT-I | 79720 | NM_024667 |
| | | | | | | | subunit | | |
| 0.0000004 | 0.000015 | <1e−07 | 38.65 | 17.18 | 2.25 | AUP1 | AUP1, lipid droplet | 550 | NM_181575 |
| | | | | | | | regulating VLDL | | |
| | | | | | | | assembly factor | | |
| 0.0000006 | 2.07E−05 | <1e−07 | 59.59 | 26.53 | 2.25 | ATG4B | autophagy related | 23192 | NM_013325 |
| | | | | | | | 4B cysteine | | |
| | | | | | | | peptidase | | |
| 0.0000019 | 0.000049 | <1e−07 | 42.82 | 19 | 2.25 | POLDIP3 | DNA polymerase | 84271 | NM_001278657 |
| | | | | | | | delta interacting | | |
| | | | | | | | protein 3 | | |
| 0.0000019 | 0.000049 | <1e−07 | 23.38 | 10.4 | 2.25 | TRAM2 | translocation | 9697 | NM_012288 |
| | | | | | | | associated | | |
| | | | | | | | mem brane protein 2 | | |
| 0.0000026 | 6.19E−05 | <1e−07 | 66.97 | 29.78 | 2.25 | DOCK10 | dedicator of | 55619 | NM_001290263 |
| | | | | | | | cytokinesis 10 | | |
| 0.0000031 | 6.98E−05 | <1e−07 | 39702.89 | 17624.61 | 2.25 | MALAT1 | metastasis | 378938 | NR_002819 |
| | | | | | | | associated lung | | |
| | | | | | | | adenocarcinoma | | |
| | | | | | | | transcript 1 (non- | | |
| | | | | | | | protein coding) | | |
| 0.0000156 | 0.000222 | <1e−07 | 27.21 | 12.11 | 2.25 | HEXA | hexosam inidase | 3073 | NM_000520 |
| | | | | | | | subunit alpha | | |
| <1e−07 | <1e−07 | <1e−07 | 19.63 | 8.67 | 2.26 | TCP11L2 | t-complex 11 like 2 | 255394 | NM_001286262 |
| <1e−07 | <1e−07 | <1e−07 | 94.46 | 41.88 | 2.26 | LPIN1 | lipin 1 | 23175 | NM_001261427 |
| 0.0000002 | 9.26E−06 | <1e−07 | 183.31 | 81.13 | 2.26 | SBF1 | SET binding factor | 6305 | NM_002972 |
| | | | | | | | 1 | | |
| 0.0000002 | 9.26E−06 | <1e−07 | 280.37 | 124.16 | 2.26 | UBR4 | ubiquitin protein | 23352 | NM_020765 |
| | | | | | | | ligase E3 | | |
| | | | | | | | component n- | | |
| | | | | | | | recognin 4 | | |
| 0.0000003 | 1.23E−05 | <1e−07 | 59.37 | 26.27 | 2.26 | GATAD2B | GATA zinc finger | 57459 | NM_020699 |
| | | | | | | | domain containing | | |
| | | | | | | | 2B | | |
| 0.0000003 | 1.23E−05 | <1e−07 | 39.92 | 17.7 | 2.26 | DUSP10 | dual specificity | 11221 | NM_007207 |
| | | | | | | | phosphatase 10 | | |
| 0.000001 | 3.07E−05 | <1e−07 | 71.05 | 31.44 | 2.26 | STX5 | syntaxin 5 | 6811 | NM_001244666 |
| 0.0000066 | 0.000118 | <1e−07 | 82.73 | 36.66 | 2.26 | DOCK8 | dedicator of | 81704 | NM_001190458 |
| | | | | | | | cytokinesis 8 | | |
| 0.0000079 | 0.000136 | <1e−07 | 104.95 | 46.46 | 2.26 | SMG1P3 | SMG1 pseudogene | 100271836 | NR_027155 |
| | | | | | | | 3 | | |
| 0.0000263 | 0.000324 | <1e−07 | 49.83 | 22.01 | 2.26 | CHST2 | carbohydrate | 9435 | NM_004267 |
| | | | | | | | sulfotransferase 2 | | |
| 0.0000976 | 0.000853 | <1e−07 | 31.91 | 14.15 | 2.26 | P2RY8 | P2Y receptor family | 286530 | NM_178129 |
| | | | | | | | member 8 | | |
| 0.0004871 | 0.00291 | 0.0001 | 25.25 | 11.19 | 2.26 | SNORD13 | small nucleolar | 692084 | NR_003041 |
| | | | | | | | RNA, C/D box 13 | | |
| 0.0006296 | 0.00357 | 0.0005 | 16.1 | 7.14 | 2.26 | CXCR5 | C-X-C motif | 643 | NM_001716 |
| | | | | | | | chemokine receptor | | |
| | | | | | | | 5 | | |
| <1e−07 | <1e−07 | <1e−07 | 74.23 | 32.64 | 2.27 | MSI2 | musashi RNA | 124540 | NM_001322250 |
| | | | | | | | binding protein 2 | | |
| <1e−07 | <1e−07 | <1e−07 | 28.28 | 12.45 | 2.27 | RANBP10 | RAN binding | 57610 | NM_001320238 |
| | | | | | | | protein 10 | | |
| <1e−07 | <1e−07 | <1e−07 | 20.64 | 9.09 | 2.27 | AC004980.1 | noncoding | | |
| | | | | | | | (Ensemble gene ID: | | |
| | | | | | | | ENSG00000214243) | | |
| <1e−07 | 5.39E−06 | <1e−07 | 37.5 | 16.54 | 2.27 | FAM3C | family with | 10447 | NM_001040020 |
| | | | | | | | sequence similarity | | |
| | | | | | | | 3 member C | | |
| 0.0000003 | 1.23E−05 | <1e−07 | 30.58 | 13.47 | 2.27 | CRTC1 | CREB regulated | 23373 | NM_001098482 |
| | | | | | | | transcription | | |
| | | | | | | | coactivator 1 | | |
| 0.0000008 | 2.61E−05 | <1e−07 | 50.88 | 22.4 | 2.27 | ZFYVE26 | zinc finger FYVE- | 23503 | NM_015346 |
| | | | | | | | type containing 26 | | |
| 0.0000014 | 3.93E−05 | <1e−07 | 115.65 | 50.94 | 2.27 | GCN1 | GCN1, eIF2 alpha | 10985 | NM_006836 |
| | | | | | | | kinase activator | | |
| | | | | | | | homolog | | |
| 0.0000021 | 5.27E−05 | <1e−07 | 49.05 | 21.62 | 2.27 | ADAM17 | ADAM | 6868 | NM_003183 |
| | | | | | | | metallopeptidase | | |
| | | | | | | | domain 17 | | |
| 0.0000044 | 8.87E−05 | <1e−07 | 129.36 | 56.95 | 2.27 | DDX39A | DExD-box helicase | 10212 | NM_001204057 |
| | | | | | | | 39A | | |
| 0.0000174 | 0.000239 | 0.0001 | 17.92 | 7.9 | 2.27 | AL021155.5 | noncoding (Clone | | |
| | | | | | | | Fragment ID: | | |
| | | | | | | | AL021155.1) | | |
| 0.0001649 | 0.00128 | <1e−07 | 30.55 | 13.49 | 2.27 | CD4 | CD4 molecule | 920 | NM_000616 |
| <1e−07 | <1e−07 | <1e−07 | 62.46 | 27.39 | 2.28 | PLEKHO1 | pleckstrin homology | 51177 | NM_001304722 |
| | | | | | | | domain containing | | |
| | | | | | | | O1 | | |
| <1e−07 | <1e−07 | <1e−07 | 117.72 | 51.63 | 2.28 | KMT2A | lysine | 4297 | NM_001197104 |
| | | | | | | | methyltransferase | | |
| | | | | | | | 2A | | |
| <1e−07 | <1e−07 | <1e−07 | 30.18 | 13.26 | 2.28 | NGLY1 | N-glycanase 1 | 55768 | NM_001145293 |
| 0.0000002 | 9.26E−06 | <1e−07 | 241.08 | 105.55 | 2.28 | ARHGEF1 | Rho guanine | 9138 | NM_004706 |
| | | | | | | | nucleotide exchange | | |
| | | | | | | | factor 1 | | |
| 0.0000009 | 2.83E−05 | <1e−07 | 43.06 | 18.87 | 2.28 | CENPC | centromere protein | 1060 | NM_001812 |
| | | | | | | | C | | |
| 0.0000072 | 0.000126 | <1e−07 | 21.23 | 9.3 | 2.28 | CABLES1 | Cdk5 and Abl | 91768 | NM_001100619 |
| | | | | | | | enzyme substrate 1 | | |
| 0.0000113 | 0.000175 | <1e−07 | 52.51 | 23.02 | 2.28 | ANKRD44 | ankyrin repeat | 91526 | NM_001195144 |
| | | | | | | | domain 44 | | |
| 0.0000149 | 0.000215 | <1e−07 | 37.97 | 16.63 | 2.28 | PITPNC1 | phosphatidylinositol | 26207 | NM_012417 |
| | | | | | | | transfer protein | | |
| | | | | | | | cytoplasmic 1 | | |
| 0.0000282 | 0.000342 | 0.0001 | 24.21 | 10.62 | 2.28 | HLA-DMA | major | 3108 | NM_006120 |
| | | | | | | | histocompatibility | | |
| | | | | | | | complex, class II, | | |
| | | | | | | | DM alpha | | |
| 0.0000301 | 0.000356 | <1e−07 | 31.03 | 13.6 | 2.28 | TMEM131L | transmembrane 131 | 23240 | NM_001131007 |
| | | | | | | | like | | |
| 0.0005291 | 0.0031 | 0.0005 | 23.31 | 10.24 | 2.28 | STAP1 | signal transducing | 26228 | NM_001317769 |
| | | | | | | | adaptor family | | |
| | | | | | | | member | | |
| <1e−07 | <1e−07 | <1e−07 | 174.09 | 75.92 | 2.29 | EHBP1L1 | EH domain binding | 254102 | NM_001099409 |
| | | | | | | | protein 1 like 1 | | |
| 0.0000003 | 1.23E−05 | <1e−07 | 25.84 | 11.26 | 2.29 | STK17A | serine/threonin | 9263 | NM_004760 |
| | | | | | | | kinase 17a | | |
| 0.0000003 | 1.23E−05 | <1e−07 | 28.34 | 12.37 | 2.29 | CNPPD1 | cyclin Pas1/PHO80 | 27013 | NM_001321389 |
| | | | | | | | domain containing 1 | | |
| 0.0000007 | 2.34E−05 | <1e−07 | 22.95 | 10.01 | 2.29 | AMZ2 | archaelysin family | 51321 | NM_001033569 |
| | | | | | | | metallopeptidase 2 | | |
| 0.0000018 | 4.73E−05 | <1e−07 | 18.36 | 8 | 2.29 | VASH1 | | 22846 | NM_014909 |
| | | | | | | | vasohibin 1 | | |
| 0.0000028 | 0.000065 | <1e−07 | 158.71 | 69.3 | 2.29 | ARID5A | AT-rich interaction | 10865 | NM_001319085 |
| | | | | | | | domain 5A | | |
| 0.0000031 | 6.98E−05 | <1e−07 | 21.31 | 9.33 | 2.29 | ESR2 | estrogen receptor 2 | 2100 | NM_001040275 |
| 0.0000037 | 0.000079 | <1e−07 | 103.09 | 45.07 | 2.29 | ANKLE2 | ankyrin repeat and | 23141 | NM_015114 |
| | | | | | | | LEM domain | | |
| | | | | | | | containing 2 | | |
| 0.0000045 | 8.96E−05 | <1e−07 | 66.24 | 28.88 | 2.29 | PRNP | | 5621 | NM_000311 |
| | | | | | | | prion protein | | |
| 0.0000063 | 0.000115 | 0.0001 | 24.28 | 10.61 | 2.29 | ADAMTSL4- | ADAMTSL4 | 574406 | NM_001025493 |
| | | | | | | AS1 | antisense RNA 1 | | |
| <1e−07 | <1e−07 | <1e−07 | 59.37 | 25.79 | 2.3 | RB1CC1 | RB1 inducible | 9821 | NM_001083617 |
| | | | | | | | coiled-coil 1 | | |
| 0.0000001 | 5.39E−06 | <1e−07 | 19.8 | 8.61 | 2.3 | TRIM39 | tripartite motif | 56658 | NM_021253 |
| | | | | | | | containing 39 | | |
| 0.0000005 | 1.78E−05 | <1e−07 | 113.4 | 49.23 | 2.3 | ITPR1 | inositol 1,4,5- | 3708 | NM_001099952 |
| | | | | | | | trisphospha | | |
| | | | | | | | receptor type 1 | | |
| 0.0000033 | 7.31E−05 | <1e−07 | 27.22 | 11.84 | 2.3 | PNPLA7 | palatin like | 375775 | NM_001098537 |
| | | | | | | | phospholipase | | |
| | | | | | | | domain containing 7 | | |
| 0.0000103 | 0.000163 | <1e−07 | 29 | 12.63 | 2.3 | AC117382.1 | noncoding (Clone | | |
| | | | | | | | Fragment ID: | | |
| | | | | | | | ACH 7382.10) | | |
| 0.0000271 | 0.000331 | <1e−07 | 58.32 | 25.33 | 23 | PIK3CD | phosphatidylinosilol- | 5293 | NM_001350234 |
| | | | | | | | 4,5-bisphosphate3- | | |
| | | | | | | | kinase catalytic | | |
| | | | | | | | subunit delta | | |
| 0.0000626 | 0.000612 | 0.0001 | 53.41 | 23.17 | 2.3 | SLC5A3 | solute carrier family | 6526 | NM_006933 |
| | | | | | | | 5 member 3 | | |
| 0.000156 | 0.00123 | 000001 | 109.55 | 47.59 | 2.3 | FER1L4 | fer-l like family | 80307 | NR_001442 |
| | | | | | | | member 4, | | |
| | | | | | | | pseudogene | | |
| 0.0000011 | 3.33E−05 | <1e−07 | 150.04 | 65.03 | 2.31 | CSNK1D | casein kinase 1 delta | 1453 | NM_001893 |
| 0.0000016 | 431E−05 | <1e−07 | 87.2 | 37.82 | 2.31 | STK17B | serine/threonine | 9262 | NM_004226 |
| | | | | | | | kinase 17b | | |
| 0.0000021 | 5.27E−05 | <1e−07 | 142.13 | 61.48 | 2.31 | STK4 | serine/threonine | 6789 | NM_001352385 |
| | | | | | | | kinase 4 | | |
| 0.0000091 | 0.00015 | <1e−07 | 106.08 | 45.85 | 2.31 | NIBAN1 | Niban Apoptosis | 116496 | |
| | | | | | | | Regulator 1 | | |
| <1e−07 | <1e−07 | <1e−07 | 137.68 | 59.28 | 2.32 | ZMIZ2 | zinc finger MIZ- | 83637 | NM_001300959 |
| | | | | | | | type containing 2 | | |
| 0.0000001 | 5.39E−06 | <1e−07 | 151.14 | 65.2 | 2.32 | SUN2 | Sad1 and UNC84 | 25777 | NM_001199579 |
| | | | | | | | domain containing 2 | | |
| 0.0000002 | 9.26E−06 | <1e−07 | 68.2 | 29.35 | 2.32 | PKN1 | protein kinase N1 | 5585 | NM_002741 |
| 0.0000013 | 3.73E−05 | <1e−07 | 118.96 | 51.23 | 2.32 | ARHGAP4 | Rho GTPase | 393 | NM_001164741 |
| | | | | | | | activating protein 4 | | |
| 0.0000051 | 9.85E−05 | 0.0001 | 62.17 | 26.83 | 2.32 | GARS1 | Glycyl-TRNA | 2617 | |
| | | | | | | | Synthetase 1 | | |
| 0.000088 | 0.00079 | 0.0001 | 138.15 | 59.47 | 2.32 | GRN | granulin precursor | 2896 | NM_001012479 |
| 0.0001291 | 0.00106 | 0.0002 | 166.78 | 71.78 | 2.32 | TRAF1 | TNF receptor | 7185 | NM_001190945 |
| | | | | | | | associated factor 1 | | |
| 0.0001746 | 0.00133 | <1e−07 | 376.32 | 162.48 | 2.32 | PSAP | prosaposin | 5660 | NM_001042465 |
| <1e−07 | <1e−07 | <1e−07 | 15.68 | 6.73 | 2.33 | WDR45 | WD repeat domain | 11152 | NM_001029896 |
| | | | | | | | 45 | | |
| <1e−07 | <1e−07 | <1e−07 | 55.94 | 24.01 | 2.33 | HDAC7 | histone deacetylase | 51564 | NM_001098416 |
| | | | | | | | 7 | | |
| 0.0000008 | 2.61E−05 | <1e−07 | 31.8 | 13.65 | 2.33 | WASHC4 | WASH complex | 23325 | NM_001293640 |
| | | | | | | | subunit 4 | | |
| 0.0000029 | 6.68E−05 | <1e−07 | 164.98 | 70.66 | 2.33 | ATP2A3 | ATPase | 489 | NM_005173 |
| | | | | | | | sarcoplasmic/ | | |
| | | | | | | | endoplasmic reticulum | | |
| | | | | | | | Ca2+ transporting 3 | | |
| 0.0000049 | 9.58E−05 | <1e−07 | 94.43 | 40.55 | 2.33 | MED12 | mediator complex | 9968 | NM_005120 |
| | | | | | | | subunit 12 | | |
| 0.0000526 | 0.00054 | 0.0002 | 37.03 | 15.91 | 2.33 | NRP2 | neuropilin 2 | 8828 | NM_003872 |
| 0.0002341 | 0.00166 | 0.0005 | 59.43 | 25.5 | 2.33 | SPHK1 | sphingosine kinase 1 | 8877 | NM_001142601 |
| <1e−07 | <1e−07 | <1e−07 | 749.5 | 319.65 | 2.34 | HNRNPH1 | heterogeneous | 3187 | NM_001257293 |
| | | | | | | | nuclear | | |
| | | | | | | | ribonucleoprotein | | |
| | | | | | | | H1 | | |
| 0.0000001 | 5.39E−06 | <1e−07 | 111.77 | 47.73 | 2.34 | ATP2B1 | ATPase plasma | 490 | NM_001001323 |
| | | | | | | | membrane Ca2+ | | |
| | | | | | | | transporting 1 | | |
| 0.0000001 | 5.39E−06 | <1e−07 | 239.51 | 102.26 | 2.34 | CCNL1 | cyclin L1 | 57018 | NM_001308185 |
| 0.0000003 | 1.23E−05 | <1e−07 | 19.2 | 8.19 | 2.34 | ZNRF1 | zinc and ring finger | 84937 | NM_032268 |
| | | | | | | | 1 | | |
| 0.0000003 | 1.23E−05 | <1e−07 | 250.77 | 107.35 | 2.34 | PER1 | period circadian | 5187 | NM_002616 |
| | | | | | | | regulator 1 | | |
| 0.0000006 | 2.07E−05 | <1e−07 | 29.55 | 12.61 | 2.34 | MTSS1 | MTSS1, I-BAR | 9788 | NM_001282971 |
| | | | | | | | domain containing | | |
| 0.0000006 | 2.07E−05 | <1e−07 | 48.92 | 20.88 | 2.34 | KDM6A | lysine demethylase | 7403 | NM_001291415 |
| | | | | | | | 6A | | |
| 0.0000011 | 3.33E−05 | <1e−07 | 22.9 | 9.8 | 2.34 | FCHO1 | FCH domain only 1 | 23149 | NM_001161357 |
| 0.0000024 | 5.78E−05 | <1e−07 | 45.02 | 19.2 | 2.34 | ARL8B | ADP ribosylation | 55207 | NM_018184 |
| | | | | | | | factor like GTPase | | |
| | | | | | | | 8B | | |
| 0.0000028 | 0.000065 | 0.0001 | 34.54 | 14.78 | 2.34 | SNHG12 | small nucleolar | 85028 | NR_024127 |
| | | | | | | | RNA host gene 12 | | |
| 0.0000036 | 7.74E−05 | <1e−07 | 87.22 | 37.34 | 2.34 | TIPARP | TCDD inducible | 25976 | NM_001184717 |
| | | | | | | | poly(ADP-ribose) | | |
| | | | | | | | polymerase | | |
| 0.0000106 | 0.000167 | <1e−07 | 32.72 | 13.96 | 2.34 | ABCG1 | ATP binding | 9619 | NM_004915 |
| | | | | | | | cassette subfamily G | | |
| | | | | | | | member 1 | | |
| 0.0000138 | 0.000203 | <1e−07 | 24.6 | 10.5 | 2.34 | ISYNA1 | inositol-3-phosphate | 51477 | NM_001170938 |
| | | | | | | | synthase 1 | | |
| 0.0001812 | 0.00137 | 0.0003 | 21.99 | 9.39 | 2.34 | NPIPB3 | nuclear pore | 23117 | NM_130464 |
| | | | | | | | complex interacting | | |
| | | | | | | | protein family | | |
| | | | | | | | member B3 | | |
| <1e−07 | <1e−07 | <1e−07 | 27.87 | 11.87 | 2.35 | NSMAF | neutral | 8439 | NM_001144772 |
| | | | | | | | sphingomyelinase | | |
| | | | | | | | activation associated | | |
| | | | | | | | factor | | |
| <1e−07 | <1e−07 | <1e−07 | 24.52 | 10.45 | 2.35 | PUS1 | pseudouridy late | 80324 | NM_001002019 |
| | | | | | | | synthase 1 | | |
| <1e−07 | 5.39E−06 | <1e−07 | 32.28 | 13.76 | 2.35 | USP16 | ubiquitin specific | 10600 | NM_001001992 |
| | | | | | | | peptidase 16 | | |
| 0.0000001 | 5.39E−06 | <1e−07 | 47.95 | 20.38 | 2.35 | ARFGAP2 | ADP ribosy lation | 84364 | NM_001242832 |
| | | | | | | | factor GTPase | | |
| | | | | | | | activating protein 2 | | |
| 0.0000015 | 0.00004 | <1e−07 | 51.89 | 22.12 | 2.35 | GMPPB | GDP-mannose | 29925 | NM_013334 |
| | | | | | | | pyrophosphorylase | | |
| | | | | | | | B | | |
| 0.0000023 | 5.65E−05 | <1e−07 | 30.99 | 13.16 | 2.35 | NPIPB4 | nuclear pore | 440345 | NM_001310148 |
| | | | | | | | complex interacting | | |
| | | | | | | | protein family | | |
| | | | | | | | member B4 | | |
| 0.0000187 | 0.000253 | 0.0002 | 201.17 | 85.69 | 2.35 | ZBTB10 | zinc finger and BTB | 65986 | NM_001105539 |
| | | | | | | | domain containing | | |
| | | | | | | | 10 | | |
| 0.000085 | 0.000769 | 0.0001 | 34.66 | 14.72 | 2.35 | TMC8 | transmembrane | 147138 | NM_152468 |
| | | | | | | | channel like 8 | | |
| 0.000445 | 0.00271 | 0.0004 | 94.79 | 40.34 | 2.35 | SLAMF7 | SLAM family | 57823 | NM_001282588 |
| | | | | | | | member 7 | | |
| <1e−07 | <1e−07 | <1e−07 | 37.22 | 15.76 | 2.36 | TJAP1 | tight junction | 93643 | NM_001146016 |
| | | | | | | | associated protein 1 | | |
| <1e−07 | <1e−07 | <1e−07 | 88.01 | 37.26 | 2.36 | RASA3 | RAS p21 protein | 22821 | NM_001320821 |
| | | | | | | | activator 3 | | |
| 0.0000003 | 1.23E−05 | <1e−07 | 120.38 | 51.1 | 2.36 | STAT6 | signal transducer | 6778 | NM_001178078 |
| | | | | | | | and activator of | | |
| | | | | | | | transcription 6 | | |
| 0.0000014 | 3.93E−05 | <1e−07 | 72.03 | 30.54 | 2.36 | EMP3 | epithelial membrane | 2014 | NM_001313905 |
| | | | | | | | protein 3 | | |
| 0.0000024 | 5.78E−05 | <1e−07 | 70.57 | 29.89 | 2.36 | CD37 | CD37 molecule | 951 | NM_001040031 |
| 0.0000106 | 0.000167 | <1e−07 | 20.07 | 8.51 | 2.36 | AC109326.1 | noncoding | | |
| | | | | | | | (Ensemble gene ID: | | |
| | | | | | | | ENSG00000225584) | | |
| 0.0000199 | 0.000265 | <1e−07 | 87.68 | 37.13 | 2.36 | YPEL5 | yippee like 5 | 51646 | NM_001127399 |
| 0.0000217 | 0.000281 | 0.0002 | 68.73 | 29.16 | 2.36 | MYO1G | myosin IG | 64005 | NM_033054 |
| 0.0000403 | 0.000442 | <1e−07 | 41.45 | 17.55 | 2.36 | TOR3A | torsin family 3 | 64222 | NM_022371 |
| | | | | | | | member A | | |
| 0.0000409 | 0.000446 | <1e−07 | 145 | 61.5 | 2.36 | KLHL21 | kelch like family | 9903 | NM_001324309 |
| | | | | | | | member 21 | | |
| 0.000052 | 0.000536 | 0.0001 | 23.83 | 10.09 | 2.36 | CHST7 | carbohydrate | 56548 | NM_019886 |
| | | | | | | | sulfotransferase 7 | | |
| 0.000062 | 0.000609 | <1e−07 | 394.01 | 166.87 | 2.36 | LENG8 | leukocyte receptor | 114823 | NM_052925 |
| | | | | | | | cluster member 8 | | |
| <1e−07 | <1e−07 | <1e−07 | 74.55 | 31.51 | 2.37 | DDB1 | damage specific | 1642 | NM_001923 |
| | | | | | | | DNA binding | | |
| | | | | | | | protein 1 | | |
| 0.0000003 | 1.23E−05 | <1e−07 | 65.89 | 27.83 | 2.37 | PNKP | polynucleotide | 11284 | NM_007254 |
| | | | | | | | kinase 3′- | | |
| | | | | | | | phosphatase | | |
| 0.0000404 | 0.000442 | <1e−07 | 446.98 | 188.67 | 2.37 | HERPUD1 | homocysteine | 9709 | NM_001010989 |
| | | | | | | | inducible ER protein | | |
| | | | | | | | with ubiquitin like | | |
| | | | | | | | domain 1 | | |
| 0.0000786 | 0.000723 | <1e−07 | 108.85 | 45.89 | 2.37 | THEMIS2 | thymocyte selection | 9473 | NM_001039477 |
| | | | | | | | associated family | | |
| | | | | | | | member 2 | | |
| 0.0005921 | 0.0034 | 0.001 | 17.68 | 7.47 | 2.37 | CD19 | CD19 molecule | 930 | NM_001178098 |
| <1e−07 | <1e−07 | <1e−07 | 22.77 | 9.57 | 2.38 | IFFO1 | intermediate | 25900 | NM_001039670 |
| | | | | | | | filament family | | |
| | | | | | | | orphan 1 | | |
| <1e−07 | <1e−07 | <1e−07 | 20.43 | 8.58 | 2.38 | ZCWPW2 | zinc finger CW-type | 152098 | NM_001040432 |
| | | | | | | | and PWWP domain | | |
| | | | | | | | containing 2 | | |
| 0.0000002 | 9.26E−06 | <1e−07 | 33.62 | 14.12 | 2.38 | MAP3K3 | mitogen-activated | 4215 | NM_001330431 |
| | | | | | | | protein kinase | | |
| | | | | | | | kinase kinase 3 | | |
| 0.0000002 | 9.26E−06 | <1e−07 | 50.08 | 21.05 | 2.38 | NR1H2 | nuclear receptor | 7376 | NM_001256647 |
| | | | | | | | subfamily 1 group H | | |
| | | | | | | | member 2 | | |
| 0.0000004 | 0.000015 | <1e−07 | 376.27 | 157.9 | 2.38 | TGFB1 | transforming growth | 7040 | NM_000660 |
| | | | | | | | factor beta 1 | | |
| 0.0000009 | 2.83E−05 | <1e−07 | 35.11 | 14.74 | 2.38 | ERO1B | endoplasmic | 56605 | NM_019891 |
| | | | | | | | reticulum | | |
| | | | | | | | oxidoreductase 1 | | |
| | | | | | | | beta | | |
| 0.000001 | 3.07E−05 | <1e−07 | 87.38 | 36.76 | 2.38 | LTBP3 | latent transforming | 4054 | NM_001130144 |
| | | | | | | | growth factor beta | | |
| | | | | | | | binding protein 3 | | |
| 0.0003145 | 0.00208 | 0.0006 | 90.26 | 37.92 | 2.38 | RRAD | RRAD, Ras related | 6236 | NM_001128850 |
| | | | | | | | glycolysis inhibitor | | |
| | | | | | | | and calcium channel | | |
| | | | | | | | regulator | | |
| <1e−07 | <1e−07 | <1e−07 | 20.68 | 8.66 | 2.39 | CFASP10 | Cilia And Fla gella | 755 | |
| | | | | | | | Associated Protein | | |
| | | | | | | | 410 | | |
| 0.0000002 | 9.26E−06 | <1e−07 | 1485 | 620.94 | 2.39 | VIM | vimentin | 7431 | NM_003380 |
| 0.0000002 | 9.26E−06 | <1e−07 | 80.01 | 33.52 | 2.39 | PCNX3 | pecanex homolog 3 | 399909 | NM_032223 |
| 0.0000024 | 5.78E−05 | <1e−07 | 30.46 | 12.75 | 2.39 | SYTL1 | synaptotagmin like | 84958 | NM_001193308 |
| | | | | | | | 1 | | |
| 0.0000025 | 0.00006 | <1e−07 | 95.7 | 40.09 | 2.39 | NKTR | natural killer cell | 4820 | NM_001012651 |
| | | | | | | | triggering receptor | | |
| 0.0000064 | 0.000116 | 0.0001 | 22.9 | 9.59 | 2.39 | A4GALT | alpha 1,4- | 53947 | NM_001318038 |
| | | | | | | | galactosyltransferase | | |
| | | | | | | | (P blood group) | | |
| 0.0000356 | 0.000404 | <1e−07 | 212.26 | 88.74 | 2.39 | MAPK8IP3 | mitogen-activated | 23162 | NM_001040439 |
| | | | | | | | protein kinase 8 | | |
| | | | | | | | interactingprotein 3 | | |
| 0.0001889 | 0.00141 | 0.0001 | 255.89 | 107.22 | 2.39 | LTBP4 | latent transforming | 8425 | NM_001042544 |
| | | | | | | | growth factor beta | | |
| | | | | | | | binding protein 4 | | |
| 0.0008179 | 0.00438 | 0.0008 | 85.08 | 35.57 | 2.39 | SSR4 | signal sequence | 6748 | NM_001204526 |
| | | | | | | | receptor subunit 4 | | |
| <1e−07 | <1e−07 | <1e−07 | 210.01 | 87.63 | 2.4 | MEF2D | myocyte enhancer | 4209 | NM_001271629 |
| | | | | | | | factor 2D | | |
| <1e−07 | <1e−07 | <1e−07 | 47.69 | 19.83 | 2.4 | PLEKHM1 | pleckstrin homology | 9842 | NM_001352825 |
| | | | | | | | and RUN domain | | |
| | | | | | | | containing M1 | | |
| 0.0000034 | 7.49E−05 | <1e−07 | 24.79 | 10.33 | 2.4 | ST6GALNAC4 | ST6 N- | 27090 | NM_014403 |
| | | | | | | | acetylgalactosaminide | | |
| | | | | | | | alpha-2,6- | | |
| | | | | | | | sialyltransferase 4 | | |
| 0.0000038 | 7.99E−05 | <1e−07 | 43.93 | 18.31 | 2.4 | ST6GAL1 | ST6 beta- | 6480 | NM_001353916 |
| | | | | | | | galactoside alpha- | | |
| | | | | | | | 2,6-sialyltransferase | | |
| | | | | | | | 1 | | |
| 0.0001008 | 0.000874 | <1e−07 | 46.66 | 19.41 | 2.4 | ITGAL | integrin subunit | 3683 | NM_001114380 |
| | | | | | | | alpha L | | |
| 0.0001551 | 0.00122 | <1e−07 | 47.96 | 19.97 | 2.4 | GM2A | GM2 ganglioside | 2760 | NM_000405 |
| | | | | | | | activator | | |
| 0.0000021 | 5.27E−05 | <1e−07 | 30.73 | 12.75 | 2.41 | AC139887.2 | noncoding | | |
| | | | | | | | (Ensemble gene ID: | | |
| | | | | | | | ENSG00000249592.6) | | |
| 0.0000064 | 0.000116 | <1e−07 | 62.93 | 26.13 | 2.41 | WDR74 | WD repeat domain | 54663 | NM_001307977 |
| | | | | | | | 74 | | |
| 0.0000171 | 0.000237 | <1e−07 | 18.55 | 7.69 | 2.41 | EBF1 | early B cell factor 1 | 1879 | NM_001290360 |
| <1e−07 | <1e−07 | <1e−07 | 29.01 | 12.01 | 2.42 | SGSH | N-sulfoglucosamine | 6448 | NM_000199 |
| | | | | | | | sulfohydrolase | | |
| <1e−07 | <1e−07 | <1e−07 | 48.29 | 19.98 | 2.42 | SRSF6 | serine and arginine | 6431 | NM_006275 |
| | | | | | | | rich splicing factor 6 | | |
| <1e−07 | 5.39E−06 | <1e−07 | 34.4 | 14.21 | 2.42 | CYBC1 | cytochrome b-245 | 79415 | NM_001033046 |
| | | | | | | | chaperone 1 | | |
| 0.0000014 | 3.93E−05 | <1e−07 | 91.75 | 37.9 | 2.42 | RHBDF2 | rhomboid 5 | 79651 | NM_001005498 |
| | | | | | | | homolog 2 | | |
| 0.0000063 | 0.000115 | <1e−07 | 90.88 | 37.59 | 2.42 | SIPA1 | signal-induced | 6494 | NM_006747 |
| | | | | | | | proliferation- | | |
| | | | | | | | associated 1 | | |
| <1e−07 | <1e−07 | <1e−07 | 57.21 | 23.51 | 2.43 | SNX29 | sorting nexin 29 | 92017 | NM_001009607 |
| <1e−07 | <1e−07 | <1e−07 | 53.14 | 21.88 | 2.43 | SLC41A1 | solute carrier family | 254428 | NM_173854 |
| | | | | | | | 41 member 1 | | |
| 0.0000001 | 5.39E−06 | <1e−07 | 46.06 | 18.97 | 2.43 | SHMT2 | serine | 6472 | NM_001166356 |
| | | | | | | | hydroxymethyl- | | |
| | | | | | | | transferase 2 | | |
| 0.0000002 | 9.26E−06 | <1e−07 | 26.76 | 10.99 | 2.43 | PPP3CC | protein phosphatase | 5533 | NM_001243974 |
| | | | | | | | 3 catalytic subunit | | |
| | | | | | | | gamma | | |
| 0.0000003 | 1.23E−05 | <1e−07 | 28.42 | 11.69 | 2.43 | LINC00342 | long intergenic non- | 150759 | NR_103734 |
| | | | | | | | protein coding RNA | | |
| | | | | | | | 342 | | |
| 0.0000005 | 1.78E−05 | <1e−07 | 15.77 | 6.48 | 2.43 | ADM2 | adrenomedullin 2 | 79924 | NM_001253845 |
| 0.0000023 | 5.65E−05 | <1e−07 | 173.56 | 71.44 | 2.43 | PIM1 | Pim-1 proto- | 5292 | NM_001243186 |
| | | | | | | | oncogene, | | |
| | | | | | | | serine/threonine | | |
| | | | | | | | kinase | | |
| 0.0000055 | 0.000104 | <1e−07 | 47.27 | 19.49 | 2.43 | SNED1 | sushi, nidogen and | 25992 | NM_001080437 |
| | | | | | | | EGF like domains 1 | | |
| 0.0000339 | 0.000389 | 0.0001 | 68.78 | 28.31 | 2.43 | SESN2 | sestrin 2 | 83667 | NM_031459 |
| <1e−07 | <1e−07 | <1e−07 | 63.77 | 26.09 | 2.44 | TTC7A | tetratricopeptide | 57217 | NM_001288951 |
| | | | | | | | repeat domain 7A | | |
| 0.0000012 | 3.52E−05 | <1e−07 | 60.96 | 25.03 | 2.44 | SLC7A1 | solute carrier family | 6541 | NM_003045 |
| | | | | | | | 7 member 1 | | |
| 0.0000045 | 8.96E−05 | <1e−07 | 112.32 | 45.97 | 2.44 | ITPKB | inositol- | 3707 | NM_002221 |
| | | | | | | | trisphosphate 3- | | |
| | | | | | | | kinase B | | |
| 0.00001 | 0.000159 | <1e−07 | 62.4 | 25.52 | 2.44 | ST3GAL1 | ST3 beta- | 6482 | NM_003033 |
| | | | | | | | galactoside alpha- | | |
| | | | | | | | 2,3-sialyltransferase | | |
| | | | | | | | 1 | | |
| <1e−07 | <1e−07 | <1e−07 | 16.77 | 6.84 | 2.45 | AC005332.4 | noncoding | | |
| | | | | | | | (Ensemble gene ID: | | |
| | | | | | | | ENSG00000274712.1) | | |
| 0.0000003 | 1.23E−05 | <1e−07 | 215.32 | 87.92 | 2.45 | MGAT1 | mannosyl (alpha- | 4245 | NM_001114617 |
| | | | | | | | 1,3-)-glycoprotein | | |
| | | | | | | | beta-1,2-N- | | |
| | | | | | | | acetylglucosaminyl- | | |
| | | | | | | | transferase | | |
| 0.0000035 | 7.64E−05 | <1e−07 | 50.42 | 20.59 | 2.45 | ITGA4 | integrin subunit | 3676 | NM_000885 |
| | | | | | | | alpha 4 | | |
| 0.0000124 | 0.000188 | <1e−07 | 33.49 | 13.67 | 2.45 | CHPF | chondroitin | 79586 | NM_001195731 |
| | | | | | | | polymerizing factor | | |
| 0.0000346 | 0.000395 | <1e−07 | 208.68 | 85.11 | 2.45 | GAK | cyclin G associated | 2580 | NM_001286833 |
| | | | | | | | kinase | | |
| 0.0000363 | 0.000409 | <1e−07 | 87.11 | 35.61 | 2.45 | OTUD1 | OTU deubiquitinase | 220213 | NM_001145373 |
| | | | | | | | 1 | | |
| 0.0001419 | 0.00114 | 0.0001 | 154.78 | 63.14 | 2.45 | ANKRD28 | ankyrin repeat | 23243 | NM_001195098 |
| | | | | | | | domain 28 | | |
| <1e−07 | <1e−07 | <1e−07 | 36.27 | 14.74 | 2.46 | IDS | iduronate 2-sulfatase | 3423 | NM_000202 |
| 0.0000002 | 9.26E−06 | <1e−07 | 79.3 | 32.26 | 2.46 | PLXND1 | plexin D1 | 23129 | NM_015103 |
| 0.0000007 | 2.34E−05 | <1e−07 | 103.54 | 42.09 | 2.46 | EDEM1 | ER degradation | 9695 | NM_014674 |
| | | | | | | | enhancing alpha - | | |
| | | | | | | | mannosidase like | | |
| | | | | | | | protein 1 | | |
| 0.0005971 | 0.00343 | 0.0003 | 135.05 | 54.88 | 2.46 | DUSP5 | dual specificity | 1847 | NM_004419 |
| | | | | | | | phosphatase 5 | | |
| <1e−07 | <1e−07 | <1e−07 | 31.7 | 12.85 | 2.47 | ATP8B2 | ATPase | 57198 | NM_001005855 |
| | | | | | | | phospholipid | | |
| | | | | | | | transporting 8B2 | | |
| 0.0000001 | 5.39E−06 | <1e−07 | 85.33 | 34.51 | 2.47 | AARS1 | Alanyl-TRNA | 16 | |
| | | | | | | | Synthetase 1 | | |
| 0.0000002 | 9.26E−06 | <1e−07 | 154.16 | 62.36 | 2.47 | FNBP4 | formin binding | 23360 | NM_001318339 |
| | | | | | | | protein 4 | | |
| 0.0000023 | 5.65E−05 | 0.0001 | 19.14 | 7.75 | 2.47 | SIK1B | salt inducible kinase | 102724428 | NM_001320643 |
| | | | | | | | 1B (putative) | | |
| 0.0000049 | 9.58E−05 | <1e−07 | 36.57 | 14.82 | 2.47 | SP140 | SP140 nuclear body | 11262 | NM_001005176 |
| | | | | | | | protein | | |
| 0.0000052 | 0.0001 | <1e−07 | 40.42 | 16.4 | 2.47 | RASAL3 | RAS protein | 64926 | NM_001348027 |
| | | | | | | | activator like 3 | | |
| 0.0000275 | 0.000335 | <1e−07 | 96.44 | 38.99 | 2.47 | HIVEP3 | human | 59269 | NM_001127714 |
| | | | | | | | immunodeficiency | | |
| | | | | | | | virus type I | | |
| | | | | | | | enhancer binding | | |
| | | | | | | | protein 3 | | |
| <1e−07 | <1e−07 | <1e−07 | 14.2 | 5.72 | 2.48 | AL353795.3 | noncoding | | |
| | | | | | | | (Ensemble gene ID: | | |
| | | | | | | | ENSG00000279608.1) | | |
| <1e−07 | <1e−07 | <1e−07 | 96.42 | 38.88 | 2.48 | ULK1 | unc-51 like | 8408 | NM_003565 |
| | | | | | | | autophagy activating | | |
| | | | | | | | kinase 1 | | |
| 0.0000009 | 2.83E−05 | <1e−07 | 99.42 | 40.05 | 2.48 | ZNF331 | zinc finger protein | 55422 | NM_001079906 |
| | | | | | | | 331 | | |
| 0.0000023 | 5.65E−05 | <1e−07 | 65.83 | 26.56 | 2.48 | OSBPL3 | oxysterol binding | 26031 | NM_015550 |
| | | | | | | | protein like 3 | | |
| 0.0000089 | 0.000147 | <1e−07 | 20.2 | 8.14 | 2.48 | RNVU1- | RNA, Variant U1 | 115409988 | |
| | | | | | | 27 | Small Nuclear 27 | | |
| 0.0000459 | 0.000489 | <1e−07 | 80.44 | 32.48 | 2.48 | RGCC | regulator of cell | 28984 | NM_014059 |
| | | | | | | | cycle | | |
| 0.0008113 | 0.00435 | 0.001 | 36.08 | 14.57 | 2.48 | DNASE1L3 | deoxyribonuclease 1 | 1776 | NM_001256560 |
| | | | | | | | like 3 | | |
| <1e−07 | <1e−07 | <1e−07 | 178.83 | 71.85 | 2.49 | IL10RA | interleukin 10 | 3587 | NM_001558 |
| | | | | | | | receptor subunit | | |
| | | | | | | | alpha | | |
| 0.0000027 | 6.33E−05 | <1e−07 | 118.24 | 47.53 | 2.49 | NCOA3 | nuclear receptor | 8202 | NM_001174087 |
| | | | | | | | coactivator 3 | | |
| 0.0000035 | 7.64E−05 | <1e−07 | 25.97 | 10.41 | 2.49 | IL411 | interleukin 4 | 259307 | NM_001258017 |
| | | | | | | | induced 1 | | |
| 0.0000146 | 0.000211 | 0.0001 | 29.98 | 12.04 | 2.49 | AC016831.1 | noncoding | | |
| | | | | | | | (Ensemble gene ID: | | |
| | | | | | | | ENSG00000226380.9) | | |
| 0.0000216 | 0.00028 | <1e−07 | 77.2 | 31 | 2.49 | ARHGAP9 | Rho GTPase | 64333 | NM_001080156 |
| | | | | | | | activating protein 9 | | |
| 0.0000278 | 0.000338 | <1e−07 | 21.05 | 8.44 | 2.49 | ADGRB1 | adhesion G protein- | 575 | NM_001702 |
| | | | | | | | coupled receptor B1 | | |
| <1e−07 | <1e−07 | <1e−07 | 28.65 | 11.47 | 2.5 | PARVB | parvin beta | 29780 | NM_001003828 |
| <1e−07 | <1e−07 | <1e−07 | 54.14 | 21.69 | 2.5 | ZNF460 | zinc finger protein | 10794 | NM_001330622 |
| | | | | | | | 460 | | |
| <1e−07 | <1e−07 | <1e−07 | 117.18 | 46.8 | 2.5 | KDM2B | lysine demethylase | 84678 | NM_001005366 |
| | | | | | | | 2B | | |
| <1e−07 | 5.39E−06 | <1e−07 | 139.07 | 55.65 | 2.5 | CRTC2 | CREB regulated | 200186 | NM_181715 |
| | | | | | | | transcription | | |
| | | | | | | | coactivator 2 | | |
| 0.0000004 | 0.000015 | <1e−07 | 49.47 | 19.81 | 2.5 | ENTPD4 | ectonucleoside | 9583 | NM_001128930 |
| | | | | | | | triphosphate | | |
| | | | | | | | diphosphohydrolase | | |
| | | | | | | | 4 | | |
| 0.0000005 | 1.78E−05 | <1e−07 | 55.35 | 22.16 | 2.5 | MAP3K1 | mitogen-activated | 4214 | NM_005921 |
| | | | | | | | protein kinase | | |
| | | | | | | | kinase kinase 1 | | |
| 0.0000012 | 3.52E−05 | <1e−07 | 42.39 | 16.96 | 2.5 | ITGB7 | integrin subunit beta | 3695 | NM_000889 |
| | | | | | | | 7 | | |
| 0.0000072 | 0.000126 | <1e−07 | 38.43 | 15.38 | 2.5 | FGD2 | FYVE, Rho GEF and | 221472 | NM_173558 |
| | | | | | | | PH domain | | |
| | | | | | | | containing 2 | | |
| | | | | | | | noncoding | | |
| 0.0000364 | 0.00041 | <1e−07 | 105.02 | 42.09 | 2.5 | AC007384.1 | (Ensemble gene ID: | | |
| | | | | | | | ENSG00000237513.2) | | |
| 0.0000534 | 0.000544 | <1e−07 | 20.24 | 8.1 | 2.5 | FCMR | Fc fragment of IgM | 9214 | NM_001142472 |
| | | | | | | | receptor | | |
| 0.0000835 | 0.00076 | 0.0002 | 122.86 | 49.12 | 2.5 | NFKBID | NFKB inhibitor | 84807 | NM_001321831 |
| | | | | | | | delta | | |
| 0.0000864 | 0.000779 | 0.0002 | 41.71 | 16.66 | 2.5 | ADAM28 | ADAM | 10863 | NM_001304351 |
| | | | | | | | metallopeptidase | | |
| | | | | | | | domain 28 | | |
| 0.0001106 | 0.000941 | 0.0001 | 88.33 | 35.29 | 2.5 | FYN | FYN proto- | 2534 | NM_001242779 |
| | | | | | | | oncogene, Src | | |
| | | | | | | | family tyrosine | | |
| | | | | | | | kinase | | |
| 0.0001771 | 0.00135 | 0.0001 | 2498.29 | 999.47 | 2.5 | FOSB | FosB proto- | 2354 | NM_001114171 |
| | | | | | | | oncogene, AP-1 | | |
| | | | | | | | transcription factor | | |
| | | | | | | | subunit | | |
| 0.0000003 | 1.23E−05 | <1e−07 | 72.97 | 29.1 | 2.51 | CARS1 | Cysteinyl-TRNA | 833 | |
| | | | | | | | Synthetase 1 | | |
| | | | | | | | Methionyl-TRNA | | |
| | | | | | | | Synthetase 1 | | |
| 0.000008 | 0.000137 | <1e−07 | 14.79 | 5.89 | 2.51 | LINC00926 | longintergenic non- | 283663 | NR_024433 |
| | | | | | | | protein coding RNA | | |
| | | | | | | | 926 | | |
| <1e−07 | <1e−07 | <1e−07 | 376.37 | 149.5 | 2.52 | FNBP1 | formin binding | 23048 | NM_015033 |
| | | | | | | | protein 1 | | |
| <1e−07 | <1e−07 | <1e−07 | 70.24 | 27.9 | 2.52 | MTA2 | meta stasis | 9219 | NM_001330292 |
| | | | | | | | associated 1 family | | |
| | | | | | | | member 2 | | |
| 0.0000018 | 4.73E−05 | <1e−07 | 38.93 | 15.4 | 2.53 | TPRA1 | transmembrane | 131601 | NM_001136053 |
| | | | | | | | protein adipocyte | | |
| | | | | | | | associated 1 | | |
| 0.000217 | 0.00157 | 0.0004 | 52.49 | 20.72 | 2.53 | HLA-DQA1 | major | 3117 | NM_002122 |
| | | | | | | | histocompatibility | | |
| | | | | | | | complex, class II, | | |
| | | | | | | | DQ alpha 1 | | |
| <1e−07 | <1e−07 | <1e−07 | 32.02 | 12.62 | 2.54 | AMFR | autocrine motility | 267 | NM_001144 |
| | | | | | | | factor receptor | | |
| <1e−07 | <1e−07 | <1e−07 | 34.13 | 13.46 | 2.54 | PNMA1 | PNMA family | 9240 | NM_006029 |
| | | | | | | | member 1 | | |
| <1e−07 | <1e−07 | <1e−07 | 26.25 | 10.32 | 2.54 | SAT2 | sperm idine/spermine | 112483 | NM_001320845 |
| | | | | | | | N1- | | |
| | | | | | | | acetyltransferase | | |
| | | | | | | | family member 2 | | |
| 0.0000001 | 5.39E−06 | <1e−07 | 105.24 | 41.37 | 2.54 | SYVN1 | synoviolin 1 | 84447 | NM_032431 |
| 0.0000036 | 7.74E−05 | <1e−07 | 47.24 | 18.62 | 2.54 | RHOH | ras homolog family | 399 | NM_001278359 |
| | | | | | | | member H | | |
| 0.0000536 | 0.000545 | 0.0002 | 125.25 | 49.25 | 2.54 | SCARNA7 | small Cajal body- | 677767 | NR_003001 |
| | | | | | | | specific RNA 7 | | |
| <1e−07 | <1e−07 | <1e−07 | 46.04 | 18.05 | 2.55 | MGAT2 | mannosyl (alpha- | 4247 | NM_001015883 |
| | | | | | | | 1,6-)-glycoprotein | | |
| | | | | | | | beta-1,2-N- | | |
| | | | | | | | acetylglucosaminyl- | | |
| | | | | | | | transferase | | |
| 0.0000056 | 0.000105 | <1e−07 | 49.12 | 19.28 | 2.55 | AMDHD2 | amidohydrolase | 51005 | NM_001145815 |
| | | | | | | | domain containing 2 | | |
| 0.0003872 | 0.00243 | 0.0003 | 40.37 | 15.83 | 2.55 | ACP5 | acid phosphatase 5, | 54 | NM_001111034 |
| | | | | | | | tartrate resistant | | |
| 0.0006423 | 0.00363 | 0.0007 | 87.62 | 34.35 | 2.55 | ADAMTS1 | ADAM | 9510 | NM_006988 |
| | | | | | | | metallopeptidase | | |
| | | | | | | | with | | |
| | | | | | | | thrombospondin | | |
| | | | | | | | type 1 motif 1 | | |
| <1e−07 | <1e−07 | <1e−07 | 138.37 | 54.05 | 2.56 | TNK2 | tyrosine kinase non | 10188 | NM_001010938 |
| | | | | | | | receptor 2 | | |
| <1e−07 | <1e−07 | <1e−07 | 127.39 | 49.76 | 2.56 | ZHX2 | zinc fingers and | 22882 | NM_014943 |
| | | | | | | | homeoboxes 2 | | |
| <1e−07 | <1e−07 | <1e−07 | 56.48 | 22.05 | 2.56 | TRPM7 | transient receptor | 54822 | NM_001301212 |
| | | | | | | | potential cation | | |
| | | | | | | | channel subfamily | | |
| | | | | | | | M member 7 | | |
| 0.0000002 | 9.26E−06 | <1e−07 | 27.47 | 10.73 | 2.56 | CARMIL2 | capping protein | 146206 | NM_001013838 |
| | | | | | | | regulator and | | |
| | | | | | | | myosin 1 linker 2 | | |
| 0.0000013 | 3.73E−05 | <1e−07 | 58.05 | 22.69 | 2.56 | SLC8B1 | solute carrier family | 80024 | NM_001330466 |
| | | | | | | | 8 member B1 | | |
| 0.0000036 | 7.74E−05 | <1e−07 | 46.65 | 18.24 | 2.56 | JAK3 | Janus kinase 3 | 3718 | NM_000215 |
| <1e−07 | <1e−07 | <1e−07 | 48.11 | 18.73 | 2.57 | SMG1P1 | SMG1 pseudogene | 641298 | NM_199284 |
| | | | | | | | 1 | | |
| 0.0000002 | 9.26E−06 | <1e−07 | 140.91 | 54.91 | 2.57 | TRA2A | transformer 2 alpha | 29896 | NM_001282757 |
| | | | | | | | homolog | | |
| 0.0000121 | 0.000185 | <1e−07 | 29.92 | 11.65 | 2.57 | AC018445.3 | noncoding | | |
| | | | | | | | (Ensemble gene ID: | | |
| | | | | | | | ENSG00000279637.1) | | |
| 0.000148 | 0.00118 | <1e−07 | 218.23 | 84.92 | 2.57 | PNISR | PNN interacting | 25957 | NM_001322405 |
| | | | | | | | serine and arginine | | |
| | | | | | | | rich protein | | |
| 0.0001764 | 0.00134 | 0.0001 | 177.99 | 69.22 | 2.57 | PLCG2 | phospholipase C | 5336 | NM_002661 |
| | | | | | | | gamma 2 | | |
| <1e−07 | <1e−07 | <1e−07 | 27.24 | 10.57 | 2.58 | DNAJC1 | DnaJ heat shock | 64215 | NM_022365 |
| | | | | | | | protein family | | |
| | | | | | | | (Hsp40) member C1 | | |
| <1e−07 | <1e−07 | <1e−07 | 58.75 | 22.76 | 2.58 | CCDC69 | coiled-coil domain | 26112 | NM_015621 |
| | | | | | | | containing 69 | | |
| 0.0000002 | 9.26E−06 | <1e−07 | 54.53 | 21.17 | 2.58 | UBE2Z | ubiquitin | 65264 | NM_023079 |
| | | | | | | | conjugating enzyme | | |
| | | | | | | | E2 Z | | |
| <1e−07 | 5.39E−05 | <1e−07 | 57.88 | 22.38 | 2.59 | IARS1 | Isoleucyl-TRNA | 3376 | |
| | | | | | | | Synthetase 1 | | |
| 0.0000274 | 0.000334 | 0.0001 | 36.25 | 14.01 | 2.59 | SPAG4 | sperm associated | 6676 | NM_001317931 |
| | | | | | | | antigen 4 | | |
| 0.0000007 | 2.34E−05 | <1e−07 | 142.42 | 54.81 | 2.6 | TYK2 | tyrosine kinase 2 | 7297 | NM_003331 |
| 0.0000074 | 0.000128 | <1e−07 | 159.49 | 61.33 | 2.6 | IRF4 | interferon regulatory | 3662 | NM_001195286 |
| | | | | | | | factor 4 | | |
| 0.0000151 | 0.000217 | 0.0001 | 148.09 | 56.65 | 2.61 | EIF2AK3 | eukaryotic | 9451 | NM_001313915 |
| | | | | | | | translation initiation | | |
| | | | | | | | factor 2 alpha kinase | | |
| | | | | | | | 3 | | |
| <1e−07 | <1e−07 | <1e−07 | 15.6 | 5.95 | 2.62 | PARP15 | poly (ADP-ribose) | 165631 | NM_001113523 |
| | | | | | | | polymerase family | | |
| | | | | | | | member 15 | | |
| <1e−07 | <1e−07 | <1e−07 | 273.91 | 104.64 | 2.62 | ERN1 | endoplasmic | 2081 | NM_001433 |
| | | | | | | | reticulum to nucleus | | |
| | | | | | | | signaling 1 | | |
| <1e−07 | <1e−07 | <1e−07 | 32.25 | 12.29 | 2.62 | MBD3 | methyl-CpG binding | 53615 | NM_001281453 |
| | | | | | | | domain protein 3 | | |
| <1e−07 | <1e−07 | <1e−07 | 280.83 | 107.12 | 2.62 | AKNA | AT-hook | 80709 | NM_001317950 |
| | | | | | | | transcription factor | | |
| 0.0000001 | 5.39E−06 | <1e−07 | 83.12 | 31.74 | 2.62 | IFFO2 | intermediate | 126917 | NM_001136265 |
| | | | | | | | filament family | | |
| | | | | | | | orphan 2 | | |
| 0.0000068 | 0.000121 | <1e−07 | 67.13 | 25.62 | 2.62 | GRASP | general receptor for | 160622 | NM_001271856 |
| | | | | | | | phosphoinositides 1 | | |
| | | | | | | | associated scaffold | | |
| | | | | | | | protein | | |
| <1e−07 | <1e−07 | <1e−07 | 17.8 | 6.76 | 2.63 | S1PR2 | sphingosine-1- | 9294 | NM_004230 |
| | | | | | | | phosphate receptor 2 | | |
| <1e−07 | <1e−07 | <1e−07 | 39.4 | 15.01 | 2.63 | SLC12A4 | solute carrier family | 6560 | NM_001145961 |
| | | | | | | | 12 member 4 | | |
| <1e−07 | <1e−07 | <1e−07 | 129.15 | 49.13 | 2.63 | PHF1 | PHD finger protein | 5252 | NM_002636 |
| | | | | | | | 1 | | |
| 0.0000057 | 0.000106 | <1e−07 | 18.65 | 7.09 | 2.63 | HLA-DMB | major | 3109 | NM_002118 |
| | | | | | | | histocompatibility | | |
| | | | | | | | complex, class II, | | |
| | | | | | | | DM beta | | |
| 0.0000073 | 0.000127 | <1e−07 | 80.01 | 30.4 | 2.63 | GPR132 | G protein-coupled | 29933 | NM_001278694 |
| | | | | | | | receptor 132 | | |
| 0.0000568 | 0.00057 | <1e−07 | 56.04 | 21.29 | 2.63 | SYTL3 | synaptota gmin like | 94120 | NM_001009991 |
| | | | | | | | 3 | | |
| 0.0004119 | 0.00255 | 0.0005 | 52.43 | 19.94 | 2.63 | MMP19 | matrix | 4327 | NM_001032360 |
| | | | | | | | metallopeptidase 19 | | |
| 0.0004946 | 0.00294 | 0.0006 | 49.98 | 19.01 | 2.63 | ZBTB16 | zinc finger and BTB | 7704 | NM_001018011 |
| | | | | | | | domain containing | | |
| | | | | | | | 16 | | |
| <1e−07 | <1e−07 | <1e−07 | 73.92 | 27.98 | 2.64 | GALNT2 | polypeptide N- | 2590 | NM_001291866 |
| | | | | | | | acetylgalactosaminyl- | | |
| | | | | | | | transferase 2 | | |
| 0.0000001 | 5.39E−06 | <1e−07 | 26.28 | 9.91 | 2.65 | BHLHE41 | basic helix-loop- | 79365 | NM_030762 |
| | | | | | | | helix family | | |
| | | | | | | | member e41 | | |
| 0.0000002 | 9.26E−06 | <1e−07 | 92.92 | 35.11 | 2.65 | PBXIP1 | PBX homeobox | 57326 | NM_001317734 |
| | | | | | | | interactingprotein 1 | | |
| 0.0005157 | 0.0030 | 0.0005 | 127.22 | 48.09 | 2.65 | ANKRD36BP2 | ankyrin repeat | 645784 | NR_015424 |
| | | | | | | | domain 36B | | |
| | | | | | | | pseudogene 2 | | |
| <1e−07 | <1e−07 | <1e−07 | 110.11 | 41.44 | 2.66 | NFATC1 | nuclear factor of | 4772 | NM_001278669 |
| | | | | | | | activated T cells 1 | | |
| <1e−07 | <1e−07 | <1e−07 | 863.28 | 324.84 | 2.66 | FLNA | filam in A | 2316 | NM_001110556 |
| <1e−07 | 5.39E−06 | <1e−07 | 31.09 | 11.68 | 2.66 | AC044849.1 | noncoding | | |
| | | | | | | | (Ensemble gene ID: | | |
| | | | | | | | ENSG00000272256.1) | | |
| 0.0000066 | 0.000118 | <1e−07 | 165.71 | 62.26 | 2.66 | KLF2 | Kruppel like factor 2 | 10365 | NM_016270 |
| 0.0000088 | 0.000146 | <1e−07 | 41.61 | 15.65 | 2.66 | P2RX1 | purinergic receptor | 5023 | NM_002558 |
| | | | | | | | P2X 1 | | |
| 0.0000146 | 0.000211 | <1e−07 | 96.23 | 36.13 | 2.66 | PIK3CG | phosphatidylinositol- | 5294 | NM_001282426 |
| | | | | | | | 4,5-bisphosphate 3- | | |
| | | | | | | | kinase catalytic | | |
| | | | | | | | subunit gamma | | |
| 0.0000005 | 1.78E−05 | <1e−07 | 24.46 | 9.17 | 2.67 | DTX1 | deltex E3 ubiquitin | 1840 | NM_004416 |
| | | | | | | | ligase 1 | | |
| 0.0000008 | 2.61E−05 | <1e−07 | 148.8 | 55.75 | 2.67 | HCLS1 | hematopoietic cell- | 3059 | NM_001292041 |
| | | | | | | | specific Lyn | | |
| | | | | | | | substrate 1 | | |
| 0.0000695 | 0.00066 | <1e−07 | 44.18 | 16.54 | 2.67 | MZB1 | marginal zone B and | 51237 | NM_016459 |
| | | | | | | | B1 cell specific | | |
| | | | | | | | protein | | |
| <1e−07 | <1e−07 | <1e−07 | 137.54 | 51.34 | 2.68 | JMY | junction mediating | 133746 | NM_152405 |
| | | | | | | | and regulatory | | |
| | | | | | | | protein, p53 cofactor | | |
| 0.0000059 | 0.000109 | <1e−07 | 57.7 | 21.51 | 2.68 | CARD11 | caspase recruitment | 84433 | NM_001324281 |
| | | | | | | | domain family | | |
| | | | | | | | member 11 | | |
| 0.0000776 | 0.000717 | <1e−07 | 64.93 | 24.22 | 2.68 | IKZF3 | IKAROS family | 22806 | NM_001257408 |
| | | | | | | | zinc finger 3 | | |
| <1e−07 | <1e−07 | <1e−07 | 104.42 | 38.85 | 2.69 | WDR6 | WD repeat domain 6 | 11180 | NM_001320546 |
| <1e−07 | <1e−07 | <1e−07 | 62 | 23.09 | 2.69 | IL6R | interleukin 6 | 3570 | NM_000565 |
| | | | | | | | receptor | | |
| 0.0000005 | 1.78E−05 | <1e−07 | 67.3 | 25 | 2.69 | ATF5 | activating | 22809 | NM_001193646 |
| | | | | | | | transcription factor | | |
| | | | | | | | 5 | | |
| <1e−07 | <1e−07 | <1e−07 | 227.62 | 84.45 | 2.7 | PABPC4 | poly (A) binding | 8761 | NM_001135653 |
| | | | | | | | protein cytoplasm ic | | |
| | | | | | | | 4 | | |
| 0.0000004 | 0.000015 | <1e−07 | 454.39 | 168.51 | 2.7 | TSC22D3 | TSC22 domain | 1831 | NM_001015881 |
| | | | | | | | family member 3 | | |
| 0.0000347 | 0.000396 | 0.0002 | 71.01 | 26.27 | 2.7 | AC131212.3 | noncoding | | |
| | | | | | | | (Ensemble gene ID: | | |
| | | | | | | | ENSG00000280287.1) | | |
| <1e−07 | <1e−07 | <1e−07 | 37.26 | 13.74 | 2.71 | ADCY7 | adenylate cycla se 7 | 113 | NM_001114 |
| <1e−07 | 5.39E−06 | <1e−07 | 84.71 | 31.25 | 2.71 | PKD1 | polycystin 1, | 5310 | NM_000296 |
| | | | | | | | transient receptor | | |
| | | | | | | | potential channel | | |
| | | | | | | | interacting | | |
| 0.0000001 | 5.39E−06 | <1e−07 | 134.13 | 49.38 | 2.72 | STAT2 | signal transducer | 6773 | NM_005419 |
| | | | | | | | and activator of | | |
| | | | | | | | transcription 2 | | |
| 0.0000062 | 0.000113 | <1e−07 | 37.91 | 13.92 | 2.72 | CD40 | CD40 molecule | 958 | NM_001250 |
| <1e−07 | <1e−07 | <1e−07 | 67.84 | 24.86 | 2.73 | VAC14 | Vac14, PIKFYVE | 55697 | NM_001351157 |
| | | | | | | | complex component | | |
| <1e−07 | <1e−07 | <1e−07 | 40.19 | 14.73 | 2.73 | NCAPH2 | non-SMC condensin | 29781 | NM_001185011 |
| | | | | | | | II complex subunit | | |
| | | | | | | | H2 | | |
| 0.0000002 | 9.26E−06 | <1e−07 | 64.7 | 23.67 | 2.73 | EMILIN2 | elastin microfibril | 84034 | NM_032048 |
| | | | | | | | interfacer 2 | | |
| 0.0000004 | 0.000015 | <1e−07 | 21.88 | 8.01 | 2.73 | FAM30A | family with | 9834 | NM_014151 |
| | | | | | | | sequence similarity | | |
| | | | | | | | 30 member A | | |
| 0.000001 | 3.07E−05 | <1e−07 | 143.22 | 52.46 | 2.73 | LINC-PINT | longintergenic non- | 378805 | NM_001085379 |
| | | | | | | | protein coding | | |
| | | | | | | | RNA, p53 induced | | |
| | | | | | | | transcript | | |
| 0.0000107 | 0.000168 | <1e−07 | 95.66 | 35.11 | 2.73 | ITGA5 | integrin subunit | 3678 | NM_002205 |
| | | | | | | | alpha 5 | | |
| <1e−07 | <1e−07 | <1e−07 | 56.34 | 20.57 | 2.74 | TMC6 | transmembrane | 11322 | NM_001127198 |
| | | | | | | | channel like 6 | | |
| <1e−07 | <1e−07 | <1e−07 | 26.34 | 9.62 | 2.74 | H1FX-AS1 | H1FX antisense | 339942 | NM_001025468 |
| | | | | | | | RNA 1 | | |
| 0.0000003 | 1.23E−05 | <1e−07 | 310.63 | 113.39 | 2.74 | PLEKHG2 | pleckstrin homology | 64857 | NM_001351693 |
| | | | | | | | and RhoGEF | | |
| | | | | | | | domain containing | | |
| | | | | | | | G2 | | |
| <1e−07 | <1e−07 | <1e−07 | 70.58 | 25.68 | 2.75 | DPP7 | dipeptidyl peptidase | 29952 | NM_013379 |
| | | | | | | | 7 | | |
| 0.0000003 | 1.23E−05 | <1e−07 | 50.24 | 18.24 | 2.75 | SLC1A4 | solute carrier family | 6509 | NM_001135581 |
| | | | | | | | 1 member 4 | | |
| 0.0003781 | 0.00239 | 0.0009 | 55.49 | 20.2 | 2.75 | AXL | AXL receptor | 558 | NM_001278599 |
| | | | | | | | tyrosine kinase | | |
| 0.0000004 | 0.000015 | <1e−07 | 107.07 | 38.82 | 2.76 | ADAM19 | ADAM | 8728 | NM_023038 |
| | | | | | | | metallopeptidase | | |
| | | | | | | | domain 19 | | |
| 0.0000389 | 0.000431 | <1e−07 | 241.79 | 87.51 | 2.76 | ABCA1 | ATP binding | 19 | NM_005502 |
| | | | | | | | cassette subfamily A | | |
| | | | | | | | member 1 | | |
| 0.000421 | 0.00259 | 0.0008 | 88.68 | 32.13 | 2.76 | MEG3 | maternally | 55384 | NR_002766 |
| | | | | | | | expressed 3 (non- | | |
| | | | | | | | protein coding) | | |
| <1e−07 | 5.39E−06 | <1e−07 | 35.79 | 12.94 | 2.77 | FKBP11 | FK506 binding | 51303 | NM_001143781 |
| | | | | | | | protein 11 | | |
| <1e−07 | <1e−07 | <1e−07 | 111.77 | 40.15 | 2.78 | INPP5D | inositol | 3635 | NM_001017915 |
| | | | | | | | polyphosphate-5- | | |
| | | | | | | | phosphatase D | | |
| 0.0000002 | 9.26E−06 | <1e−07 | 166.82 | 59.88 | 2.79 | TNFRSF14 | TNF receptor | 8764 | NM_001297605 |
| | | | | | | | superfamily member | | |
| | | | | | | | 14 | | |
| 0.0000024 | 5.78E−05 | <1e−07 | 61.81 | 22.19 | 2.79 | TBC1D9 | TBC1 domain | 23158 | NM_015130 |
| | | | | | | | family member 9 | | |
| <1e−07 | <1e−07 | <1e−07 | 154.99 | 55.41 | 2.8 | RIPOR1 | RHO family | 79567 | NM_001193522 |
| | | | | | | | interacting cell | | |
| | | | | | | | polarization | | |
| | | | | | | | regulator 1 | | |
| 0.0003501 | 0.00226 | 0.0004 | 44.38 | 15.85 | 2.8 | CD79A | CD79a molecule | 973 | NM_001783 |
| <1e−07 | <1e−07 | <1e−07 | 105.09 | 37.36 | 2.81 | RFTN1 | raftlin, lipid raft | 23180 | NM_015150 |
| | | | | | | | linker 1 | | |
| 0.0000195 | 0.000261 | <1e−07 | 50.45 | 17.95 | 2.81 | DUSP4 | dual specificity | 1846 | NM_001394 |
| | | | | | | | phosphatase 4 | | |
| 0.0003538 | 0.00228 | 0.0004 | 46.07 | 16.42 | 2.81 | CD163 | CD163 molecule | 9332 | NM_004244 |
| <1e−07 | <1e−07 | <1e−07 | 32.35 | 11.47 | 2.82 | MAP4K1 | mitogen-activated | 11184 | NM_001042600 |
| | | | | | | | protein kinase | | |
| | | | | | | | kinase kinase kinase | | |
| | | | | | | | 1 | | |
| <1e−07 | <1e−07 | <1e−07 | 457.87 | 162.39 | 2.82 | RAPGEF1 | Rap guanine | 2889 | NM_001304275 |
| | | | | | | | nucleotide exchange | | |
| | | | | | | | factor 1 | | |
| 0.0000003 | 1.23E−05 | <1e−07 | 52.96 | 18.76 | 2.82 | SNX8 | sorting nexin 8 | 29886 | NM_013321 |
| 0.0000024 | 5.78E−05 | <1e−07 | 24.61 | 8.74 | 2.82 | BLK | BLK proto- | 640 | NM_001330465 |
| | | | | | | | oncogene, Src | | |
| | | | | | | | family tyrosine | | |
| | | | | | | | kinase | | |
| 0.0000194 | 0.00026 | <1e−07 | 64.95 | 23.04 | 2.82 | POU2AF1 | POU class 2 | 5450 | NM_006235 |
| | | | | | | | associating factor 1 | | |
| <1e−07 | <1e−07 | <1e−07 | 225.48 | 79.68 | 2.83 | FMNL1 | form in like 1 | 752 | NM_005892 |
| <1e−07 | <1e−07 | <1e−07 | 318.76 | 112.6 | 2.83 | HYOU1 | hypoxia up- | 10525 | NM_001130991 |
| | | | | | | | regulated 1 | | |
| <1e−07 | <1e−07 | <1e−07 | 15.57 | 5.51 | 2.83 | AC092068.3 | noncoding | | |
| | | | | | | | (Ensemble gene ID: | | |
| | | | | | | | ENSG00000267749.1) | | |
| 0.0000002 | 9.26E−06 | <1e−07 | 93.97 | 33.2 | 2.83 | LPXN | leupaxin | 9404 | NM_001143995 |
| 0.0000072 | 0.000126 | <1e−07 | 49.01 | 17.35 | 2.83 | SNHG22 | small nucleolar | 103091864 | NR_117096 |
| | | | | | | | RNA host gene 22 | | |
| 0.0000107 | 0.000168 | <1e−07 | 3340.15 | 1176.19 | 2.84 | NEAT1 | nuclear paraspeckle | 283 131 | NR_002802 |
| | | | | | | | assembly transcript | | |
| | | | | | | | 1 (non-protein | | |
| | | | | | | | coding) | | |
| 0.0001429 | 0.00115 | 0.0001 | 1152.66 | 405.22 | 2.84 | AC020916.1 | noncoding | | |
| | | | | | | | (Ensemble gene ID: | | |
| | | | | | | | ENSG00000267519.6) | | |
| <1e−07 | <1e−07 | <1e−07 | 65.15 | 22.85 | 2.85 | EML3 | echinoderm | 256364 | NM_001300793 |
| | | | | | | | microtubule | | |
| | | | | | | | associated protein | | |
| | | | | | | | like 3 | | |
| 0.000001 | 3.07E−05 | <1e−07 | 27.49 | 9.61 | 2.86 | IGLL5 | immunoglobulin | 100423062 | NM_001178126 |
| | | | | | | | lambda like | | |
| | | | | | | | polypeptide 5 | | |
| 0.0000567 | 0.000569 | <1e−07 | 134.99 | 47.21 | 2.86 | AEBP1 | AE binding protein | 165 | NM_001129 |
| | | | | | | | 1 | | |
| <1e−07 | <1e−07 | <1e−07 | 42.3 | 14.71 | 2.88 | POU2F2 | POU class 2 | 5452 | NM_001207025 |
| | | | | | | | homeobox 2 | | |
| <1e−07 | <1e−07 | <1e−07 | 46.92 | 16.1 | 2.91 | FADS3 | fatty acid desaturase | 3995 | NM_021727 |
| | | | | | | | 3 | | |
| <1e−07 | <1e−07 | <1e−07 | 41.25 | 14.2 | 2.91 | ARMC5 | armadillo repeat | 79798 | NM_001105247 |
| | | | | | | | containing 5 | | |
| <1e−07 | 5.39E−06 | <1e−07 | 73.93 | 25.42 | 2.91 | ARFGAP1 | ADP ribosylation | 55738 | NM_001281482 |
| | | | | | | | factor GTPase | | |
| | | | | | | | activating protein 1 | | |
| 0.0000159 | 0.000225 | <1e−07 | 172.38 | 58.73 | 2.93 | HLA-DRB1 | major | 3123 | NM_001243965 |
| | | | | | | | histocompatibility | | |
| | | | | | | | complex, class II, | | |
| | | | | | | | DR beta 1 | | |
| <1e−07 | <1e−07 | <1e−07 | 48.4 | 16.48 | 2.94 | RBM38 | RNA binding motif | 55544 | NM_001291780 |
| | | | | | | | protein 38 | | |
| 0.0000146 | 0.000211 | <1e−07 | 108.18 | 36.83 | 2.94 | SEMA4A | semaphorin 4A | 64218 | NM_001193300 |
| <1e−07 | <1e−07 | <1e−07 | 41.91 | 14.23 | 2.95 | TIAM1 | T cell lymphoma | 7074 | NM_001353684 |
| | | | | | | | invasion and | | |
| | | | | | | | metastasis 1 | | |
| <1e−07 | <1e−07 | <1e−07 | 89.98 | 30.41 | 2.96 | BCL2 | BCL2, apoptosis | 596 | NM_000633 |
| | | | | | | | regulator | | |
| 0.0000005 | 1.78E−05 | <1e−07 | 127.28 | 43.04 | 2.96 | OGT | O-linked N- | 8473 | NM_003605 |
| | | | | | | | acetylglucosamine | | |
| | | | | | | | (GlcNAc) | | |
| | | | | | | | transferase | | |
| <1e−07 | <1e−07 | <1e−07 | 21.89 | 7.36 | 2.97 | N4BP3 | NEDD4 binding | 23138 | NM_015111 |
| | | | | | | | protein 3 | | |
| 0.0000032 | 7.14E−05 | <1e−07 | 90.44 | 30.45 | 2.97 | LAMA5 | laminin subunit | 3911 | NM_005560 |
| | | | | | | | alpha 5 | | |
| 0.0001875 | 0.00141 | 0.0001 | 97.19 | 32.77 | 2.97 | SPOCK2 | SPARC | 9806 | NM_001134434 |
| | | | | | | | (osteonectin), cwcv | | |
| | | | | | | | and kazal like | | |
| | | | | | | | domains | | |
| | | | | | | | proteoglycan 2 | | |
| 0.000091 | 0.00081 | 0.0001 | 66.54 | 22.25 | 2.99 | CHAC1 | ChaC glutathione | 79094 | NM_001142776 |
| | | | | | | | specific gamma- | | |
| | | | | | | | glutamylcyclo- | | |
| | | | | | | | transferase 1 | | |
| 0.0000003 | 1.23E−05 | <1e−07 | 44.39 | 14.81 | 3 | RASGRP3 | RAS guanyl | 25780 | NM_001139488 |
| | | | | | | | releasing protein 3 | | |
| <1e−07 | <1e−07 | <1e−07 | 80.17 | 26.55 | 3.02 | CBX6 | chromobox 6 | 23466 | NM_001303494 |
| <1e−07 | <1e−07 | <1e−07 | 41.47 | 13.72 | 3.02 | PARVG | parvin gamma | 64098 | NM_001137605 |
| <1e−07 | <1e−07 | <1e−07 | 213.56 | 70.39 | 3.03 | AC004151.1 | noncoding | | |
| | | | | | | | (Ensemble gene ID: | | |
| | | | | | | | ENSG00000180448) | | |
| <1e−07 | <1e−07 | <1e−07 | 115.79 | 38.17 | 3.03 | RUBCN | RUN and cysteine | 9711 | NM_001145642 |
| | | | | | | | rich domain | | |
| | | | | | | | containing beclin 1 | | |
| | | | | | | | interacting protein | | |
| 0.0000004 | 0.000015 | <1e−07 | 33.87 | 11.15 | 3.04 | TNFRSF18 | TNF receptor | 8784 | NM_004195 |
| | | | | | | | superfamily member | | |
| | | | | | | | 18 | | |
| <1e−07 | <1e−07 | <1e−07 | 117.5 | 38.48 | 3.05 | SYNE3 | spectrin repeat | 161176 | NM_024633 |
| | | | | | | | containing nuclear | | |
| | | | | | | | envelope family | | |
| | | | | | | | member 3 | | |
| <1e−07 | <1e−07 | <1e−07 | 2535.56 | 831.48 | 3.05 | AHNAK | AHNAK | 79026 | NM_001346445 |
| | | | | | | | nucleoprotein | | |
| 0.0000065 | 0.000117 | <1e−07 | 47.62 | 15.64 | 3.05 | TNRC18P1 | trinucleotide repeat | 644962 | NR_077215 |
| | | | | | | | containing 18 | | |
| | | | | | | | pseudogene 1 | | |
| <1e−07 | <1e−07 | <1e−07 | 23.2 | 7.58 | 3.06 | C13orf46 | chromosome 13 | 100507747 | XM_006719994 |
| | | | | | | | open reading frame | | |
| | | | | | | | 46 | | |
| 0.0000004 | 0.000015 | <1e−07 | 139.26 | 45.3 | 3.07 | MICAL1 | microtubule | 64780 | NM_001159291 |
| | | | | | | | associated | | |
| | | | | | | | monooxygenase, | | |
| | | | | | | | calponin and LIM | | |
| | | | | | | | domain containing 1 | | |
| <1e−07 | <1e−07 | <1e−07 | 55.82 | 18.14 | 3.08 | SH3TC1 | SH3 domain and | 54436 | NM_001318480 |
| | | | | | | | tetratricopeptide | | |
| | | | | | | | repeats 1 | | |
| 0.0000002 | 9.26E−06 | <1e−07 | 108 | 34.99 | 3.09 | PLXNA1 | plexin A1 | 5361 | NM_032242 |
| | | | | | | | immunoglobulin | | |
| | | | | | | | lambda variable 3- | | |
| | | | | | | | 21 | | |
| 0.0000005 | 1.78E−05 | <1e−07 | 31.25 | 9.95 | 3.14 | TNFRSF13B | TNF receptor | 23495 | NM_012452 |
| | | | | | | | superfamily member | | |
| | | | | | | | 13B | | |
| 0.0000274 | 0.000334 | <1e−07 | 43.55 | 13.81 | 3.15 | CD22 | CD22 molecule | 933 | NM_001185099 |
| 0.0000004 | 0.000015 | <1e−07 | 24.21 | 7.63 | 3.17 | CD72 | CD72 molecule | 971 | NM_001782 |
| 0.000003 | 6.83E−05 | <1e−07 | 35.01 | 11.03 | 3.17 | MIR155HG | MIR155 host gene | 114614 | NR_001458 |
| 0.0000002 | 9.26E−06 | <1e−07 | 72.57 | 22.74 | 3.19 | ACAP1 | ArfGAP with | 9744 | NM_014716 |
| | | | | | | | coiled-coil, ankyrin | | |
| | | | | | | | repeat and PH | | |
| | | | | | | | domains 1 | | |
| 0.0000128 | 0.000192 | <1e−07 | 35.11 | 11 | 3.19 | IFITM10 | interferon induced | 402778 | NM_001170820 |
| | | | | | | | transmembrane | | |
| | | | | | | | protein 10 | | |
| <1e−07 | <1e−07 | <1e−07 | 48.24 | 15.04 | 3.21 | NLRP1 | NLR family pyrin | 22861 | NM_001033053 |
| | | | | | | | domain containing 1 | | |
| 0.0000065 | 0.000117 | <1e−07 | 80.15 | 24.98 | 3.21 | WHRN | whirlin | 25861 | NM_001083885 |
| <1e−07 | <1e−07 | <1e−07 | 40.05 | 12.43 | 3.22 | TTYH2 | tweety family | 94015 | NM_001330453 |
| | | | | | | | member 2 | | |
| 0.0000001 | 5.39E−06 | <1e−07 | 65.96 | 20.31 | 3.25 | PTPRS | protein tyrosine | 5802 | NM_002850 |
| | | | | | | | phosphatase, | | |
| | | | | | | | receptor type S | | |
| 0.0000003 | 1.23E−05 | <1e−07 | 44.68 | 13.71 | 3.26 | LY9 | lymphocyte antigen | 4063 | NM_001033667 |
| | | | | | | | 9 | | |
| <1e−07 | <1e−07 | <1e−07 | 109.03 | 33.16 | 3.29 | CLCN7 | chloride voltage- | 1186 | NM_001114331 |
| | | | | | | | gated channel 7 | | |
| <1e−07 | <1e−07 | <1e−07 | 82.43 | 24.9 | 3.31 | AP5Z1 | adaptor related | 9907 | NM_014855 |
| | | | | | | | protein complex 5 | | |
| | | | | | | | zeta 1 subunit | | |
| 0.0000108 | 0.000169 | <1e−07 | 58.69 | 17.64 | 3.33 | CCR7 | C-C motif | 1236 | NM_001301714 |
| | | | | | | | chemokine receptor | | |
| | | | | | | | 7 | | |
| <1e−07 | <1e−07 | <1e−07 | 82.81 | 24.73 | 3.35 | GGA2 | golgi associated, | 23062 | NM_015044 |
| | | | | | | | gamma adaptin ear | | |
| | | | | | | | containing, ARF | | |
| | | | | | | | binding protein 2 | | |
| 0.0000725 | 0.000682 | 0.0001 | 42.92 | 12.82 | 3.35 | SNORA20 | small nucleolar | 677806 | NR_002960 |
| | | | | | | | RNA, H/ACA box | | |
| | | | | | | | 20 | | |
| 0.0000288 | 0.000346 | 0.0001 | 72.53 | 21.39 | 3.39 | TRPV3 | transient receptor | 162514 | NM_001258205 |
| | | | | | | | potential cation | | |
| | | | | | | | channel subfamily V | | |
| | | | | | | | member 3 | | |
| <1e−07 | <1e−07 | <1e−07 | 107.45 | 31.59 | 3.4 | ARL4C | ADP ribosy lation | 10123 | NM_001282431 |
| | | | | | | | factor like GTPase | | |
| | | | | | | | 4C | | |
| <1e−07 | <1e−07 | <1e−07 | 186.65 | 54.42 | 3.43 | FAM214A | family with | 56204 | NM_001286495 |
| | | | | | | | sequence similarity | | |
| | | | | | | | 214 member A | | |
| 0.0000252 | 0.000314 | <1e−07 | 133.58 | 38.97 | 3.43 | SEMA6B | semaphorin 6B | 10501 | NM_020241 |
| 0.0000857 | 0.000774 | 0.0001 | 44.14 | 12.83 | 3.44 | CD163L1 | CD163 molecule | 283316 | NM_001297650 |
| | | | | | | | like 1 | | |
| <1e−07 | <1e−07 | <1e−07 | 71.28 | 20.3 | 3.51 | UAPIL1 | UDP-N- | 91373 | NM_207309 |
| | | | | | | | acetylglucosamine | | |
| | | | | | | | pyrophosphorylase 1 | | |
| | | | | | | | like 1 | | |
| <1e−07 | <1e−07 | <1e−07 | 37.12 | 10.37 | 3.58 | RASGRP2 | RAS guanyl | 10235 | NM_001098670 |
| | | | | | | | releasing protein 2 | | |
| 0.0000001 | 5.39E−06 | <1e−07 | 44.05 | 12.22 | 3.6 | BACH2 | BTB domain and | 60468 | NM_001170794 |
| | | | | | | | CNC homolog 2 | | |
| 0.0000235 | 0.000298 | 0.0001 | 56.98 | 15.81 | 3.6 | SNORD3B-2 | small nucleolar | 780852 | NR_003924 |
| | | | | | | | RNA, C/D box 3B-2 | | |
| 0.0000331 | 0.000384 | <1e−07 | 43.02 | 11.88 | 3.62 | IGHG4 | immunoglobulin | 3503 | |
| | | | | | | | heavy constant | | |
| | | | | | | | gamma 4 (G4m | | |
| | | | | | | | marker) | | |
| <1e−07 | <1e−07 | <1e−07 | 104.8 | 28.66 | 3.66 | LSS | lanosterol synthase | 4047 | NM_001001438 |
| 0.0000106 | 0.000167 | <1e−07 | 39.83 | 10.75 | 3.71 | SNORD3B-1 | small nucleolar | 26851 | NR_003271 |
| | | | | | | | RNA, C/D box 3B-1 | | |
| <1e−07 | <1e−07 | <1e−07 | 45.71 | 12.09 | 3.78 | SLC26A11 | solute carrier family | 284129 | NM_001166347 |
| | | | | | | | 26 member 11 | | |
| <1e−07 | <1e−07 | <1e−07 | 26.87 | 7.04 | 3.82 | TNFRSF13C | TNF receptor | 115650 | NM_052945 |
| | | | | | | | superfamily member | | |
| | | | | | | | 13C | | |
| <1e−07 | <1e−07 | <1e−07 | 73.43 | 19.14 | 3.84 | AC004687.1 | Homo sapiens | | |
| | | | | | | | transcribed RNA | | |
| | | | | | | | MIR142 gene for | | |
| | | | | | | | microRNA 142, | | |
| | | | | | | | complete sequence | | |
| | | | | | | | (EnsEMBL Gene | | |
| | | | | | | | ID | | |
| | | | | | | | (ENST00000579003.1) | | |
| <1e−07 | <1e−07 | <1e−07 | 35.37 | 9.17 | 3.86 | LILRB1 | leukocyte | 10859 | NM_001081637 |
| | | | | | | | immunoglobulin | | |
| | | | | | | | like receptor B1 | | |
| <1e−07 | <1e−07 | <1e−07 | 61.29 | 15.71 | 3.9 | SLC17A9 | solute carrier family | 63910 | NM_001302643 |
| | | | | | | | 17 member 9 | | |
| <1e−07 | <1e−07 | <1e−07 | 66.87 | 16.87 | 3.96 | WDFY4 | WDFY family | 57705 | NM_020945 |
| | | | | | | | member 4 | | |
| <1e−07 | 5.39 | <1e−07 | 86.58 | 21.54 | 4.02 | NPIPB5 | nuclear pore | 100132 | NM_0011 |
| | | | | | | | complex interacting | | |
| | | | | | | | protein family | | |
| | | | | | | | member B5 | | |
| 0.0000035 | 7.64E−05 | <1e−07 | 1863.62 | 461.63 | 4.04 | SNORD17 | small nucleolar | 692086 | NR_003045 |
| | | | | | | | RNA, C/D box 17 | | |
| <1e−07 | <1e−07 | <1e−07 | 46.63 | 11.04 | 4.22 | GNG7 | G protein subunit | 2788 | NM_0528 |
| | | | | | | | gamma 7 | | |
| 0.0000003 | 1.23E−05 | <1e−07 | 114.12 | 25.59 | 4.46 | SNORD89 | small nucleolar | 692205 | NR_003070 |
| | | | | | | | RNA, C/D box 89 | | |
| 0.0000001 | 5.39E−06 | <1e−07 | 174.93 | 32.55 | 5.37 | STAB1 | | 23166 | NM_015136 |
| | | | | | | | stabilin 1 | | |

TABLE 2B
Exemplary Biomarkers of a Transcriptomic Signature

| Unique ID | Name | HGNC ID | Location |
|---|---|---|---|
| ABCA13 | ATP binding cassette subfamily A member 13 | HGNC: 14638 | 7p12.3 |
| AC103691.1 | long non-coding RNAs | | |
| ADGRV1 | adhesion G protein-coupled receptor V1 | HGNC: 17416 | 5q14.3 |
| AGR2 | anterior gradient 2, protein disulphide isomerase family member | HGNC: 328 | 7p21.1 |
| AL356534.1 | long non-coding RNAs | | |
| ALDOB | aldolase, fructose-bisphosphate B | HGNC: 417 | 9q31.1 |
| ANXA4 | annexin A4 | HGNC: 542 | 2p13.3 |
| ARFGEF3 | ARFGEF family member 3 | HGNC: 21213 | 6q23.3-q24.1 |
| ATP10B | ATPase phospholipid transporting 10B (putative) | HGNC: 13543 | 5q34 |
| ATP8B1 | ATPase phospholipid transporting 8B1 | HGNC: 3706 | 18q21.31 |
| B3GALT5 | beta-1,3-galactosyltransferase 5 | HGNC: 920 | 21q22.2 |
| BAIAP2L2 | BAR/IMD domain containing adaptor protein 2 like 2 | HGNC: 26203 | 22q13.1 |
| C1orf115 | chromosome 1 open reading frame 115 | HGNC: 25873 | 1q41 |
| CACNA1A | calcium voltage-gated channel subunit alpha 1 A | HGNC: 1388 | 19p13.13 |
| CACNA1D | calcium volta ge-gated channel subunit alpha 1 D | HGNC: 1391 | 3p21.1 |
| CAMSAP3 | calmodulin regulated spectrin associated protein family member 3 | HGNC: 29307 | 19p13.2 |
| CARD10 | caspase recruitment domain family member 10 | HGNC: 16422 | 22q13.1 |
| CCDC26 | CCDC26 long non-coding RNA | HGNC: 28416 | 8q24.21 |
| CCND1 | cyclin D1 | HGNC: 1582 | 11q13.3 |
| CDH1 | cadherin 1 | HGNC: 1748 | 16q22.1 |
| CDH17 | cadherin 17 | HGNC: 1756 | 8q22.1 |
| CDHR2 | cadherin related family member 2 | HGNC: 18231 | 5q35.2 |
| CDHR5 | cadherin related family member 5 | HGNC: 7521 | 11p15.5 |
| CFTR | CF transmembrane conductance regulator | HGNC: 1884 | 7q31.2 |
| CGN | cingulin | HGNC: 17429 | 1q21.3 |
| CHDH | choline dehydrogenase | HGNC: 24288 | 3p21.1 |
| CLCA1 | chloride channel accessory 1 | HGNC: 2015 | 1p22.3 |
| CLDN3 | claudin 3 | HGNC: 2045 | 7q11.23 |
| CLDN7 | claudin 7 | HGNC: 2049 | 17p13.1 |
| CPS1 | carbamoyl-phosphate synthase 1 | HGNC: 2323 | 2q34 |
| CRACD | capping protein inhibiting regulator of actin dynamics | HGNC: 29219 | 4q12 |
| CREB3L1 | cAMP responsive element binding protein 3 like 1 | HGNC: 18856 | 11p11.2 |
| CYP3A5 | cytochrome P450 family 3 subfamily A member 5 | HGNC: 2638 | 7q22.1 |
| DDC | dopa decarboxylase | HGNC: 2719 | 7p12.2-p12.1 |
| DEFA5 | defensin alpha 5 | HGNC: 2764 | 8p23.1 |
| DHRS11 | dehydrogenase/reductase 11 | HGNC: 28639 | 17q12 |
| DMBT1 | deleted in malignant brain tumors 1 | HGNC: 2926 | 10q26.13 |
| DNAH7 | dynein axonemal heavy chain 7 | HGNC: 18661 | 2q32.3 |
| ENPEP | glutamyl aminopeptidase | HGNC: 3355 | 4q25 |
| EPCAM | epithelial cell adhesion molecule | HGNC: 11529 | 2p21 |
| EPHX2 | epoxide hydrolase 2 | HGNC: 3402 | 8p21.2-p21.1 |
| ERBB3 | erb-b2 receptor tyrosine kinase 3 | HGNC: 3431 | 12q13.2 |
| FAM83E | family with sequence similarity 83 member E | HGNC: 25972 | 19q13.33 |
| FOXP2 | forkhead box P2 | HGNC: 13875 | 7q31.1 |
| FOXQ1 | forkhead box Q1 | HGNC: 20951 | 6p25.3 |
| FREM1 | FRAS1 related extracellular matrix 1 | HGNC: 23399 | 9p22.3 |
| GATA6 | GATA binding protein 6 | HGNC: 4174 | 18q11.2 |
| H1-0 | H1.0 linker histone | HGNC: 4714 | 22q13.1 |
| HELLPAR | HELLP associated long non-coding RNA | HGNC: 43984 | 12q23.2 |
| HMGN5 | high mobility group nucleosome binding domain 5 | HGNC: 8013 | Xq21.1 |
| HNF4A | hepatocyte nuclear factor 4 alpha | HGNC: 5024 | 20q13.12 |
| INAVA | innate immunity activator | HGNC: 25599 | 1q32.1 |
| ITGB4 | integrin subunit beta 4 | HGNC: 6158 | 17q25.1 |
| KIAA1324 | endosome-lysosome associated apoptosis and a utophagy regulator 1 | HGNC: 29618 | 1p13.3 |
| KLF5 | Kruppel like factor 5 | HGNC: 6349 | 13q22.1 |
| KRT20 | keratin 20 | HGNC: 20412 | 17q21.2 |
| KRT8 | keratin 8 | HGNC: 6446 | 12q13.13 |
| LGALS4 | galectin 4 | HGNC: 6565 | 19q13.2 |
| LIPG | lipase G, endothelial type | HGNC: 6623 | 18q21.1 |
| LPIN3 | lipin 3 | HGNC: 14451 | 20q12 |
| LRATD1 | LRAT domain containing 1 | HGNC: 20743 | 2p24.3 |
| MAGI1 | membrane associated guanylate kinase, WW and PDZ domain containing 1 | HGNC: 946 | 3p14.1 |
| MALRD1 | MAM and LDL receptor class A domain containing 1 | HGNC: 24331 | 10p12.31 |
| MAOA | monoamine oxidase A | HGNC: 6833 | Xp11.3 |
| MAP7 | microtubule associated protein 7 | HGNC: 6869 | 6q23.3 |
| MDK | midkine | HGNC: 6972 | 11p11.2 |
| MECOM | MDS1 and EVI1 complex locus | HGNC: 3498 | 3q26.2 |
| MGAM2 | malta se-glucoamylase 2 (putative) | HGNC: 28101 | 7q34 |
| MLXIPL | MLX interacting protein like | HGNC: 12744 | 7q11.23 |
| MUC17 | mucin 17, cell surface associated | HGNC: 16800 | 7q22.1 |
| MUC19 | mucin 19, oligomeric | HGNC: 14362 | 12q12 |
| MUC2 | mucin 2, oligomeric mucus/gel-forming | HGNC: 7512 | 11p15.5 |
| MUC3A | mucin 3A, cell surface associated | HGNC: 7513 | 7q22.1 |
| MYH14 | myosin heavy chain 14 | HGNC: 23212 | 19q13.33 |
| MYO1A | myosin IA | HGNC: 7595 | 12q13.3 |
| MYO5B | myosin VB | HGNC: 7603 | 18q |
| MYO5C | myosin VC | HGNC: 7604 | 15q21.2 |
| MYRF | myelin regulatory factor | HGNC: 1181 | 11q12.2 |
| NFIB | nuclear factor I B | HGNC: 7785 | 9p23-p22.3 |
| NGEF | neuronal guanine nucleotide exchange factor | HGNC: 7807 | 2q37.1 |
| NHSL1 | NHS like 1 | HGNC: 21021 | 6q24.1 |
| NR1I2 | nuclear receptor subfamily 1 group I member 2 | HGNC: 7968 | 3q13.33 |
| OLFM4 | olfactomedin 4 | HGNC: 17190 | 13q14.3 |
| PARD3B | par-3 family cell polarity regulator beta | HGNC: 14446 | 2q33.3 |
| PARP3 | poly (ADP-ribose) polymerase family member 3 | HGNC: 273 | 3p21.2 |
| PBLD | phenazine biosynthesis like protein domain containing | HGNC: 23301 | 10q21.3 |
| PCLO | piccolo presynaptic cytomatrix protein | HGNC: 13406 | 7q21.11 |
| PCSK5 | proprotein convertase subtilisin/kexin type 5 | HGNC: 8747 | 9q21.13 |
| PIGR | polymeric immunoglobulin receptor | HGNC: 8968 | 1q32.1 |
| PKP2 | plakophilin 2 | HGNC: 9024 | 12p11.21 |
| PLA2R1 | phospholipase A2 receptor 1 | HGNC: 9042 | 2q23-q24 |
| PLCB4 | phospholipase C beta 4 | HGNC: 9059 | 20p12.3-p12.2 |
| PLCE1 | phospholipase C epsilon 1 | HGNC: 17175 | 10q23.33 |
| PLEKHG6 | pleckstrin homology and RhoGEF domain containing G6 | HGNC: 25562 | 12p13.31 |
| PLEKHH1 | pleckstrin homology, My TH4 and FERM domain containing H1 | HGNC: 17733 | 14q24.1 |
| PMFBP1 | polyamine modulated factor 1 binding protein 1 | HGNC: 17728 | 16q22.2 |
| PPARGC1B | PPARG coactivator 1 beta | HGNC: 30022 | 5q32 |
| PRDM16 | PR/SET domain 16 | HGNC: 14000 | 1p36.32 |
| PRELID2 | PRELI domain containing 2 | HGNC: 28306 | 5q32 |
| PROM1 | prom inin 1 | HGNC: 9454 | 4p15.32 |
| PRSS8 | serine protease 8 | HGNC: 9491 | 16p11.2 |
| PTPRH | protein tyrosine phosphatase receptor type H | HGNC: 9672 | 19q13.42 |
| RAP1GAP | RAP1 GTPase activating protein | HGNC: 9858 | 1p36.12 |
| REG4 | regenerating family member 4 | HGNC: 22977 | 1p12 |
| SELENBP1 | selenium binding protein 1 | HGNC: 10719 | 1q21.3 |
| SEMA4G | semaphorin 4G | HGNC: 10735 | 10q24.31 |
| SEMA6A | semaphorin 6A | HGNC: 10738 | 5q23.1 |
| SH3D19 | SH3 domain containing 19 | HGNC: 30418 | 4q31.3 |
| SH3RF2 | SH3 domain containing ring finger 2 | HGNC: 26299 | 5q32 |
| SHANK2 | SH3 and multiple ankyrin repeat domains 2 | HGNC: 14295 | 11q13.3-q13.4 |
| SHROOM3 | shroom family member 3 | HGNC: 30422 | 4q21.1 |
| SI | sucra se -isom a lta se | HGNC: 10856 | 3q26.1 |
| SLC4A4 | solute carrier family 4 member 4 | HGNC: 11030 | 4q13.3 |
| SMIM24 | small integral membrane protein 24 | HGNC: 37244 | 19p13.3 |
| SOX6 | SRY-box transcription factor 6 | HGNC: 16421 | 11p15.2 |
| SPIRE2 | spire type actin nucleation factor 2 | HGNC: 30623 | 16q24.3 |
| ST6GALNAC1 | ST6 N-acetylgalactosaminide alpha-2,6-sialyltransferase 1 | HGNC: 23614 | 17q25.1 |
| TCEA3 | transcription elongation factor A3 | HGNC: 11615 | 1p36.12 |
| TJP3 | tight junction protein 3 | HGNC: 11829 | 19p13.3 |
| TMC4 | transmembrane channel like 4 | HGNC: 22998 | 19q13.42 |
| TMC5 | transmembrane channel like 5 | HGNC: 22999 | 16p12.3 |
| TMEM184A | transmembrane protein 184A | HGNC: 28797 | 7p22.3 |
| TMPRSS2 | transmembrane serine protease 2 | HGNC: 11876 | 21q22.3 |
| TRIM2 | tripartite motif containing 2 | HGNC: 15974 | 4q31.3 |
| UACA | uvealautoantigen with coiled-coil domains and ankyrin repeats | HGNC: 15947 | 15q23 |
| VIL1 | villin 1 | HGNC: 12690 | 2q35 |
| WWC1 | WW and C2 domain containing 1 | HGNC: 29435 | 5q34 |
| XDH | xanthine dehydrogenase | HGNC: 12805 | 2p23.1 |

In embodiments where more than one biomarker is detected, the differences in expression between a patient having an inflammatory MNP signature or a resident mucosal MNP signature and a reference subject (e.g., non-IBD subject) may be different for each marker, e.g., each of the biomarkers detected is at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold up-modulated as compared to the expression level of the respective biomarker in the reference sample. In some cases, at least about 80%, 85%, 90%, 95%, 96%, 97%, 98%, or 99% of the biomarkers detected in a transcriptomic signature is at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold up-modulated as compared to the expression level of the respective biomarker in the reference sample. In some embodiments, the reference sample is obtained from a subject that does not have the disease or the condition disclosed herein. In some embodiments, the reference sample is obtained from a subject that has the disease or the condition, but does not have the subtype of the disease of the condition described herein.

### Expression and RNA Sequencing Methods

In some embodiments, the gene expression levels in from the MNP in the biological samples may be measured by an array. In some cases, the array comprises a microarray, sequencing, and qPCR. In some instances, the array comprises RNA sequencing (RNA-Seq). In some examples, RNA-Seq data may be analyzed using the BRB array tools. BRB array tools may comprise tools for visualization and statistical analysis of microarray gene expression, copy number, methylation and/or RNA-Seq data. In some examples, the analysis tools may be run using an R-program. In some embodiments, the analysis tools described herein may be used for analyzing genes through differential gene expressions, hierarchical clustering and/or principal component analysis (PCA) plots.

Any suitable method can be utilized to assess (directly or indirectly) the level of expression of a biomarker in a sample. Non-limiting examples of such methods include analyzing the sample using nucleic acid hybridization methods, nucleic acid reverse transcription methods, nucleic acid amplification methods, array analysis, and combinations thereof. In some embodiments, the level of expression of a biomarker in a sample is determined by detecting a transcribed polynucleotide, or portion thereof, e.g., mRNA, or cDNA, of the biomarker gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, quantitative PCR analysis, RNase protection assays, Northern blotting and in situ hybridization. Other suitable systems for RNA sample analysis include microarray analysis (e.g., using Affymetrix's microarray system or Illumina's BeadArray Technology).

Isolated RNA can be used in hybridization or amplification assays that include, but are not limited to, Southern or Northern analyses, polymerase chain reaction (PCR) analyses and probe arrays. An exemplary method for the determination of RNA levels involves contacting RNA with a nucleic acid molecule (e.g., probe) that can hybridize to the biomarker mRNA. The nucleic acid molecule can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the biomarker genomic DNA. In some embodiments, the RNA is immobilized on a solid surface and contacted with a probe, for example by running the isolated RNA on an agarose gel and transferring the RNA from the gel to a membrane, such as nitrocellulose. In some embodiments, the probe(s) are immobilized on a solid surface, for example, in an Affymetrix gene chip array, and the probe(s) are contacted with RNA.

The level of expression of the biomarker in a sample can also be determined using methods that involve the use of nucleic acid amplification and/or reverse transcriptase, e.g., by RT-PCR, ligase chain reaction, self-sustained sequence replication, transcriptional amplification system, Q-Beta Replicase, rolling circle replication or any other nucleic acid amplification method, followed by the detection of the amplified molecules. These approaches may be useful for the detection of nucleic acid molecules if such molecules are present in very low numbers. In some embodiments, the level of expression of the biomarker is determined by quantitative fluorogenic RT-PCR (e.g., the TaqMan™ System). Such methods may utilize pairs of oligonucleotide primers that are specific for the biomarker.

In some embodiments, biomarker expression is determined by sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modem sequencing technologies such as Illumina sequencing (e.g., Solexa), Roche 454 sequencing, Ion torrent sequencing, SOLiD sequencing, PacBio sequencing (e.g., SMRT), oxford nanopore technologies sequencing, and sequencing by transient binding (Life Tech). In some cases, next-generation sequencing involves high-throughput sequencing methods. Additional sequencing methods available to one of skill in the art may also be employed.

The expression levels of biomarker RNA can be monitored using a membrane blot (such as used in hybridization analysis such as Northern, Southern, dot, and the like), microwells, sample tubes, gels, beads, fibers, or any solid support comprising bound nucleic acids. The determination of biomarker expression level may also comprise using nucleic acid probes in solution.

In some embodiments, microarrays are used to detect the level of expression of a biomarker. DNA microarrays provide one method for the simultaneous measurement of the expression levels of large numbers of genes. Each array consists of a reproducible pattern of capture probes attached to a solid support. Labeled nucleic acid is hybridized to complementary probes on the array and then detected, e.g., by laser scanning. Hybridization intensities for each probe on the array are determined and converted to a quantitative value representing relative gene expression levels. High-density oligonucleotide arrays may be useful for determining the gene expression profile for a large number of RNA's in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like.

Other methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some instances, a method of detecting an expression profile in a subject comprises contacting nucleic acids from a sample of the subject with a nucleic acid polymer that hybridizes to a region of a biomarker nucleic acid sequence. Hybridization may occur at standard hybridization temperatures, e.g., between about 35° C. and about 65° C. in a standard PCR buffer. In some cases, the biomarker nucleic acid sequence is a sequence comprising at least about 30, 40, 50, 60, 70, 80, 90, or 100 nucleobases of a biomarker listed in Tables 1, or 2A-2B. The nucleic acid polymer can comprise an oligonucleotide of at least or about 5, 10, 15, 20, 25, 30, 35, 40, 45, 50, 75, 100 or more nucleobases in length and sufficient to specifically hybridize to a biomarker of Tables 1 or 2A-B. In some instances, the nucleic acid polymer comprises between about 10 and about 100 nucleobases, between about 10 and about 75 nucleobases, between about 10 and about 50 nucleobases, between about 15 and about 100 nucleobases, between about 15 and about 75 nucleobases, between about 15 and about 50 nucleobases, between about 20 and about 100 nucleobases, between about 20 and about 75 nucleobases, between about 20 and about 50 nucleobases, between about 25 and about 100 nucleobases, between about 25 and about 75 nucleobases, or between about 25 and about 50 nucleobases.

Nucleic acid polymers include primers useful for amplifying a nucleic acid of biomarker provided in Tables 1 or 2A-2B. For example, for use in an amplification assay such as qPCR. Nucleic acid polymers also include probes comprising a detectable label for detecting and/or quantifying a biomarker of Tables 1 or 2A-2B. In some cases, the probes are reporters that comprise a dye label on one end and a quencher on the other end. When the probes are hybridized to a biomarker nucleic acid, an added DNA polymerase may cleave those hybridized probes, separating the reporter dye from the quencher, and thus increasing fluorescence by the reporter. In some cases, provided is a probe comprising a nucleic acid polymer described herein.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3,4-ij: 5,6,7-i‘j’]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3,6,7,12,13,16,17-octahydro-inner salt (TR or Texas Red); or BODIPY™ dyes. In some cases, the probe comprises FAM as the dye label.

In some instances, primers and/or probes described herein for hybridization to a biomarker of Tables 1 or 2A-2B are used in an amplification reaction. In some instances, the amplification reaction is qPCR. An exemplary qPCR is a method employing a TaqMan™ assay.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In one aspect, the methods provided herein for determining an expression profile in a subject comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In an exemplary qPCR assay, the nucleic acid sample is combined with primers and probes specific for a biomarker nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a biomarker nucleic acid.

In some embodiments, provided herein are primers/probes for the various biomarkers described herein, including in Tables 1, 2A, and/or 2B. In one embodiment, the primer/probe comprises a nu-cleotide sequence that hybridizes to contiguous 12-45 nucleotides within the nucleic acid sequences of the biomarkers provided in Tables 1, 2A, and/or 2B, wherein the nucleic acid sequences of the bi-omarkers can be found in and are hereby incorporated by reference from the public databases using the references in Tables 1, 2A, and/or 2B. In another embodiment, the primer/probe comprises a nu-cleotide sequence that hybridizes to a contiguous 12-45 nucleotides within the nucleic acid sequences of the biomarkers provided in Tables 1, 2A, and/or 2B under stringent conditions (e.g., hybridization to filter-bound DNA in 6× sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art. See, e.g., Current Protocols in Molecular Biology Vol. I, 6.3.1-6.3.6 and 2.10.3 (Ausubel et al. eds., 1989); Sambrook et al., Molecular Cloning: A Laboratory Manual (2001). In a further embodi-ment, the primer/probe is hybridizable to contiguous 12-45 nucleotides within the reverse comple-ment of the nucleic acid sequences of the biomarkers provided in Tables 1, 2A, and/or 2B. In yet an-other embodiment, the primer/probe comprises a nucleotide sequence that hybridizes to a contigu-ous 12-45 nucleotides within the reverse complement of the nucleic acid sequences of the biomarkers provided in Tables 1, 2A, and/or 2B under stringent conditions (e.g., hybridization to filter-bound DNA in 6× sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art. See, e.g., Current Protocols in Molecular Biology Vol. I, 6.3.1-6.3.6 and 2.10.3 (Ausubel et al. eds., 1989); Sambrook et al., Molecular Cloning: A Laboratory Manual (2001). The contiguous 12-45 nucleotides of the nucleic acid sequences or the reverse complement of the nucleic sequences of the biomarkers provided in Tables 1, 2A, and/or 2B are referred to as the target hybridization nucleotides. In some embodiments of the primers/probes provided herein, including in this paragraph, the target hybridiza-tion nucleotides comprise 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides.

### Profile Analysis

The expression profile of a patient sample (test sample) may be compared to a reference sample, e.g., a sample from a subject who does not have IBD such as CD (normal sample), or a sample from a subject who has a non-inflammatory MNP signature or a resident mucosal MNP signature. In some cases, a normal sample is that which is or is expected to be free of IBD disease or condition, or a sample that would test negative for any IBD disease or condition. The reference sample may be assayed at the same time, or at a different time from the test sample. In some cases, the expression profile of a reference sample is obtained and stored in a database for comparison to the test sample.

The results of an assay on the test sample may be compared to the results of the same assay on a reference sample. In some cases, the results of the assay on the normal sample are from a database. In some cases, the results of the assay on the normal sample are a known or generally accepted value by those skilled in the art. In some cases, the comparison is qualitative. In other cases, the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, gene product expression levels, gene product expression level changes, alternative exon usage, changes in alternative exon usage, protein levels, DNA polymorphisms, coy number variations, indications of the presence or absence of one or more DNA markers or regions, and/or nucleic acid sequences.

In some embodiments, the gene expression profile of a test sample is evaluated using methods for correlating gene product expression levels with a specific phenotype of CD, such as the subtype described herein. In some cases, a specified statistical confidence level may be determined in order to provide a diagnostic confidence level. For example, it may be determined that a confidence level of greater than 90% may be a useful predictor of an inflammatory MNP signature or a resident mucosal MNP signature.

In other embodiments, more or less stringent confidence levels may be chosen. For example, a confidence level of approximately 70%, 75%, 80%, 85%, 90%, 95%, 97.5%, 99%, 99.5%, or 99.9% may be chosen as a useful phenotypic predictor. The confidence level provided may in some cases be related to the quality of the sample, the quality of the data, the quality of the analysis, the specific methods used, and the number of gene expression products analyzed. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operator Curve analysis (ROC), binormal ROC, principal component analysis, partial least squares analysis, singular value decomposition, least absolute shrinkage and selection operator analysis, least angle regression, and the threshold gradient directed regularization method.

Raw gene expression level data may in some cases be improved through the application of algorithms designed to normalize and or improve the reliability of the data. In some embodiments of the present invention the data analysis requires a computer or other device, machine or apparatus for application of the various algorithms described herein due to the large number of individual data points that are processed. A “machine learning algorithm” refers to a computational-based prediction methodology, also known as a “classifier”, employed for characterizing a gene expression profile. The signals corresponding to certain expression levels, which are obtained by, e.g., microarray-based hybridization assays or sequencing, are typically subjected to the algorithm in order to classify the expression profile. Supervised learning generally involves “training” a classifier to recognize the distinctions among classes and then “testing” the accuracy of the classifier on an independent test set. For test samples the classifier can be used to predict the class in which the samples belong.

In some cases, the robust multi-array Average (RMA) method may be used to normalize the raw data. The RMA method begins by computing background-corrected intensities for each matched cell on a number of microarrays. The background corrected values are restricted to positive values as described by Irizarry et al. Biostatistics 2003 Apr. 4 (2): 249-64. The back-ground corrected, log-transformed, matched intensity on each microarray is then normalized using the quantile normalization method in which for each input array and each probe expression value, the array percentile probe value is replaced with the average of all array percentile points, this method is more completely described by Bolstad et al. Bioinformatics 2003. Following quantile normalization, the normalized data may then be fit to a linear model to obtain an expression measure for each probe on each microarray. Tukey's median polish algorithm (Tukey, J. W., Exploratory Data Analysis. 1977) may then be used to determine the log-scale expression level for the normalized probe set data.

Data may further be filtered to remove data that may be considered suspect. In some embodiments, data deriving from microarray probes that have fewer than about 4, 5, 6, 7 or 8 guanosine+ cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+ cytosine nucleotides may be considered unreliable due to their aberrant hybridization propensity or secondary structure issues.

In some cases, unreliable probe sets may be selected for exclusion from data analysis by ranking probe-set reliability against a series of reference datasets. For example, RefSeq or Ensembl (EMBL) are considered very high-quality reference datasets. Data from probe sets matching RefSeq or Ensembl sequences may in some cases be specifically included in microarray analysis experiments due to their expected high reliability. Similarly, data from probe-sets matching less reliable reference datasets may be excluded from further analysis, or considered on a case by case basis for inclusion. In some cases, the Ensembl high throughput cDNA (HTC) and/or mRNA reference datasets may be used to determine the probe-set reliability separately or together. In other cases, probe-set reliability may be ranked. For example, probes and/or probe-sets that match perfectly to all reference datasets such as for example RefSeq, HTC, and mRNA, may be ranked as most reliable (1). Furthermore, probes and/or probe-sets that match two out of three reference datasets may be ranked as next most reliable (2), probes and/or probe-sets that match one out of three reference datasets may be ranked next (3) and probes and/or probe sets that match no reference datasets may be ranked last (4). Probes and or probe-sets may then be included or excluded from analysis based on their ranking. For example, one may choose to include data from category 1, 2, 3, and 4 probe-sets; category 1, 2, and 3 probe-sets; category 1 and 2 probe-sets; or category 1 probe-sets for further analysis. In another example, probe-sets may be ranked by the number of base pair mismatches to reference dataset entries. It is understood that there are many methods understood in the art for assessing the reliability of a given probe and/or probe-set for molecular profiling and the methods of the present invention encompass any of these methods and combinations thereof.

The results of the expression profile may be analyzed to classify a subject as having or lacking an IBD disease or condition, such as an inflammatory MNP signature or a resident mucosal MNP signature. In some cases, a diagnostic result may indicate a certain molecular pathway involved in the IBD disease or condition, or a certain grade or stage of a particular IBD disease or condition. In some cases, a diagnostic result may inform an appropriate therapeutic intervention, such as a specific drug regimen like a molecule that targets a biomolecule in a pathway of any biomarker in Tables 1, 2A-2B, or a surgical intervention. In some cases, a diagnostic result indicates suitability or non-suitability of a patient for treatment with anti-TNFα.

In some embodiments, results are classified using a trained algorithm. Trained algorithms include algorithms that have been developed using a reference set of samples with a known IBD phenotype, such as PBT and CD-PBmu, or with an inflammatory MNP signature or a resident mucosal MNP signature. Algorithms suitable for categorization of samples include but are not limited to k-nearest neighbor algorithms, concept vector algorithms, naive Bayesian algorithms, neural network algorithms, hidden Markov model algorithms, genetic algorithms, and mutual information feature selection algorithms or any combination thereof. In some cases, trained algorithms may incorporate data other than gene expression such as DNA polymorphism data, sequencing data, scoring or diagnosis by cytologists or pathologists, information provided by the pre-classifier algorithm, or information about the medical history of the subject.

Compositions and Methods of Diagnosis and/or Treatment

Provided herein are compositions and methods of diagnosing, selecting for treatment, monitoring the treatment or treating an individual having a disease or condition. Non-limiting examples of inflammatory diseases include diseases of the gastrointestinal tract, liver, and/or gallbladder, including Crohn's disease (CD) and ulcerative colitis, systemic lupus erythematosus (SLE), and rheumatoid arthritis. In some embodiments, the subject has a certain phenotype of IBD, such as perianal disease/fistula, stricturing disease, recurrence, or increased immune reactivity to a microbial antigen, or a combination thereof. Compositions include any therapeutic agent that modulates expression and/or activity of a biomolecule in a pathway of one or more markers in Tables 1, or 2A-2B.

In some embodiments, described herein are methods for diagnosing a disease or a condition disclosed herein. In some embodiments, methods comprise: (a) detecting an increase or a decrease in expression of one or more genes provided in Table 1 and/or 2A-2B; and (b) diagnosing the subject with the disease or the condition based on the expression of the one or more genes that is detected. In some embodiments, methods comprise detecting a transcriptomic signature of the subject, wherein the transcriptomic signature comprises an increase or a decrease in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 1 and/or Table 2A or Table 2B. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold as compared to a reference sample. In some embodiments, the increase or the decrease in the expression of the one or more genes disclosed herein is an increase or a decrease of at least about 80%, 85%, 90%, 95%, 96%, 97%, 98%, or 99% of a level of expression in a reference sample. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 relative to a reference sample. In some embodiments, the reference sample is obtained from a subject that does not have the disease or the condition disclosed herein. In some embodiments, the reference sample is obtained from a subject that has the disease or the condition, but does not have the subtype of the disease of the condition described herein.

### Therapeutic Agents

In certain embodiments, described herein are methods for evaluating an effect of a treatment described herein. In some instances, the treatment comprises administration with a therapeutic agent provided herein, and optionally one or more additional therapeutic agents. In some instances, the treatment is monitored by evaluating the gene expression profile of a subject for expression of one or more genes in Tables 1, or 2A-2B. The gene expression profile may be determined prior to and/or after administration of a therapeutic agent. Gene expression profiling may also be used to ascertain the potential efficacy of a specific therapeutic intervention prior to administering to a subject. In some embodiments, methods comprise detecting a transcriptomic signature of the subject, wherein the transcriptomic signature comprises an increase or a decrease in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 1 and/or Table 2A or Table 2B. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold as compared to a reference sample. In some embodiments, the increase or the decrease in the expression of the one or more genes disclosed herein is an increase or a decrease of at least about 80%, 85%, 90%, 95%, 96%, 97%, 98%, or 99% of a level of expression in a reference sample. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 relative to a reference sample. In some embodiments, the reference sample is obtained from a subject that does not have the disease or the condition disclosed herein. In some embodiments, the reference sample is obtained from a subject that has the disease or the condition, but does not have the subtype of the disease of the condition described herein.

### MicroRNA Modulators

In some embodiments, methods disclosed herein comprise treating a disease or a condition in a subject by administering a therapeutic agent to the subject, provided that a transcriptomic signature disclosed herein is detected in a sample obtained from the subject. In some embodiments, the transcriptomic signature comprises an increase or a decrease in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 1 and/or Table 2A or Table 2B. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold as compared to a reference sample. In some embodiments, the increase or the decrease in the expression of the one or more genes disclosed herein is an increase or a decrease of at least about 80%, 85%, 90%, 95%, 96%, 97%, 98%, or 99% of a level of expression in a reference sample. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 relative to a reference sample. In some embodiments, the reference sample is obtained from a subject that does not have the disease or the condition disclosed herein. In some embodiments, the reference sample is obtained from a subject that has the disease or the condition, but does not have the subtype of the disease of the condition described herein. In some embodiments, the therapeutic agent disclosed herein, comprise modulators of miR-181a, miR-92a or miR-124, or any combination thereof. In some embodiments, the therapeutic agents are useful for the treatment of a disease or condition, or symptom of the disease or condition, disclosed herein. For example, the disease or condition comprises a subtype disclosed here, such as, for example, an inflammatory MNP signature or a resident mucosal MNP signature of Crohn's disease. In some embodiments, the disease or the condition is an inflammatory bowel disease (IBD). In some embodiments, the IBD is Crohn's disease (CD) or ulcerative colitis (UC).

In some embodiments, the therapeutic agents comprise a modulator of miR-181a. In some cases, the modulator of miR-181a is an antagonist, partial antagonist, agonist, or partial agonist. In some embodiments, the miR-181a modulator modulates the expression of one or more genes comprising PLAGI, PTPN11, HRAS, BCL2, FOS, DDIT4, BCL2L11, ATM, NOTCHI, MCL1, GATA6, DUSP6, DUSP5, PTPN22, PROXI, KAT2B, CDKN1B, XIAP, MTMR3, SRT1, NLK, KLF6, HPK2, RALA, or a combination thereof.

In certain embodiments, an miR-181a modulator comprises a molecule that upregulates expression of miR-181a. In certain embodiments, an miR-181a modulator comprises a molecule that downregulates or otherwise inhibits miR-181a. In some embodiments, the modulator of miR-181a is an oligomer. In some embodiments, the modulator of miR-181a is a microRNA inhibitor. In some embodiments, the modulator of miR-181a is a microRNA mimic. In a non-limiting exemplary embodiment, the microRNA is microRNA-181a or a precursor thereof, such as a mammalian microRNA-181a. Mammalian microRNA-181a includes human and mouse microRNA-181a.

In some embodiments, the therapeutic agents comprise a modulator of miR-92a. In some cases, the modulator of miR-92a is an antagonist, partial antagonist, agonist, or partial agonist. In some embodiments, the miR-92a modulator modulates the expression of one or more genes comprising BMPR2, PCGF5, TGFBR2, ITGAS, ESR2, CPEB2, OSBPL2, KLF2, KAT2B, HIPK3, MAPREl, RFFL, OSBPL8, TP63, ARD48, MYLP, or a combination thereof.

In certain embodiments, an miR-92a modulator comprises a molecule that upregulates expression of miR-92a. In certain embodiments, an miR-92a modulator comprises a molecule that downregulates or otherwise inhibits miR-92a. In some embodiments, the modulator of miR-92a is an oligomer. In some embodiments, the modulator of miR-92a is a microRNA inhibitor. In some embodiments, the modulator of miR-92a is a microRNA mimic. In a non-limiting exemplary embodiment, the microRNA is microRNA-92a or a precursor thereof, such as a mammalian microRNA-92a. Mammalian microRNA-92a includes human and mouse microRNA-92a.

In some embodiments, the therapeutic agents comprise a modulator of miR-124. In some cases, the modulator of miR-124 is an antagonist, partial antagonist, agonist, or partial agonist. In some embodiments, the miR-124 modulator modulates the expression of one or more genes comprising CEBPA, BACE1, SMYD3, RELA, AR, MECP2, E2F6, FXN, PEA15, IL6R, SLC116A1, NR3C2, ITGB1, NFKBIZ, CTDSP1, IQGAP1, HMGA1, LAMC1, CDK4, ROCK2, CDK2, RDH10, NR3C1, ELK3, CCL2, AHR, EZH2, MTPN, CDK8, EFNB1, VM, ADPOR2 or a combination thereof.

In certain embodiments, an miR-124 modulator comprises a molecule that upregulates expression of miR-124. In certain embodiments, an miR-124 modulator comprises a molecule that downregulates or otherwise inhibits miR-124. In some embodiments, the modulator of miR-124 is an oligomer. In some embodiments, the modulator of miR-124 is a microRNA inhibitor. In some embodiments, the modulator of miR-124 is a microRNA mimic. In a non-limiting exemplary embodiment, the microRNA is microRNA-124 or a precursor thereof, such as a mammalian microRNA-124. Mammalian microRNA-124 includes human and mouse microRNA-124.

### TL1A Therapy

In some embodiments, the therapeutic agents comprise a Tumor necrosis factor-like cytokine TA (TL1A) therapy. In some embodiments, the TL1A therapy is a modulator of TL1A activity or expression. In some embodiments, the TL1A therapy is an inhibitor of TL1A activity or expression. In some embodiments, the TL1A therapy is an allosteric modulator of TL1A. Non-limiting examples of an inhibitor of TL1A expression include RNA to protein translation inhibitors, antisense oligonucleotides targeting the TNFSF15 mRNA (such as miRNAs, or siRNA), epigenetic editing (such as, for example, targeting the DNA-binding domain of TNFSF15, or post-translational modifications of histone tails and/or DNA molecules). Non-limiting examples of an inhibitor of TL1A activity include antagonists to the TL1A binding partners (e.g., Decoy Receptor 3 (DcR3)), antagonists to TL1A antigen, and antagonists to gene expression products involved in TL1A mediated disease. Antagonists as disclosed herein, may include, but are not limited to, an anti-TL1A antibody or antigen-binding fragment thereof, or a small molecule drug. In some embodiments, the TL1A therapy comprises an anti-TL1A therapy. In one embodiment, the anti-TL1A therapy is a small molecule drug. In one embodiment, the anti-TL1A therapy is a biologic drug. In some embodiments, the anti-TL1A therapy comprises an antibody and antigen-binding fragment thereof. In some embodiments, an antibody comprises an antigen-binding fragment that refers to a portion of an antibody having antigenic determining variable regions of an antibody. Examples of antigen-binding fragments include, but are not limited to, Fab, Fab′, F(ab′)2, and Fv fragments, linear antibodies, single chain antibodies, and multispecific antibodies formed from antibody fragments. In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′)2, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, or the like.

In some embodiments, the anti-TL1A antibody or antigen-binding fragment thereof is humanized. In some embodiments, a humanized antibody refers to forms of non-human (e.g., murine) antibodies having specific immunoglobulin chains, chimeric immunoglobulins, or fragments thereof that contain minimal non-human (e.g., murine) sequences. In a non-limiting example, a humanized antibody comprises less than about 40% non-human sequence in the variable region. In some cases, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 110%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability. These humanized antibodies may contain one or more non-human species mutations, e.g., the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region.

In some embodiments, the anti-TL1A antibody or antigen-binding fragment thereof is is a chimeric antibody or antigen-binding fragment thereof. In some embodiments, chimeric antibodies refer to antibodies wherein the sequence of the immunoglobulin molecule is derived from two or more species. As a non-limiting example, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

In some embodiments, the anti-TL1A antibody or antigen-binding fragment thereof specifically binds to TL1A (e.g., Entrez Gene: 9966; UniProtKB: 095150). In some embodiments, the anti-TL1A antibody or antigen-binding fragment thereof specifically binds to soluble TL1A. In some embodiments, the anti-TL1A antibody or antigen-binding fragment thereof specifically binds to membrane bound TL1A In some embodiments, an anti-TL1A antibody or antigen-binding fragment thereof comprises a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4. In some embodiments, the anti-TL1A antibody or antigen-binding fragment thereof is or comprises PRA023. In some embodiments, the anti-TL1A antibody or antigen-binding fragment thereof is or comprises tulisokibart. In some embodiments, the anti-TL1A antibody or antigen-binding fragment thereof is or comprises PF-06480605. In some embodiments, the anti-TL1A antibody or antigen-binding fragment thereof is or comprises PF-06480605 (such as, for example, disclosed in clinical trial NCT04090411, NCT05471492, or NCT02840721). In some embodiments, the anti-TL1A antibody or antigen-binding fragment thereof is or comprises TEV-48574 (such as, for example, disclosed in clinical trial NCT05499130). In some embodiments, the anti-TL1A antibody or antigen-binding fragment thereof is or comprises an antibody or antigen-binding fragment disclosed in U.S. Pat. Nos. 10,322,174; 10,689,439; 11,440,954; 11,136,386; 11,292,848; 9,683,998; 10,968,279; 8,642,741; 10,822,422; 8,263,743; 8,728,482; 10,138,296; 9,290,576; or 11,104,745; or any combination thereof; each of which is hereby incorporated by reference in its entirety.

### CD30L Therapy

In some embodiments, the therapeutic agent comprises a TNF Superfamily Member 8 (CD30L) therapy. In some embodiments, the CD30L therapy comprises an modulator of CD30L activity or expression. In some embodiments, the CD30L therapy comprises an inhibitor of CD30L activity or expression. In some embodiments, the CD30L therapy comprise an anti-CD30L therapy. In some embodiments, the anti-CD30L therapy is a small molecule drug. In some embodiments, the anti-CD30L therapy is a biologic drug. In some embodiments, the anti-CD30L therapy comprises an antibody or antigen-binding fragment thereof (anti-CD30L antibody or antigen-binding fragment thereof). In some embodiments, the antibody or antigen-binding fragment thereof binds CD30 ligand (CD30L). In some embodiments, CD30L that is the target of the anti-CD30L therapy provided herein can be an isoform of CD30L. Such CD30L isoforms can be results of an alternate splice site in the coding region of the last exon. In some embodiments of the isoform of CD30L is isoform 1 of CD30L. In some embodiments, the isoform of CD30L is isoform 2 of CD30L. Isoform 1 of CD30L is described in further detail in NM_001244.4 (mRNA and protein sequence) and NP_001235.1 (protein sequence), each of which is incorporated by reference herein in its entirety. Isoform 2 of CD30L is described in further detail in NM_001252290.1 (mRNA and protein sequence) and NP_001239219.1 (protein sequence), each of which is incorporated herein by reference in its entirety. In some embodiments, the the inhibitor of CD30L activity or expression blocks an interaction between CD30L and CD30. In some embodiments, the the inhibitor of CD30L activity or expression inhibits pro-inflammatory cytokine release. In some embodiments, the inhibitor of CD30L activity or expression is effective to inhibit CD30L-CD30 binding. In some embodiments, the inhibitor of CD30L activity or expression comprises an allosteric modulator of CD30L. An allosteric modulator of CD30L may indirectly influence the effects of CD30L on CD30. The inhibitor of CD30L activity or expression may be a direct inhibitor or indirect inhibitor. Non-limiting examples of an inhibitor of CD30L expression include RNA to protein CD30L translation inhibitors, antisense oligonucleotides targeting the TNFSF8 mRNA (such as miRNAs, or siRNA), epigenetic editing (such as targeting the DNA-binding domain of TNFSF8, or post-translational modifications of histone tails and/or DNA molecules). Non-limiting examples of an inhibitor of CD30L activity include antagonists to the CD30L receptors, (e.g., CD30), antagonists to CD30L antigen, and antagonists to gene expression products involved in CD30L mediated disease. Antagonists as disclosed herein, may include, but are not limited to, an anti-CD30L antibody or antigen-binding fragment thereof, or a small molecule. The small molecule may be a small molecule that binds to CD30L or CD30. The anti-CD30L antibody may be monoclonal or polyclonal. The anti-CD30L antibody may be humanized. The anti-CD30L antibody may be chimeric. The anti-CD30L antibody may be a fusion protein. The anti-CD30L antibody may be a blocking anti-CD30L antibody. In some embodiments, a blocking antibody blocks binding between two proteins, e.g., a ligand and its receptor. Therefore, an anti-CD30L blocking antibody includes an antibody that prevents binding of CD30L to CD30L receptors (e.g., CD30). In a non-limiting example, the anti-CD30L blocking antibody binds to CD30. In some cases, the CD30L antibody is an anti-CD30L antibody that specifically binds to CD30L. In some cases, the CD30L antibody specifically binds to an epitope of the CD30L protein.

In some embodiments, the anti-CD30L antibody is or comprises KPL-045. In some embodiments, the anti-CD30L antibody is or comprises PRA052. In some embodiments, the anti-CD30L antibody or antigen-binding fragment thereof is or comprises an antibody or antigen-binding fragment thereof disclosed in U.S. patent application Ser. No. 17/822,598, which is incorporated by reference herein in its entirety. In some embodiments, the anti-CD30L antibody or antigen-binding fragment thereof is or comprises an antibody or antigen-binding fragment thereof disclosed in U.S. Pat. No. 9,926,373, which is incorporated by reference herein in its entirety. In one embodiment, the anti-CD30L antibody or antigen binding fragment thereof comprises an anti-CD30L antibody or antigen binding fragment selected from those described in U.S. Pat. No. 9,926,373, or U.S. patent application Ser. No. 17/822,598; at a formulation and dose as described in U.S. Pat. No. 9,926,373 or U.S. patent application Ser. No. 17/822,598, each of which is hereby incorporated by reference in its entirety.

### Pharmaceutical Compositions, Formulations, and Methods of Administration

In one aspect, methods of treating a subject, e.g., a subject having an inflammatory MNP signature, or a resident mucosal MNP signature, or any combination thereof, involve administration of a pharmaceutical composition comprising a therapeutic agent described herein, e.g., a modulatory of expression and/or activity of a biomarker in Tables 1, or 2A-2B, or a modulator of miR-181a, miR-92a or miR-124, or a combination thereof, in therapeutically effective amounts to said subject. In some embodiments, the subject has perianal disease/fistula, stricturing disease, recurrence, or increased immune reactivity to a microbial antigen, or a combination thereof. In some embodiments, a therapeutic agent described herein is used in the preparation of medicaments for treating an inflammatory disease, such as Crohn's Disease.

In certain embodiments, the compositions containing the therapeutic agent described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial. In some cases, a therapeutic agent is administered to a patient suffering from an inflammatory disease such as CD, and optionally comprises an inflammatory MNP signature or a resident mucosal MNP signature.

In prophylactic applications, compositions containing a therapeutic agent described herein are administered to a patient susceptible to or otherwise at risk of a particular disease, disorder or condition, e.g., an inflammatory disease. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition.

In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given therapeutic agent that corresponds to such an amount varies depending upon factors such as the particular therapeutic agent, disease condition and its severity, the identity (e.g., weight, sex, age) of the subject in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In some embodiments, as a patient is started on a regimen of a therapeutic agent, the patient is also weaned off (e.g., step-wise decrease in dose) a second treatment regimen.

In one embodiment, the daily dosages appropriate for a therapeutic agent herein are from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In some embodiments, the daily dosage is administered in extended release form. In certain embodiments, suitable unit dosage forms for oral administration comprise from about 1 to 500 mg active ingredient. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED50 with minimal toxicity. In certain embodiments, the daily dosage range and/or the unit dosage amount varies within this range depending upon the dosage form employed and the route of administration utilized.

Disclosed herein are therapeutic agents formulated into pharmaceutical compositions. Pharmaceutical compositions are formulated in a conventional manner using one or more pharmaceutically acceptable inactive ingredients that facilitate processing of the active therapeutic agent into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided herein are pharmaceutical compositions that include a therapeutic agent described herein, and at least one pharmaceutically acceptable inactive ingredient. In some embodiments, the therapeutic agents described herein are administered as pharmaceutical compositions in which the therapeutic agents are mixed with other active ingredients, as in combination therapy. In some embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In some embodiments, the pharmaceutical compositions include other therapeutically valuable substances.

A pharmaceutical composition, as used herein, refers to a mixture of a therapeutic agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other steroisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, atherapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e. g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH>5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of atherapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

### Kits

The disclosure also provides kits for detecting expression of one or more genes in Tables 1, or 2A-2B. Exemplary kits include nucleic acids configured for specific hybridization to one or more genes in Tables 1, or 2A-2B. In some cases a kit comprises a plurality of such nucleic acids immobilized on a substrate, such as a microarray, welled plate, chip, or other material suitable for microfluidic processing.

In some embodiments, the kit includes nucleic acid and/or polypeptide isolation reagents. In some embodiments, the kit includes one or more detection reagents, for example probes and/or primers for amplification of, or hybridization to, a gene in Tables 1, or 2A-2B. In some embodiments, the kit includes primers and probes for control genes, such as housekeeping genes. In some embodiments, the primers and probes for control genes are used, for example, in ΔCt calculations. In some embodiments, the probes or primers are labeled with an enzymatic, florescent, or radionuclide label.

In some instances, a kit comprises a nucleic acid polymer (e.g., primer and/or probe) comprising at least about 10 contiguous nucleobases having at least about 70%, 80%, 85%, 90%, 91%, 92%, 93%, 94%, 95%, 96%, 97%, 98% or 99% sequence identity or homology to a biomarker of Tables 1, or 2A-2B.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, a kit includes one or more additional containers, each with one or more of various materials (such as reagents, optionally in concentrated form, and/or devices) desirable from a commercial and user standpoint for use of described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

### Systems

Disclosed herein, in some embodiments, is a system for detecting a particular subtype of IBD or CD in a subject. In some embodiments, the subtype comprises an inflammatory MNP signature. In some embodiments, the subtype comprises a resident mucosal MNP signature. In some embodiments, the subtype is CD-PBmu. In some embodiments, the subtype is CD PBT (peripheral blood T cells). In some embodiments, the subtype is monocyte 2 subtype. In some embodiments, the subtype is monocyte 1 subtype. The system is configured to implement the methods described in this disclosure, including, but not limited to, detecting the presence of a particular CD subtype to determine whether the subject is suitable for treatment with a particular therapy.

In some embodiments, disclosed herein is a system for detecting a IBD subtype in a subject, comprising: (a) a computer processing device, optionally connected to a computer network; and (b) a software module executed by the computer processing device to analyze a target nucleic acid sequence of a transcriptomic profile in a sample from a subject. In some instances, the system comprises a central processing unit (CPU), memory (e.g., random access memory, flash memory), electronic storage unit, computer program, communication interface to communicate with one or more other systems, and any combination thereof. In some instances, the system comprises a genotype device, such as a sequencer, polymerase chain reaction (PCR) machine or a genotype array configured for detecting the increase or the decrease in the expression of the one or more genes in Table 1 and/or Table 2A or Table 2B. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof.

The computer-implemented platforms or systems disclosed herein for determining a Crohn's Disease (CD) subtype status in a subject having CD, wherein the status comprises identifying a CD13+ mononuclear phagocytic (MNP) subtype, comprise: (a) one or more processors collectively or individually programmed to implement a method comprising: (i) analyzing genotype data of the subject to detect a level of expression of one or more genes provided in Table 1 and/or Table 2A or Table 2B to produce an expression profile of the subject; and (ii) determining the CD subtype status of the subject based upon the expression profile, wherein an increased level of the expression of the one or more genes as compared to a reference expression profile indicates that the CD subtype status of the subject comprises a CD-MNP subtype; and (b) a database for storing the genotype data of the subject and/or the expression profile. In some embodiments, the non-CD-MNP subtype comprises a CD subtype without MNP signature.

In some embodiments, the subtype is predicted with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the subtype of the disease or condition using the system may be calculated as the percentage of samples identified or classified as having the subtype of the disease or condition that correspond to subjects that truly have the subtype.

In some embodiments, the subtype is predicted with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the subtype of the disease or condition using the system may be calculated as the percentage of samples identified or classified as not having the subtype of the disease or condition that correspond to subjects that truly do not have the subtype.

In some embodiments, the subtype is predicted with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the subtype of the disease or condition using the system disclosed herein may be calculated as the percentage of independent test samples associated with presence of the subtype (e.g., subjects known to have the subtype) that are correctly identified or classified as having the subtype.

In some embodiments, the subtype is predicted with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the subtype of the disease or condition using the system may be calculated as the percentage of independent test samples associated with absence of the subtype (e.g., subjects with negative clinical test results for the subtype) that are correctly identified or classified as not having the subtype.

In some embodiments, the system is configured to identify the presence (e.g., positive test result) or absence (e.g., negative test result) of the subtype with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the algorithm that classifies the samples as having or not having the subtype. The AUC may range from a value of 0 to 1, where an AUC of 0.5 is indicative of a completely random classifier (e.g., a coin flip) and an AUC of 1 is indicative of a perfectly accurate classifier (with sensitivity of 100% and specificity of 100%).

### Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. In some instances, a computer program includes one or more executable complied applications.

### Web Browser Plug-In

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems.

Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The subject matter described herein, including methods for detecting a particular CD subtype, are configured to be performed in one or more facilities at one or more locations. Facility locations are not limited by country and include any country or territory. In some instances, one or more steps are performed in a different country than another step of the method. In some instances, one or more steps for obtaining a sample are performed in a different country than one or more steps for detecting the presence or absence of a particular CD subtype from a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy, or the like, in the same or different location or country.

### Business Methods Utilizing a Computer

The gene expression profiling methods may utilize one or more computers. The computer may be used for managing customer and sample information such as sample or customer tracking, database management, analyzing molecular profiling data, analyzing cytological data, storing data, billing, marketing, reporting results, storing results, or a combination thereof. The computer may include a monitor or other graphical interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a gene expression profile may enter sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

As used herein percentage identity or homology may generally refer to percentage of nucleotides that are identical between two or more sequences of DNA or RNA.

As used herein the term “gene” may generally refer to a sequence of nucleotides that comprise a part of a chromosome.

As used herein the term “profile” may generally refer to a compilation of data associated with an individual or a population comprising information specific to that individual or population. In some instances, the information comprises genetic information such as genetic variations or gene expressions specific to that individual or population.

As used herein the term “signature” may generally refer to a single or combined group of genes that are a uniquely characteristic pattern of gene expression associated with a population or subpopulation.

In some instances, the characteristic pattern of gene expressions is associated with a phenotype expressed by the population or subpopulation.

As used herein the term “control” or “reference” may generally refer to a group that can be used to in a scientific experiment in which the independent variable cannot influence the outcome.

### EXAMPLES

The following illustrative examples are representative of embodiments of the systems and methods described herein and are not meant to be limiting in any way.

### Example 1—Stratifying Intestinal Mononuclear Phagocyte Expression Profiles Subjects

Human subjects were recruited through the MIRIAD IBD Biobank at the F. Widjaja Foundation Inflammatory Bowel and Immunobiology Research Institute at Cedars-Sinai Medical Center. Informed consent (approved by the Institutional Review Board at Cedars-Sinai Medical Center) was obtained from all participating subjects. Clinical information was obtained from Crohn's disease (CD) patients (n=48) prior to undergoing surgical resection after which patients were followed prospectively. Subjects without inflammatory bowel disease (IBD) (n=11) had no known history of IBD and underwent surgery for colon cancer (n=3), colon polyps (n=2), colonic inertia (n=1), diverticulitis (n=2), rectal cancer (n=1), familial adenomatous polyposis (n=1), and rectal polyps (n=1). All CD and non-IBD samples were collected from surgical resections performed by a single provider.

### Methods and Results

CD13+ mononuclear phagocytic (MNP) cells were purified from lamina propria mucosal tissue from the 48 CD patients and 11 non-IBD individuals requiring surgery. CD14+ peripheral monocyte cells were purified from lamina propria mucosal tissue from 47 CD patients subjects and 9 non-IBD patients at the time of surgery, as well as after surgery for 42 of those subjects. Whole RNA was extracted from the CD13+ and CD14+MNP cell subsets. Libraries for RNA-Seq were prepared with an updated version of the kit (Nugen Universal RNA-seq with NuQuant (part number: 0364 Nugen, Tecan) to generate strand-specific RNA-seq libraries. The workflow consists of poly(A) RNA selection, RNA fragmentation and double-stranded cDNA generation using a mixture of random and oligo(dT) priming, followed by end repair to generate blunt ends, adaptor ligation, strand selection, and PCR amplification to produce the final library. Different index adaptors were used for multiplexing samples in one sequencing lane. RNA sequencing was performed using the Illumina NovaSeq™ 6000 (2×150 output) at 30 million (M) reads/sample from each direction. All libraries were prepared using a single lot or reagents, equipment and processed by same technical staff. Samples were processed in two runs with technical and sample duplicates with negligible batch differences. Data quality check was done on Illumina SAV. Demultiplexing was performed with Illumina Bcl2fastq2 v 2.19.1.403 program. STAR aligner software version 2.7.2a was used to align sequenced reads to Human Genome version GRCh38. Reads per gene was quantified using STAR version 2.7.2a with Ensembl GRCh38.98 GTF file. Normalization of reads per gene data was performed with TMM normalization method in Partek Flow software. Samples that had less than 10 million reads mapping to gen exons were eliminated from final normalization. Clean, processed data along with respective meta-data was available in-house. The differential gene expression data from the RNA-Seq is provided in Table 1.

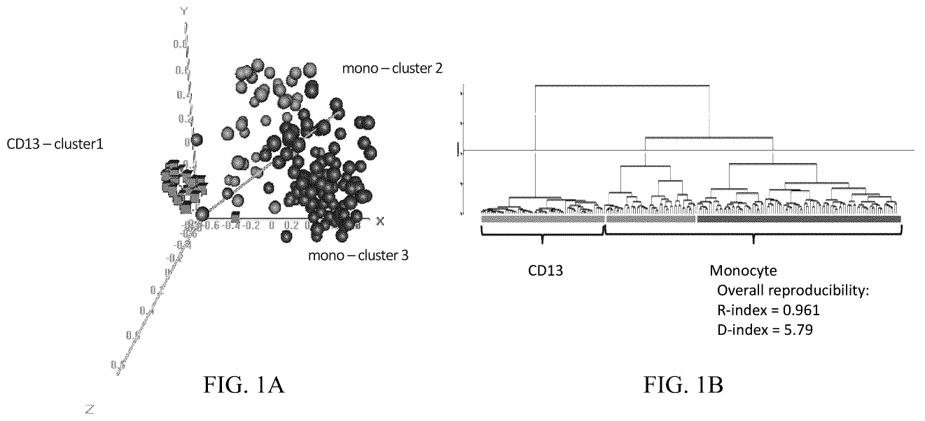

A hierarchical cluster analysis was performed on the RNA-Seq data for the CD13+ and CD14+ subsets, and the results are provided in FIG. 1B, showing three distinct clusters. A principle component analysis (PCA) analysis was performed based on the clustering analysis, which is shown in FIG. 1B (squares corresponding to CD13+, and light and dark circles corresponding with other monocyte populations, clusters 3 and 2 respectively). FIG. 1B shows a dendrogram illustrating the results from this PCA analysis, and illustrates that CD13+ subset clusters separately from the other monocyte population.

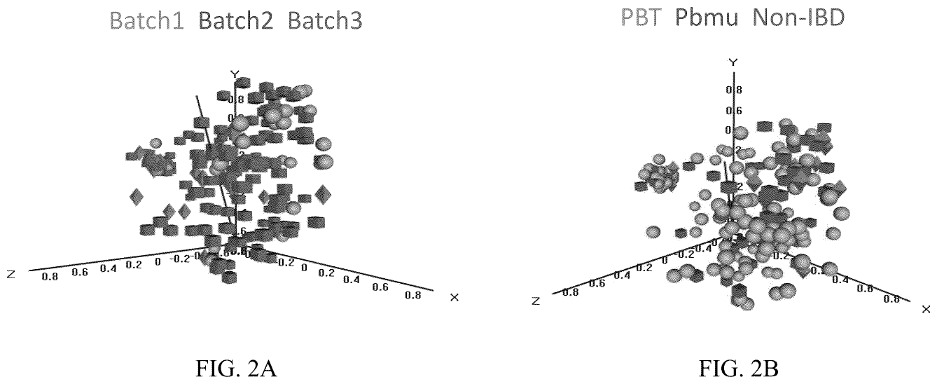

The results shown in FIG. 1A and FIG. 1B were not the result of batch effects, as shown in FIG. 2A. A hierarchical clustering analysis was also performed to see whether the previously reported CD-PBmu subtype carried over to monocytes, which it does not, as shown in FIG. 2B. The CD-PBmu subtype was previous reported in U.S. patent application Ser. No. 17/334,109, and International Application No. PCT/US2021/035217, each of which is hereby incorporated by reference in its entirety.

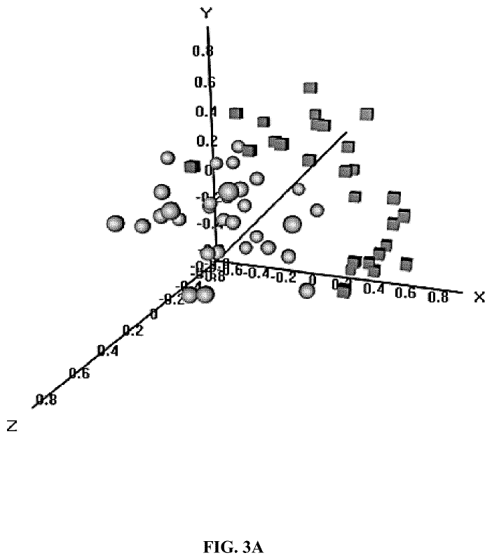

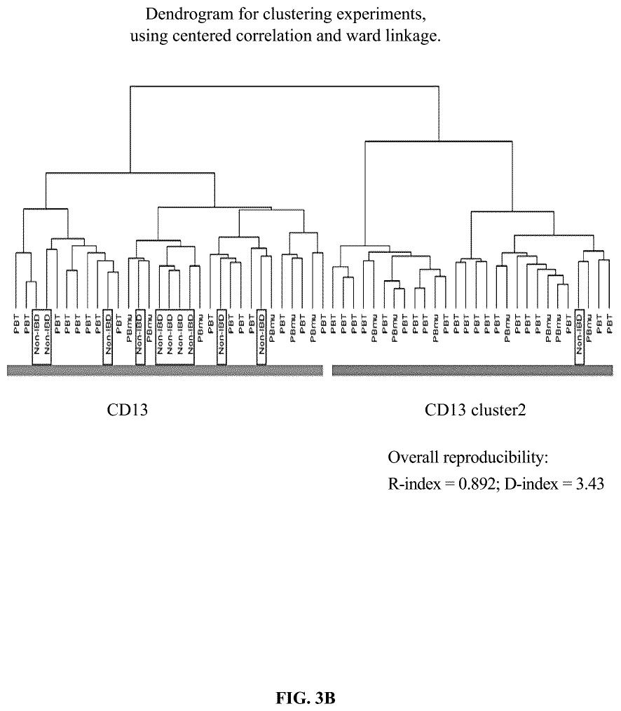

Unsupervised hierarchical clustering analysis was performed on the RNA-Seq data for the CD13+ cluster only, revealing two transcriptomic signatures within this subset: Expression from CD subgroup 1 (53%) (“CD13 cluster 1”), clustered tightly with the non-IBD group, and was defined by an inflammatory macrophage signature with upregulated expression overlapping with pathways regulating Th1/Th2/Th17 cell differentiation, inflammation mediated cytokine/chemokine signaling and IBD GWAS associations. CD subgroup 2 (“CD13 cluster 2”), clustered tightly with the non-IBD group, and was defined by a resident macrophage signature with upregulated expression overlapping with a strong mucosal and mitochondrial dysfunction signature and with abnormal crypt and intestinal physiology in murine animal models. FIG. 3A shows the same cluster analysis in a plot, with squares corresponding with CD13 cluster 2 and circles corresponding to CD13 cluster 1. FIG. 3B provides a dendrogram illustrating the results from this clustering analysis, and illustrates that CD13 cluster 1 clustered tightly with the non-IBD. Table 2A provides the input for CD13 cluster 1. Table 2B provides the input for CD13 cluster 2.

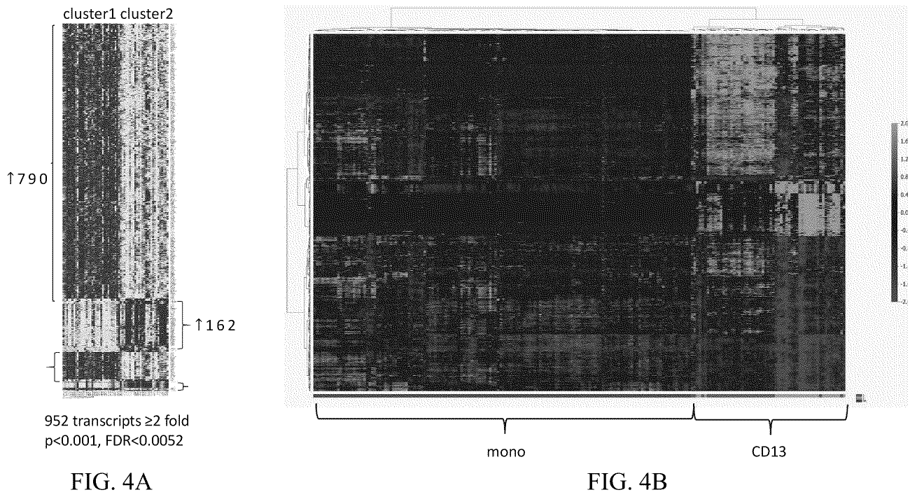

Differential gene expression between CD13 cluster 1 and CD13 cluster 2 can be seen in the heatmap provided in FIG. 4A (also listed in Table 1), which illustrates an upregulation of 162 genes in the CD13 cluster 2 as compared with CD13 cluster 1 (listed in Table 2B); and an upregulation of 790 genes in CD13 cluster 1 as compared with CD13 cluster 2 (listed in Table 2A). FIG. 4B provides a heatmap of the differential gene expression of the 952 transcripts in FIG. 4A in the entire monocyte dataset (“mono”) as compared with the CD13 cluster 1 and CD13 cluster 2, and illustrates that the CD13 cluster 1 and CD13 cluster 2 differ in gene expression from each other, as well as the rest of the monocyte dataset.

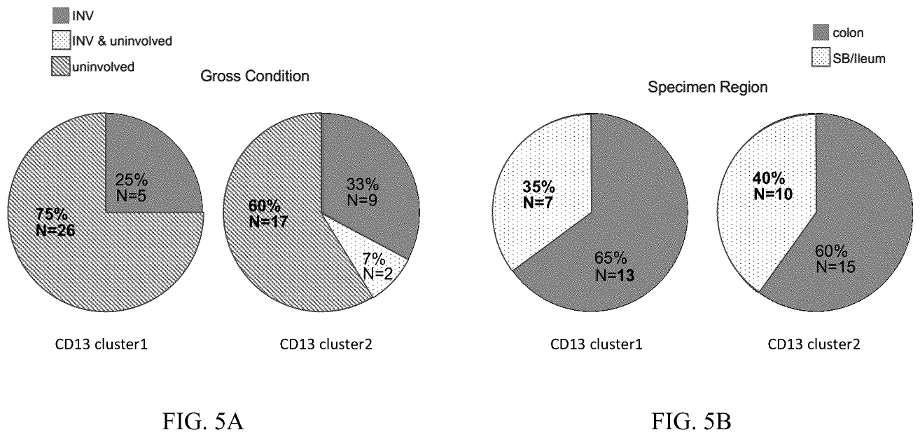

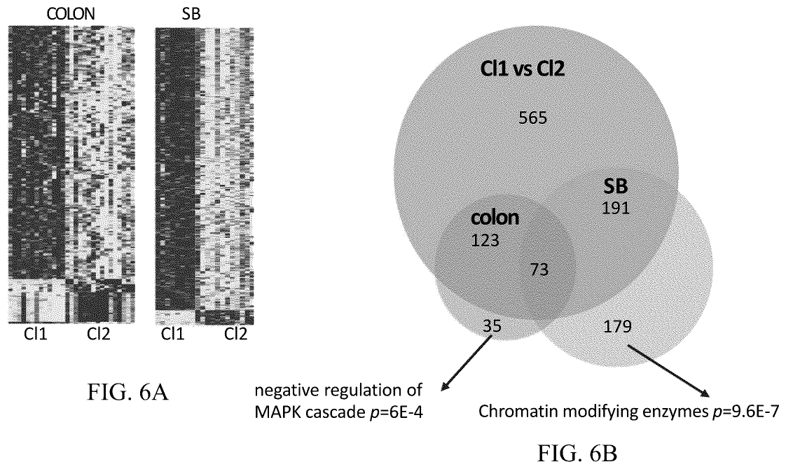

The samples corresponding with CD13 cluster 1 and CD13 cluster 2 were analyzed to see whether there was an association between either cluster and the “gross condition” of the tissue as determined by a pathologist looking at whether there was inflammation in the tissue (“INV”), no inflammation in the tissue (“uninvolved”) or a mix of inflammation an uninflamed tissue (“INV & uninvolved”). FIG. 5A shows that there was no significant association between the gross condition of the tissue and the CD13 cluster 1 and CD13 cluster 2. A similar analysis was done to determine whether the CD13 cluster 1 and CD13 cluster 2 are associated with disease location (“colon” or the “SB/ileum,” “SB” refers to small bowel). FIG. 5B shows that there is no significant association between disease location and the CD13 cluster 1 and CD13 cluster 2. When the differential gene expression between the CD13 cluster 1 and CD13 cluster 2 in the colon tissue versus the small bowel, the differences observed were due to differences in gene expression between CD13 cluster 1 and CD13 cluster 2 and not necessarily based on disease location, as shown in the heat map provided in FIG. 6A and pictorially represented in the Venn diagram provided in FIG. 6B.

Pathway enrichment analyses were performed on the RNA-Seq data to identify associations between cellular pathways and the differential gene expression in the CD13+ subsets. Comparing the CD13+ gene expression in non-IBD subjects compared with CD subjects, enrichment in apoptosis, TNF/stress signaling and Toll receptor pathways, as shown in Table 3.

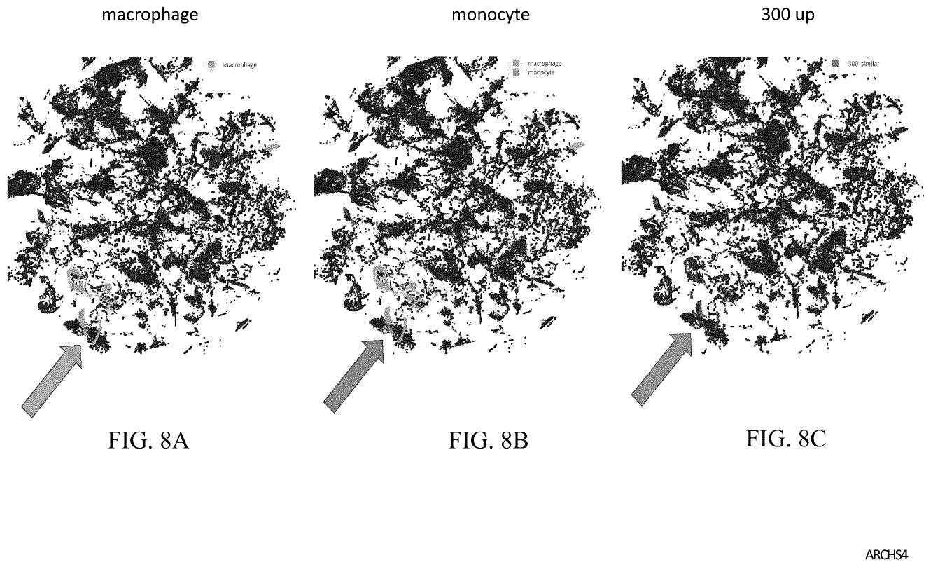

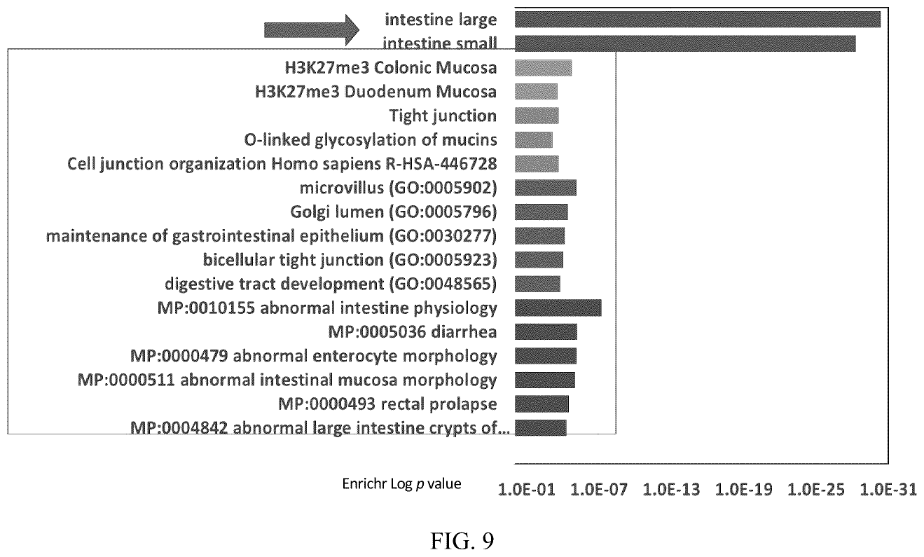

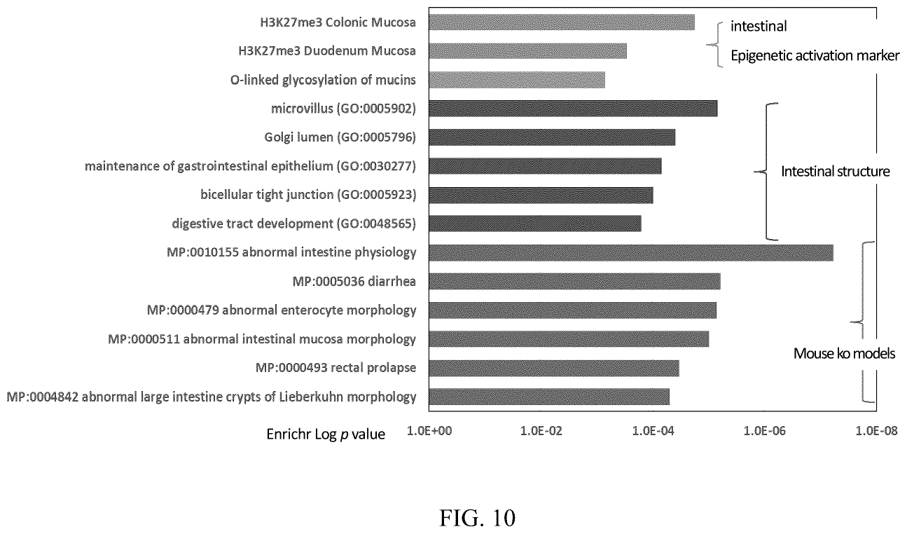

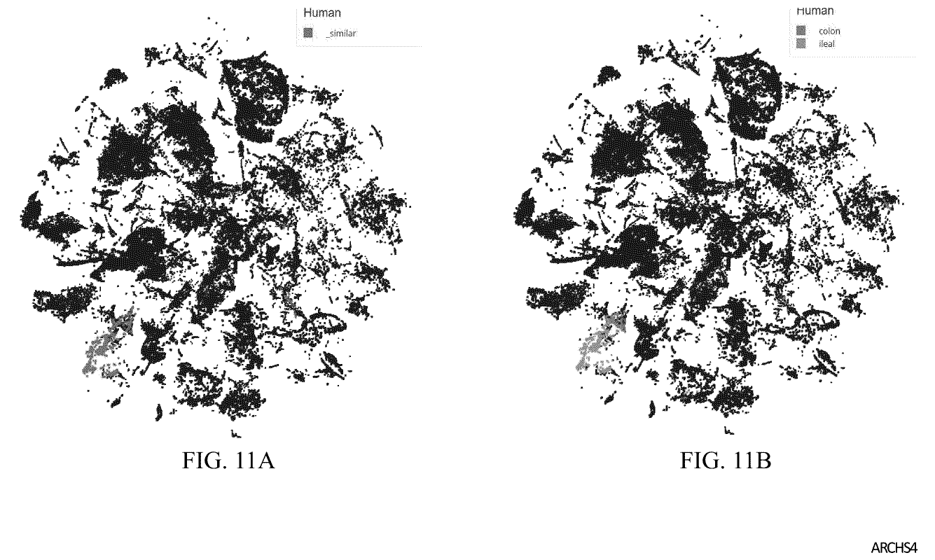

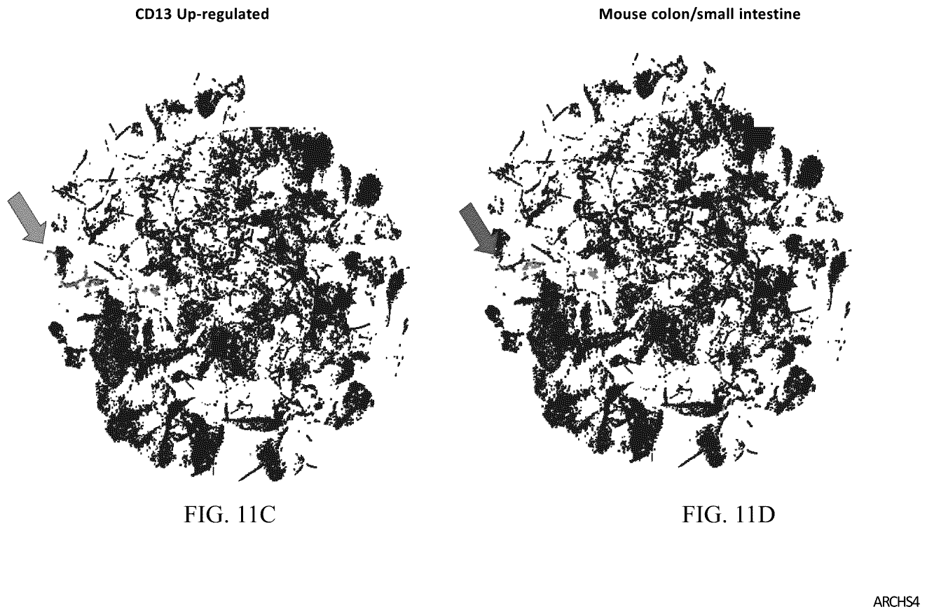

FIG. 7 shows an upregulation of immune system pathways, inflammatory bowel disease, apoptosis, tumor necrosis factor (TNF)/stress signaling and Toll receptor pathways in the CD13 cluster 1. ARCHS⁴ (https://amp.pharm.mssm.edu/archs4) database tool was used to identify tissue gene expression signatures in common with the RNA-Seq data from CD13 cluster 1, which is shown in FIG. 8A-8B. FIG. 8A shows that a macrophage gene expression signature (FIG. 8A), monocyte gene expression signature (FIG. 8B) and the first ranked 300 genes upregulated in cluster 1 (FIG. 8C) from other datasets all overlap with the gene expression signature of CD13 cluster 1. By contrast, pathways enriched in the CD13 cluster 2 include the large and small intestine pathways (FIG. 9), as well as intestinal epigenic activation, intestinal structure, and mouse knock out (“ko”) models (FIG. 10). ARCHS⁴ was used to identify tissue gene expression signatures in human datasets (FIG. A) and human ileal and colonic datasets (FIG. 11B), as well as mouse datasets (FIG. 11C) and mouse datasets in the colon and small intestine (FIG. 11D), all of which overlap with CD13 cluster 2.

TABLE 3
Transcriptomic pathways enriched in non-IBD vs CD MNP

| | |
|---|---|
| Apoptosis signaling pathway | 5.6E−04 |
| Toll receptor cascades | 2.9E−03 |
| TNF/stress-related signaling | 4.5E−03 |

Transcriptomic pathways enriched in CD13
cluster 1 - pro-inflammatory MNP

| | |
|---|---|
| IL-2 Signaling | 8.4E−08 |
| Th17 cell differentiation | 2.7E−06 |
| Chemokine signaling pathway | 7.3E−06 |
| Th1 and Th2 cell differentiation | 1.1E−05 |
| IL 4 signaling pathway | 9.4E−05 |
| IL22 Soluble Receptor Signaling Pathway | 3.4E−04 |
| Inflammatory Bowel Diseases | 9.7E−04 |

Transcriptomic pathways enriched in CD13
cluster 2- resident mucosal MNP

| | |
|---|---|
| Mitochondrial myopathy | 7.8E−24 |
| Small intestine | 4.4E−15 |
| Ileum | 1.4E−13 |
| Colon | 3.7E−12 |
| MGI Mammalian Phenotype | |
| intestinal obstruction MP: 0003270 | 4.0E−05 |
| abnormal large intestine crypts of Lieberkuhn morphology | 4.2E−04 |
| MP: 0004842 | |
| abnormal intestine physiology MP: 0010155 | 7.9E−04 |
| abnormal intestinal mucosa morphology MP: 0000511 | 1.2E−03 |
| Enriched in pro-inflammatory vs. resident subtype MNP | |
| macrophage | 3.0E−02 |
| M1-like | 1.1E−02 |
| M2-like | 2.0E−03 |
| activated DC | 9.4E−03 |
| Enriched in resident vs pro-inflammatory subtype MNP | |
| anti-Saccharomyces cerevisiae glycan antibodies | 1.0E−02 |
| anti-neutrophil cytoplasmic antibodies | 4.0E−02 |
| expression of serotonin receptor HTR2a | 2.0E−04 |
| expression of serotonin receptor HTR4 | 2.8E−05 |

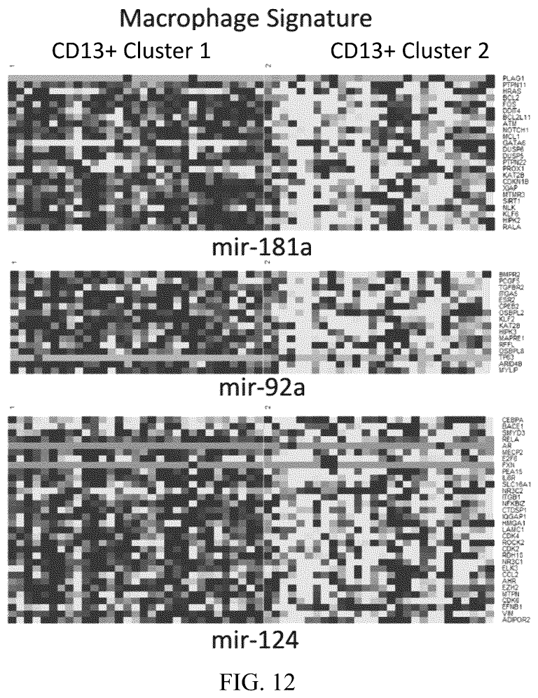

Differentially expressed genes between CD13 cluster 1 and CD13 cluster 2 are targets for CD associated miRNA, including mir-181a, mir-92a, and mir-124. Without being bound by any particular theory, such miRNA such as those provided in FIG. 12 may serve as therapeutic targets for the treatment of these CD patient subsets.

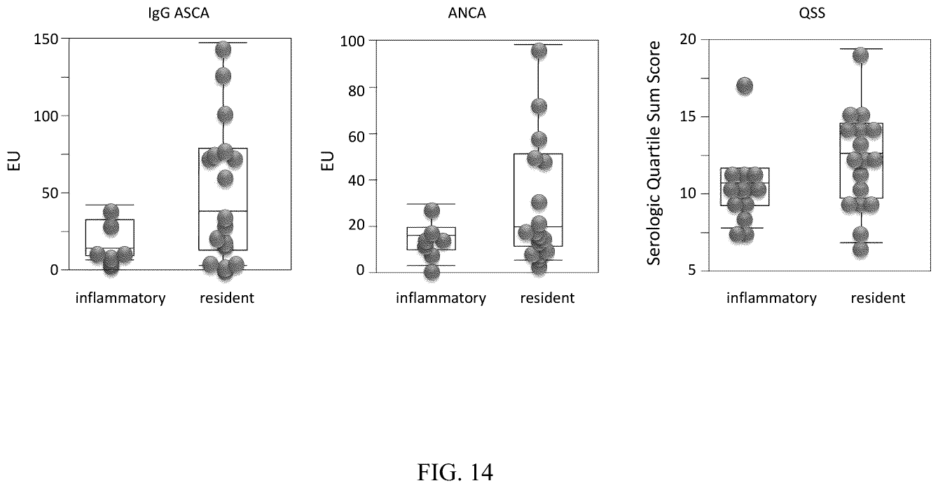

A cell type enrichment analysis was performed on the RNA-seq data to characterize the CD13+MNP cell subset composition with inferred by xCell. FIG. 13 shows that the CD13 cluster 1 (“inflammatory”) subset is associated with enhanced macrophage and activated dendritic cells enrichment scores. FIG. 14 shows that the CD13 cluster 2 (“resident”) subset is associated with elevated levels of antibodies against Saccharomyces cerevisiae (ASCA), antibodies against neutrophils (ANCA), and the quartile sum score (QSS). The QSS was determined based on the following serological markers: ASCA, Outer Membrane Porin C (OMPC), and associated sequence 12 (I2).

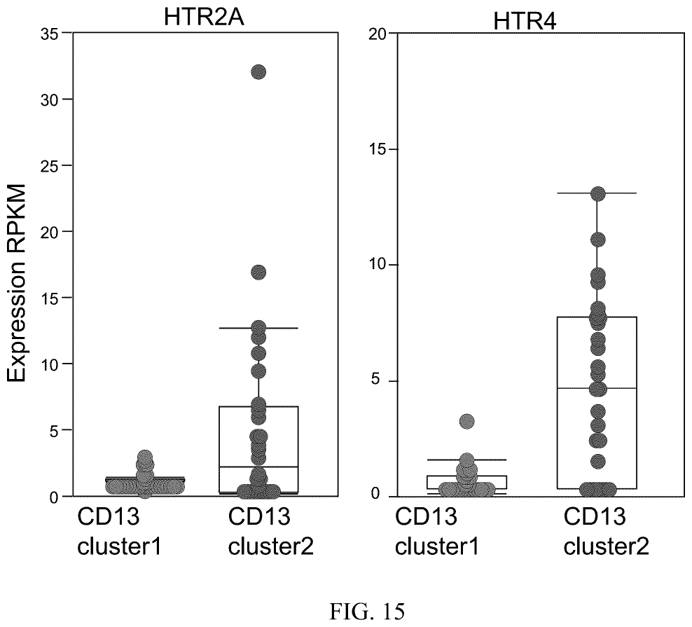

An enrichment analysis was performed to see whether CD13 cluster 1 (“immune-like”) and CD13 cluster 2 (“mucosal-like”) were associated with expression of serotonin receptors, 5-hydroxytryptamine receptor 2A (HTR2A) and 5-Hydroxytryptamine Receptor 4 (HTR4). FIG. 15 shows that the CD13 cluster 2 is significantly associated with increased expression of both HTR2A and HTR4, suggesting a role of gut-mediated serotonin expression/signaling in this subset of CD patients.

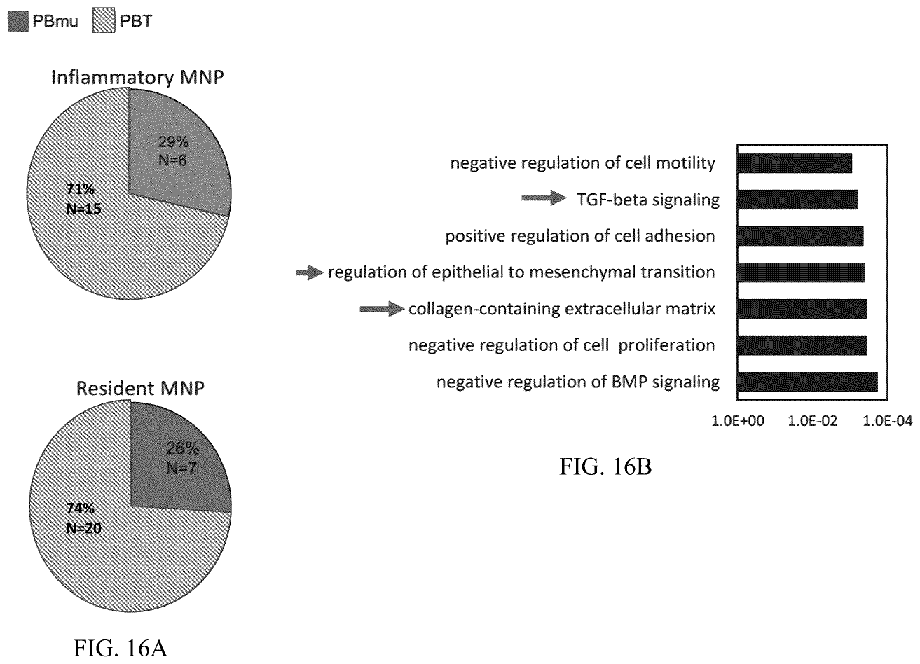

The RNA-Seq data for the CD13 cluster 1 (“inflammatory”) and CD13 cluster 2 (“resident”) subsets was compared with RNA-Seq data from the previously reported CD-PBmu subtype to determine whether there is any overlap between the two. FIG. 16A shows that there is very little association between the PBmu subtype and either of the CD13+ subsets provided herein. However, the genes that do overlap are involved in TGF-beta signaling, regulation of epithelial to mesenchymal transition, and collagen-containing extracellular matrix, according to a pathway analysis, as shown in FIG. 16B. The overlapping genes are shown in Table 4.

TABLE 4
Overlapping genes between molecular
pathways in PBmu and MNP signatures

| Gene | p-value | Fold | Name |
|---|---|---|---|
| SFRP2 | 2E−05 | 5.1 | secreted frizzled related protein2 |
| GREM1 | 3E−05 | 2.4 | gremlin 1 |
| COL5A3 | 3E−05 | 3.9 | collagen type V alpha 3 chain |
| MN1 | 4E−05 | 2.4 | MN1 proto-oncogene |
| CHRD | 4E−05 | 1.9 | chordin |
| ZUP1 | 6E−05 | 1.2 | zinc finger ubiquitin peptidasel |
| PALM | 7E−05 | 3.4 | paralemmin |

Without being bound by any particular theory, subjects having a fold-change increase in any one of the above genes is believed to have the CD-PBmu and either of the CD13 cluster 1 (“inflammatory”) and CD 13 cluster 2 (“resident”) subtypes.

Transcriptomic signatures were not associated with gender, age, disease location/behavior or therapeutic treatment. There were no significant differences reflecting a larger burden of mucosal inflammatory disease were noted between the subgroups, as shown in Table 5.

TABLE 5

| | CD13 Cluster 1 | CD13 Cluster 2 |
|---|---|---|
| | (“Inflammatory”) | (“Mucosal”) resident |
| Patient demographics | MNP signature | MNP signature |
| Gender Female (%) | 45 | 50 |
| Age at diagnosis (median and IQR), yr. | 18 (10-26) | 26 (16-46) |
| Montreal classification | | |
| ≤16 years (A1) | 42 | 27 |
| 17-40 years (A2) | 42 | 41 |
| >40 years (A3) | 16 | 32 |
| Disease duration (median and IQR), yrs. | 3(1-16) | 8(2-12) |
| Age at surgery (median and IQR), yr. | 31(27-45) | 41(28-53) |
| Anemia | 50 | 53 |
| elevated CRP at surgery | 67 | 60 |
| Steroids | 58 | 78 |
| 5-ASA | 64 | 67 |
| anti-TNF | 57 | 63 |
| immunomodulators | 60 | 73 |
| CD disease location ileocolonic | 95 | 78 |
| CD Disease Behavior | | |
| Stricturing Disease | 63 | 68 |
| Internal penetrating | 25 | 33 |
| resected bowel length (median and IQR), cm. | 33(23-52) | 40(31-62) |

### CONCLUSIONS

Patients with severe CD can be stratified into 2 functionally diverse MNP expression profiles with an inflammatory or mucosal-resident signature. The inflammatory subgroup displays enrichment across a phenotypical spectrum of both pro-inflammatory (M1) or anti-inflammatory (M2) phenotypes features, with elevated expression of pro-inflammatory interleukin 6 (IL6), tumor necrosis factor (TNFα) and healing Transforming growth factor beta (TGFβ) cytokines as well as CD163, CCL22, CD40, CCL3, CCL5 markers. The resident mucosal CD subgroup was associated with elevated response to microbial ASCA and ANCA and increased expression of serotonin receptors HTR2a and HTR4. Enriched serotonin receptor expression and sero-positivity to microbial antigens in the resident CD subgroup is intriguing In light of serotonin mediated host-microbiota interactions. Understanding the distinct differentiation programs of mucosal MNPs could aid in the design of novel CD subtype-specific therapeutics.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1. A method of determining a Crohn's disease (CD) subtype status in a subject having CD, wherein the status comprises a CD13+ mononuclear phagocytic (MNP) subtype, the method comprising:
detecting expression of one or more genes from Tables 1 or 2A-2B in a biological sample from the subject to obtain an expression profile comprising the expression levels of each of the one or more genes in the biological sample, and
determining the CD subtype status of the subject based upon the expression profile,
wherein an increased level of expression in the one or more genes in the biological sample as compared to a reference expression profile indicates status of CD-MNP subtype.

2. A method of selecting a treatment for a subject having a Crohn's disease (CD) CD13+ mononuclear phagocytic (MNP) subtype, the method comprising:
(a) determining a level of expression of one or more genes from Tables 2A-2B in a biological sample obtained from the subject having CD;
(b) detecting an expression profile comprising an increase in the level of expression of the one or more genes in the biological sample, relative to a reference expression profile; and
(c) identifying the subject as a candidate for treatment of Crohn's disease based upon the expression profile that is detected in (b).

3. (canceled)

4. A method of treating moderate to severe Crohn's disease (CD) in a subject, the method comprising: administering a therapeutically effective amount of a therapeutic agent for treatment of the CD to the subject, provided the subject is determined to have a CD13+ mononuclear phagocytic (MNP) subtype (CD-MNP subtype) based, at least in part, on an increased expression of one or more genes from Tables 2A-2B in a biological sample obtained from the subject, relative to a reference expression profile.

5. The method of claim 1, wherein CD-MNP subtype is characterized by an inflammatory MNP transcriptomic signature, resident mucosal MNP transcriptomic signature, or any combination thereof.

6.-9. (canceled)

10. The method of claim 5, wherein the resident mucosal MNP transcriptomic signature is associated with an increase or a decrease in a serological marker of an immune reactivity to a microbial antigen.

11. The method of claim 10, wherein the serological marker comprises antineutrophil cytoplasmic antibodies (ANCA), antibodies (IgG) against the yeast Saccharomyces cerevisiae (ASCA), or a combination thereof.

12.-16. (canceled)

17. The method of claim 1, wherein the reference expression profile is derived from gene expression levels measured in samples obtained from one or more individuals that:
(a) does not have the CD; or
(b) has a different CD13+MNP subtype of the CD.

18.-20. (canceled)

21. The method of claim 1, wherein determining or detecting a level of expression of one or more genes comprises utilizing an assay selected from the group consisting of an RNA sequencing method, a microarray method, and quantitative polymerase chain reaction (qPCR).

22. The method of claim 1, wherein determining or detecting a level of expression of one or more genes comprises:
(a) contacting the biological sample with a nucleic acid primer and/or detectable nucleic acid probe; and
(b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes.

23. The method of claim 1, further comprising administering to the subject a therapeutic agent against Crohn's disease based upon the expression profile.

24. The method of claim 23, wherein the therapeutic agent comprises a modulator of miR-181a, miR-92a, miR-124, Tumor necrosis factor-like cytokine 1A (TL1A), Tumor necrosis factor ligand superfamily member 8 (CD30L), or any combination thereof.

25. The method of claim 24, wherein the therapeutic agent comprises an antibody or antigen-binding fragment thereof.

26. The method of claim 25, wherein antibody or antigen-binding fragment thereof comprises a TL1A antibody or antigen-binding fragment thereof.

27. The method of claim 26, wherein the TL1A antibody or antigen-binding fragment thereof comprises PRA023, tulisokibart, PF-06480605, or TEV-48574.

28.-29. (canceled)

30. The method of claim 24, wherein the antibody or antigen-binding fragment thereof comprises a CD30L antibody or antigen-binding fragment thereof.

31. The method of claim 30, wherein the CD30L antibody or antigen-binding fragment thereof comprises KPL-045, PRA052, or any combination of one or more thereof.

32.-35. (canceled)

36. The method of claim 1, provided the biological sample comprises a blood sample or is purified from a blood sample of the subject.

37. The method of claim 1, wherein the subject is not responsive to anti-TNF therapy.

38. (canceled)

39. The method of claim 1, wherein the subject has or is susceptible to having increased length of bowel resection.

40. The method of claim 1, wherein the CD is associated with perianal disease/fistula, stricturing disease, recurrence, or immune reactivity to a microbial antigen, or any combination of one or more thereof.

41.-85. (canceled)
